SEQUENCE LISTING (24 sequences)

SEQ ID NO: 1 - DNA, 21 bp, Homo sapiens
caacaagatgaagagcaccaa

SEQ ID NO: 2 - DNA, 21 bp, Homo sapiens
gagaagatgattgcgatggat

SEQ ID NO: 3 - DNA, 21 bp, Homo sapiens
cctcaagccatccaacatatt

SEQ ID NO: 4 - protein, 46 aa, Homo sapiens
FLRCINYVFFPSLKPSSSIDEYFSEQPLKNLLLSTSEEQIEKCFII

SEQ ID NO: 5 - protein, 46 aa, Pan troglodytes
FLRCINYVFFPSLKPSSNIDEYFSEQPLKNLLLSTSEEQIEKCFII

SEQ ID NO: 6 - protein, 46 aa, Papio anubus
LLRCINYVFFPSLKPSSNIDEYFSEQSLKNLLLSTSEEQIEKCLII

SEQ ID NO: 7 - protein, 46 aa, Macaca fascicularis; identical to SEQ ID NO: 6

SEQ ID NO: 8 - protein, 46 aa, Bos taurus
FLRCVKYVFFPSSKPPSSVDEYFSDQPLPNLLLSTSEEQTERCFII

SEQ ID NO: 9 - protein, 47 aa, Equus caballus
ELWRCINYVFFPSSKPPSAVDEYFSEHLLKNLLLSTSAEQIERCFVI

SEQ ID NO: 10 - protein, 46 aa, Ovis aries
FLRCVKYVFFPSSKPPSSVDQYFSDQPLPNLLLSTSEEQTERCFII

SEQ ID NO: 11 - protein, 46 aa, Sus scrofa
VSRCINYVFFPSSKPPSTIDEYFAEQPLKNLLLSTSEEQTEICFIV

SEQ ID NO: 12 - protein, 46 aa, Canis familiaris
LLRCISYVFFPSSKPPSTIDEYFSEPPLWNLLLLTSEEQTERCFII

SEQ ID NO: 13 - protein, 46 aa, Rattus norvegicus
SLQKYLYYVFSPPLKPPCSIDEFFSELPSKSLLLLTAEEHTERCFV

SEQ ID NO: 14 - protein, 46 aa, Mus musculus
VWKYLCHVCFPPLKPPRSIDEFFSEPPSKNLVLLTAEEHTERCFII

SEQ ID NO: 15 - protein, 46 aa, Gallus gallus
VYNKIKYMFFPSCQTPLNIEGFGAQLFSSPFVPTVEEPVEICYIIE

SEQ ID NO: 16 - protein, 13 aa, Homo sapiens
DIKPDNFLMGIGR

SEQ ID NO: 17 - protein, 14 aa, Homo sapiens
YASINAHLGIEQSR

SEQ ID NO: 18 - protein, 9 aa, Homo sapiens
TSLPWQGLK

SEQ ID NO: 19 - protein, 11 aa, Homo sapiens
KMSTPVEVLCK

SEQ ID NO: 20 - protein, 11 aa, Homo sapiens
FEEAPDYMYLR

SEQ ID NO: 21 - DNA, 23 bp, Photinus pyralis
cuuacgcugaguacuucgadtdt

SEQ ID NO: 22 - DNA, 23 bp, Homo sapiens
ccaggcauccccaguugcudtdt

SEQ ID NO: 23 - DNA, 23 bp, Homo sapiens
ccaggcuaggccaguugcudtdt

SEQ ID NO: 24 - protein, 23 aa, Artificial Sequence
  Chemically synthesized
EDHKKYSSQTPSQDSGNYSNEDE

(19) United States
(12) Patent Application Publication
(10) Pub. No.: US 2012/0014911 A1
(43) Pub. Date: Jan. 19, 2012

(54) Regulators of the Interferon-Alpha Receptor 1 (IFNAR1) Chain of the Interferon Receptor

(76) Inventors: Serge Fuchs, Media, PA (US); J. Alan Diehl, Ardmore, PA (US)

(21) Appl. No.: 13/142,814

(22) PCT Filed: Jan. 8, 2010
(86) PCT No.: PCT/US10/20489

§ 371 Date: Sep. 26, 2011

Related U.S. Application Data
(60) Provisional application No. 61/143,627, filed on Jan. 9, 2009.

(51) Int. Cl. A61K 31/7088; A61K 38/02; A61K 31/7064; A61K 31/675; C12N 5/071; A61K 38/21; A61P 35/00; A61P 25/00; A61P 37/00; A61P 31/12; A61K 39/395; A61K 31/663

(52) U.S. Cl. 424/85.4; 514/44 A; 514/44 R; 424/130.1; 514/1.1; 514/49; 514/82; 514/141; 435/375; 435/325

(57) ABSTRACT

The present invention includes compositions and methods for modulating a regulator of IFNAR1. The invention includes inhibitors and activators of PERK, PTP1B, and/or PKD2 wherein inhibition, or activation, of at least one of PERK, PTP1B, and PKD2 modulates the stability of IFNAR1.

### BACKGROUND OF THE INVENTION

Animal hosts defend themselves against infectious agents or tumor growth by utilizing the mechanisms of innate and adaptive immunity. Importantly, diverse pathways of innate immunity converge on the induction of cytokines that belong to a family of Type I interferons (IFN) including various types of IFN-α and IFN-β that play a major role in host defenses against the viruses. Unlike IFNγ, which belongs to Type II IFN group, all members of the Type I family act on cells via the same cognate receptor that consists of two sub-units: IFNAR1 and IFNAR2c (reviewed in Pestka, 2000, Biopolymers 55:254-287).

Dimerization of receptor chains in response to the ligands results in the activation of Janus kinase (Jak) family members Jak1 and Tyk2 that phosphorylate each other, the aforementioned receptor subunits and the recruited signal transducers and activators of transcription (Stat1 and Stat2) at specific tyrosines. Phosphorylated Stat proteins translocate to the nucleus, bind to IFN-stimulated regulatory elements (ISRE) and activate transcription of a large number of IFN-stimulated genes (ISGs, reviewed in Stark et al., 1998, Annu. Rev, Biochem, 67:227-264). ISGs mediate a plethora of IFNa effects that play key roles in anti-viral defense (Brassard, et al, 2002,J Leukoc Biol 71(4):565-581; Katze, et al., 2002, Nat Rev Immunol 2(9):675-687), inhibition of cell proliferation (Brassard, et al, 2002, J Leukoc Biol 71(4):565-581; Kirkwood, 2002, Semin Oncol 29(3 Suppl 7):18-26; Stark, et al., 1998, Annu Rev Biochem 67:227-264) and modulation of immune responses (Biron, 2001, Immunity 14:661-664; Brassard, et al, 2002, J Leukoc Biol 71(4):565-581). The ability of IFNα to evoke these outcomes makes it an attractive therapeutic agent extensively used for treatment of patients with neoplastic diseases, i.e. cancers (Kirkwood, 2002, Semin Oncol 29(3 Suppl 7):18-26), chronic viral infections (Brassard, et al, 2002, J Leukoc Biol 71(4):565-581; Katze, et al., 2002, Nat Rev Immunol 2(9):675-687), and multiple sclerosis (Karp, et al., 2000, Immunol Today 21(1):24-28).

Studies in cell culture revealed that anti-viral effects of IFN are best seen when it is added to cells prior to the infection (Blalock, et al., 1979, J Gen Virol 42:363-372; Pfeffer, et al., 1991, Pharmacol Ther 52(2):149-157). While decreased efficacy of IFN added to already infected cells is largely explained by insufficient time to transcribe and translate ISG products (reviewed in (Friedman, et al., 1970, Arch Intern Med 126(1):51-63; Pfeffer, et al., 1991, Pharmacol Ther 52(2):149-157)), additional mechanisms such as a negative effect of virus on IFN action have been also postulated (Lockart, 1963, J Bacteriol 85:556-566; Lockart, 1963, J Bacteriol 85:996-1002). Indeed, many viruses evolved to employ a multitude of specific mechanisms to protect themselves against Type I IFN. These mechanisms usually involve a rapid synthesis of numerous virus type-specific proteins that impede diverse elements of pathways converging on either IFN production or IFN signaling (reviewed in (Katze, et al., 2002, Nat Rev Immunol 2(9):675-687)).

A need for the robust synthesis of viral polypeptides, however, poses additional problems for the virus as it challenges the capacity of the host cell to properly fold and activate proteins. Accumulation of sub-optimally folded proteins in the ER of the host cell induces a series of signaling events known as the ER stress or the unfolded protein response (UPR) (Welihinda, et al., 1999, Gene Expr 7:293-300). While the ER protein chaperone BiP is central to initiating virtually all branches of the response, subsequent signaling proceeds via a number of defined mechanisms that include other transmembrane sensors including ATF6, IRE1 and PKR-like ER kinase (PERK). The activation of PERK and ensuing phosphorylation of eIF2α restricts translation to alleviate the load of unfolded proteins The latter is necessary in order to protect the host cells from ER stress-mediated death, to enable (reviewed in (Malhotra, et al., 2007, Semin Cell Dev Biol 18(6):716-731; Ron, et al., 2007, Nat Rev Mol Cell Biol 8(7):519-529)). Viruses are known to both induce UPR and produce the means of inhibiting these responses translation of viral proteins and to continue virus production (He, 2006, Cell Death Differ 13(3):393-403; Schroder, et al., 2006, Curr Mol Med 6(1):5-36; Wang, et al., 2006, J Gastroenterol Hepatol 21 Suppl 3:S34-37; Waris, et al., 2002, Biochem Pharmacol 64:1425-1430).

While investigating the mechanisms that govern proteolytic degradation of Type I IFN receptor it was found that IFNAR1 undergoes ligand-induced Tyk2 activity-dependent phosphorylation on specific Ser residues (Ser535 in humans and Ser526 in mice). This phosphorylation leads to the recruitment of βTrcp E3 ubiquitin ligase followed by IFNAR1 ubiquitination, internalization, and lysosomal degradation (Kumar, et al., 2007, J Cell Biol 179(5):935-950; Kumar, et al., 2004, J Biol Chem 279(45):46614-46620; Kumar, et al., 2003, Embo J 22(20):5480-5490; Marijanovic, et al., 2006, Biochem J 397(1)31-38). Intriguingly, there is also a ligand- and Jak-independent pathway resulting in phosphorylation and turnover of IFNAR1 in cells that over-expressed this receptor (Liu, et al., 2008, Biochem Biophys Res Commun 367(2):388-393).

There have been many attempts made to use various agents to target the IFN signaling pathway for treating diseases associated with this pathway. There is a need in the art for the development of successful therapeutic agents to increase the efficacy of either endogenous IFN or IFN-based drug in patients with viral infections, tumors and multiple sclerosis. The present invention satisfies the need in the art for development of new approaches for efficient means to increase IFN efficacy.

### BRIEF SUMMARY OF THE INVENTION

The present invention provides a method of modulating the stability of IFNAR1 in a cell. The method comprises contacting a cell with an effective amount of a composition comprising an inhibitor of a regulator of IFNAR1, including any one or more of PERK, PTP1B, or PKD2. In various embodiments, the inhibitor is at least one of an siRNA, a microRNA, an antisense nucleic acid, a ribozyme, a transdominant negative mutant, an intracellular antibody, a peptide or a small molecule.

The invention also includes a method of treating a disease or disorder associated with a dysfunctional IFN response. For diseases or disorders associated with an abnormally diminished IFN response, the method comprises administering to a mammal in need thereof, a therapeutically effective amount of a composition or pharmaceutical composition comprising an inhibitor of a regulator of IFNAR1, including any one or more of PERK, PTP1B, or PKD2. In one embodiment, the composition or the pharmaceutical composition is administered in combination with a therapeutic agent. Preferably, the therapeutic agent is IFN. In various embodiments, the disease is a viral infection, cancer or an autoimmune disease. One nonlimiting example of an autoimmune disease amenable to the methods of the invention is multiple sclerosis.

The invention also includes a method of treating a disease or disorder associated with a dysfunctional IFN response. For diseases or disorders associated with an pathologically increased IFN response, the method comprises administering to a mammal in need thereof, a therapeutically effective amount of a composition or pharmaceutical composition comprising an activator of a regulator of IFNAR1, including any one or more of PERK, PTP1B, or PKD2. In one embodiment, the composition or the pharmaceutical composition is administered in combination with a therapeutic agent. In various embodiments, the disease is a viral infection, cancer or an autoimmune disease. Two nonlimiting examples of autoimmune diseases amenable to the methods of the invention is systemic lupus erythematosus and psoriasis.

The invention also provides a method of increasing the efficacy of endogenous IFN in a mammal. The method comprises administering to a mammal in need thereof, a therapeutically effective amount of a composition or pharmaceutical composition comprising an inhibitor of a regulator of IFNAR1, including any one or more of PERK, PTP1B, or PKD2. The invention further provides a method of increasing the efficacy of IFN-based drug treatment in a mammal. The method comprises administering to a mammal in need thereof, a therapeutically effective amount of a composition or pharmaceutical composition comprising an inhibitor of a regulator of IFNAR1, including of any one or more of PERK, PTP1B, or PKD2.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1, comprising FIGS. 1A through 1F, is a series of images demonstrating that ER stress induces IFNAR1 Ser535 kinase activity and promotes phosphorylation of IFNAR1 within its destruction motif in a manner that does not require Tyk2 activity but relies on activity of PERK.

FIG. 1A is an image depicting immunoblotting (IB) analysis of the lysates from KR-2 cells (lacking catalytic activity of Tyk2) transfected with Flag-IFNAR1 plasmid (0-3.0 μg) subjected to immunoprecipitation (IP) using anti-Flag antibody followed by immunoblotting (IB) using the indicated antibodies. Relative intensity of bands in IB using either anti-phospho-S535 (black squares) or IFNAR1 (R1, gray squares) or Flag (black squares) was quantified and plotted in the right panel.

FIG. 1B is an image depicting analysis of the lysates from KR-2 cells transfected with Flag-IFNAR1 (1.5-3.0 μg) or empty vector used as a source of kinase activity in an in vitro kinase assay using GST-IFNAR1 as a substrate. The reactions were analyzed by IB using anti-phospho-S535 antibody (upper panel; both shorter and longer exposures shown) and by Ponceau staining to detect the substrate levels (middle panel). Levels of Flag-IFNAR1 in the cell lysates were analyzed by IB using anti-Flag antibody (lower panel).

FIG. 1C is an image depicting analysis of the 293T cells treated with thapsigargin (TG, 1 μM) for 30 min. Endogenous IFNAR1 was immunoprecipitated and analyzed by IB using indicated antibodies. Aliquots of the whole cell lysates were analyzed for levels of phospho- and total eIF2α.

FIG. 1D is an image depicting analysis of the cells harboring wild type (WT-5) or the kinase dead Tyk2 (KR-2) treated with IFNα (1000 IU/ml) or TG at indicated concentrations for 30 min. Endogenous IFNAR1 was analyzed by IP-IB as in FIG. 1C.

FIG. 1E is an image depicting analysis of the WT or PERK^{-/-} MEFs treated with TG (1 μM) or murine IFNβ (1000 U/ml) for 30 min. Mouse endogenous IFNAR1 was analyzed for its phosphorylation and levels using the indicated antibodies.

FIG. 1F is an image depicting analysis of the MEFs from PERKfl/fl mice that received an empty vector (Mock) or vector for expression of Cre recombinase (Cre), Whole cell lysates from these cells were also analyzed by IB using the indicated antibodies,

FIG. 2, comprising FIGS. 2A through 2F, is a series of images demonstrating that ER stress promotes IFNAR1 ubiquitination and degradation in a ligand/Jak-independent manner

FIG. 2A is an image depicting analysis of the levels of endogenous IFNAR1 in 293T cells pre-treated or not with methylamine HCl (MA, 20 mM) for 1 h and then treated with TG (1 μM) for indicated time were analyzed by IP-IB. Levels of β-actin in whole cell lysates are also shown.

FIG. 2B is an image depicting analysis of the cells harboring the WT Tyk2 (WT-5) or the kinase dead Tyk2 (KR-2) treated with TG as indicated and ubiquitination and levels of endogenous IFNAR1 were analyzed by IP-IB. Aliquots of whole cell lysates were also analyzed by IB using anti-β-actin antibody (lower panel).

FIG. 2C is an image depicting analysis of the 293T cells pre-treated or not with MA for 1 h and then treated with cycloheximide (Chx, 50 μg/ml) alone or together with TG (1 μM) for indicated times. Levels of endogenous IFNAR1 were analyzed by IP-IB. Levels of c-Jun and β-actin in whole cell lysates were also determined by IB using indicated antibodies.

FIG. 2D is an image depicting analysis of the ubiquitination of Flag-tagged IFNAR1 co-expressed with the indicated shRNA constructs in 293T cells was analyzed by IP using anti-Flag antibody followed by IB using anti-ubiquitin and anti-Flag antibodies as indicated. Aliquots of whole cell lysates were also analyzed by IB using anti-β-actin antibody (lower panel).

FIG. 2E is an image depicting analysis of the PERK^{fl/fl} MEF that either underwent acute deletion of PERK (Cre) or not (Mock) treated with 1 μM of TG (together with Chx, 10 μg/ml) for 45 min as indicated. Endogenous mouse IFNAR1 was analyzed by IP-IB using the indicated antibodies. Ig: heavy chain immunoglobulins. Whole cell lysates were also subjected to IB analysis to determine levels of phosphorylated β-catenin and eIF2α as well as total levels of PERK and eIF2α using respective antibodies. NS: non-specific band.

FIG. 2F is an image depicting analysis of the mouse Flag-IFNAR1 expressed in PERK^{fl/fl} MEFs that either underwent acute deletion of PERK (Cre) or not (Mock) analyzed by IB using anti-Flag antibody. Levels of PERK are shown in lower panel. NS: non-specific band that serves as a loading control.

FIG. 3, comprising FIGS. 3A through 3D, is a series of images demonstrating that ER stress ER stress promotes IFNAR1 degradation in a manner depending on IFNAR1 phosphorylation within its phospho-degron.

FIG. 3A is an image depicting a vector and a targeting strategy that were used to generate an S526A allele in mouse ES cells (C57/BL6). Position of mutated Ser residue, resistance markers, loxP sites as well as restriction sites and the probe used for Southern analysis are also shown.

FIG. 3B is an image depicting Southern analysis of several selected ES clones that underwent homologous recombination (marked by an asterisk) performed on genomic DNA digested with KpnI. Correct targeting yielded a 10.6 kb band in clones 106 and 252 (besides the 8.5 kb band indicative of the WT allele).

FIG. 3C is an image depicting analysis of the embryoid bodies (EB) derived from the WT (WT/WT) or the mutant (S526A/WT) ES cells. Cells were pre-treated or not with MA (20 mM for 1 h) and then treated with TG (1 μM, 15 min) as indicated. Endogenous mouse IFNAR1 was immunoprecipitated and analyzed by IB using the indicated antibodies. Phosphorylation of eIF2□ and levels of PKR were also determined in aliquots of whole cell lysates by IB.

FIG. 3D is an image depicting analysis of the EB-derived cells treated with TG (1 μM) for indicated times and analyzed for total levels of endogenous IFNAR1 (by IP-IB). Levels of eIF2α phosphorylation and total β-catenin were shown as stress and loading controls, respectively.

FIG. 4, comprising FIGS. 4A through 4E, is a series of images demonstrating that viral infection promotes phosphorylation-dependent ubiquitination and downregulation of IFNAR1 in a Tyk2-independent and S535/526-dependent manner.

FIG. 4A is an image depicting analysis of the ubiquitination, phosphorylation and total levels of endogenous IFNAR1 from KR-2 cells infected with VSV (for 16, 18 and 20 h) were analyzed by IP using anti-IFNAR1 antibody followed by IB using the indicated antibodies. Viral protein accumulation is shown by the levels of VSV-M.

FIG. 4B is an image depicting analysis of the levels of endogenous IFNAR1 in the lysates from Huh7 cells (parental or harboring either a full length or subgenomic HCV) were analyzed by IP-IB. Levels of β-actin in the lysates aliquots are also shown.

FIG. 4C is an image depicting analysis of the endogenous IFNAR1 proteins immunopurified from the indicated cells and loaded onto the gel to yield comparable levels of total IFNAR1 (lower panel). Phosphorylation of IFNAR1 was then analyzed by IB using indicated antibody (upper panel).

FIG. 4D is an image depicting analysis of the MEFs from IFNAR1−/−mice stably reconstituted with murine Flag-IFNAR1 (either wild type or S526A mutant) and then infected with VSV (for 16-18 h). Levels of IFNAR1, VSV-M and β-actin were analyzed by IB.

FIG. 4E is an image depicting analysis of the EB-derived cells of WT (WT/WT) or mutant (S526A/WT) genotype infected (or not) with VSV for 12 h and lysed. Under these conditions, levels of VSV-M become saturated at 10 hr post-infection. Levels of endogenous mouse IFNAR1 were determined by IP-IB. Levels of β-actin and VSV-M in the lysates were also determined.

FIG. 5, comprising FIGS. 5A through 5D, is a series of images demonstrating the role of PERK in virus-induced degradation of TFNAR1.

FIG. 5A is an image depicting analysis of the phosphorylation and levels of endogenous IFNAR1 in 2fTGH cells that received indicated shRNA constructs and then were infected with VSV (for 16, 18 and 20 h) was analyzed by IP-IB using the indicated antibodies. Aliquots of IP supernatants were used for analysis of VSV-M, p-eIF2α and eIF2α levels by IB.

FIG. 5B is an image depicting analysis of the control or PERK-depleted 2fTGH cells (as in FIG. 5A) infected with VSV (for 17 h) and then treated with Chx (1 or 10 μg/ml for 1.5 h). Total levels of IFNAR1 were determined by IP-IB.

FIG. 5C is an image depicting analysis of the levels of cell surface IFNAR1 analyzed by FACS using monoclonal anti-mIFNAR1 antibody in MEFs from PERK^{fl/fl} mice (transduced with either empty vector (Mock) or construct for expression of Cre) either left untreated (black line) infected with VSV (for 17 h, red line) or treated with TG (1 μM for 4 h, green line). Blue line represents the isotype Ig control.

FIG. 5D is an image depicting analysis of the levels of IFNAR1 and actin in Huh7 cells harboring the full-length or subgenomic HCV that were co-transfected with Flag-IFNAR1 and indicated shRNA constructs were analyzed using indicated antibodies.

FIG. 6, comprising FIGS. 6A through 6D, is a series of images demonstrating that viral infection inhibits Type I IFN signaling via accelerating Ser526 phosphorylation-dependent degradation of IFNAR1.

FIG. 6A is an image depicting analysis of the 2fTGH cells infected or not with VSV (for 20 h) treated with 50 IU/ml of IFNα□ or IFNβ for 30 min. Phosphorylation of Stat1 and total levels of Stat1, actin and VSV-M were analyzed by IB.

FIG. 6B is an image depicting analysis of the Huh7 cell line derivatives co-transfected with Flag-STAT1 and either empty vector (pcDNA3) or Flag-IFNAR1 (WT or S535A) as indicated. Lysates from these cells treated or not with IFNα (50 IU/ml) were immunoprecipitated using anti-Flag antibody and these reactions were analyzed by IB using the indicated antibodies.

FIG. 6C is an image depicting analysis of the EB-derived cells of wild type (WT/WT) or mutant (S526A/WT) genotype infected with VSV (for 12 h) and then treated with murine IFN-β (100 μl/ml) or IFN-γ (5 ng/ml) for 30 min, Phosphorylation of Stat1 and total levels of Stat1 and actin were analyzed by IB.

FIG. 6D is an image depicting analysis of the titer of VSV produced in EB-derived cells 14 h after infection (an incubation of cells with VSV at MOI 1.0 for 1 h), The effect of IFN-β (20 IU/ml) added either 16 h prior to the infection (pre-treat) or immediately after infection (co-add) was determined. Data shown (the mean±SD) are representative of two independent experiments (each in triplicate). Asterisk denotes p<0.01 in comparison with untreated cells.

FIG. 7, comprising FIGS. 7A through 7G, is a series of images demonstrating the role of PERK in virus-induced suppression of Type I IFN signaling.

FIG. 7A is an image depicting analysis of the control or PERK-depleted derivatives of 2fTGH cells infected with VSV (for 20 h) and treated with IFN-α or IFN-γ□(50 IU/ml for 30 min). Phosphorylation and total levels of Stat1 and eIF2α were analyzed by

FIG. 7B is an image depicting analysis of the MEFs from PERK^{fl/fl} mice transduced with either empty vector (Mock) or construct for expression of Cre were infected with VSV (for 20 h) and then treated with IFN-β□(100 IU/ml) or IFN-γ (5 ng/ml) for 30 min. Phosphorylation and total levels of Stat1 were analyzed by IB.

FIG. 7C is an image depicting analysis of the MEFs from PERK^{fl/fl} mice transduced as indicated infected with VSV at MOI 0.1 (+) or 0.5 (++) for 20 h and treated with IFN-β for 30 min. IB analyses using indicated antibodies are shown.

FIG. 7D is an image depicting analysis of the derivatives of Huh7 cells co-transfected with indicated shRNA constructs and Flag-STAT 1 and treated with IFN-a (50 IU/ml) for 30 min. Stat1 proteins were immunoprecipitated using anti-Flag antibody and analyzed by IB using anti-phospho-Stat1 and anti-Stat1 antibody.

FIG. 7E is an image depicting analysis of the titer of VSV produced in control or PERK-depleted derivatives of 2fTGH cells 14h after infection (an incubation of cells with VSV at MOI 1.0 for 1 h). The effect of IFN-α (20 IU/ml) added either 16 h prior to the infection (pre-treat) or immediately after infection (co-add) was determined. Data shown (the mean±SD) are representative of two independent experiments (each in triplicate). Asterisk denotes p<0.01 in comparison with untreated cells.

FIG. 7F is an image depicting analysis of the MEFs from PERK^{fl/fl} mice transduced as indicated infected with VSV (MIO 1.0). 20 h after infection, viral titer in the culture supernatant was determined. Values represent the mean±SD of three independent experiments each performed in triplicate. VSV-M protein levels analyzed by IB in cell lysates are also shown in the inset.

FIG. 7G is an image depicting analysis of the 2fTGH and isogenic IFNAR2-deficient U5a cells transduced with indicated shRNA constructs and then infected with VSV (MOI 1.0) for 18-20 h. Levels of VSV-M, ISG15 and β-actin were determined by IB. In a parallel experiment, these cells were treated with TG (1mM for 30 min) and analyzed for PERK levels by IP-IB (lower panel).

FIG. 8 is an image depicting analysis of the 293T cells transfected with Flag-IFNAR1 or empty vector (pcDNA3) and the lysates analyzed by immunoblotting using the indicated antibodies.

FIG. 9 is an image depicting analysis of the 2fTGH cells transfected with Flag-IFNAR1 or empty vector (pcDNA3) and whole cell lysates (WCE). Endogenous IFNAR1 was immunoprecipitated and analyzed by IB using anti-pS535 and anti-IFNAR1 (R1) antibodies. WCE were analyzed by immunoblotting using the indicated antibodies,

FIG. 10 is an image depicting characterization of shRNA against human PERK. 293T cells were transfected with control shRNA plasmid or shPERK plasmid. 48 h after transfection, cells were harvested and the cells lysates were subjected to analysis for PERK levels by IP-IB. Levels of IRE1 in the lysates serve as loading control.

FIG. 11 is an image depicting analysis of the 293T cells co-transfected with Flag-IFNAR1 along with control shRNA plasmid or shPERK plasmid as indicated. 48 h after transfection, cells were harvested and the cells lysates were subjected to immunoblotting analysis using the indicated antibodies.

FIG. 12 is an image depicting analysis of the 293T cells pre-treated with methylamine (20 mM) for 1 h and then with 5 mM of DTT for 30 min. Lysates were subjected to IP-IB analysis for pS535 and total IFNAR1 levels.

FIG. 13 is an image depicting analysis of the 293T cells transduced with lentiviruses encoding control shRNA (shCon) or shIRE1α (shIRE1). The cells were treated with TG (1 μM) or IFN-α (1000 IU/ml) for 30 min. pS535 and total IFNAR1 levels were examined using IP-IB. The effect of IREla knockdown was also determined by direct immunoblot.

FIG. 14 is an image depicting analysis of the 293T cells transfected with a control shRNA construct or shRNA against PERK treated with TG (1 μM) or IFN-α (1000 IU/ml) for 30 min. Phosphorylation and levels of endogenous IFNAR1 and eIF2α were analyzed by immunoblotting.

FIG. 15 is an image depicting analysis of the 293T cells pre-treated with methylamine HCl (MA, 20 mM) for 1 h and then treated with TG (1 μM) for indicated time. Ubiquitination of endogenous IFNAR1 was analyzed by IP-IB using the indicated antibodies.

FIG. 16 is an image depicting analysis of the MEF cells derived from IFNAR1-null mice stably transduced with either empty retrovirus (“Vector) or with retroviruses for expression of mouse Flag-tagged IFNAR1 (wild type or S526A mutant, “SA”). Effect of TG treatment on IFNAR1 ubiquitination was analyzed by IP-IB using the indicated antibodies.

FIG. 17 is an image depicting analysis of the 2fTGH cells left untreated or infected with VSV (MOI 0.1 and 0.3 respectively) for 19 h. Levels of indicated proteins were analyzed by immunoblotting using indicated antibodies.

FIG. 18 is an image depicting analysis of the 2fTGH cells infected with MOI 0.1 VSV for 16, 18 and 20 h. pS535 and total IFNAR1 levels were determined.

FIG. 19 is an image depicting analysis of the 2fTGH cells transduced with control virus (shCON) or virus encoding shPERK (shPERK) treated with TG (1 μM) for 30 min. PERK levels were determined by IPIB. Levels of p-eIF2α or total eIF2a were determined by direct immunoblot.

FIG. 20 is an image depicting analysis of the 2fTGH transduced with empty virus (pLK), virus encoding shPERK (shPERK), irrelevant control shRNA (shCon) or shIRE1 were infected with MOI 0.1 of VSV for 20 h. Total IFNAR1 levels were determined by IP-IB. Position of mature IFNAR1 is indicated by arrow. Asterisks points to a non-specific band that serves as loading control.

FIG. 21 is an image depicting analysis of the 293T cells treated with TG (1 μM) for 4 h and then the cells were re-fed with fresh medium overnight. After that incubation, cells were treated with 50 IU/ml of IFNα or IFNγ for 30 min. pSTAT1 and STAT1 levels were determined by immunoblotting.

FIG. 22 is an image depicting analysis of the parental Huh7 cells or cells harboring the subgenomic (Sub-HCV) or the full length (FL-HCV) HCV genome treated with 50 IU/ml of IFNα (0.5 h and 1 h) or IFNγ (0.5 h). pSTAT1, STAT1 and β-actin levels were analyzed.

FIG. 23 is an image depicting analysis of the WT or PKR^{-/-}MEFs infected with MOI 0.1 of VSV for 20 h. Cells were then treated with mIFNβ (50 IU/ml) for 30 min. pSTAT1 and total STAT1 levels were determined, VSV-M, p-eIF2α and levels of two short-lived proteins p-β-catenin and c-Jun were used to show similar levels of viral and stress load in either cell line, Total eIF2α levels were used as loading control.

FIG. 24 is an image depicting analysis of the WT or conventional PERK^{-/-}MEFs or MEFs stably expressing exogenous PERK infected with VSV. Twenty hours later, virus-containing culture supernatant was harvested and viral titer was determined. Levels of PERK expression are shown in FIG. 25.

FIG. 25 is an image depicting analysis of the WT or conventional PERK^{-/-}MEFs or MEFs stably expressing exogenous PERK transfected with ISRE-luciferase reporter along with Renilla luciferase reporter plasmid. IFN-β-induced ISRE-driven transcription was analyzed as previously described (Kumar, et al., 2003, Embo J 22(20):5480-5490). Average activity (in arbitrary units normalized per renilla luciferase) from three independent experiments is shown. Lower panel depicts IB analysis of the lysates from these cells using anti-PERK antibody. Position of PERK is indicated by an arrow. A non-specific (NS) band serves as a loading control.

FIG. 26 is an image depicting analysis of the 21TGH cells harboring either shPERK or irrelevant control shRNA (shCon) infected with MOI 0.1 of VSV. Blocking antibodies against IFN-α and IFN-β (at 500 U) were added to the medium at that time as indicated. Cells were harvested 16 h later and the levels of VSV-M and β-actin were analyzed by IB.

FIG. 27, comprising FIGS. 27A through 27D, is a series of images demonstrating that PTP1B regulates the extent of IFNAR1 endocytosis. FIG. 27A is a graph demonstrating that overexpression of PTP1B (blue line) increases the efficacy of internalization of IFNAR1 measured by the high throughput fluorescence assay; expression of catalytically inactive PTP1B mutant (D181A) decreases the rate of IFNAR1 endocytosis. FIG. 27B is a graph demonstrating that knockdown of PTP1B by shRNA decreases the rate of IFNAR1 endocytosis. FIG. 27C is an image presenting commercially available inhibitors of PTP1B, FIG. 27D is a graph demonstrating that inhibitors of PTP1B prevent efficient endocytosis of IFNAR1. FIG. 28, comprising FIGS. 28A and 28B, is a graph demonstrating pre-treatment of human 21TGH cells with inhibitors of PTP1B potentiates the protective effect of IFN-α against viruses. Cells were pre-treated or not with SSG and then treated with different doses of IFN-α (25-100 U/ml). After that, cells were infected with vesicular stomatitis virus (VSV) and the titer of this virus (as a measure of its replication) was measured 24 hr after treatments.

FIG. 29 is an image depicting PTP1B inhibitors.

FIG. 30 is an image demonstrating that inhibition of PKD2 (but not related kinases PKD1 or PKD3) by siRNA prevents phosphorylation of IFNAR1 on Ser535 in HeLa cells treated with IFN-α. Lower panel shows total levels of IFNAR1,

FIG. 31 is an image demonstrating that inhibition of PKD2 (but not related kinases PKD1 or PKD3) by shRNA prevents phosphorylation of IFNAR1 on Ser535 in HeLa cells treated with IFN-α. Lower panel shows total levels of IFNAR1.

FIG. 32 is an image depicting the analysis of the degradation of IFNAR1 in shCOO2 and ShPKD2 stable cells after treatment with interferon alpha.

FIG. 33 is an image demonstrating that knockdown of PKD2 prevents downregulation of cell surface levels of IFNAR1 as measured y the FACS analysis. Green line=untreated cells; blue and brown lines=treated with IFN-α (for 1 and 2 hr respectively). Red=negative control (isotype antibody).

FIG. 34 is an image depicting the analysis of the effects of knockdown of PKD2 in HeLa cells on IFN-α signaling measured by activating tyrosine phosphorylation of Stat1 protein, Cells were treated with a pulse (15 min) of IFN-α and when incubated in IFN-free media. Stat1 phosphorylation (upper panel) and total levels (lower panel) was assessed at different time points (in minutes) as indicated.

FIG. 35 is an image depicting the analysis of the knockdown of PKD2 in human 2fTGH cells stimulates expression of interferon-inducible genes such as PKR and Stat1 Levels of these proteins and levels of actin (as loading control) are analyzed as indicated.

FIG. 36 is an image depicting the analysis of the replication of vesicular stomatitis virus (VSV) of 2fTGH-shCON and 2fTGH-shPKD2 cells. Upper panels show the assessment of viral replication by expression of viral VSV-M protein. Lower panel actually depicts the results of measurement of the titer of VSV in cells treated with indicated doses of IFN-α.

FIG. 37, comprising FIGS. 37A-37E, depicts the results of example experiments demonstrating that Sangivamycin (SGM) inhibits PKD2 and increases the efficiency of IFN signaling.

FIG. 38, comprising FIGS. 38A-38D, depicts the results of example experiments demonstrating that sangivamycin inhibits PKD2 and increases the efficiency of IFN signaling.

FIG. 39, comprising FIGS. 39A-39E, depicts the results of example experiments conducting inhibitory analysis of the degron phosphorylation of IFNAR1 and its interaction with O-Trcp2. (A) In vitro binding of 35S-labeled β-Trcp2 to GST-IFNAR1 (wild type or S535,539A mutant, SA) upon its phosphorylation with CK1α-depleted lysates from cells treated with IFN-α as indicated; (B) Effect of various kinase inhibitors on phosphorylation of GST-IFNAR1 by the CK1α-depleted lysate from IFN-α-treated (for 10 min) cells analyzed by subsequent binding of 35S-labeled β-Trcp2; (C) Immunoblot analysis of Flag-IFNAR1 immunopurified from cells pre-treated with kinase inhibitors and then treated with IFN-α as indicated; (D, E) Immunoblot analysis of endogenous IFNAR1 immunopurified from cells pre-treated with kinase inhibitors and treated with IFN-α as indicated.

FIG. 40, comprising FIGS. 40A-40E, depicts the results of example experiments demonstrating that PKD2 mediates degron phosphorylation of IFNAR1 (A) Immunoblot analysis of endogenous IFNAR1 immunopurified from HeLa cells that received indicated siRNA oligos. Levels of PKD species and β-actin in whole cell lysates (WCL) were also analyzed; (B) Immunoblot analysis of Flag-IFNAR1 immunopurified from U3A cells that received indicated siRNA oligos; (C) In vitro phosphorylation of GST-IFNAR1 on Ser535 by purified GST-PKD species was analyzed by immunoblotting; (D) Effect of Gö6976 (20-200 nM) on in vitro phosphorylation of GST-IFNAR1 by purified GST-PKD2 (wild type or kinase dead, KD) analyzed as in panel C; (E) Immunoblot analysis of endogenous IFNAR1 immunopurified from cells stably transduced with shRNA against GFP (shCON) or PKD2 (shPKD2) and then treated with IFN-α or thapsigargin (TG) was carried out as described in panel A.

FIG. 41, comprising FIGS. 41A-41E, depicts the results of example experiments demonstrating that PKD2 regulates ubiquitination, endocytosis and degradation of IFNAR1. (A) Immunoblot analysis of IFNAR1 immunopurified from HeLa cells that received indicated shRNA oligos. Levels of PKD2 and β-actin in whole cell lysates (WCL) were also analyzed; (B) Effect of PKD2 knockdown (open squares) on the rate of internalization of endogenous IFNAR1 measured by a fluorescence-based assay are presented as % of total cell surface IFNAR1 level (Mean±S.E.M.); (C) FACS analysis of IFNAR1 levels on the surface of cells that received indicated shRNA. Green, blue, and brown signals represent cell surface expression of IFNAR1 after 0, 1, and 2 h of IFN-α treatment, respectively (red-isotype control); (D) Immunoblot analysis of endogenous IFNAR1 in cells untreated or pre-treated with the PKD inhibitor CID755673, and then subjected to a cycloheximide (CHX) chase in the presence of IFN-α for the indicated times; (E) Degradation of IFNAR1 in cells that received indicated shRNA was assessed as in panel D.

FIG. 42, comprising FIGS. 42A-42D, depicts the results of example experiments demonstrating the ligand-induced recruitment of PKD2 to IFNAR1 and stimulation of PKD2 kinase activity. (A) Immunoblot analysis of IFNAR1 immunopurified from 293T cells treated with IFN-α for the indicated times. Left lane of the upper panel represents the whole cell lysate from untreated cells; (B) Immunoblot analysis of phospho-Ser710 levels of GST-PKD2 and purified from HeLa cells treated with IFN-α as indicated; (C) Immunoblot analysis of phospho-Ser710 levels of endogenous PKD2 immunopurified from HeLa cells treated with IFN-α as indicated; (D) In vitro immunokinase activity of endogenous PKD2 purified from HeLa cells (treated with IFN-α as indicated) towards GST-IFNAR1 as a substrate in the presence of γ-32P-ATP as analyzed by autoradiography (upper panel), Coomassie staining (middle panel), and immunoblotting with an antibody against PKD (lower panel).

FIG. 43, comprising FIGS. 43A-43E, depicts the results of example experiments assessing the role of Tyk2 and tyrosine phosphorylation of PKD2 in its activation and IFNAR1 degron phosphorylation. (A) Activity of GST-PKD2 expressed in cells harboring wild type or kinase dead Tyk2, and left untreated or treated with IFN-α, was analyzed by in vitro Ser535 phosphorylation of GST-IFNAR1 as assessed by immunoblotting using a phospho-S535 specific antibody. Levels of substrate and kinase were also analyzed by immunoblotting; (B) Tyrosine phosphorylation and levels of endogenous IFNAR1 immunopurified from HeLa cells treated with IFN-α as indicated were analyzed by immunoblot; (C) In vitro tyrosine phosphorylation of recombinant PKD2 by recombinant Src or HA-tagged Tyk2 purifed from cells untreated or treated with IFN-α as indicated, was analyzed by immunoblotting using phospho-Tyr specific antibody. Immunoblot analysis of levels of HA-Tyk2 and PKD2 in the reaction is also provided in the middle and lower panels; (D) Activity of GST-PKD2 (wild type or Y438F mutant) expressed in HeLa cells treated as indicated was analyzed as in panel A; (E) Immunoblot analysis of phosphorylation and levels of endogenous IFNAR1 in HeLa cells that received indicated shRNA and expression constructs. Levels of endogenous (lower panel) and exogenous PKD2 in whole cell lysates are also shown.

FIG. 44, comprising FIGS. 44A-44F, depicts the results of example experiments demonstrating that PKD2 regulates the extent of cellular responses to IFNa. (A) Stat1 Tyr phosphorylation and levels in cells untreated or pre-treated with the PKD inhibitor CID755673 for 1 h and then pulse-treated with IFN-α for 15 min (followed by removal of cytokine and inhibitor and incubation of cells for the indicated times) was analyzed by immunoblotting; (B) Analysis in cells that received indicated shRNA was carried out as in panel A; (C) Relative activity of ISRE-driven firefly luciferase activity normalized to renilla luciferase activity in 2fTGH cells. Cells were pre-treated with the PKD inhibitor CID755673 (for 1 h) and pulse-treatment with IFN-α (for 0-2 h) as indicated and the activity of luciferase was assessed 24 h later. Data from four independent experiments (each in triplicate) are presented as Mean±S.E.M.; (D) Analysis of ISRE-driven transcription in 2fTGH cells that received the indicated shRNA and were pulse treated with IFN-α for indicated time was carried out as described in panel C; (B) Immunoblot analysis of PKR, Stat1 and β-actin levels in 2fTGH cells that received the indicated shRNA, were pulse-treated with IFN-α for indicated times, and were analyzed 24 h thereafter; (F) Cells were untreated or pre-treated with IFN-α (at the indicated doses) and then infected with VSV. Viral titers from three independent experiments (each in quadruplicate) are shown as mean±S.E.M.

FIG. 45, comprising FIGS. 45A-45F, depicts the results of example experiments assessing the role of phosphorylation-dependent degradation of IFNAR1 in VEGF-stimulated angiogenesis. (A) Immunoblot analysis of ubiquitination, phosphorylation and levels of Flag-IFNAR1 stably expressed in U3A cells treated with VEGF as indicated. Phosphorylation and levels of PKD and Erk were also analyzed; (B) Immunoblot analysis of phosphorylation and levels of Flag-IFNAR1 from U3A cells that received indicated shRNA and were treated with VEGF as indicated; (C) Degradation of Flag-IFNAR1 in U3A cells treated with VEGF as indicated; (D) Immunoblot analysis of Stat1 phosphorylation and levels in human umbilical vein endothelial cells pre-treated with VEGF (100 ng/ml for 2 h) and treated with indicated dose of IFN-α (for 15 min). The relative ratio of phospho-Stat1/total Stat1 signal (arbitrary rated at 1.0 in untreated cells) is depicted below the bottom panel; (E) Cell surface levels of mIFNAR1 in mVEGF-treated (red line) or not (blue line) CD31-positive bone marrow cells from indicated mice; control Ig—green line; (F) In vivo angiogenesis assay in mice of indicated genotype was carried out as outlined in Experimental Procedures. Pictures of retrieved plugs and graph that depicts measurement of hemoglobin (mean±S.E.M.) are shown in the left panels. Immunohistochemical analysis using anti-CD31 Ab is shown on the right.

FIG. 46 depicts the results of example experiments assessing the phosphorylation of endogenous IFNAR1 in 293T cells transfected with siRNA against PKD1 or 293T cells PKD2. Levels of PKD species were also analyzed by immunoblot in WCL.

FIG. 47 depicts the results of example experiments assessing indicated GST-tagged PKD proteins expressed in 293T cells and purified by pull down with glutathione beads were incubated with myelin basic protein (MBP) and radiolabeled γ32P-ATP. A control reaction (“CON”) was carried out using the lysates from untransfected cells. Incorporation of labeled phosphate into PKD and MBP was analyzed by autoradiography. Levels of MBP (Coomassie staining) and GST-tagged PKD species (immunoblot with antibody against GST) are also shown.

FIG. 48 depicts the results of example experiments assessing the co-immunoprecipitation of endogenous IFNAR1 and PKD2 from the lysates of 293T cells. Control reaction utilized an irrelevant monoclonal IgG antibody (anti-Flag).

FIG. 49 depicts the results of example experiments assessing the phosphorylation of and levels of endogenous IFNAR1 immunopurified from 11.1-derived cells that harbor wild type (WT) or catalytically inactive (KR) Tyk2 treated with IFN-α as indicated.

FIG. 50 depicts the results of example experiments assessing the cytopathogenic effect of VSV manifested in the appearance of rounded, poorly attached, dying cells upon infection of human 2fTGH cells that received indicated shRNAs and pre-treated with IFN-α as indicated and then infected with VSV (at M.O.I. of 0.5). A comparable viral load in untreated cells was verified by expression of VSV-M viral protein (analyzed by immunoblot shown in the lower panels). The extent of PKD2 knockdown is also shown.

FIG. 51 depicts the results of example immunoblot analyses of endogenous IFNAR1 immunopurified from cells treated with IFN-α, H2O2 or TPA as indicated.

FIG. 52 depicts the results of example cychoheximide chase analyses of turnover of endogenous IFNAR1 in human umbilical vein endothelial cells untreated or treated with VEGF, Equal loading was verified by analysis of β-actin in these samples. The graph depicts % of remaining IFNAR1 at the indicated time points.

FIG. 53 depicts the results of example PCR analyses of DNA from tails of back-crossed chimeric S526A mice of the indicated gender. Presence of the SA alleles in 3 founders is indicated by a PCR product that migrates slower due to a remaining loxP site.

FIG. 54, comprising FIGS. 54A-54C, depicts the results of example experiments demonstrating the purification of cellular Ser535 kinase activity. (A) Purification scheme and results from in vitro kinase activity assays that used immunoblotting with phospho-specific antibody or [˜−32P]ATP incorporation into the GST-IFNAR1 substrate as indicated. (B) Phosphorylation of bacterium-produced GST-IFNAR1 (wild type or Ser535,539Ala mutant [SA]) by the starting fractions before loading onto either SP Sepharose (SP) or hydroxyappatite (HA) columns in the presence of radioactive [˜−32P]ATP was analyzed by SDS-PAGE and autoradiography. Mutant IFNAR1 migrates slower due to the presence of additional amino acids in the linker between GST and the cytoplasmic domain of IFNAR1 (as outlined in references 31 and 32). (C) Active fractions were pooled after the last purification step. Proteins were precipitated and separated on an SDS-PAGE gel followed by silver staining. Five indicated major bands were cut out for mass spectrometry analysis. The identities of the bands and the sequences of identified Ck1α-derived peptides are shown on the tight.

FIG. 55, comprising FIGS. 55A-55E, depicts the results of example experiments demonstrating that Ck1α represents the major Ser535 kinase in the cell lysates. (A) HeLa lysates were immunoprecipitated (IP) with control immunoglobulin Gs (IgGs) or antibodies against Ck1α or CK1s and protein G beads. The supernatants of these reaction mixtures were analyzed for their S535 kinase activity by an in vitro kinase assay (KA) with GST-IFNAR1 as a substrate, detected by immunoblotting using anti-pS535 and anti-GST antibodies (upper panels). Efficacy of immunodepletion was verified by immunoblotting using antibodies for Ck1α and CK1s (lower panels). (B) Lysates from HeLa cells transfected with the indicated siRNAs were used in the kinase assay. Phosphorylation of GST-IFNAR1 and levels of Ck1α in whole-cell lysates (WCL) were analyzed by IB using the indicated antibodies. Ponceau S staining of the membrane to detect GST-IFNAR1 is also depicted. (C) 293T cells were transfected with empty vector or Myc-tagged human Ck1α, and lysates were prepared. These lysates were analyzed for their IFNAR1 Ser535 kinase activity (as for panels A and B). In addition, phosphorylation of GST-IFNAR1 in the immunokinase assay (as well as the levels of Myc-Ck1α and GST-IFNAR1) was assessed via IP using anti-Myc antibody; results are depicted in the lower panels. (D) 293T cells were treated with DMSO or TG (1 μM) for 30 min. Lysates were subjected to Ck1α IP, followed by analysis of Ser535 activity in vitro using GST-IFNAR1 as a substrate. Induction of ER stress was shown by phosphorylation of p-eIF2α as assessed by IB using phosphor-specific antibody. (E) 293T cells were untreated or treated with TG (1 μM for 30 min) and harvested. Lysates from these cells were immunodepleted of Ck1α as outlined for panel A. Increasing amounts (0.12 to 0.5 μg) of bacterium-produced recombinant GST-Ck1α were incubated with the substrate (GST-IFNAR1) and ATP (except in lane 1) at 30° C. for 30 min without any lysates (lanes 4 to 6) or in the presence of 4 μg of immunodepleted lysates from untreated (UN; lanes 7 to 9) or TG-treated (lanes 10 to 12) cells. Phosphorylation of GST-IFNAR1 on Ser535, levels of GST-IFNAR1 (using anti-GST antibody), and levels of Ck1α were analyzed by IB.

FIG. 56, comprising FIGS. 56A-56F, depicts the results of example experiments demonstrating that Ck1α mediates basal IFNAR1 phosphorylation, ubiquitination, and downregulation in cells. (A) 293T cells were cotransfected with Flag-IFNAR1 and Myc-hCk1α or an empty vector. Ser535 phosphorylation of Flag-IFNAR1 was analyzed by Flag immunoprecipitation (IP) followed by IB of pS535. The total levels of IFNAR1 were determined by reprobing the blot with an anti-Flag antibody. Myc-Ck1α levels in the whole-cell lysates (WCL) are shown in the lower panel. (B) 293T cells expressing Flag-IFNAR1 were treated with CKI-7 (400 μM) for the indicated times. Immunopurified IFNAR1 was analyzed by IB using the indicated antibodies. (C) HeLa cells were cotransfected with Flag-IFNAR1 and siRNA against Ck1α or a control siRNA. At 48 h after transfection, lysates were harvested and were subjected to IP using anti-Flag antibody followed by IB analysis using the indicated antibodies. Levels of Ck1α and Erk1/2 (as a loading control) in WCL were assessed by IB using the indicated antibodies. (D) HeLa cells were transfected with RNAi against Ck1α or luciferase and analyzed for the surface levels of endogenous IFNAR1 by FACS using the AA3 monoclonal antibody. Control using irrelevant immunoglobulin (Ig) is also shown. (E) HeLa cells were transfected with siRNA as for panel C and then treated with the low dose of IFN-a (5 IU/ml) for 15 min as indicated. Activation and levels of Stan were analyzed by immunoblotting using the indicated antibodies. The ratio between pStat1 and Stat1 signals was calculated using Li-Cor's Odyssey infrared fluorescence-based quantification system. (F) Human 2fTGH cells were cotransfected with shRNA against GFP (shCON) or against Ck1α (shCk1α) and with pBABE-puro vector. After 4 days of selection in medium containing puromycin (4 μg/ml), the cells were plated into 96-well plates (5×104/well) and treated (+) or not treated (−) with IFN-a (250 IU/ml for 48 h) as indicated. Numbers of cells as a function of absorbance were measured using the CellTiter 96 cell proliferation assay kit (Promega) and are presented in optical density (OD) units as depicted on the graph. Averages of a total of six experiments are shown.

FIG. 57, comprising FIGS. 57A-57E, depicts the results of example experiments demonstrating that Ck1α is required for efficient IFNAR1 downregulation in response to ER stress. (A) HeLa cells were transfected with control siRNA or siRNA against Ck1α. After 48 h, cells were treated with vehicle control, TO (1 μM), or IFN-a (1,000 IJ/ml) for 30 min, and lysates were harvested. The lysates were subjected to IFNAR1 immunoprecipitation (IP) followed by 1B of pSer535 and total IFNAR1. The efficiency of Ck1α knockdown is shown in the lower panel. (B) 2fTGH cells were pretreated with 15 μM of D4476 or DMSO for 1 h and then treated with vehicle control, TG (1 μM), or IFN-a (1,0001J/ml) for 30 min. IFNAR1Ser535 phosphorylation and total levels were determined by IP-IB. Total levels of eIF2a and its phosphorylation (as a marker of the PERK-dependent effect of TG) in whole-cell lysates (WCL) were also analyzed. (C) HeLa cells were transfected with control siRNA (siCon) or siRNA against Ck1α (siCk1α). At 48 h after transfection, cells were treated with DMSO or TG (1 μM) for the indicated times. Levels of total IFNAR1 were determined by IP-IB. Levels of Ck1α and actin in total cell lysate were examined by IB. (D) 2fTGH cells were infected with VSV (MOI, 0.1) for 13 h. The infected cells were then treated with DMSO or 20 μM of D4476, and cells were further incubated for 0.5, 1.0, or 2.0 h. At these time points, cells were harvested. Endogenous IFNAR1 from these cells was analyzed by IP-IB using the indicated antibodies, Levels of viral protein VSV-M and phosphorylation of eIF2a (indicative of ER stress) were also assessed by 1B in WCL. The nonspecific band (NS) is indicative of the loading of the gel. (E) Lysates from experiments shown panels B and D were analyzed for Stat1 phosphorylation and Stat1 levels by 1B using the indicated antibodies.

FIG. 58, comprising FIGS. 58A-58F, depicts the results of example experiments characterizing the S535 kinase activity of several human CK1 isoforms and CK1-like proteins from other organisms, (A) 293T cells were transfected with an empty vector (Vec) or Myc-tagged Ck1α (a), CK1 S(S), or CK1 s (s) or, as shown in the right panel, with HA-tagged vaccinia virus B1 kinase (vvB1), the kinase-dead vvB1(1(D-B1), L. major CK1 (L-CK1), or human Ck1α. These transfected kinases were IPed with Myc or HA and were subjected to in vitro immunokinase assay (KA) to determine Ser535 phosphorylation of GST-IFNAR1. Levels of the substrate as well as kinases expression were also analyzed. (B) Autophosphorylation of HA-tagged vvB1 (expressed in and immunopurified from 293T cells) was carried out in the presence of labeled [˜−32P]ATP and detected by SDS-PAGE and autoradiography. Immunoprecipitation (IP) reactions from the lysates of the vector-transfected cells or cells expressing catalytically inactive KD-vvB 1 mutant were used as a negative control. (C) In vitro phosphorylation of GST-IFNAR1 using lysates from cells transfected with indicated kinases as a source of the kinase activity was measured by immunoblotting using phospho-specific anti-pS535 antibody (upper panel). Expression of CK1 species was analyzed by IB using anti-HA antibody (lower panel). pEF and pSG indicate cells transfected with the indicated empty vectors. (D) 293T cells were cotransfected with Flag-IFNAR1 together with an empty vector (Vec) or Myc-tagged Ck1α (a), CK1S(S), or CK1s (s) or, as shown in the right panel, with HA-tagged L-CK1. Phospho-S535 and total IFNAR1 signals were analyzed by IP-IB. Ectopic expression levels of the kinases were determined by Myc or HA IB. In the left panel, phosphorylation and total eIF2α levels are indicative of comparable levels of ER stress in cells transfected with different CK1 isoforms. (E) In vitro phosphorylation of GST-IFNAR1 with supernatant from L. major promastigote culture. Buffer lacking Leishmania was used as a control (Con), These fractions were incubated with ATP and GST-IFNAR1 (5 μg) at 30° C. for 30 min. The products of this kinase reaction were analyzed by IB for 0535 and GST. (F) In vitro phosphorylation of GST-IFNAR1 by concentrated supernatant of cultured amastigotes of L. mexicana (obtained upon treatment with buffers with indicated pHs that mimicked the phagosomal or cytosolic environments) was measured by incorporation of radioactive phosphate as described elsewhere herein.

FIG. 59, comprising FIGS. 59A-59D, depicts the results of example experiments demonstrating that expression of L-CK1 promotes phosphorylation-dependent IFNAR1 ubiquitination, and degradation. (A) 293T cells were cotransfected with Flag-IFNAR1 and HA-tagged L-CK1 (WT or kinase-dead K40R mutant) or an empty vector. The levels of pS535 and total IFNAR1 were determined by IP-IB. The levels of L-CKI in whole-cell lysates (WCL) were determined by IB using anti-HA antibody. (B) 293T cells were cotransfected with IFNAR1 (WT or S535A mutant) and L-CK1 or an empty vector. The levels of ubiquitinated, S535-phosphorylated, and total IFNAR1 were analyzed by IP-IB. The levels of L-CK1 were assessed by HA IB. (C) MEFs derived from IFNAR1′ mice reconstituted with WT or S526A mouse IFNAR1 were transfected with L-CK1 or an empty vector. The levels of IFNAR1 and L-CK1 were determined by Flag and HA IB, respectively. N.S., nonspecific band. (D) Human blood monocyte-derived dendritic cells were infected with L. major promastigote culture (containing 50% metacyclics at an MOI of 10) or left uninfected as a control (Con). After overnight incubation, cells were subjected to FACS analysis of cell surface IFNAR1 using AA3 monoclonal antibody.

FIG. 60, comprising FIGS. 60A-60D, depicts the results of example experiments demonstrating that L. major infection or L-CK1 expression suppresses Type I IFN signaling. (A) Mouse bone marrow-derived macrophages were infected or not with L. major parasites (as outlined for FIG. 59D) at the indicated ratios. After overnight incubation, cells were treated with mouse IFN-a (200 IU/ml) or IFN—y (10 ng/ml) for 30 min. Levels of phosphorylated and total Stat1 were determined by IB. (B) 293T cells transfected with the indicated plasmids were subjected to pulse treatment with human IFN-a (500 IU for 15 min), Cells were harvested at the indicated time points after beginning of treatment and analyzed for Stat1 activation using the indicated antibodies. Levels of L-CK1 were analyzed by IB. (C) MEFs from IFNAR1mice reconstituted as described for FIG. 59C were transfected with empty vector or L-CK1 as indicated. After 24 h, cells were trypsinized and equal numbers of cells were plated into 12-well plates. After overnight incubation, cells were pulsed with murine IFN-a (50 IU/ml) for 30 min and then chased with fresh medium for the indicated times (relative to the initial addition of IFN). Lysates were harvested, and the levels of pStatl, total Stat1, and Flag-IFNAR1 were determined by 113. (D) Lysates from untreated cells from the experiment shown in panel C were analyzed for expression of HA-tagged L-CK1 using IP-IB with anti-HA antibody.

FIG. 61, comprising FIGS. 61A-61B, depicts the conserved priming site within IFNAR1 regulates the intrinsic stability of the protein. A. Alignment of primary sequences of IFNAR1 from indicated species. The phospho-degron sequences are shaded and serine residues within the degron are underlined. The conserved putative priming site (Ser532 in human IFNAR1) is denoted by an asterisk. B. Degradation of Flag-IFNAR1 (wild type or S532A mutant) overexpressed in 293T cells was analyzed by cycloheximide (CHX, 2 mM) chase for the indicated times followed by immunoblotting using anti-Flag antibody. Levels of f3-actin were also analyzed as a loading control.

FIG. 62, comprising FIGS. 62A-62D, depicts the results of example experiments demonstrating that priming phosphorylation is required for the ligand-independent phosphorylation of IFNAR1 degron. A. Degron phosphorylation of Flag-IFNAR1 (wild type or Ser532A mutant) co-expressed in 293T cells with Myc-tagged human CK1α or empty vector (Vec) and treated or not with IFNα (1000 IU/mL for 30 min as indicated) was analyzed by Flag immunoprecipitation followed by immunoblotting using the indicated antibodies. Levels of Myc-CK1α in whole cell lysates were also determined. B. Flag-IFNAR1 (wild type or Ser532A mutant) was co-expressed in 293T cells with HA-tagged Leishmania CK1 (HA-L-CK1, wild type or kinase-dead, KD) and purified by Flag immunoprecipitation. Phosphorylation of the IFNAR1 degron and levels of IFNAR1 were analyzed by immunoblotting using the indicated antibodies. Levels of HA-L-CK1 in whole cell lysates (WCL) were also determined. C. Characterization of anti-pS532 antibody. Flag-IFNAR1 proteins (wild type, S535A or S532A mutants) were expressed in 293T cells, immunopurified, and analyzed using the indicated antibodies, Vec-reactions from cells transfected with empty vector (pcDNA3). D. 293T cells were untreated (UN) or treated with thapsigargin (TG, 1 μM for 30 min) and harvested. Lysates from these cells were twice immunodepleted with antibodies against CK1α and the CK1α-free supernatants (4 μg) were used alone (lanes 2-3) or together with 0.5 μg of bacterially produced recombinant GST-CK1α (lanes 1 and 4-9) for in vitro phosphorylation of GST-IFNAR1 (wild type, lanes 1-6, or S532A mutant, lanes 7-9) in the presence of ATP (except in lane 1) at 30° C. for 30 min as indicated. Phosphorylation of GST-IFNAR1 on Ser532, Ser535, levels of GST-IFNAR1 (using anti-GST antibody) and levels of CK1α were analyzed by immunoblotting.

FIG. 63, comprising FIGS. 63A-63D, depicts the results of example experiments demonstrating that UPR induces phosphorylation of the priming site that is required for increased ubiquitination and degradation of IFNAR1. A. Endogenous IFNAR1 proteins immunopurified from HeLa cells that were untreated (Mock), or were treated with TG (1 μM for 30 min), IFNα (6000 IU/mL for 30 min), or infected with VSV (0.1 MOI for 1 hr followed by additional 10 hr incubation in virus-free medium) as indicated, were analyzed by immunoblotting using the indicated antibodies. B. Endogenous IFNAR1 proteins immunopurified from 11.1-Tyk2-null derivative cell lines reconstituted with wild type Tyk2 (WT) or catalytically deficient Tyk2 (KR) or WT cells were treated with TG or IFNα (in doses indicated in panel A) for 30 min. C. Flag-IFNAR1 proteins (wild type or S532A mutant) were expressed in 293T cells. The cells were pre-treated with a lysosomal inhibitor (methylamine HCl, 10 mM) for 1 hr to prevent degradation of ubiquitinated receptors. Then the cells were treated with TG (1 μM for the indicated times) and Flag-IFNAR1 proteins were immunopurified under denaturing conditions and analyzed by immunoblotting using antibodies against ubiquitin (upper panel) and Flag (lower panel). D. Downregulation of the levels of Flag-IFNAR1 (wild type or S532A mutant) expressed in 293T cells treated with TG (1 μM for indicated times) was analyzed by immunoblotting using Flag antibody. Comparable loading was verified by anti-β-actin immunoblot (lower panel).

FIG. 64, comprising FIGS. 64A-64D, depicts the results of example experiments demonstrating the role of PERK in UPR-induced phosphorylation of priming site of IFNAR1. A. HeLa cells transfected with smRNA against PERK or against GFP (shCon) were treated with TG (1 μM for 30 min) and endogenous IFNAR1 proteins were immunopurified and analyzed for phosphorylation on the priming site, and for total levels by immunoblotting using the indicated antibodies. Phosphorylation of a known PERK substrate eIF2α (as well as its total levels) and the levels of PERK itself were also determined in whole cell lysates (WCL). B. Mouse embryo fibroblasts from wild type or PERK knockout animals were treated with TG as indicated. Levels and priming phosphorylation of endogenous murine IFNAR1 on Ser523 (analogue of human Ser532) was analyzed by immunoblotting using indicated antibodies. Phosphorylation and levels of eIF2α and the levels of PERK in whole cell lysates (WCL) was also determined. C. Levels of endogenous IFNAR1 in 293T cells transfected with wild type or the catalytically deficient mutant (K618A) of PERK were analyzed by immunoprecipitation followed by immunoblotting using an anti-IFNAR1 antibody. Levels of PERK, phosphorylated PERK, and levels of eIF2α were also examined. D. Whole cell extracts from 293T cells or recombinant bacterially produced constitutively active ΔN-PERK were incubated alone or with GST-IFNAR1 in the presence of radiolabeled y-ATP as indicated. Resulting phosphorylation of GST-IFNAR1 or contaminants and autophosphorylation of PERK was determined by SDS-PAGE followed by Coomassie staining and autoradiography. Positions of PERK, GST-IFNAR1, and some irrelevant contaminants (denoted by asterisks) are indicated.

FIG. 65, comprising FIGS. 65A-65D, depicts the results of example experiments demonstrating that priming phosphorylation of IFNAR1 contributes to regulation of the extent of IFNα/β signaling. A. Control human Huh7 cells, and those expressing the HCV replicon, were analyzed for IFNAR1 levels by immunoprecipitation-immunoblotting (upper panel). The lower three panels depict the experiments where gel loading was normalized to achieve comparable levels of immunopurified IFNAR1 in each lane. Phosphorylation of IFNAR1 on Ser532 and Ser535 was determined by immunoblotting using the indicated antibodies. B. Control human Huh7 cells, and those expressing the HCV replicon, were transfected with Flag-tagged Stat1 alone with empty vector (Vec) or Flag-IFNAR1 (wild type or S532A mutant), and were untreated or treated with IFN-α (60 IU/mL for 30 min) as indicated. Lysates of these cells were immunoprecipitated using anti-Flag antibody and analyzed by immunoblotting using antibodies against phospho-Stat1, total Stat1, and IFNAR1. C. Mouse embryo fibroblasts from IFNAR1-null animals were reconstituted with murine Flag-IFNAR1 (wild type or S523A mutant, which is a mouse analogue of human S532A mutant). Cells were treated with indicated doses of murine IFN-β for 1 h, incubated for 8 h in fresh medium, and then infected with VSV (MOI 0.1). Expression of VSV-M protein was analyzed 16 h later by immunoblotting. Levels of β-actin were also determined (lower panel). D. Model for ligand-dependent and ligand-independent ubiquitination and degradation of IFNAR1. Both pathways converge at the level of degron phosphorylation (pS535). Signaling induced by IFN and dependent on the activity of Tyk2 does not require either CK1α (24) or priming phosphorylation (this study). Ligand-independent pathway initiated by inducers of UPR does not need either ligand or Tyk2 activity but requires CK1α (26) and PERK-dependent priming phosphorylation, as disclosed herein.

FIG. 66, comprising FIGS. 66A-66E, depicts the results of example experiments demonstrating that PERK expression is maintained in cancer cells wherein it regulates tumor expansion in vivo. (A) PERK protein levels were measured by immunoprecipitation (IP) followed by Western blot analysis in the following cell lines: MCF10A (1), MCF7 (2), T47D (3), MDA-MB231 (4), MDA-MB468 (5), TE3 (6), TE7 (7), KYSE 520 (8). (B) PERK protein levels following shRNA targeting of PERK. (C) Parental MDA-MB468 cell line, shPERK-transduced cells (shPERK), and shPERK-transduced cells reconstituted with mouse Myc-PERK (+mPERK) were treated with 2 g/ml tunicamycin for the indicated intervals. Western analysis for ATF4, CHOP, or -actin. (D) Volume of orthotopic tumors formed from the mouse mammary tumor-derived cells transduced in vitro with empty vector virus (Neu/PERKloxP/loxP) or Cre-expressing retrovirus)(Neu/PERK/°) 28 days post-transplant (n=4). Representative image of tumors are provided. All p-values determined by Student t-test. (E) Western analysis of transgenic ErbB2 and PERK expression following infection of mouse mammary tumor-derived cells with control (Neu/PERKloxP/loxP) or Cre-expressing retrovirus (Neu/PERK/). Thapsigargin treatment (50 nM, 1 h) was used to demonstrate that PERK is functional.

FIG. 67, comprising FIGS. 67A-67D, depicts the results of example experiments demonstrating that PERK knockdown triggers a G2/M delay. (A) MDA-MB468 cells were infected with control shRNA or anti-PERK shRNA for the indicated intervals. Cells were pulsed with BrdU 45 min prior to harvest for FACS analysis. (B) Kinetics of growth of the MDA-MB468 parental cell line, control shRNA-(shControl) or shPERK-transduced cells (shPERK), and shPERK-transduced cells reconstituted with mouse Myc-PERK (+InPERK), PERK protein levels following expression of shRNA targeting human PERK and reconstitution with mouse Myc-PERK are shown. (C) Proliferation rates in mammary gland sections from control (PERKloxP/loxP) and mammary gland-specific PERK knockout mice)(PERK/° on pregnancy day 16 (P16) and lactation day 3 (L3) were determined by immunohistochemistry for BrdU (animals were injected with BrdU 1 h prior to being euthanized). (D) Quantification of BrdU-positive cells from (C) is shown; error bars indicate S.D. among 3 animals, 5 acini were counted per animal.

FIG. 68, comprising FIGS. 68A-68D, depicts the results of example experiments demonstrating that PERK knockdown triggers DNA damage response signaling pathway. (A) Immunofluorescence staining for DNA damage-induced foci containing phospho-ATM and phospho-Chk2 following acute PERK knockdown (72 h after infection) in MDA-MB468 cells. (B) Quantification of phospho-ATM positive cells (>3 foci) is shown; error bars indicate S.D. from 3 slides, 5 fields were counted per slide. p-value was determined by Student t-test. (C) Western analysis of DNA damage response-associated markers following PERK knockdown. (D) IP/kinase assays assessing CDK2-dependent phosphorylation of histone H1 (bottom panel). CDK2 complexes were immunoprecipitated from MDA-MB 468 cells treated as indicated. PERK levels were assessed by IP/immunoblot and CDK2 recovery in precipitates was assessed by CDK2 immunoblot (middle panel).

FIG. 69, comprising FIGS. 69A-69C, depicts the results of example experiments demonstrating that increased levels of Reactive Oxygen Species (ROS) in PERK knockdown cells contribute to reduced kinetics of cell growth. (A) DCF fluorescence measured by FACS using CM-H2DCFDA dye. (B) Mean values of ROS determined from three independent experiments. Uninfected (black), stably infected with empty vector (blue) or shPERK (red) in both (A) and (B). (C) Growth analysis of MDA-MB468 parental cell line, cells transduced with control shRNA, shPERK, or shPERK and reconstituted with mouse Myc-PERK in the presence of ROS scavenger N-acetylcysteine (NAC).

FIG. 70, comprising FIGS. 70A-70D, depicts the results of example experiments demonstrating that ROS accumulation triggers oxidative DNA lesions in PERK-deficient breast cancer cells and tumors. (A) Detection of 8-oxoguanine oxidized DNA adduct (8-OxoG) using a FITC conjugated 8-OxoG binding peptide in parental MDA-MB468 cells, MDA-MB468 cells transduced with control shRNA, shPERK, or shPERK and reconstituted with mouse Myc-PERK. (B) Quantification of 8-oxoG positive cells from 3 independent experiments. (C) 8-OxoG in paraffin sections from tumors formed by parental MDA-MB468 cells, MDA-MB468 cells transduced with control shRNA, or shPERK. (D) Quantification of 8-OxoG positive cells shown in (C) is provided and error bars indicate S.D. from 4 animals. (E) Detection of 8-OxoG in paraffin sections from orthotopic tumors formed by mouse mammary tumor-derived cells transduced with empty vector (Neu/PERKloxP/loxP) or retrovirus expressing Cre recombinase (Neu/PERKP). (F) Quantification of 8-OxoG positive cells from (E); error bars indicate S.D. from 4 animals. All p-values determined by Student t-test.

FIG. 71, comprising FIGS. 71A-71C, depicts the results of example experiments demonstrating that ROS accumulation triggers DNA double strand breaks in PERK-deficient breast cancer cells and tumors, (A) Immunofluorescent staining of—H2AX foci in control or PERK knockdown MDA-MB468 cells. (B) Quantification of -H2AX positive (>5 foci) cells under standard tissue culture conditions. Error bars represent S.D. from 3 independent experiments performed in triplicate. (C) Quantification of the COMET tail moment in control or PERK knockdown cells under standard tissue culture conditions. Error bars indicate S.D. from 3 experiments.

FIG. 72, comprising FIGS. 72A-72F, depicts the results of example experiments demonstrating that reduced activity of Nrf2 causes increased oxidative stress in PERK knockdown cells. (A) Quantitative real time PCR analysis of Nrf2 target genes NQO1 and GCLC in the indicated cell lines asynchronously proliferating under standard conditions. (B) Purified recombinant Nrf2-Neh2 domain of WT, T80A, S40A or T80A/S40A, was incubated with purified recombinant AN-PERK in the in vitro kinase assay. Phosphorylated Nrf2-Neh2 was detected by autoradiography (upper panel). (C) 293T cells were transfected with WT Nrf2 or Nrf2-T80A. 24 hours after transfection, cells were left untreated (C) or treated with tunicamycin (Tu) for 2 hours followed by immunoprecipitation with anti-Nrf2 antibody. Threonine phosphorylation was detected using a phospho-Thr reactive antibody. Nrf2 in the IP and the whole cell lysate (WCL) was detected with Nrf2 specific antibody. (D) Proliferation of the indicated cell lines was assessed by a 6-day growth curve under standard tissue culture conditions as described in materials and methods. PERK levels were detected by IP/Western blot analysis. (E) Oxidized guanine in damaged DNA was detected by a FITC-conjugated 8-OxoG binding peptide in PERK knockdown cells infected with pBabe control vector (shPERK) or Nrf2-HA (shPERK Nr12-HA). Quantification of 8-OxoG positive cells is provided. Error bars represent S.D. from 3 experiments. (F) 8-OxoG was detected in PERK knockdown cells transfected with scramble siRNA (Serm), or keapl siRNA (sikeapi). Error bars in graphs represent S.D. from 3 experiments. Western blot panels demonstrate levels of Nrf2-HA and Keap1 in PERK knockdown cells.

FIG. 73, comprising FIGS. 73A-731, depicts the results of example experiments demonstrating that PERK loss attenuates MMTV-Neu-driven mammary tumorigenesis in mice, but promotes spontaneous mammary tumor formation in aged mammary gland-specific PERK knockout mice. (A) Kaplan-Meier analysis of tumor-free survival for cohorts of MMTV-Neu/PERKloxP/loxP (n-21) and MMTV-Neu/PERK/(n=27) mice. (B) Hematoxylin and eosin staining demonstrating histology of control (Neu/PERKloxP/loxP) and PERK knockout (Neu)PERK/° mammary gland tumors. (C) Western analysis for PERK, ErbB2, and eIF4E levels on whole protein extracts from control (Neu/PERKloxP/loxP) and PERK knockout (Neu/PERK/) mammary gland tumors or mammary gland from lactating PERKloxP/loxP darn (L10). (D) Nrf2 was precipitated from tumor lysates prepared from MMTV-Neu/PERK/° or control mice and blotted for phospho-Thr and Nrf2. PERK expression was determined by immunoblot. (E) PERK excision delays development of Neu-driven hyperplastic lesions. Representative mammary glands from 9- to 14-months old control (Neu/PERKloxP/loxP) and PERK knockout (Neu/PERK/) mice revealing pre-malignant lesions are shown. (F) Hematoxylin and eosin staining on lungs from control (Neu/PERKloxP/loxP, n=24) and PERK knockout (Neu/PERK/, n=27) mice revealing metastatic lesions. (G) Troma-1 (cytokeratin-8) staining on lung specimens containing metastatic foci. LT=lung tissue; Met=metastasis; OL=overlay. (H) Hematoxylin and eosin staining for tumor histology and whole mount of hyperplastic lesions in mammary glands of PERK/° aged females. (I) qRT-PCR for ErbB2 on genomic DNA from PERK/° tumors and FISH analysis on paraffin sections from the same animals. Levels of ErbB2 in tumors were compared to matched spleen tissues.

FIG. 74 depicts the results of example experiments demonstrating that PERK deficiency inhibits expansion of MDA-MB468-derived orthotopic tumors. Tumor volume 36 days post-orthotopic injection of MDA-MB468 parental cell line, control shRNA-(shControl) or shPERK-transduced cells (shPERK) (4 mice were used per cell line), Representative images of tumors are provided.

FIG. 75, comprising FIGS. 75A-75C, depicts the results of example experiments demonstrating that PERK knockdown triggers a G2/M delay and attenuates growth of cancer cells in vitro. (A) T47D cells were infected with control shRNA or anti-PERK shRNA and then pulsed with BrdU 45 min prior to being collected for FACS analysis 48 h after infection. (B) Kinetics of growth of T47D parental cell line, control shRNA-(shControl) or shPERK-transduced cells (shPERK), (C) Kinetics of growth of TE3 parental cell line, control shRNA-(shControl) or shPERK-transduced cells (shPERK).

FIG. 76, comprising FIGS. 76A-76C, depicts the results of example experiments demonstrating that PERK knockdown triggers DNA damage response signaling pathway. (A) Immunofluorescence staining for DNA damage-induced foci containing phospho-ATM and phospho-Chk2 72 h post-PERK knockdown in T47D cells. (B) Quantification of phospho-ATM positive cells (>3 foci) is shown; error bars indicate S.D. from 3 slides, 5 fields were counted per slide. p-value was determined by Student t-test. (C) Down-regulation of CDK2-dependent kinase activity in PERK null tumors. CDK2 complexes were immuno-purified from MMTV-NeuJPERKloxP/loxP control and MMTV-Neu/PERK/PERK-null tumors and its activity was monitored using recombinant histone H1 as a substrate. The efficiency of PERK excision was evaluated by RT-PCR and IP/Western blot analysis.

FIG. 77, comprising FIGS. 77A-77C, depicts the results of example experiments demonstrating that PERK deficiency results in accumulation of DNA DSBs in orthotopic tumors in vivo. (A) Immunohistochemistry for -H2AX in orthotopic tumors formed by uninfected MDA-MB468 cells, MDA-MB468 cells transduced with control shRNA, or with anti-PERK shRNA. Quantification of -H2AX-positive cells is provided. Error bars represent S.D. among 4 animals. (B) Immunohistochemistry for -H2AX in orthotopic tumors formed by mouse mammary tumor-derived cells transduced with empty vector pBabe retrovirus or retrovirus expressing Cre recombinase. Quantification of -H2AX-positive cells is provided. Error bars represent S.D. among 4 animals. (C) Immunofluorescence staining for markers of senescence, p19/Arf and try-methylated lysine 9 in histone H3 (H3K9) on orthotopic tumors formed by mouse mammary tumor-derived cells transduced with empty vector pBabe retrovirus or retrovirus expressing Cre recombinase.

FIG. 78 depicts the results of example experiments assessing the efficiency pf PERK excision in mouse mammary gland tumors. Efficiency of PERK allele excision was determined by semiquantitative RT-PCR on genomic DNA extracted from tumors of Neu PERKloxP/loxP (control) and Neu PERK/mice. Ratio of the intensity of recombinant allele band to wild type (wt) allele band was determined.

FIG. 79 depicts the results of example experiments demonstrating that DNA damage-induced checkpoint activation can be averted in PERK-deficient mouse mammary gland tumors. Western blot analysis of PERK, ErbB2, and DNA damage signaling-related markers in tumors derived from control (Neu/PERKlo7P/lo7P) or mammary gland-specific PERK-null (Neu/PERK/) tumor-prone mice. eIF4E levels were used as a loading control.

### DETAILED DESCRIPTION OF THE INVENTION

The invention provides compositions and methods for regulating the IFNAR1 chain of Type I interferon (IFN) receptor. In one embodiment, the invention relates to regulating phosphorylation-dependent ubiquitination and degradation of IFNAR1. In another embodiment, the invention relates to stabilizing IFNAR1. The invention is based on the discovery that inhibiting regulators of IFNAR1 and thereby inhibiting degradation of IFNAR1 serves to relieve the suppression of Type 1 IFN signaling and, therefore, provide a therapeutic benefit. This is because increasing the stability of IFNAR1 and Type I IFN receptor leads to augmentation of response to IFN. Conversely, activating the degradation of IFNAR1 can provide relief in diseases or disorders having pathologically increased IFN signaling, such as, by way of non-limiting examples, systemic lupus erythematosus and psoriasis.

The present invention relates to modulating the stability of IFNAR1 and Type I IFN receptor by modulating a regulator of IFNAR1 in a cell. The invention provides compositions and methods for inhibiting degradation of IFNAR1 and Type I IFN in a cell by modulation of a regulator of IFNAR1 such as PKR-like ER-localized eIF2α kinase (PERK), PTP1B (a tyrosine phosphatase), protein kinase D2 (PKD2), or any combination thereof. Therefore, the present invention provides a therapeutic benefit of interfering with a negative regulator of IFNAR1 during treatment of diseases or disorders associated with dysfunctional IFN responses, such as cancer, autoimmune diseases, multiple sclerosis, and viral infections.

### DEFINITIONS

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

The term “about” will be understood by persons of ordinary skill in the art and will vary to some extent on the context in which it is used.

“Allogeneic” refers to a graft derived from a different animal of the same species.

“Alloantigen” is an antigen that differs from an antigen expressed by the recipient.

The term “antibody” as used herein, refers to an immunoglobulin molecule, which is able to specifically bind to a specific epitope on an antigen.

Antibodies can be intact immunoglobulins derived from natural sources or from recombinant sources and can be immunoactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies (Harlow et al., 1988; Houston et al., 1988; Bird et al., 1988).

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA, A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded soley by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

“Antisense” refers particularly to the nucleic acid sequence of the non-coding strand of a double stranded DNA molecule encoding a polypeptide, or to a sequence which is substantially homologous to the non-coding strand. As defined herein, an antisense sequence is complementary to the sequence of a double stranded DNA molecule encoding a polypeptide. It is not necessary that the antisense sequence be complementary solely to the coding portion of the coding strand of the DNA molecule. The antisense sequence may be complementary to regulatory sequences specified on the coding strand of a DNA molecule encoding a polypeptide, which regulatory sequences control expression of the coding sequences.

As used herein, the term “autologous” is meant to refer to any material derived from the same subject to which it is later to be re-introduced into the subject.

The term “autoimmune disease” as used herein is defined as a disorder that results from an autoimmune response. An autoimmune disease is the result of an inappropriate and excessive response to a self-antigen. Examples of autoimmune diseases include but are not limited to, Addision's disease, alopecia greata, ankylosing spondylitis, autoimmune hepatitis, autoimmune parotitis, Crohn's disease, diabetes (Type I), dystrophic epidermolysis bullosa, epididymitis, glomerulonephritis, Graves' disease, Guillain-Barr syndrome, Hashimoto's disease, hemolytic anemia, systemic lupus erythematosus, multiple sclerosis, myasthenia gravis, pemphigus vulgaris, psoriasis, rheumatic fever, rheumatoid arthritis, sarcoidosis, scleroderma, Sjogren's syndrome, spondyloarthropathies, thyroiditis, vasculitis, vitiligo, myxedema, pernicious anemia, ulcerative colitis, among others.

The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like,

The term “DNA” as used herein is defined as deoxyribonucleic acid.

As used herein, an “effector cell” refers to a cell which mediates an immune response against an antigen. An example of an effector cell includes, but is not limited to a T cell and a B cell.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system.

As used herein, the term “exogenous” refers to any material introduced from or produced outside an organism, cell, tissue or system.

The term “epitope” as used herein is defined as a small chemical molecule on an antigen that can elicit an immune response, inducing B and/or T cell responses. An antigen can have one or more epitopes. Most antigens have many epitopes; i.e., they are multivalent. In general, an epitope is roughly five amino acids and/or sugars in size. One skilled in the art understands that generally the overall three-dimensional structure, rather than the specific linear sequence of the molecule, is the main criterion of antigenic specificity and therefore distinguishes one epitope from another.

The term “expression” as used herein is defined as the transcription and/or translation of a particular nucleotide sequence driven by its promoter.

The term “expression vector” as used herein refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules, siRNA, ribozymes, and the like. Expression vectors can contain a variety of control sequences, which refer to nucleic acid sequences necessary for the transcription and possibly translation of an operatively linked coding sequence in a particular host organism. In addition to control sequences that govern transcription and translation, vectors and expression vectors may contain nucleic acid sequences that serve other functions as well.

The term “heterologous” as used herein is defined as DNA or RNA sequences or proteins that are derived from the different species.

“Homologous” as used herein, refers to the subunit sequence similarity between two polymeric molecules, e.g., between two nucleic acid molecules, e.g., two

DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two composition sequences are homologous then the two sequences are 50% homologous, if 90% of the positions, e.g., 9 of 10, are matched or homologous, the two sequences share 90% homology. By way of example, the DNA sequences 5′-ATTGCC-3′ and 5′-TATGGC-3′ share 50% homology.

As used herein, “homology” is used synonymously with “identity.”

As used herein, “immunogen” refers to a substance that is able to stimulate or induce a Immoral antibody and/or cell-mediated immune response in a mammal.

The term “immunoglobulin” or “Ig”, as used herein is defined as a class of proteins, which function as antibodies. The five members included in this class of proteins are IgA, IgG, IgM, IgD, and IgE, IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most mammals. It is the most efficient immunoglobulin in agglutination, complement fixation, and other antibody responses, and is important in defense against bacteria and viruses. IgD is the immunoglobulin that has no known antibody function, but may serve as an antigen receptor. IgE is the immunoglobulin that mediates immediate hypersensitivity by causing release of mediators from mast cells and basophils upon exposure to allergen.

An “isolated nucleic acid” refers to a nucleic acid segment or fragment which has been separated from sequences which flank it in a naturally occurring state, i.e., a DNA fragment which has been removed from the sequences which are normally adjacent to the fragment, i.e., the sequences adjacent to the fragment in a genome in which it naturally occurs. The term also applies to nucleic acids which have been substantially purified from other components which naturally accompany the nucleic acid, i.e., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes a recombinant DNA which is part of a hybrid gene encoding additional polypeptide sequence.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

As used herein, the term “modulate” is meant to refer to any change in biological state, i.e. increasing, decreasing, and the like. For example, the term “modulate” refers to the ability to regulate positively or negatively the expression, stability or activity of IFNAR1, including but not limited to transcription of IFNAR1 mRNA, stability of IFNAR1 mRNA, translation of IFNAR1 mRNA, stability of IFNAR1 polypeptide, IFNAR1 post-translational modifications, or any combination thereof. Further, the term modulate can be used to refer to an increase, decrease, masking, altering, overriding or restoring of activity, including but not limited to, IFNAR1 activity.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCRT™, and the like, and by synthetic means.

The term “polypeptide” as used herein is defined as a chain of amino acid residues, usually having a defined sequence. As used herein the term polypeptide is mutually inclusive of the terms “peptide” and “protein”.

“Proliferation” is used herein to refer to the reproduction or multiplication of similar forms of entities, for example proliferation of a cell. That is, proliferation encompasses production of a greater number of cells, and can be measured by, among other things, simply counting the numbers of cells, measuring incorporation of ³H-thymidine into the cell, and the like.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a cell substantially only if the cell is a cell of the tissue type corresponding to the promoter.

The term “RNA” as used herein is defined as ribonucleic acid.

The term “recombinant DNA” as used herein is defined as DNA produced by joining pieces of DNA from different sources.

The term “recombinant polypeptide” as used herein is defined as a polypeptide produced by using recombinant DNA methods.

The term “self-antigen” as used herein is defined as an antigen that is expressed by a host cell or tissue. Self-antigens may be tumor antigens, but in certain embodiments, are expressed in both normal and tumor cells. A skilled artisan would readily understand that a self-antigen may be overexpressed in a cell.

As used herein, a “substantially purified” cell is a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some embodiments, the cells are culture in vitro. In other embodiments, the cells are not cultured in vitro.

The term “T-cell” as used herein is defined as a thymus-derived cell that participates in a variety of cell-mediated immune reactions.

The term “B-cell” as used herein is defined as a cell derived from the bone marrow and/or spleen. B cells can develop into plasma cells which produce antibodies.

“Therapeutically effective amount” is an amount of a composition of the invention, that when administered to a patient, ameliorates a symptom of the disease. The amount of a composition of the invention which constitutes a “therapeutically effective amount” will vary depending on the composition, the disease state and its severity, the age of the patient to be treated, and the like. The therapeutically effective amount can be determined routinely by one of ordinary skill in the art having regard to his own knowledge and to this disclosure.

“Patient” for the purposes of the present invention includes humans and other animals, particularly mammals, and other organisms. Thus the methods are applicable to both human therapy and veterinary applications. In a preferred embodiment the patient is a mammal, and in a most preferred embodiment the patient is human.

The terms “treat,” “treating,” and “treatment,” refer to therapeutic or preventative measures described herein. The methods of “treatment” employ administration to a subject, in need of such treatment, a composition of the present invention, for example, a subject having a disorder mediated by IFNAR1 or a subject who ultimately may acquire such a disorder, in order to prevent, cure, delay, reduce the severity of, or ameliorate one or more symptoms of the disorder or recurring disorder, or in order to prolong the survival of a subject beyond that expected in the absence of such treatment.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The phrase “under transcriptional control” or “operatively linked” as used herein means that the promoter is in the correct location and orientation in relation to a polynucleotide to control the initiation of transcription by RNA polymerase and expression of the polynucleotide.

The term “vaccine” as used herein is defined as a material used to provoke an immune response after administration of the material to a mammal.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compositions, plasmids, and viruses. Thus, the term “vector” includes an autonomously replicating plasmid or a virus. The term should also be construed to include non-plasmid and non-viral compositions which facilitate transfer of nucleic acid into cells, such as, for example, polylysine compositions, liposomes, and the like. Examples of viral vectors include, but are not limited to, adenoviral vectors, adeno-associated virus vectors, retroviral vectors, and the like.

“Xenogeneic” refers to a graft derived from an animal of a different species.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual and partial numbers within that range, for example, 1, 2, 3, 4, 5, 5.5 and 6. This applies regardless of the breadth of the range.

### DESCRIPTION

The present invention provides compositions and methods for modulating

IFNAR1 and methods of treating diseases that are amenable to therapeutic effects of endogenous IFN or pharmaceutical IFN-based drugs whose effects are mediated by IFNAR1 using the compositions of the invention. Diseases that are treated by IFN (whose actions are mediated by IFNAR1) include, but are not limited to, cancer, multiple sclerosis and other autoimmune diseases, and viral infections.

The present invention relates to the discovery that UPR triggers activation of PERK to promote ligand- and Jak-independent phosphorylation of IFNAR1 within its phospho-degron, leading to IFNAR1 ubiquitination and degradation as well as to suppress Type I IFN signaling. In some instances, UPR triggers activation of PERK in the context of a viral infection. Accordingly, the invention includes compositions and methods of targeting PERK in treatment of viral infections or other diseases that benefit from IFN.

The present invention also relates to the discovery that activity of PKD2 is required for ligand- and Jak-dependent phosphorylation of IFNAR1 within its phospho-degron, leading to IFNAR1 ubiquitination and degradation as well as to suppress Type I IFN signaling. Accordingly, the invention includes compositions and methods of targeting PKD in treatment of viral infections or other diseases that benefit from IFN.

The present invention also relates to the discovery that regardless of how phosphorylation-dependent ubiquitination of IFNAR1 proceeds, the endocytosis and degradation of IFNAR1 (as well as the extent of Type I IFN signaling) requires activity of PTP 1B. Accordingly, the invention includes compositions and methods of targeting PTP1B in treatment of viral infections or other diseases that benefit from IFN.

Also included in the invention are compositions and methods for increasing the efficacy of endogenous IFN and/or enhancement of efficacy of an IFN-based treatment.

### Compositions

As described elsewhere herein, the invention is based on the discovery that inhibition of a regulator of IFNAR1, such as, for example, PERK, PTP1B, or PKD2, can modulate the stability of IFNAR1 and provide a therapeutic benefit by increasing the efficacy of endogenous IFN and/or enhancement of efficacy of an IFN-based treatment.

The present invention relates to the discovery that inhibition of regulator of IFNAR1, such as, for example, PERK, PTP1B, or PKD2, provides a therapeutic benefit. Thus, the invention comprises compositions and methods for modulating any of these proteins in a cell thereby enhancing IFN response in the cell.

Based on the disclosure herein, the present invention includes a generic concept for inhibiting a negative regulator of stability and signaling of IFNAR1 or a functional equivalent thereof. Preferably, the negative regulator is PERK, PTP 1 B, and/or PKD2. Inhibiting any one or more of these proteins is associated with increasing the stability of IFNAR1. Accordingly, the invention includes inhibiting at least one of the aforementioned targets to increase the efficacy of endogenous IFN and/or enhancement of efficacy of an IFN-based treatment.

In one embodiment, the invention comprises a composition comprising an inhibitor of any one or more of the following regulators: PERK, PTP 1 B, or PKD2. The composition comprising the inhibitor is selected from the group consisting of a small interfering RNA (siRNA), a microRNA, an antisense nucleic acid, a ribozyme, an expression vector encoding a transdominant negative mutant, an intracellular antibody, a peptide and a small molecule.

One non-limiting example of an inhibitor useful in the methods of the invention is sangivamyein. Other non-limiting example of inhibitors useful in the methods of the invention are quinoline-difluoromethylphosphonate and naphthalene-difluoromethylphosphonate, and derivatives thereof, such as those described in Han et al. (2008, Bioorganic & Medicinal Chemistry Letters 18:3200-3205). Still other non-limiting examples of inhibitors useful in the methods of the invention are trifluoromethyl sulfonyl and derivatives thereof, benzooxathiazonle and derivatives thereof, cinnamic acid and derivatives thereof, hydroxyphenyl azole and derivatives thereof, pyrrol phenoxy propionic acid and derivatives thereof; phenylalanine and derivatives thereof, 3′-carboxy-4′-(β-carboxymethyl)-tyrosine and derivatives thereof, ertiprotfib and derivatives thereof NNC-52-1236 and derivatives thereof, A-366901 and derivatives thereof, A-321842 and derivatives thereof, 1,2-naphtoquinone and derivatives thereof, 4′-phosphenyldifluoromethyl-phenylanaline and derivatives thereof, aryldifluoromethylphosphonic acid and derivatives thereof.

One skilled in the art will readily appreciate that as a result of the degeneracy of the genetic code, many different nucleotide sequences may encode the same polypeptide. That is, an amino acid may be encoded by one of several different codons, and a person skilled in the art can readily determine that while one particular nucleotide sequence may differ from another, the polynucleotides may in fact encode polypeptides with identical amino acid sequences. As such, polynucleotides that vary due to differences in codon usage are specifically contemplated by the present invention.

One skilled in the art will appreciate, based on the disclosure provided herein, that one way to decrease the mRNA and/or protein levels of PERK, PTPIB, and/or PKD2 in a cell is by reducing or inhibiting expression of the nucleic acid encoding the regulator. Thus, the protein level of the regulator in a cell can also be decreased using a molecule or composition that inhibits or reduces gene expression such as, for example, an antisense molecule or a ribozyme.

In a preferred embodiment, the modulating sequence is an antisense nucleic acid sequence which is expressed by a plasmid vector. The antisense expressing vector is used to transfect a mammalian cell or the mammal itself, thereby causing reduced endogenous expression of a desired regulator in the cell. However, the invention should not be construed to be limited to inhibiting expression of a regulator by transfection of cells with antisense molecules. Rather, the invention encompasses other methods known in the art for inhibiting expression or activity of a protein in the cell including, but not limited to, the use of a ribozyme, the expression of a non-functional regulator (i.e. transdominant negative mutant) and use of an intracellular antibody.

Antisense molecules and their use for inhibiting gene expression are well known in the art (see, e.g., Cohen, 1989, In: Oligodeoxyribonucleotides, Antisense Inhibitors of Gene Expression, CRC Press). Antisense nucleic acids are DNA or RNA molecules that are complementary, as that term is defined elsewhere herein, to at least a portion of a specific mRNA molecule (Weintraub, 1990, Scientific American 262:40). In the cell, antisense nucleic acids hybridize to the corresponding mRNA, forming a double-stranded molecule thereby inhibiting the translation of genes.

The use of antisense methods to inhibit the translation of genes is known in the art, and is described, for example, in Marcus-Sakura (1988, Anal. Biochem. 172:289). Such antisense molecules may be provided to the cell via genetic expression using DNA encoding the antisense molecule as taught by Inoue, 1993, U.S. Pat. No. 5,190,931.

Alternatively, antisense molecules of the invention may be made synthetically and then provided to the cell. Antisense oligomers of between about 10 to about 30, and more preferably about 15 nucleotides, are preferred, since they are easily synthesized and introduced into a target cell. Synthetic antisense molecules contemplated by the invention include oligonucleotide derivatives known in the art which have improved biological activity compared to unmodified oligonucleotides (see U.S. Pat. No. 5,023,243).

Ribozymes and their use for inhibiting gene expression are also well known in the art (see, e.g., Cech et al., 1992, J. Biol. Chem. 267:17479-17482; Hampel et al., 1989, Biochemistry 28:4929-4933; Eckstein et al., International Publication No. WO 92/07065; Altman et al., U.S. Pat. No. 5,168,053). Ribozymes are RNA molecules possessing the ability to specifically cleave other single-stranded RNA in a manner analogous to DNA restriction endonucleases. Through the modification of nucleotide sequences encoding these RNAs, molecules can be engineered to recognize specific nucleotide sequences in an RNA molecule and cleave it (Cech, 1988, J. Amer. Med. Assn. 260:3030). A major advantage of this approach is the fact that ribozymes are sequence-specific.

There are two basic types of ribozymes, namely, tetrahymena-type (Hasselhoff, 1988, Nature 334:585) and hammerhead-type. Tetrahymena-type ribozymes recognize sequences which are four bases in length, while hammerhead-type ribozymes recognize base sequences 11-18 bases in length. The longer the sequence, the greater the likelihood that the sequence will occur exclusively in the target mRNA species. Consequently, hammerhead-type ribozymes are preferable to tetrahymena-type ribozymes for inactivating specific mRNA species, and 18-base recognition sequences are preferable to shorter recognition sequences which may occur randomly within various unrelated mRNA molecules.

Ribozymes useful for inhibiting the expression of a regulator may be designed by incorporating target sequences into the basic ribozyme structure which are complementary to the mRNA sequence of the desired regulator of the present invention, including but are not limited to, PERK, PTP1B, PKD2 and equivalents thereof. Ribozymes targeting the desired regulator may be synthesized using commercially available reagents (Applied Biosystems, Inc., Foster City, Calif.) or they may be genetically expressed from DNA encoding them.

In another aspect of the invention, the regulator can be inhibited by way of inactivating and/or sequestering the regulator, As such, inhibiting the effects of a regulator can be accomplished by using a transdominant negative mutant. Alternatively an antibody specific for the desired regulator, otherwise known as an antagonist to the regulator may be used. In one embodiment, the antagonist is a protein and/or composition having the desirable property of interacting with a binding partner of the regulator and thereby competing with the corresponding wild-type regulator, In another embodiment, the antagonist is a protein and/or composition having the desirable property of interacting with the regulator and thereby sequestering the regulator.

### Antibodies

As will be understood by one skilled in the art, any antibody that can recognize and bind to an antigen of interest is useful in the present invention. That is, the antibody can inhibit a regulator of IFNAR1 such as PERK, PTP1B, and/or PKD2 provides a beneficial effect.

Methods of making and using antibodies are well known in the art. For example, polyclonal antibodies useful in the present invention are generated by immunizing rabbits according to standard immunological techniques well-known in the art (see, e.g., Harlow et al., 1988, In: Antibodies, A Laboratory Manual, Cold Spring Harbor, N.Y.). Such techniques include immunizing an animal with a chimeric protein comprising a portion of another protein such as a maltose binding protein or glutathione (GSH) tag polypeptide portion, and/or a moiety such that the antigenic protein of interest is rendered immunogenic (e.g., an antigen of interest conjugated with keyhole limpet hemocyanin, KLH) and a portion comprising the respective antigenic protein amino acid residues. The chimeric proteins are produced by cloning the appropriate nucleic acids encoding the marker protein into a plasmid vector suitable for this purpose, such as but not limited to, pMAL-2 or pCMX.

However, the invention should not be construed as being limited solely to methods and compositions including these antibodies or to these portions of the antigens. Rather, the invention should be construed to include other antibodies, as that term is defined elsewhere herein, to antigens, or portions thereof. Further, the present invention should be construed to encompass antibodies, inter alia, bind to the specific antigens of interest, and they are able to bind the antigen present on Western blots, in solution in enzyme linked immunoassays, in fluorescence activated cells sorting (FACS) assays, in magnetic-activated cell sorting (MACS) assays, and in immunofluorescence microscopy of a cell transiently transfected with a nucleic acid encoding at least a portion of the antigenic protein, for example.

One skilled in the art would appreciate, based upon the disclosure provided herein, that the antibody can specifically bind with any portion of the antigen and the full-length protein can be used to generate antibodies specific therefor. However, the present invention is not limited to using the full-length protein as an immunogen. Rather, the present invention includes using an immunogenic portion of the protein to produce an antibody that specifically binds with a specific antigen. That is, the invention includes immunizing an animal using an immunogenic portion, or antigenic determinant, of the antigen.

Once armed with the sequence of a specific antigen of interest and the detailed analysis localizing the various conserved and non-conserved domains of the protein, the skilled artisan would understand, based upon the disclosure provided herein, how to obtain antibodies specific for the various portions of the antigen using methods well-known in the art or to be developed.

The skilled artisan would appreciate, based upon the disclosure provided herein, that that present invention includes use of a single antibody recognizing a single antigenic epitope but that the invention is not limited to use of a single antibody. Instead, the invention encompasses use of at least one antibody where the antibodies can be directed to the same or different antigenic protein epitopes.

The generation of polyclonal antibodies is accomplished by inoculating the desired animal with the antigen and isolating antibodies which specifically bind the antigen therefrom using standard antibody production methods such as those described in, for example, Harlow et al. (1988, In: Antibodies, A Laboratory Manual, Cold Spring Harbor, N.Y.).

Monoclonal antibodies directed against full length or peptide fragments of a protein or peptide may be prepared using any well known monoclonal antibody preparation procedures, such as those described, for example, in Harlow et al. (1988, In: Antibodies, A Laboratory Manual, Cold Spring Harbor, N.Y.) and in Tuszynski et al. (1988, Blood, 72:109-115). Quantities of the desired peptide may also be synthesized using chemical synthesis technology. Alternatively, DNA encoding the desired peptide may be cloned and expressed from an appropriate promoter sequence in cells suitable for the generation of large quantities of peptide. Monoclonal antibodies directed against the peptide are generated from mice immunized with the peptide using standard procedures as referenced herein.

Nucleic acid encoding the monoclonal antibody obtained using the procedures described herein may be cloned and sequenced using technology which is available in the art, and is described, for example, in Wright et al. (1992, Critical Rev. Immunol. 12:125-168), and the references cited therein. Further, the antibody of the invention may be “humanized” using the technology described in, for example, Wright et al., and in the references cited therein, and in Gu et al. (1997, Thrombosis and Hematocyst 77:755-759), and other methods of humanizing antibodies well-known in the art or to be developed.

The present invention also includes the use of humanized antibodies specifically reactive with epitopes of an antigen of interest. The humanized antibodies of the invention have a human framework and have one or more complementarity determining regions (CDRs) from an antibody, typically a mouse antibody, specifically reactive with an antigen of interest. When the antibody used in the invention is humanized, the antibody may be generated as described in Queen, et al. (U.S. Pat. No. 6,180,370), Wright et al., (supra) and in the references cited therein, or in On et (1997, Thrombosis and Hematocyst 77(4):755-759). The method disclosed in Queen et al. is directed in part toward designing humanized immunoglobulins that are produced by expressing recombinant DNA segments encoding the heavy and light chain complementarity determining regions (CDRs) from a donor immunoglobulin capable of binding to a desired antigen, such as an epitope on an antigen of interest, attached to DNA segments encoding acceptor human framework regions. Generally speaking, the invention in the Queen patent has applicability toward the design of substantially any humanized immunoglobulin. Queen explains that the DNA segments will typically include an expression control DNA sequence operably linked to the humanized immunoglobulin coding sequences, including naturally-associated or heterologous promoter regions. The expression control sequences can be eukaryotic promoter systems in vectors capable of transforming or transfecting eukaryotic host cells or the expression control sequences can be prokaryotic promoter systems in vectors capable of transforming or transfecting prokaryotic host cells. Once the vector has been incorporated into the appropriate host, the host is maintained under conditions suitable for high level expression of the introduced nucleotide sequences and as desired the collection and purification of the humanized light chains, heavy chains, light/heavy chain dimers or intact antibodies, binding fragments or other immunoglobulin forms may follow (Beychok, Cells of Immunoglobulin Synthesis, Academic Press, New York, (1979), which is incorporated herein by reference).

The invention also includes functional equivalents of the antibodies described herein. Functional equivalents have binding characteristics comparable to those of the antibodies, and include, for example, hybridized and single chain antibodies, as well as fragments thereof. Methods of producing such functional equivalents are disclosed in PCT Application WO 93/21319 and PCT Application WO 89/09622.

Functional equivalents include polypeptides with amino acid sequences substantially the same as the amino acid sequence of the variable or hypervariable regions of the antibodies. “Substantially the same” amino acid sequence is defined herein as a sequence with at least 70%, preferably at least about 80%, more preferably at least about 90%, even more preferably at least about 95%, and most preferably at least 99% homology to another amino acid sequence (or any integer in between 70 and 99), as determined by the PASTA search method in accordance with Pearson and Lipman, 1988 Proc. Nat'l. Acad. Sci. USA 85: 2444-2448. Chimeric or other hybrid antibodies have constant regions derived substantially or exclusively from human antibody constant regions and variable regions derived substantially or exclusively from the sequence of the variable region of a monoclonal antibody from each stable hybridoma.

Single chain antibodies (scFv) or Fv fragments are polypeptides that consist of the variable region of the heavy chain of the antibody linked to the variable region of the light chain, with or without an interconnecting linker. Thus, the Fv comprises an antibody combining site.

Functional equivalents of the antibodies of the invention further include fragments of antibodies that have the same, or substantially the same, binding characteristics to those of the whole antibody. Such fragments may contain one or both Fab fragments or the F(ab′)₂ fragment. The antibody fragments contain all six complement determining regions of the whole antibody, although fragments containing fewer than all of such regions, such as three, four or five complement determining regions, are also functional. The functional equivalents are members of the IgG immunoglobulin class and subclasses thereof, but may be or may combine with any one of the following immunoglobulin classes: IgM, IgA, IgD, or IgE, and subclasses thereof. Heavy chains of various subclasses, such as the IgG subclasses, are responsible for different effector functions and thus, by choosing the desired heavy chain constant region, hybrid antibodies with desired effector function are produced. Exemplary constant regions are gamma 1 (IgG 1), gamma 2 (IgG2), gamma 3 (IgG3), and gamma 4 (IgG4). The light chain constant region can be of the kappa or lambda type.

The immunoglobulins of the present invention can be monovalent, divalent or polyvalent. Monovalent immunoglobulins are dimers (HL) formed of a hybrid heavy chain associated through disulfide bridges with a hybrid light chain. Divalent immunoglobulins are tetramers (H₂L₂) formed of two dimers associated through at least one disulfide bridge.

### Modification of Nucleic Acid Molecules

Inhibition of PERK, PTP1B, and/or PKD2 or their functional equivalents, resulting in modulation of IFNAR1 stability can be accomplished using a nucleic acid molecule. For example, the inhibitor is selected from the group consisting of a small interfering RNA (siRNA), a microRNA, an antisense nucleic acid, a ribozyme, an expression vector encoding a transdominant negative mutant, and the likes.

By way of example, modification of nucleic acid molecules is described in the context of an siRNA molecule. However, the methods of modifying nucleic acid molecules can be applied to other types of nucleic acid based inhibitors of the invention.

As a non-limiting example, an siRNA polynucleotide is an RNA nucleic acid molecule that interferes with RNA activity that is generally considered to occur via a post-transcriptional gene silencing mechanism. An siRNA polynucleotide preferably comprises a double-stranded RNA (dsRNA) but is not intended to be so limited and may comprise a single-stranded RNA (see, e.g., Martinez et al., 2002 Cell 110:563-74). The siRNA polynucleotide included in the invention may comprise other naturally occurring, recombinant, or synthetic single-stranded or double-stranded polymers of nucleotides (ribonucleotides or deoxyribonucleotides or a combination of both) and/or nucleotide analogues as provided herein (e.g., an oligonucleotide or polynucleotide or the like, typically in 5′ to 3′ phosphodiester linkage). Accordingly it will be appreciated that certain exemplary sequences disclosed herein as DNA sequences capable of directing the transcription of the siRNA polynucleotides are also intended to describe the corresponding RNA sequences and their complements, given the well established principles of complementary nucleotide base-pairing.

Preferred siRNA polynucleotides comprise double-stranded polynucleotides of about 18-30 nucleotide base pairs, preferably about 18, about 19, about 20, about 21, about 22, about 23, about 24, about 25, about 26, or about 27 base pairs, and in other preferred embodiments about 19, about 20, about 21, about 22 or about 23 base pairs, or about 27 base pairs, whereby the use of “about” indicates that in certain embodiments and under certain conditions the processive cleavage steps that may give rise to functional siRNA polynucleotides that are capable of interfering with expression of a selected polypeptide may not be absolutely efficient. Hence, siRNA polynucleotides, may include one or more siRNA polynucleotide molecules that may differ (e.g., by nucleotide insertion or deletion) in length by one, two, three, four or more base pairs as a consequence of the variability in processing, in biosynthesis, or in artificial synthesis of the siRNA. The siRNA polynucleotide of the present invention may also comprise a polynucleotide sequence that exhibits variability by differing (e.g., by nucleotide substitution, including transition or transversion) at one, two, three or four nucleotides from a particular sequence. These differences can occur at any of the nucleotide positions of a particular siRNA polynucleotide sequence, depending on the length of the molecule, whether situated in a sense or in an antisense strand of the double-stranded polynucleotide. The nucleotide difference may be found on one strand of a double-stranded polynucleotide, where the complementary nucleotide with which the substitute nucleotide would typically form hydrogen bond base pairing, may not necessarily be correspondingly substituted. In preferred embodiments, the siRNA polynucleotides are homogeneous with respect to a specific nucleotide sequence.

Based on the present disclosure, it should be appreciated that the siRNAs of the present invention may effect silencing of the target polypeptide expression to different degrees. The siRNAs thus must first be tested for their effectiveness. Selection of siRNAs are made therefrom based on the ability of a given siRNA to interfere with or modulate the expression of the target polypeptide. Accordingly, identification of specific siRNA polynucleotide sequences that are capable of interfering with expression of a desired target polypeptide requires production and testing of each siRNA. The methods for testing each siRNA and selection of suitable siRNAs for use in the present invention are fully set forth herein the Examples. Since not all siRNAs that interfere with protein expression will have a physiologically important effect, the present disclosure also sets forth various physiologically relevant assays for determining whether the levels of interference with target protein expression using the siRNAs of the invention have clinically relevant significance.

Polynucleotides of the siRNA may be prepared using any of a variety of techniques, which are useful for the preparation of specifically desired siRNA polynucleotides. For example, a polynucleotide may be amplified from a cDNA prepared from a suitable cell or tissue type. Such a polynucleotide may be amplified via polymerase chain reaction (PCR). Using this approach, sequence-specific primers are designed based on the sequences provided herein, and may be purchased or synthesized directly. An amplified portion of the primer may be used to isolate a full-length gene, or a desired portion thereof; from a suitable DNA library using well known techniques. A library (cDNA or genomic) is screened using one or more polynucleotide probes or primers suitable for amplification, Preferably, the library is size-selected to include larger polynucleotide sequences. Random primed libraries may also be preferred in order to identify 5′ and other upstream regions of the genes. Genomic libraries are preferred for obtaining introns and extending 5′ sequences. The siRNA polynucleotide contemplated by the present invention may also be selected from a library of siRNA polynucleotide sequences.

For hybridization techniques, a partial polynucleotide sequence may be labeled (e.g., by nick-translation or end-labeling with ³²P) using well known techniques. A bacterial or bacteriophage library may then be screened by hybridization to filters containing denatured bacterial colonies (or lawns containing phage plaques) with the labeled probe (see, e.g., Sambrook et al., Molecular Cloning: A Laboratory Manual, Cold Spring Harbor Laboratories, Cold Spring Harbor, N.Y., 2001). Hybridizing colonies or plaques are selected and expanded, and the DNA is isolated for further analysis.

Alternatively, numerous amplification techniques are known in the art for obtaining a full-length coding sequence from a partial cDNA sequence. Within such techniques, amplification is generally performed via PCR. One such technique is known as “rapid amplification of cDNA ends” or RACE (see, e.g., Sambrook et al., Molecular Cloning: A Laboratory Manual, Cold Spring Harbor Laboratories, Cold Spring Harbor, N.Y., 2001).

A number of specific siRNA polynucleotide sequences useful for interfering with target polypeptide expression are presented in the Examples, the Drawings, and in the Sequence Listing included herein. siRNA polynucleotides may generally be prepared by any method known in the art, including, for example, solid phase chemical synthesis. Modifications in a polynucleotide sequence may also be introduced using standard mutagenesis techniques, such as oligonucleotide-directed site-specific mutagenesis. Further, siRNAs may be chemically modified or conjugated with other molecules to improve their stability and/or delivery properties. Included as one aspect of the invention are siRNAs as described herein, wherein one or more ribose sugars has been removed therefrom.

Alternatively, siRNA polynucleotide molecules may be generated by in vitro or in vivo transcription of suitable DNA sequences (e.g., polynucleotide sequences encoding a target polypeptide, or a desired portion thereof), provided that the DNA is incorporated into a vector with a suitable RNA polymerase promoter (such as for example, T7, U6, H1, or SP6 although other promoters may be equally useful). In addition, an siRNA polynucleotide may be administered to a mammal, as may be a DNA sequence (e.g., a recombinant nucleic acid construct as provided herein) that supports transcription (and optionally appropriate processing steps) such that a desired siRNA is generated in vivo.

In one embodiment, an siRNA polynucleotide, wherein the siRNA polynucleotide is capable of interfering with expression of a target polypeptide can be used to generate a silenced cell. Any siRNA polynucleotide that, when contacted with a biological source for a period of time, results in a significant decrease in the expression of the target polypeptide is included in the invention. Preferably the decrease is greater than about 10%, more preferably greater than about 20%, more preferably greater than about 30%, more preferably greater than about 40%, about 50%, about 60%, about 70%, about 75%, about 80%, about 85%, about 90%, about 95% or about 98% relative to the expression level of the target polypeptide detected in the absence of the siRNA. Preferably, the presence of the siRNA polynucleotide in a cell does not result in or cause any undesired toxic effects, for example, apoptosis or death of a cell in which apoptosis is not a desired effect of RNA interference.

In another embodiment, the siRNA polynucleotide that, when contacted with a biological source for a period of time, results in a significant decrease in the expression of the target polypeptide. Preferably the decrease is about 10%-20%, more preferably about 20%-30%, more preferably about 30%-40%, more preferably about 40%-50%, more preferably about 50%-60%, more preferably about 60%-70%, more preferably about 70%-80%, more preferably about 80%-90%, more preferably about 90%-95%, more preferably about 95%-98% relative to the expression level of the target polypeptide detected in the absence of the siRNA. Preferably, the presence of the siRNA polynucleotide in a cell does not result in or cause any undesired toxic effects.

In yet another embodiment, the siRNA polynucleotide that, when contacted with a biological source for a period of time, results in a significant decrease in the expression of the target polypeptide. Preferably the decrease is about 10% or more, more preferably about 20% or more, more preferably about 30% or more, more preferably about 40% or more, more preferably about 50% or more, more preferably about 60% or more, more preferably about 70% or more, more preferably about 80% or more, more preferably about 90% or more, more preferably about 95% or more, more preferably about 98% or more relative to the expression level of the target polypeptide detected in the absence of the siRNA. Preferably, the presence of the siRNA polynucleotide in a cell does not result in or cause any undesired toxic effects.

Any polynucleotide of the invention may be further modified to increase its stability in vivo. Possible modifications include, but are not limited to, the addition of flanking sequences at the 5′ and/or 3′ ends; the use of phosphorothioate or T O-methyl rather than phosphodiester linkages in the backbone; and/or the inclusion of nontraditional bases such as inosine, queosine, and wybutosine and the like, as well as acetyl-methyl-, thio- and other modified forms of adenine, cytidine, guanine, thymine, and uridine.

### Genetic Modification

In other related aspects, the invention includes an isolated nucleic acid encoding an inhibitor, preferably an siRNA, that inhibits a regulator of IFNAR1, operably linked to a nucleic acid comprising a promoter/regulatory sequence such that the nucleic acid is preferably capable of directing expression of the protein encoded by the nucleic acid. In some embodiments, the isolated nucleic acid is an antisense nucleic acid encoding an antisense inhibitor. Thus, the invention encompasses expression vectors and methods for the introduction of exogenous DNA into cells with concomitant expression of the exogenous DNA in the cells such as those described, for example, in Sambrook et al. (2001, Molecular Cloning: A Laboratory Manual, Cold Spring Harbor Laboratory, New York), and in Ausubel et al. (1997, Current Protocols in Molecular Biology, John Wiley & Sons, New York).

The desired polynucleotide can be cloned into a number of types of vectors. However, the present invention should not be construed to be limited to any particular vector. Instead, the present invention should be construed to encompass a wide plethora of vectors which are readily available and/or well-known in the art. For example, a desired polynucleotide of the invention can be cloned into a vector including, but not limited to a plasmid, a phagemid, a phage derivative, an animal viruse, and a cosmid. Vectors of particular interest include expression vectors, replication vectors, probe generation vectors, and sequencing vectors.

In specific embodiments, the expression vector is selected from the group consisting of a viral vector, a bacterial vector and a mammalian cell vector. Numerous expression vector systems exist that comprise at least a part or all of the compositions discussed above. Prokaryote- and/or eukaryote-vector based systems can be employed for use with the present invention to produce polynucleotides, or their cognate polypeptides. Many such systems are commercially and widely available.

Further, the expression vector may be provided to a cell in the form of a viral vector. Viral vector technology is well known in the art and is described, for example, in Sambrook et al. (2001), and in Ausubel et al. (1997), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. (See, e.g., WO 01/96584; WO 01/29058; and U.S. Pat. No. 6,326,193.

For expression of the desired polynucleotide, at least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 genes, a discrete element overlying the start site itself helps to fix the place of initiation.

Additional promoter elements, i.e., enhancers, regulate the frequency of transcriptional initiation. Typically, these are located in the region 30-110 by upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 by apart before activity begins to decline. Depending on the promoter, it appears that individual elements can function either co-operatively or independently to activate transcription.

A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCRThi, in connection with the compositions disclosed herein (U.S. Pat. No. 4,683,202, U.S. Pat. No. 5,928,906). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know how to use promoters, enhancers, and cell type combinations for protein expression, for example, see Sambrook et al. (2001). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

A promoter sequence exemplified in the experimental examples presented herein is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, Moloney virus promoter, the avian leukemia virus promoter, Epstein-Barr virus immediate early promoter, Rous sarcoma virus promoter, as well as human gene promoters such as, but not limited to, the actin promoter, the myosin promoter, the hemoglobin promoter, and the muscle creatine promoter. Further, the invention should not be limited to the use of constitutive promoters. Inducible promoters are also contemplated as part of the invention. The use of an inducible promoter in the invention provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter. Further, the invention includes the use of a tissue specific promoter, which promoter is active only in a desired tissue. Tissue specific promoters are well known in the art and include, but are not limited to, the HER-2 promoter and the PSA associated promoter sequences.

In order to assess the expression of the siRNA, the expression vector to be introduced into a cell can also contain either a selectable marker gene or a reporter gene or both to facilitate identification and selection of expressing cells from the population of cells sought to be transfected or infected through viral vectors. In other embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neo and the like.

Reporter genes are used for identifying potentially transfected cells and for evaluating the functionality of regulatory sequences. Reporter genes that encode for easily assayable proteins are well known in the art. In general, a reporter gene is a gene that is not present in or expressed by the recipient organism or tissue and that encodes a protein whose expression is manifested by some easily detectable property, e.g., enzymatic activity. Expression of the reporter gene is assayed at a suitable time after the DNA has been introduced into the recipient cells.

Suitable reporter genes may include genes encoding luciferase, beta-galactosidase, chloramphenicol acetyl transferase, secreted alkaline phosphatase, or the green fluorescent protein gene (see, e.g., Ui-Tei et al., 2000 FEBS Lett. 479:79-82). Suitable expression systems are well known and may be prepared using well known techniques or obtained commercially. Internal deletion constructs may be generated using unique internal restriction sites or by partial digestion of non-unique restriction sites. Constructs may then be transfected into cells that display high levels of siRNA polynucleotide and/or polypeptide expression. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

In the context of an expression vector, the vector can be readily introduced into a host cell, e.g., mammalian, bacterial, yeast or insect cell by any method in the art. For example, the expression vector can be transferred into a host cell by physical, chemical or biological means. It is readily understood that the introduction of the expression vector comprising the polynucleotide of the invention yields a silenced cell with respect to a regulator.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York), and in Ausubel et al. (1997, Current Protocols in Molecular Biology, John Wiley & Sons, New York).

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. A preferred colloidal system for use as a delivery vehicle in vitro and in vivo is a liposome (i.e., an artificial membrane vesicle). The preparation and use of such systems is well known in the art.

Regardless of the method used to introduce exogenous nucleic acids into a host cell or otherwise expose a cell to the inhibitor of the present invention, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

Any DNA vector or delivery vehicle can be utilized to transfer the desired polynucleotide to a cell in vitro or in vivo, In the case where a non-viral delivery system is utilized, a preferred delivery vehicle is a liposome. The above-mentioned delivery systems and protocols therefore can be found in Gene Targeting Protocols, 2ed., pp 1-35 (2002) and Gene Transfer and Expression Protocols, Vol. 7, Murray ed., pp 81-89 (1991).

“Liposome” is a generic term encompassing a variety of single and multilamellar lipid vehicles formed by the generation of enclosed lipid bilayers or aggregates. Liposomes may be characterized as having vesicular structures with a phospholipid bilayer membrane and an inner aqueous medium, Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991, Targeted Diagn Ther 4:87-103). However, the present invention also encompasses compositions that have different structures in solution than the normal vesicular structure. For example, the lipids may assume a micellar structure or merely exist as nonuniform aggregates of lipid molecules. Also contemplated are lipofectamine-nucleic acid complexes.

### Therapeutic Application

The present invention includes an inhibitor of a regulator of IFNAR1, including an inhibitor of any one or more of PERK, PTP1B, PKD2, or a functional equivalent of any of these proteins. The present invention also provides compositions and methods to augment the efficacy of Type I IFN. Thus, the invention provides compositions and methods of treating diseases or disorders associated with dysfunctional IFN responses, such as cancer, autoimmune diseases, multiple sclerosis, and viral infections,

The present invention provides a use of an agent that is capable of inhibiting a regulator of IFNAR1, including an inhibitor of any one or more of PERK, PTP1B, or PKD2, as a means to augment the efficacy of Type I IFN. As such, a vaccine useful for in vivo immunization comprises at least an inhibitor component, wherein the inhibitor component is able to inhibit degradation of IFNAR1. Based on the present disclosure, administration of an inhibitor of one or more of PERK, PTP1B, or PKD2 enhances the stability of IFNAR1.

In another embodiment, the compositions of the present invention may be used in combination with existing therapeutic agents used to treat diseases or disorders associated with dysfunctional IFN responses, such as cancer, autoimmune diseases, multiple sclerosis, and viral infections. In some instances, the compositions of the invention may be used in combination these therapeutic agents to enhance the efficacy of Tvpe I IFN.

In some embodiments, an effective amount of a composition of the invention and a therapeutic agent is a synergistic amount. As used herein, a “synergistic combination” or a “synergistic amount” of a composition of the invention and a therapeutic agent is a combination or amount that is more effective in the therapeutic or prophylactic treatment of a disease than the incremental improvement in treatment outcome that could be predicted or expected from a merely additive combination of (0 the therapeutic or prophylactic benefit of the composition of the invention when administered at that same dosage as a monotherapy and (ii) the therapeutic or prophylactic benefit of the therapeutic agent when administered at the same dosage as a monotherapy.

### Methods of Treatment

In various embodiments, the methods of the invention comprise administering a therapeutically effective amount of at least one composition that is an inhibitor of a regulator of IFNAR1, including an inhibitor of any one or more of PERK, PTP1B, PKD2, or a functional equivalent of any of these proteins, to a cell, or to a subject in need thereof. In other embodiments, the methods of the invention comprise administering a therapeutically effective amount of at least one composition that is an activator of a regulator of IFNAR1, including an activator of any one or more of PERK, PTP1B, PKD2, or a functional equivalent of any of these proteins, to a cell, or to a subject in need thereof. In preferred embodiments the subject is a mammal. In more preferred embodiments the subject is a human.

The composition comprising the inhibitor is selected from the group consisting of a small interfering RNA (siRNA), a microRNA, an antisense nucleic acid, a ribozyme, an expression vector encoding a transdominant negative mutant, an intracellular antibody, a peptide and a small molecule. The present invention should in no way be construed to be limited to the inhibitors described herein, but rather should be construed to encompass any inhibitor of any modulator of IFNAR1. The methods of the invention comprise administering a therapeutically effective amount of at least one inhibitor to a subject.

The composition comprising the activator is selected from the group consisting of a small interfering RNA (siRNA), a microRNA, an antisense nucleic acid, a ribozyme, an expression vector encoding a transdominant negative mutant, an intracellular antibody, a peptide and a small molecule. The present invention should in no way be construed to be limited to the activators described herein, but rather should be construed to encompass any activator of any modulator of IFNAR1. The methods of the invention comprise administering a therapeutically effective amount of at least one activator to a subject.

The methods of the invention comprise administering a therapeutically effective amount of at least one inhibitor either alone or in combination with other therapeutic agents.

In various embodiments, inhibitors of the invention can be delivered to a cell in vitro or in vivo using vectors comprising one or more isolated inhibitor nucleic acid sequences. In some embodiments, the nucleic acid sequence has been incorporated into the genome of the vector. The vector comprising a nucleic acid inhibitor described herein can be contacted with a cell in vitro or in vivo and infection or transfection can occur. The cell can then be used experimentally to study, for example, the effect of a nucleic acid inhibitor in vitro. The cell can be present in a biological sample obtained from a subject (e.g., blood, bone marrow, tissue, biological fluids, organs, etc.) and used in the treatment of disease, or can be obtained from cell culture.

Various vectors can be used to introduce an isolated nucleic acid inhibitor into animal cells. Examples of viral vectors have been discussed elsewhere herein and include retrovirus, adenovirus, parvovirus (e.g., adeno-associated viruses), coronavirus, negative-strand RNA viruses such as orthomyxovirus (e.g., influenza virus), rhabdovirus (e.g., rabies and vesicular stomatitis virus), paramyxovirus (e.g. measles and Sendai), positive-strand RNA viruses such as picornavirus and alphavirus, and double stranded DNA viruses including adenovirus, herpesvirus (e.g., herpes simplex virus types 1 and 2, Epstein-Barr virus, cytomegalovirus), and poxvirus (e.g. vaccinia, fowlpox and canarypox). Other viruses include Norwalk virus, togavirus, flavivirus, reoviruses, papovavirus, hepadnavirus and hepatitis virus, for example, Examples of retroviruses include: avian leukosis-sarcoma, mammalian C-type, B-type viruses, D-type viruses, HTLV-BLV group, lentivirus (e.g. human immunodeficiency virus), and spumavirus (Coffin, J. M., Retroviridae: The viruses and their replication, In Fundamental Virology, Third Edition, B. N. Fields et al., Eds., Lippincott-Raven Publishers, Philadelphia, 1996). Other examples include murine leukemia viruses, murine sarcoma viruses, mouse mammary tumor virus, bovine leukemia virus, feline leukemia virus, feline sarcoma virus, avian leukemia virus, human T-eell leukemia virus, baboon endogenous virus, Gibbon ape leukemia virus, Mason Pfizer monkey virus, simian immunodeficiency virus, simian sarcoma virus, Rous sarcoma virus, lentiviruses and baculoviruses.

In addition, an engineered viral vector can be used to deliver an isolated nucleic acid inhibitor of the present invention. These vectors provide a means to introduce nucleic acids into cycling and quiescent cells, and have been modified to reduce cytotoxicity and to improve genetic stability. The preparation and use of engineered Herpes simplex virus type 1 (Krisky et al., 1997, Gene Therapy 4:1120-1125), adenoviral (Amalfitanl et al., 1998, Journal of Virology 72:926-933) attenuated lentiviral (Zufferey et al., 1997, Nature Biotechnology 15:871-875) and adenoviral/retroviral chimeric (Feng et al., 1997, Nature Biotechnology 15:866-870) vectors are known to the skilled artisan.

In addition to delivery through the use of vectors, a nucleic acid inhibitor can be delivered to cells without vectors, e.g. as “naked” nucleic acid delivery using methods known to those of skill in the art. See, for example, U.S. Pat. Nos. 5,350,674 and 5,585,362. Physical methods for introducing a nucleic acid into a host cell include, by way of examples, transfection, calcium phosphate precipitation, lipofection, particle bombardment, microinjection, electroporation and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York), and in Ausubel et al. (2001, Current Protocols in Molecular Biology, John Wiley & Sons, New York).

Chemical means for introducing a nucleic acid inhibitor into a host cell include, by way of examples, colloidal dispersion systems, such as macromolecule complexes, nanocapsules, microspheres, beads, and lipid-based systems including oil-in-water emulsions, micelles, mixed micelles and liposomes. A preferred colloidal system for use as a delivery vehicle in vitro and in vivo is a liposome (i.e., an artificial membrane vesicle). The preparation and use of such systems is well known in the art.

Various forms of a nucleic acid inhibitor, as described herein, can be administered or delivered to an animal cell (e.g., by virus, direct injection, or liposomes, or by any other suitable methods known in the art or later developed). The methods of delivery can be modified to target certain cells, and in particular, cell surface receptor molecules. As an example, the use of cationic lipids as a carrier for nucleic acid constructs provides an efficient means of delivering the nucleic acid inhibitor of the present invention.

Various formulations of cationic lipids have been used to deliver nucleic acids to cells (WO 91/17424; WO 91/16024; U.S. Pat. Nos. 4,897,355; 4,946,787; 5,049,386; and 5,208,036). Cationic lipids have also been used to introduce foreign nucleic acids into frog and rat cells in vivo (Holt et al., 1990, Neuron 4:203-214; Hazinski et al., 1991, Am. J. Respr. Cell. Mol. Biol. 4:206-209). Therefore, cationic lipids may be used, generally, as pharmaceutical carriers to provide biologically active substances (for example, see WO 91/17424; WO 91/16024; and WO 93/03709). Thus, cationic liposomes can provide an efficient carrier for the introduction of nucleic acids into a cell.

Further, liposomes can be used as carriers to deliver a nucleic acid inhibitor to a cell, tissue or organ. Liposomes comprising neutral or anionic lipids do not generally fuse with the target cell surface, but are taken up phagocytically, and the nucleic acids are subsequently subjected to the degradative enzymes of the lysosomal compartment (Straubinger et al., 1983, Methods Enzymol. 101:512-527; Mannino et al., 1988, Biotechniques 6:682-690). However, an isolated nucleic acid of the present invention can be a stable nucleic acid, and thus, may not be susceptible to degradative enzymes. Further, despite the fact that the aqueous space of typical liposomes may be too small to accommodate large macromolecules, the isolated nucleic acid inhibitor of the present invention is relatively small, and therefore, liposomes are a suitable delivery vehicle for some embodiments of the present invention. Methods of delivering a nucleic acid to a cell, tissue or organism, including liposome-mediated delivery, are known in the art and are described in, for example, Feigner (Gene Transfer and Expression Protocols Vol. 7, Murray, E. J. Ed., Humana Press, New Jersey, (1991)).

In other related aspects, the invention includes an isolated nucleic acid inhibitor operably linked to a nucleic acid comprising a promoter/regulatory sequence such that the nucleic acid is preferably capable of delivering a nucleic acid inhibitor. Thus, the invention encompasses expression vectors and methods for the introduction of an isolated nucleic acid inhibitor into or to cells.

Such delivery can be accomplished by generating a plasmid, viral, or other type of vector comprising an isolated nucleic acid inhibitor operably linked to a promoter/regulatory sequence which serves to introduce the nucleic acid inhibitor into cells in which the vector is introduced. Many promoter/regulatory sequences useful for the methods of the present invention are available in the art and include, but are not limited to, for example, the cytomegalovirus immediate early promoter enhancer sequence, the SV40 early promoter, as well as the Rous sarcoma virus promoter, and the like. Moreover, inducible and tissue specific expression of an isolated nucleic acid inhibitor may be accomplished by placing an isolated nucleic acid inhibitor, with or without a tag, under the control of an inducible or tissue specific promoter/regulatory sequence. Examples of tissue specific or inducible promoter/regulatory sequences which are useful for his purpose include, but are not limited to the MMTV LTR inducible promoter, and the SV40 late enhancer/promoter. In addition, promoters which are well known in the art which are induced in response to inducing agents such as metals, glucocorticoids, and the like, are also contemplated in the invention. Thus, it will be appreciated that the invention includes the use of any promoter/regulatory sequence, which is either known or unknown, and which is capable of driving expression of the desired protein operably linked thereto.

Selection of any particular plasmid vector or other vector is not a limiting factor in the invention and a wide plethora of vectors are well-known in the art. Further, it is well within the skill of the artisan to choose particular promoter/regulatory sequences and operably link those promoter/regulatory sequences to a DNA sequence encoding a desired polypeptide. Such technology is well known in the art and is described, for example, in Sambrook et al. (2001, Molecular Cloning: A Laboratory Manual, Cold Spring Harbor Laboratory, New York), and in Ausubel et al. (2001, Current Protocols in Molecular Biology, John Wiley & Sons, New York) and elsewhere herein.

### Pharmaceutical Compositions and Therapies

Administration of an inhibitor composition of the invention comprising one or more of a small interfering RNA (siRNA), a microRNA, an antisense nucleic acid, a ribozyme, an expression vector encoding a transdominant negative mutant, an intracellular antibody, a peptide and a small molecule of the invention in a method of treatment can be achieved in a number of different ways, using methods known in the art. Such methods include, but are not limited to, providing exogenous nucleic acids, antisense nucleic acids, polynucleotides, or oligonucleotides to a subject or expressing a recombinant nucleic acid, antisense nucleic acid, polynucleotide, or oligonucleotide expression cassette.

The pharmaceutical compositions useful for practicing the invention may be administered to deliver a dose of between 1 ng/kg/day and 100 mg/kg/day. In one embodiment, the invention envisions administration of a dose which results in a concentration of the compound of the present invention between 1 μM and 10 μM in a mammal. In another embodiment, the invention envisions administration of a dose which results in a concentration of the compound of the present invention between 1 μM and 10 μM in a cell of a mammal.

Typically, dosages which may be administered in a method of the invention to a subject, preferably a human, range in amount from 0.5 μg to about 50 mg per kilogram of body weight of the animal. While the precise dosage administered will vary depending upon any number of factors, including but not limited to, the type of subject and type of disease state being treated, the age of the subject and the route of administration. Preferably, the dosage of the compound will vary from about 1 μg to about 10 mg per kilogram of body weight of the animal. More preferably, the dosage will vary from about 3 μg to about 1 mg per kilogram of body weight of the animal.

The inhibitor of the invention may be administered to a subject, or to a part of a subject such as a cell of an animal, as frequently as several times daily, or it may be administered less frequently, such as once a day, once a week, once every two weeks, once a month or even less frequently, such as once every several months or even once a year or less. The frequency of the dose will be readily apparent to the skilled artisan and will depend upon any number of factors, such as, but not limited to, the type and severity of the disease being treated, the type and age of the subject, etc. The formulations of the pharmaceutical compositions described herein may be prepared by any method known or hereafter developed in the art of pharmacology, In general, such preparatory methods include the step of bringing the active ingredient into association with a carrier or one or more other accessory ingredients, and then, if necessary or desirable, shaping or packaging the product into a desired single- or multi-dose unit,

Although the description of pharmaceutical compositions provided herein are principally directed to pharmaceutical compositions which are suitable for ethical administration to humans, it will be understood by the skilled artisan that such compositions are generally suitable for administration to animals of all sorts, including mammals. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation, Subjects to which administration of the pharmaceutical compositions of the invention is contemplated include, but are not limited to, birds, humans and other primates, mammals including commercially relevant mammals such as non-human primates, cattle, pigs, horses, sheep, cats, and dogs.

Pharmaceutical compositions that are useful in the methods of the invention may be prepared, packaged, or sold in formulations suitable for ophthalmic, oral, rectal, vaginal, parenteral, topical, pulmonary, intranasal, buccal, or another route of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. A unit dose is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents.

Controlled- or sustained-release formulations of a pharmaceutical composition of the invention may be made using conventional technology.

Parenteral administration of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intraocular, intravitreal, subcutaneous, intraperitoneal, intramuscular, intrasternal injection, and intratumoral.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, creams, lotions, gels, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition.

Formulations of a pharmaceutical composition suitable for topical (including mucosal) administration comprise the active ingredient combined with a pharmaceutically acceptable carrier, such as sterile water or sterile isotonic saline. Such formulations may be prepared, packaged, or sold in a form suitable for bolus administration or for continuous administration. Formulations may be prepared, packaged, or sold in unit dosage form, such as in ampules or in multi-dose containers containing a preservative. Formulations for topical administration include, but are not limited to, suspensions, solutions, emulsions in oily or aqueous vehicles, pastes, creams, lotions, gels, and implantable sustained-release or biodegradable formulations. Such formulations may further comprise one or more additional ingredients including, but not limited to, suspending, stabilizing, or dispersing agents. In one embodiment of a formulation for topical administration, the active ingredient is provided in dry (i.e. powder or granular) form for reconstitution with a suitable vehicle (e.g. sterile pyrogen-free water) prior to administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the invention.

Another formulation suitable for intranasal administration is a coarse powder comprising the active ingredient and having an average particle from about 0.2 to 500 micrometers. Such a formulation is administered in the manner in which snuff is taken i.e. by rapid inhalation through the nasal passage from a container of the powder held close to the nares.

Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the invention may be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein.

Pharmaceutical compositions of the invention may also provide the active ingredient in the form of gels, hydrogels, creams, solutions or suspensions. Gels and hydrogels may include but not limited to HydroxyEthyl Cellulose (HEC) gel, alginate gels or other gels or hydrogels. Such formulations may be prepared, packaged, or sold as gels, hydrogels, creams, solutions, suspensions, optionally sterile, comprising the active ingredient, and may conveniently be administered using any suitable applicator device. Such formulations may further comprise one or more additional ingredients including, but not limited to, a flavoring agent such as saccharin sodium, a buffering agent, a surface active agent, or a preservative such as sorbic acid or methylhydroxybenzoate.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Remington's Pharmaceutical Sciences (1985, Genaro, ed., Mack Publishing Co., Easton, Pa.), which is incorporated herein by reference.

### Kits

The invention also includes a kit comprising an inhibitor composition of the invention, or combinations thereof, and an instructional material which describes, for instance, administering the inhibitor composition of the invention, or combinations thereof; to a subject as a therapeutic treatment, or as a non-treatment use as described elsewhere herein. In an embodiment, the kit further comprises a (preferably sterile) pharmaceutically acceptable carrier suitable for dissolving or suspending the inhibitor composition of the invention, or combinations thereof; for instance, prior to administering the composition to a subject. Optionally, the kit comprises an applicator for administering the inhibitor composition.

### EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

### Example 1

### Virus-Induced Unfolded Protein Response Attenuates Anti-Viral Defenses Via Phosphorylation-Dependent Degradation of the Type I Interferon Receptor

Phosphorylation-dependent ubiquitination and degradation of the IFNAR1 chain of Type I interferon (IFN) receptor is regulated by two different pathways, one of which is ligand-independent. The results presented herein demonstrate that this pathway is activated by inducers of the endoplasmic reticulum (ER) stress, including viral infection, in a PERK-dependent manner. Upon infection, activation of this pathway promotes phosphorylation-dependent ubiquitination and degradation of IFNAR1, and specifically inhibits Type I IFN signaling and antiviral defenses. Either knock-in of an IFNAR1 mutant insensitive to virus-induced turnover or inhibition of PERK via either conditional knockout or knockdown by RNAi prevented ER stress- and virus-induced IFNAR1 degradation while restoring cellular responses to Type I IFN and resistance to viruses. The role of this novel mechanism in pathogenesis of viral infections and therapeutic approaches to their treatment is discussed below.

The Materials and Methods used in the experiments presented in this Example are now described.

### Plasmids and Reagents

Vectors for bacterial expression of GST-ctIFNAR1 and mammalian expression of human and mouse Flag-IFNAR1 were described previously (Kumar, et al., 2004, J. Biol Chem 279(45):46614-46620; Kumar et al., 2007, Cancer Biol Ther 6(9):1437-1441; Kumar, et al., 2003, Embo J 22(20):5480-5490); other plasmids were generous gifts (e.g., Flag-STAT1, HCV constructs, and Cre). All smRNA constructs used were based on pLKO.1. Recombinant human IFNα2 was purchased from Roche Diagnostics. Recombinant human and mouse IFNγ and mouse IFNβ were purchased from PBL. Thapsigargin, cycloheximide and methylamine HCl were purchased from Sigma.

Plasmid and viruses used are as follows:

shCon (CAACAAGATGAAGAGCACCAA; SEQ ID NO: 1), shIRE1α (GAGAAGATGATTGCGATGGAT; SEQ ID NO: 2) and shPERK (CCTCAAGCCATCCAACATATT; SEQ ID NO: 3) plasmids based on pLK0.1-puro vector (Sigma) were used in either transient transfection experiments or were used to generate lentivinises encoding the short hairpin sequences to infect 293T or 2fTGH cells.

### Cell Culture and Virus

All cell lines were maintained in Dulbecco's modified Eagle's medium (DMEM) supplemented with 10% (v/v) fetal bovine serum (Hyclone) and various selection antibiotics when indicated. To acutely delete PERK in MEFs, MEFs harboring PERK^{fl/fl} were infected with control retrovirus or retrovirus expressing Cre. The transduced cells were selected by puromycin for 72 hour. The surviving clones were pooled and used for further analysis. IFNAR1-null MEFs reconstituted with pBABE-puro-based retroviral vector encoding Flag-tagged mIFNARIwt and mIFNAR1S526A (Kumar, et al., 2003, Embo J 22(20):5480-5490) were generated and cultured in the presence of 4 μg/ml of puromycin. Huh7-derivative cells introduced with a complete HCV genome or a subgenomic genome were described in detail in (Luquin, et al., 2007, Antiviral Res 76(2):194-197) and were cultured in the presence of 500 μg/ml of G418.

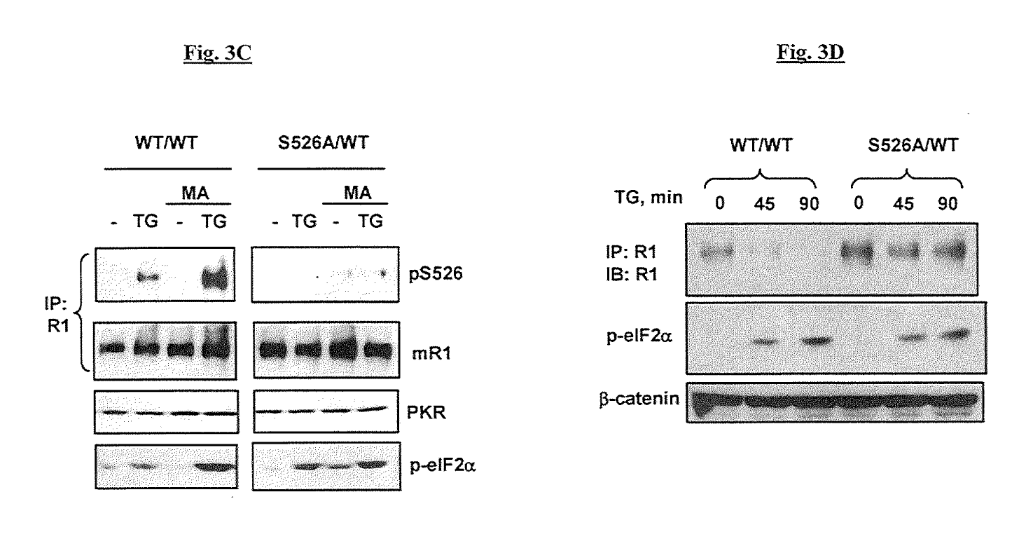

Mouse ES clone harboring a S526A mutation were obtained by homologous recombination, The targeting vector containing this mutation (FIG. 3A) was introduced via electroporation into the C57/BL6 ES cells. The cells were subjected to neomycin selection and DNA samples from survived clones were analyzed by Southern blotting using the indicated probes to identify the homologous recombinants. For experiments, ES cells were differentiated into embryonic bodies according to ATCC recommendations established protocol (Maatman, et al., 2003, Methods Mol Bol 209:201-230). The embryonic bodies were trypsinized and plated in gelatinized plates using IMDM containing 10% FBS. VSV (Indiana serotype) was propagated in HeLa cells.

### Transfections and Lentiviral Vector-Mediated Gene Knockdown:

Transfection of 293T cells and KR-2 cells using LIPOfectamine Plus and of Huh7-derivatives using LIPOfecatimine-2000 (Invitrogen) was carried out according to manufacturer's recommendations. Replication-deficient lentiviral particles encoding shRNA against GFP (shCON), hPERK and hIRE1α, or the empty virus control were prepared via co-transfecting 293T cells with three other helper vectors as previously described (Dull, et al, 1998, J Virol 72(11):8463-8471). Viral supernatant were concentrated by PEG8000 precipitation and were used to infect 2fTGH and U5A lines in the presence of 3 μg/ml of polybrene (Sigma). Cells were selected and maintained in the presence of 1.5 μg/ml of puromycin.

PERK-deficient MEFs and its WT counterparts were generous gifts from David Ron (New York University). PKR^{-/-}MEFs and their WT counterparts were generous gifts of R. Kaufman (University of Michigan). 293T cells were transfected with shRNA plasmids using LIPOfectamine Plus reagent (Invitrogen) according to manufacture's instructions. Studies on HCV were carried out using HCV genomic and subgenomic replicon system. Stable derivatives of Huh7 human hepatic cell line that express either incomplete genome of HCV (lacking structural proteins) or complete HCV genome (that expresses structural proteins as well) were generated and characterized as previously described (Luquin, et al., 2007, Antiviral Res 76(2):194-197).

### Cell Treatment and Viral Infection

For examining the signaling event occurring after initiation of ER stress, cells were treated with vehicle (DMSO) or TG (1 μM, unless otherwise indicated) for 0.5-2 hour as shown in the figure legends. Unless otherwise specified, cells were inoculated with VSV at an initial MOI of 0.1-1.0 for 1 hour. After removing the virus inoculum, cells were then fed with fresh medium. Cells were harvested at different time points afterwards; most of the effects were observed when the cells were uniformly infected and viral markers were at saturation. In some experiments, virus-infected cells were pulsed with IFNs for 30 min and then harvested. To examine the anti-viral effect of IFN in relation to the time of its addition, 20 IU/ml of IFN was either added overnight prior to VSV infection or was added after the initial virus inoculation/removal. Culture supernatant was generally harvested 20 hour after the initial inoculation for analysis of viral titer. VSV titer determination was performed as described elsewhere (Sharma, et al., 2003, Science 300(5622):1148-1151).

### Viral Titer Determination:

MEFs were infected with an apparent MOI0.1 of VSV for 1 h before the initial inoculum was removed and the cell layer was fed with medium after washed once with PBS. 20 h after infection, the virus-containing culture supernatant was harvested and the viral titer is determined according to previously published report (Sharma, et al., 2003, Science 300(5622):1148-1151). At the time of harvesting cells for biochemical analyses, cells were infected almost uniformly judging by saturation in the levels of viral markers.

Virus-Mediated shRNA Knockdown:

Virus packaging was done in 293T cells as described elsewhere (Dull et al., 1998). Target cells were infected with concentrated virus in the presence of 3 μg/ml of polybrene. 48 h after transduction, 2fTGH and 293T cells were selected in medium containing 1.5 and 3 μg/ml of puromycin, respectively.

### Immunotechniques

Antibodies against pSTAT1, p-eIF2, p-β-catenin, β-catenin, IRE1α (Cell Signaling), STAT1 (Cell Signaling), eIF2α (Biosources), hIFNAR1, PKR, c-Jun, IKBa (Santa Cruz), mIFNAR1 (R&D Systems), Flag tag, β-actin (Sigma) and ubiquitin (clone FK2, Biomol), ISG15 and PERK (Rockland) were used for immunoprecipitation and immunoblotting. Monoclonal antibody 23H12, specific for the M protein of VSV (VSV-M), was kindly provided by D. S. Lyles (Wake Forest University School of Medicine, Winston-Salem, N.C.). Antibody against IFNAR1 phosphorylated on Ser535 (in human receptor) or Ser526 (in murine receptor) were described previously (Kumar, et al., 2004, J Biol Chem 279(45):46614-46620). Cells lysis, immunoprecipitation and immunoblotting procedures were described earlier (Kumar, et al., 2004, J Biol Chem 279(45):46614-46620). Kinase assay with cell lysates and GST-ctIFNAR1 as a substrate was previously described (Liu, et al., 2008, Biochem Biophys Res Commun 367(2):388-393).

### In Vitro Kinase Assay

In vitro PERK kinase assay using GST-cIFNAR1 as a substrate was performed using kinase buffer containing 20 mM HEPES 7.4, 50 mM KCl, 2 mM MgOAC, 2 mM MnCl2, 20 μM ATP and 1.5 mM DTT. Recombinant PER^{ΔN} and GST-IFNAR1 were described previously (Cullinan et al., 2003, Mol Cell Biol 23:7198-7209; Kumar, et al., 2003, Embo J 22(20):5480-5490). 5 ng of PERK^{ΔN} and 1 μg of GST-IFNAR1 was mixed in the kinase buffer containing 1 μCi of γ-ATP. The reaction mix was incubated at 30° C. for 30 min. The samples were separated on SDS-PAGE and analyzed by auto-radiography.

### FACS Analysis

Measurements of surface levels of IFNAR1 in MEFs of various genetic background was carried out using an anti-mIFNAR1 antibody (R&D Services) as previously described elsewhere (Sheehan et al., J. Interferon Cytokine Res. 26:804).

The results of the experiments presented in this Example are now described.

### The UPR Induces Perk-Dependent Phosphorylation of IFNAR1 Degron

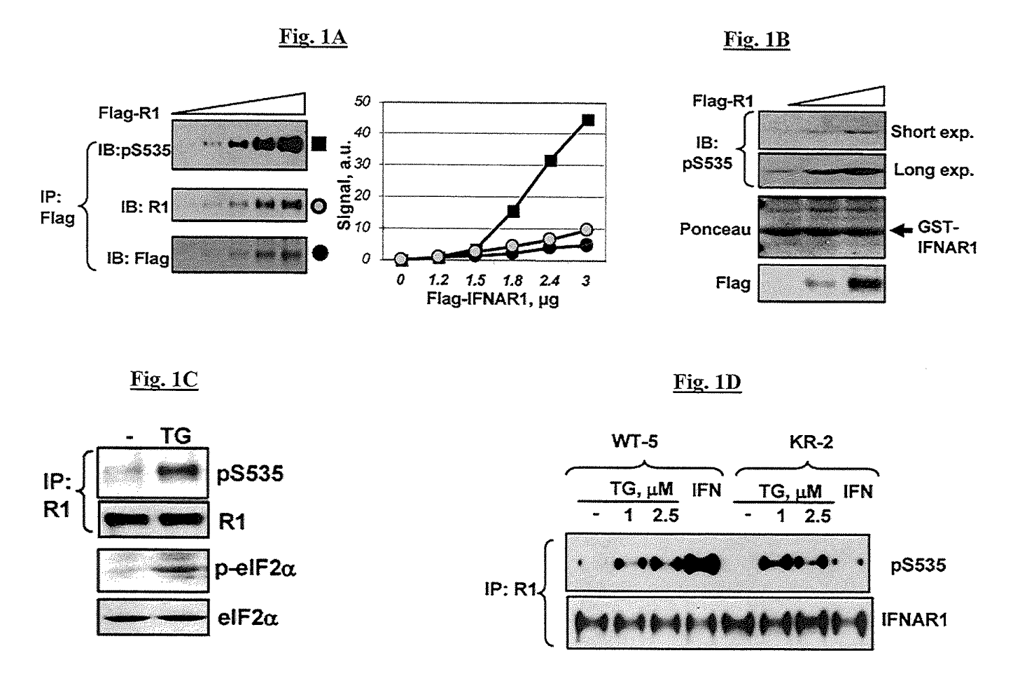

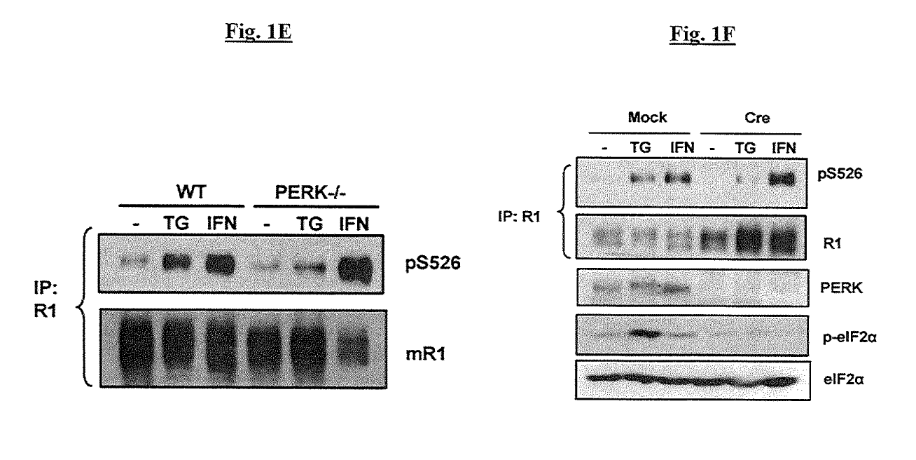

Overexpressed IFNAR1 undergoes ligand- and Jak-independent degron phosphorylation followed by ubiquitination and degradation of this receptor (Liu, et al., 2008, Biochem Biophys Res Commun 367(2):388-393). Increasing the amount of transfected IFNAR1 plasmid led to a disproportionate increase in phospho-IFNAR1 signal that cannot be explained solely by higher levels of total IFNAR1 expressed in these cells (FIG. 1A). Furthermore, lysates from these transfected cells displayed an elevated ability to phosphorylate bacterially produced GST-IFNAR1 protein on Ser535 (FIG. 1B) indicating that forced expression of IFNAR1 activates a signal transduction pathway inducing an unknown protein kinase activity that phosphorylates IFNAR1 within its degron.

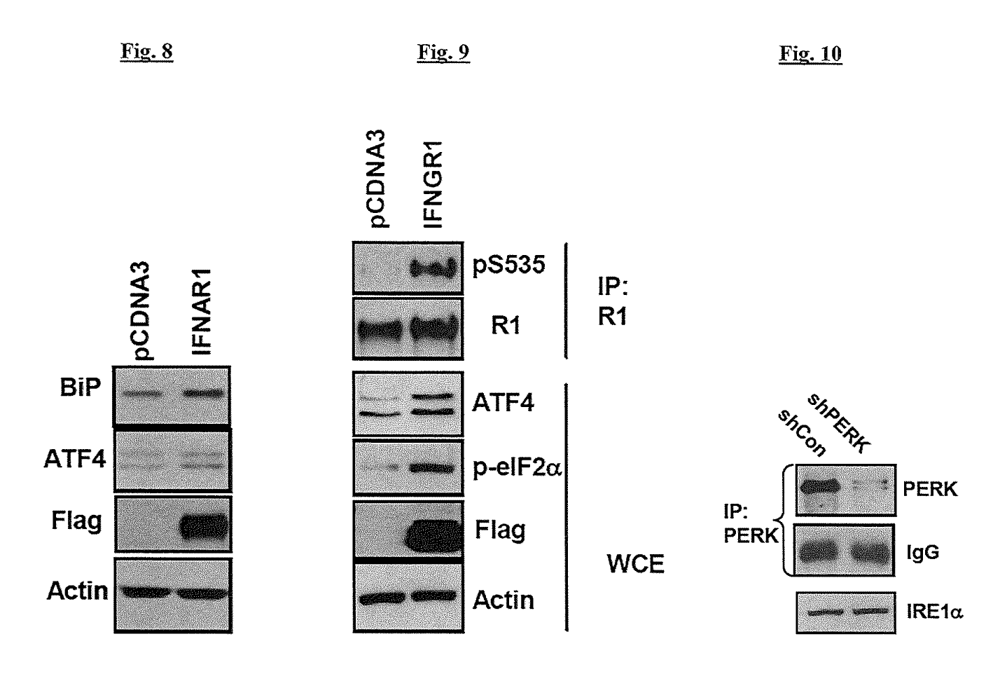

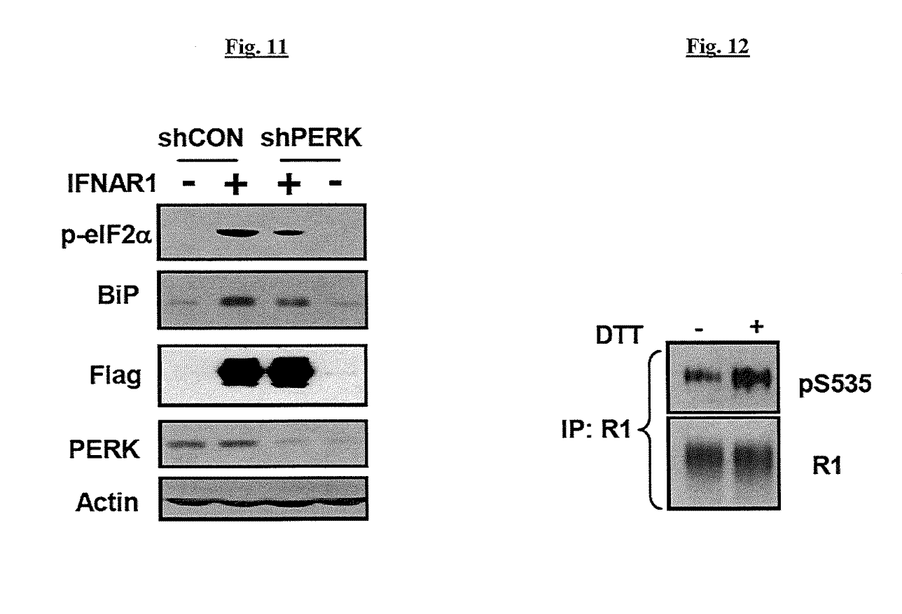

Overexpression of secretory and transmembrane proteins (such as IFNAR1) may overpower the ability of a cell to properly fold these proteins in the ER and, therefore initiate the UPR (Welihinda, et al., 1999, Gene Expr 7:293-300). As disclosed elsewhere herein, in FIGS. 8 and 11, forced expression of IFNAR1 induced the markers of the UPR such as BiP and ATF4 and promoted phosphorylation of eIF2α. Similar results along with phosphorylation of endogenous IFNAR1 on Ser535 were obtained upon overexpression of unrelated IFNγ receptor IFNAR1 (FIG. 9). It appears that eIF2α phosphorylation was dependent on PERK as evident from experiments using RNAi approach to knock down this kinase (FIGS. 10-11). Remarkably, thapsigargin, a known inducer of UPR stimulated phosphorylation of endogenous IFNAR1 on Ser535 in the absence of IFN (FIG. 1C). Similar results were obtained using other known UPR stimuli such as DTT (FIG. 12) and tunicamycin (not shown).

The next set of experiments was designed to investigate whether activity of Tyk2, which is required for IFNAR1 phosphorylation in response to IFN (Liu, et al., 2008, Biochem Biophys Res Commun 367(2):388-393; Marijanovic, et al., 2006, Biochem J 397(1):31-38), plays a role in the ligand-independent pathway. To this end, derivatives of human fibrosarcoma 2fTGH-derived cell lines originally sensitive to Type I IFN (John, et al., 1991, Mol Cell Biol 11(8):4189-4195) but then having lost Tyk2 expression was utilized (Velazquez, et al., 1992, Cell 70(2):313-322). These cells were reconstituted with either wild type (WT) Tyk2 or its catalytically inactive (KR) mutant (Marijanovic, et al., 2006, Biochem J 397(1):31-38). In line with the latter report, IFN-a-stimulated phosphorylation was inhibited in KR cells; however, thapsigargin induced comparable levels of Ser535 phosphorylation of IFNAR1 in both cell lines (FIG. 1D). These data suggest that UPR mediates phosphorylation of IFNAR1 in a ligand- and Tyk2 kinase-independent manner.

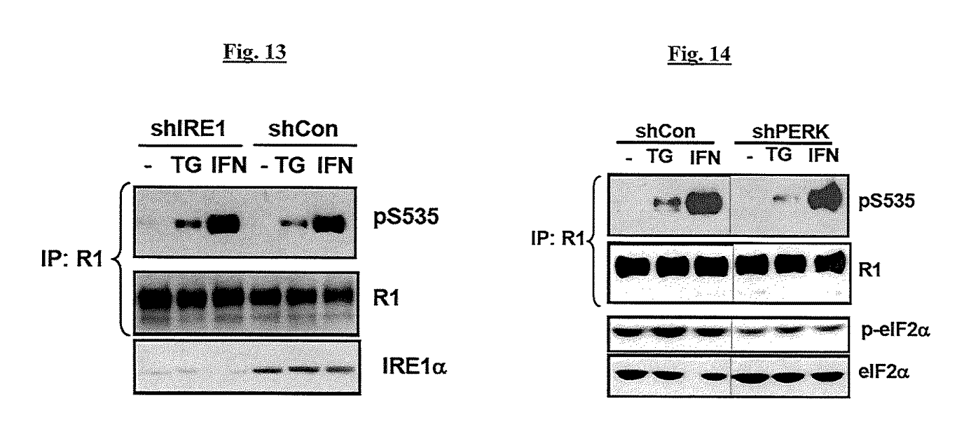

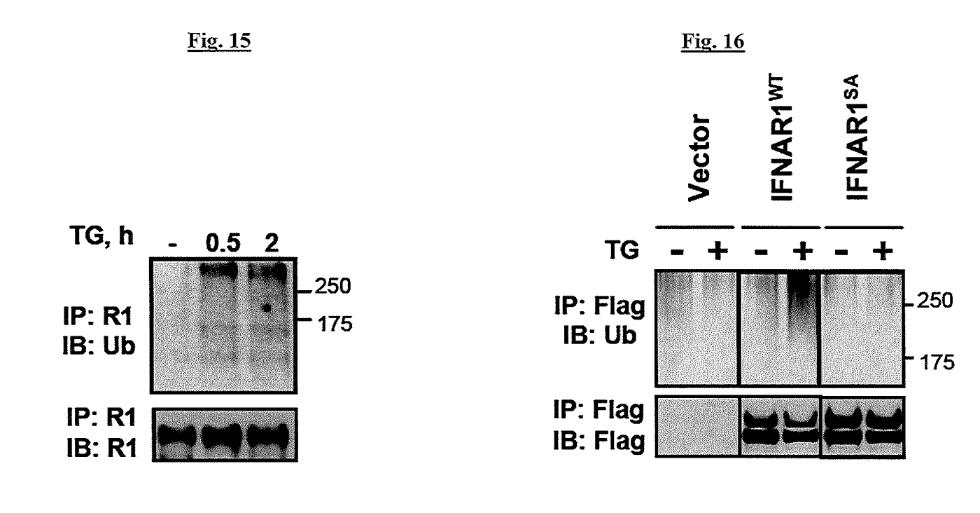

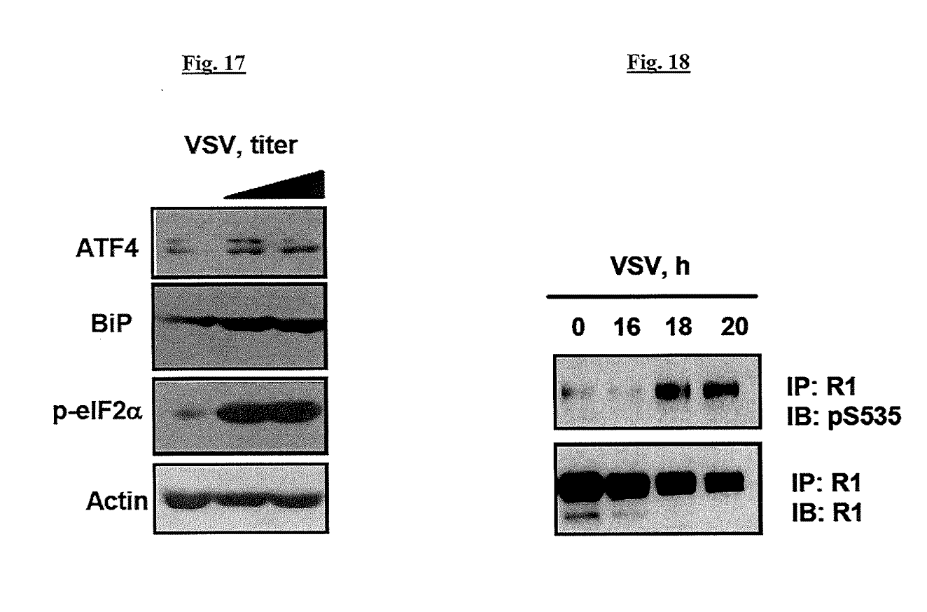

The next set of experiments was designed to investigated which branch of UPR signaling is involved in regulating IFNAR1 phosphorylation. Embryo fibroblasts derived from PERK-null mice exhibited attenuated IFNAR1 phosphorylation in response to thapsigargin but not to murine (FIG. 1E), This data was corroborated using the fibroblasts from mice harboring a conditional knockout allele of PERK (PERKfllfl) where PERK is acutely excised upon transduction with retrovirus encoding the Cre recombinase (Zhang, et al., 2002, Mol Cell Biol 22:3864-3874). The acute deletion of PERK inhibited IFNAR1 phosphorylation induced by thapsigargin without affecting IFN-triggered phosphorylation (FIG. 1F). Phosphorylation of IFNAR1 in response to thapsigargin in human cells was not inhibited by either knockdown of IRE1 or the expression of a dominant negative mutant of IRE1 (FIG. 13). Conversely, the knockdown of PERK noticeably decreased the efficacy of IFNAR1 phosphorylation induced by thapsigargin but not by IFN-α in human cells (FIG. 14).

Collectively, these data suggest that PERK is required for IFNAR1 degron phosphorylation stimulated by UPR. Given that activated PERK was not capable of phosphorylating IFNAR1 in vitro it is likely that a kinase(s) downstream of PERK is responsible for the direct phosphorylation of IFNAR1 degron.

### The UPR Promotes IFNAR1 Ubiquitination and Degradation by Inducing Degron Phosphorylation in a Ligand- and Tyk2-Independent Manner

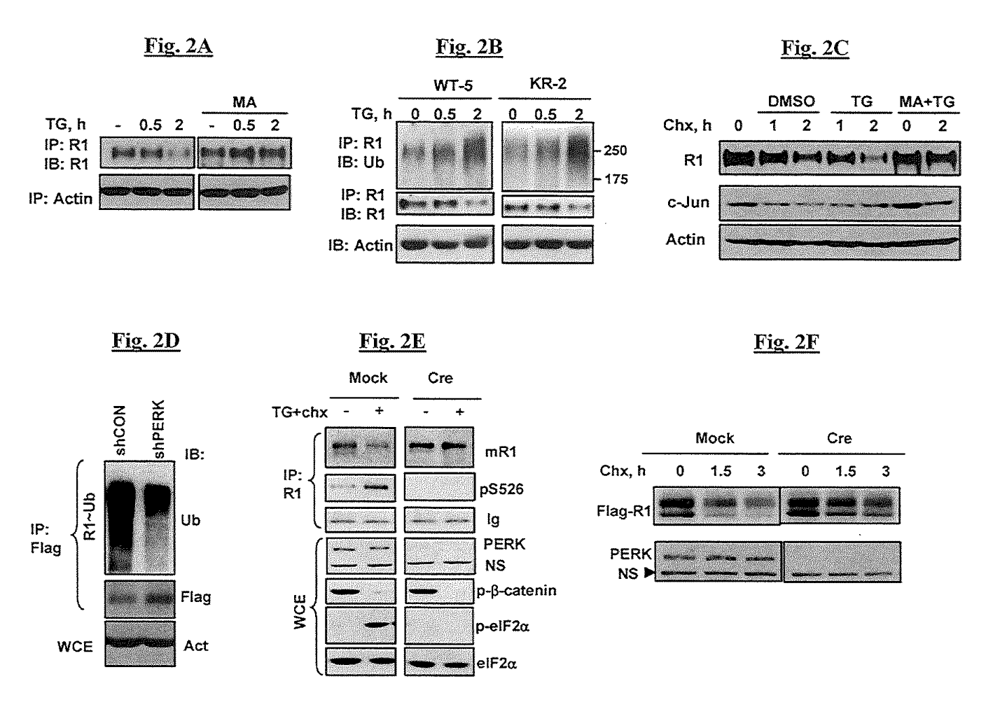

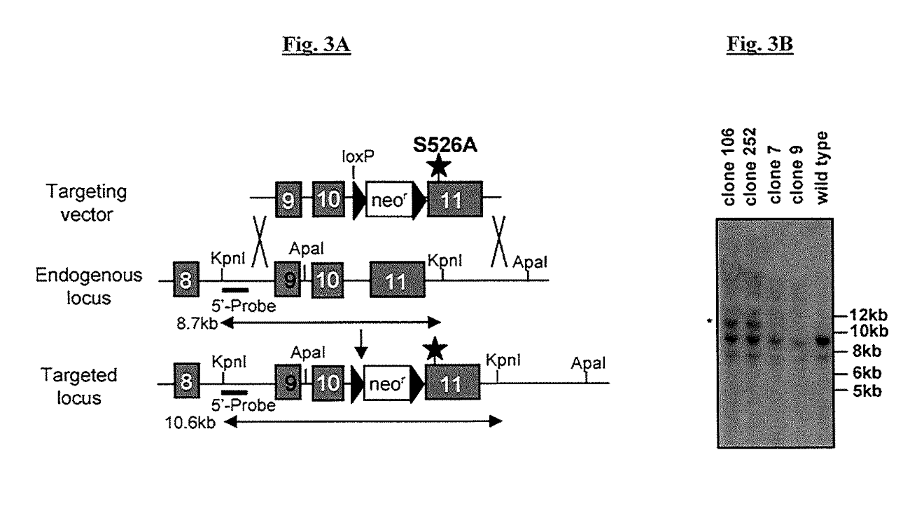

Phosphorylation within the IFNAR1 degron is expected to promote ubiquitination of this receptor and its degradation in the lysosome (Kumar, et al., 2004, J Biol Chem 279(45):46614-46620; Kumar, et al., 2003, Embo J 22(20):5480-5490; Marijanovic, et al., 2006, Biochem J 397(0:31-38). Indeed, treatment of cells with thapsigargin decreased the levels of IFNAR1 in human cells within two hours even in the absence of IFN. This decrease was prevented by pre-treating cells with methylamine HCl (MA), an inhibitor of the lysosomal pathway (FIG. 2A). Furthermore, thapsigargin treatment induced ubiquitination of IFNAR1 and downregulated this receptor in human fibrosarcoma cells that express either wild type (WT) or catalytically inactive (KR) Tyk2 (FIG. 2B) as well as in 293T cells (FIG. 15). Ligand-independent stimulation of IFNAR1 ubiquitination by thapsigargin was also seen in IFNAR1-null mouse fibroblasts reconstituted with IFNAR1WT but not with IFNAR1SA mutant lacking phosphorylation site (FIG. 16). These results indicate that induction of the UPR promotes phosphorylation-dependent ubiquitination of IFNAR1 and downregulates its levels in a manner independent of Tyk2 and of exogenous IFN.

Treatment of cells with thapsigargin decreased the half life of IFNAR1 but not of an unrelated short lived protein, c-Jun, in 293T cells treated with cycloheximide to inhibit translation (FIG. 2C). Knockdown of PERK decreased the ubiquitination of exogenously overexpressed IFNAR1 and noticeably increased its level in human cells (FIG. 2D). Similarly, thapsigargin-induced ubiquitination of IFNAR1 was alleviated in PERK-deficient cells mouse cells. In addition, acute Cre-mediated ablation of PERK slowed down UPR-induced turnover of both endogenous (FIG. 2E) and exogenously expressed mouse IFNAR1 (FIG. 2F). In contrast to that, degradation of another P-Trcp substrate, phosphorylated Jβ-catenin, was not affected under these conditions (FIG. 2E). Collectively, these data suggest that UPR promotes ubiquitination and degradation of IFNAR1 in a PERK-dependent manner.

The next set of experiments was designed to investigate whether UPR-stimulated IFNAR1 degradation is mediated via phosphorylation of serine residues within the IFNAR1 degron. To this end, mouse embryonic stem (ES) cells that harbor one mutant IFNAR1S526A allele introduced via a homologous recombination approach was generated (FIG. 3A-B). These cells were grown as embryoid bodies (EB) and differentiated into fibroblast-like cells for analysis. Although treatment with thapsigargin induced a comparable level of eIF2α phosphorylation in both wild type and S526A knock-in cells, the latter displayed a grossly reduced phosphorylation of IFNAR1 on Ser526 (FIG. 3C). Furthermore, thapsigargin-stimulated degradation of IFNAR1 was clearly inhibited in the S526A knock-in cells (FIG. 3D). These data indicate that UPR-induced acceleration of proteolytic turnover of IFNAR1 depends on its phosphorylation within the specific degron.

### VSV and HCV Accelerate the Degradation of IFNAR1 Via Induction of PERK-Dependent IFNAR1 Degron Phosphorylation

While IFN-α/β play a major role in the defense against viruses, pre-treatment of yet uninfected cells with these cytokines are often required to obtain the protective effect. Numerous viruses including hepatitis C virus (HCV, (Ciccaglione, et al., 2007, Virus Res 126(1-2):128-138; Wang, et al., 2006, J Gastroenterol Hepatol 21 Suppl 3:S34-37; Zheng, et al., 2005, J Microbiol 43:529-536)) are known to massively express their proteins and cause ER stress. Therefore, the next set of experiments was designed to investigate whether virus-induced UPR might also affect IFNAR1 phosphorylation and stability that may also lead to inhibiting IFN responsiveness of already infected cells.

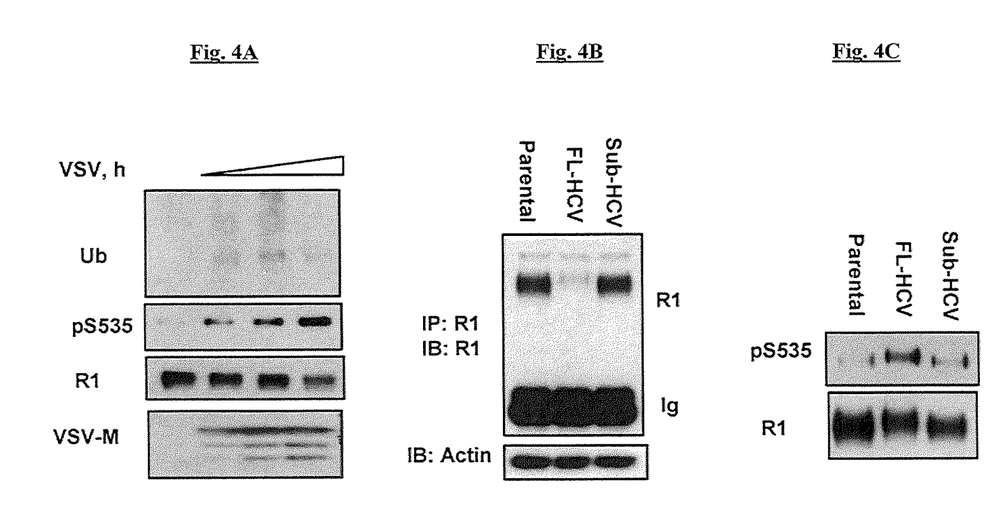

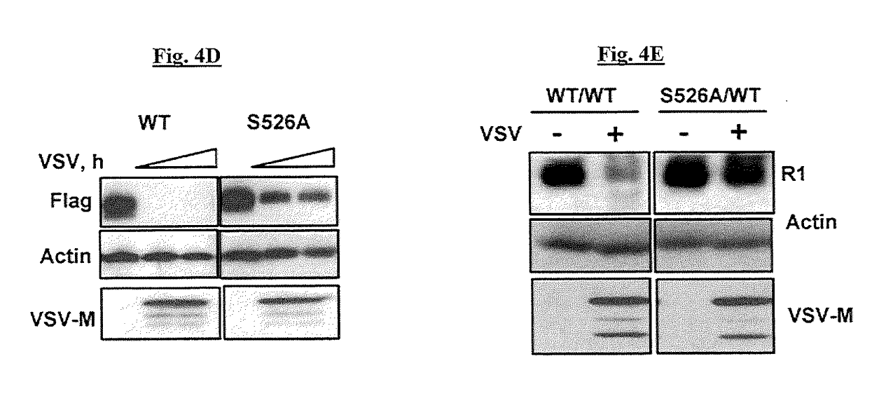

Infection of 2fTGH human fibrosarcoma cells with vesicular stomatitis virus (VSV) induced the expression of UPR markers (FIG. 17). Intriguingly, this infection also stimulated IFNAR1 phosphorylation on Ser535 and decreased total levels of IFNAR1 (FIG. 18). Similar results and an increase in the extent of IFNAR1 ubiquitination were observed in the KR derivative cell line (FIG. 4A) indicating that VSV infection promotes IFNAR1 phosphorylation, ubiquitination and degradation in a Tyk2-independent manner. Furthermore, given that Tyk2 activity is essential for IFN-induced IFNAR1 phosphorylation (Marijanovic, et al., 2006, Biochem J 397(1):31-38), this result suggests that the effects of VSV on IFNAR1 downregulation could not be attributed solely to induction of endogenous IFN.

Infection with hepatitis C virus (HCV) promotes the ER stress (Tardif, et al., 2005, Trends Microbiol 13(4):159-163; Waris, et al., 2002, Biochem Pharmacol 64:1425-1430) that is robustly stimulated by the synthesis of structural proteins (Ciccaglione, et al., 2005, Arch Virol 150(7):1339-1356) known to reside in ER lumen of infected cells (Wu, 2001, IUBMB Life 51:19-23). In human hepatoblastoma Huh7 cells, total levels of endogenous IFNAR1 were dramatically down regulated by stable transfection of a complete HCV genome (FIG. 4B). When the levels of IFNAR1 taken into immunoprecipitation were normalized to yield comparable total levels of IFNAR1, a robust phosphorylation of IFNAR1 degron was detected in Huh7 cells expressing the complete HCV genome (FIG. 4C). Thus, it is plausible that the effects of viral infection/expression of viral proteins on IFNAR1 levels could be mediated via IFNAR1 degron phosphorylation and ensuing degradation.

Indeed, while VSV infection dramatically down regulated murine Flag-tagged IFNAR1 (re-expressed in MEFs from IFNAR1-null mice), a noticeably lesser effect was observed on mutant IFNAR1S526A (FIG. 4D). This result was further corroborated in IFNAR1S526A knock-in cells that were more resistant in decreasing IFNAR1 levels in response to VSV (FIG. 4E). These data indicate that phosphorylation of IFNAR1 degron is implicated in the receptor downregulation stimulated by virus.

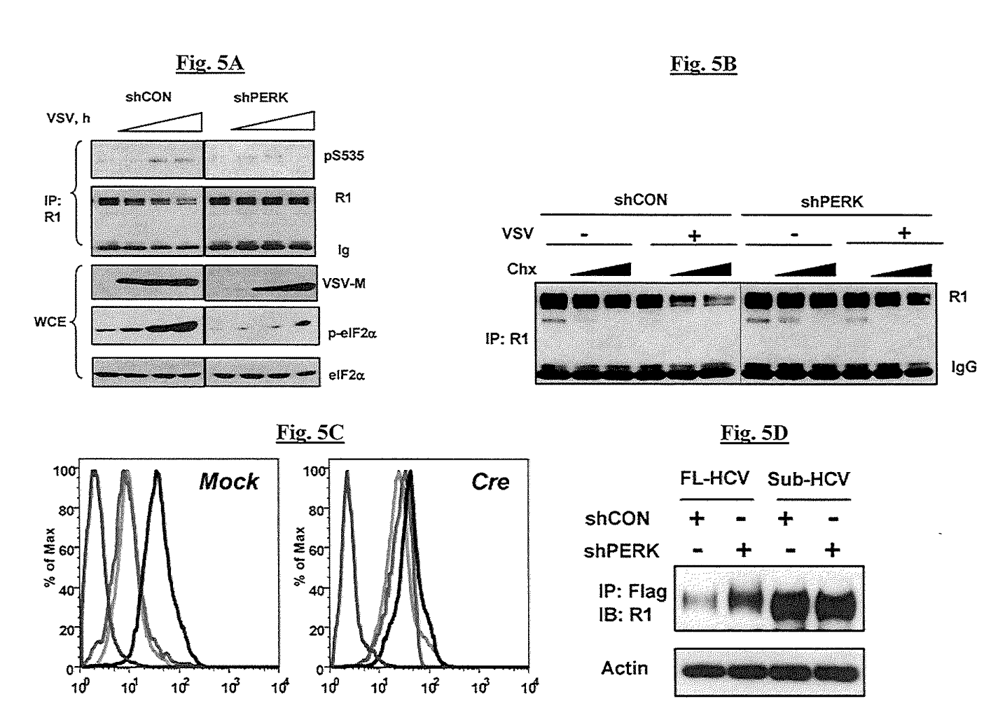

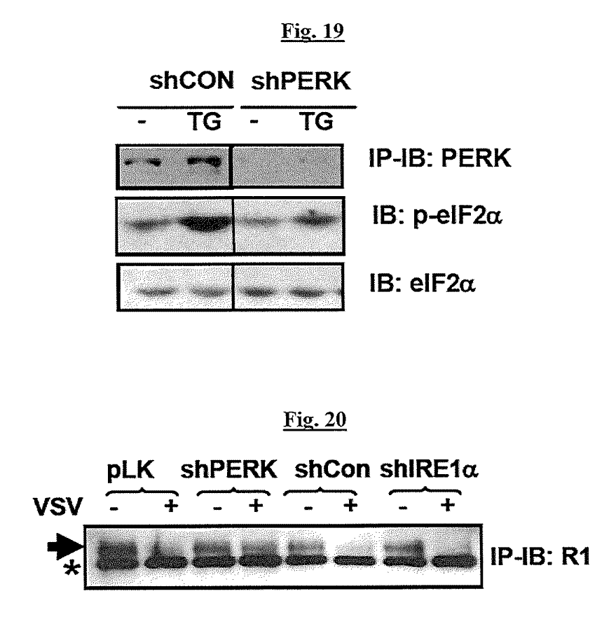

Knock-down of PERK in human 2fTGH cells (FIG. 19) attenuated degron phosphorylation and downregulation of IFNAR1 in cells infected with VSV (FIG. 5A). Similarly, downregulation of IFNAR1 in VSV-infected 21TGH cells was prevented by shRNA against PERK but not by a number of irrelevant shRNAs or shRNA against IRE1 (FIG. 20). Accordingly, a much lesser extent of degradation of IFNAR1 promoted by VSV infection (measured in cycloheximide-treated cells) was detected in PERK knockdown cells (FIG. 5B). Furthermore, CRE-mediated ablation of PERK decreased the extent of downregulation of cell surface IFNAR1 levels in response to either TG treatment or VSV infection (FIG. 5C). In addition, knockdown of PERK partially rescued a decrease in IFNAR1 observed in Huh7 cells expressing the complete HCV genome (FIG. 5D). These data suggest that viral infection and expression of structural viral proteins promote downregulation and degradation of IFNAR1 via a PERK-dependent signaling.

### VSV and HCV Attenuate Cellular Responses to Type I IFN Via PERK-Dependent Phosphorylation and Downregulation of IFNAR1

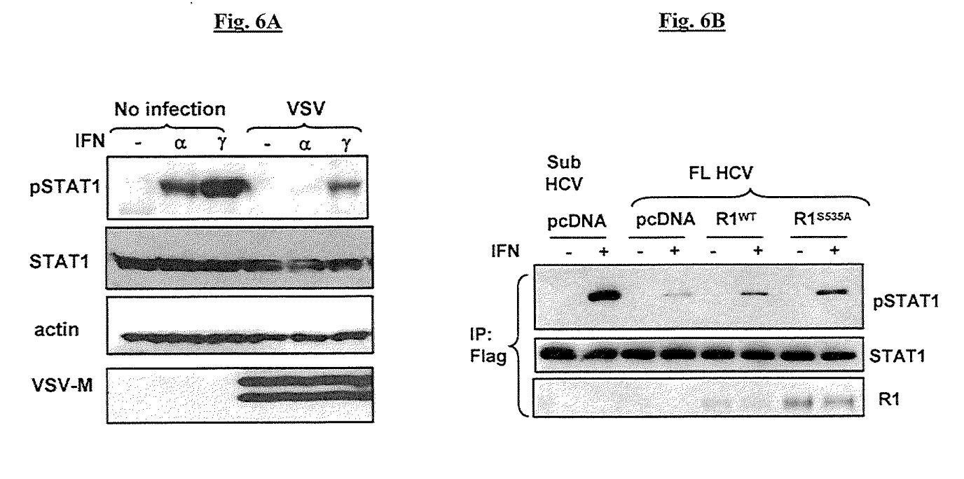

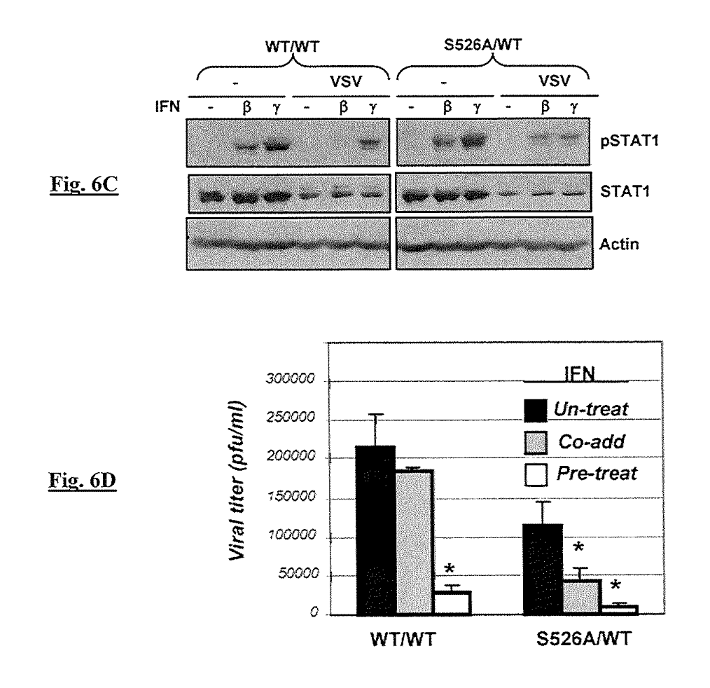

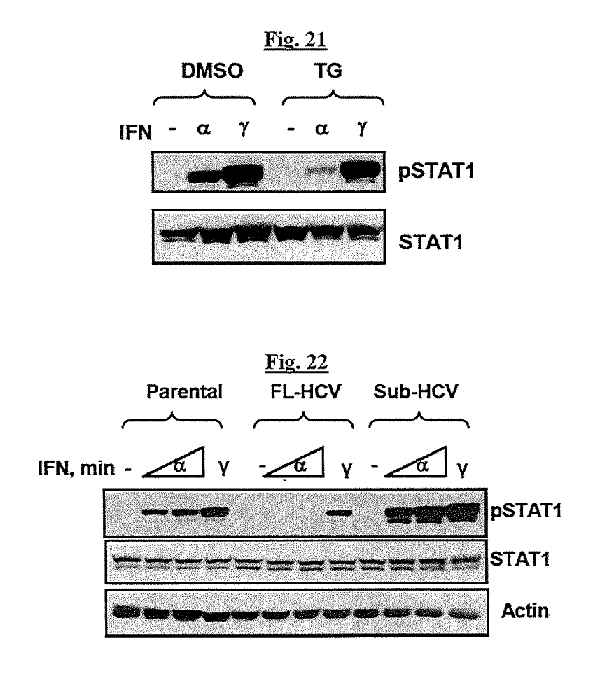

Attenuated anti-viral defense observed in cells from IFNAR1+/−mice suggests that levels of IFNAR1 are important for Type I IFN signaling (Hwang et al., 1995, Proc Natl Acad Sci USA 92(24):11284-11288; Muller, et al, 1994, Science 264(5167):1918-1921). Therefore, IFNAR1 downregulation triggered by UPR activation is expected to inhibit cellular responses to IFN-α/(3. Indeed, either infection of cells with VSV (FIG. 6A) or pre-treatment of cells with thapsigargin (FIG. 21) noticeably decreased tyrosine phosphorylation of Stat1 induced by IFN-a in human cells. Much lesser inhibition was seen in cells treated with IFNγ that utilizes an entirely different receptor (Pestka, 2000, Biopolymers 55(4):254-287; Schreiber, et al., 1993, Gastroenterol Jpn 28(Suppl 4):88-94), although yet requiring IFNAR1 for maximal signaling (Takaoka, et al., 2000, Science 288(5475):2357-2360).

The expression of the complete HCV genome in Huh7 cells dramatically inhibited Stat1 phosphorylation induced by IFN-a while IFNγ signaling was only modestly affected ((Luquin, et al., 2007, Antiviral Res 76(2):194-197) and FIG. 22). Remarkably, expression of IFNAR1 proteins in these cells partially rescued Type I IFN signaling; this effect was especially pronounced when a stabilized IFNAR1S535A mutant (that lacks Ser responsible for UPR-driven degradation, FIG. 4) was used (FIG. 6B). Similarly, a lesser inhibitory effect of VSV infection on Type I (but not Type II) IFN signaling was observed in cells derived from the IFNAR1S526A knock-in ES cells (FIG. 6C). These results indicate that ER stress and viral infection inhibit Type I IFN signaling via phosphorylation-dependent downregulation of IFNAR1.

The next set of experiments was designed to investigate the role of this regulation in anti-viral defense. While pre-treatment of wild type cells with IFN-β exhibited an anti-viral effect, this cytokine was inefficient when added immediately after the virus. However, under the latter conditions, cells that harbored the knocked-in IFNAR1S526A mutant were capable of utilizing IFN-β to significantly reduce VSV propagation (FIG. 6D). These results indicate that viruses at least temporarily benefit from the induced phosphorylation-dependent degradation of IFNAR1 and the ensuing suppression of the anti-viral defenses.

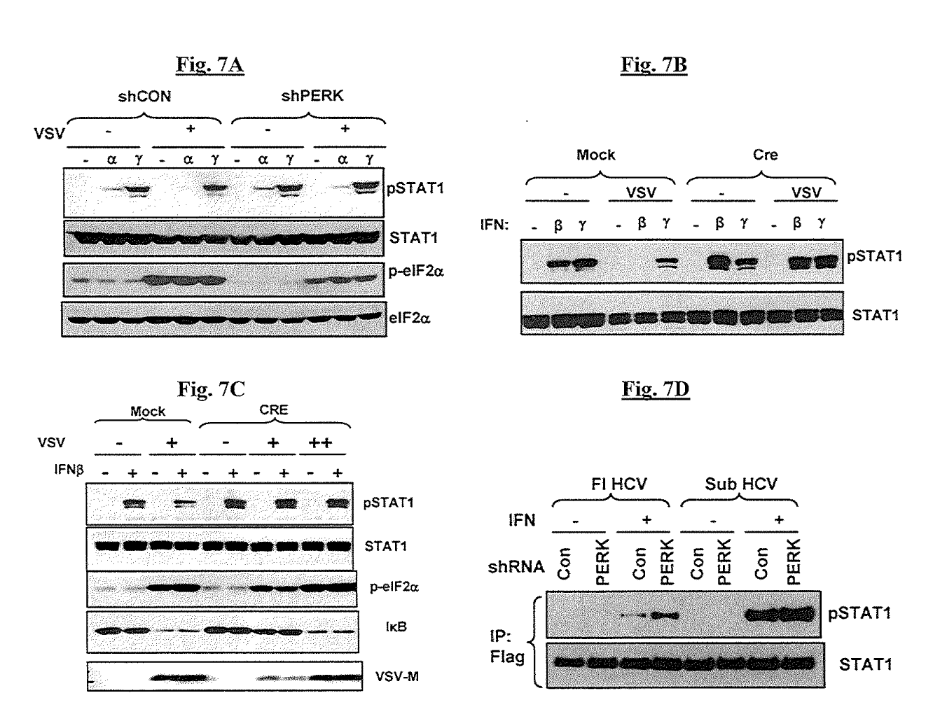

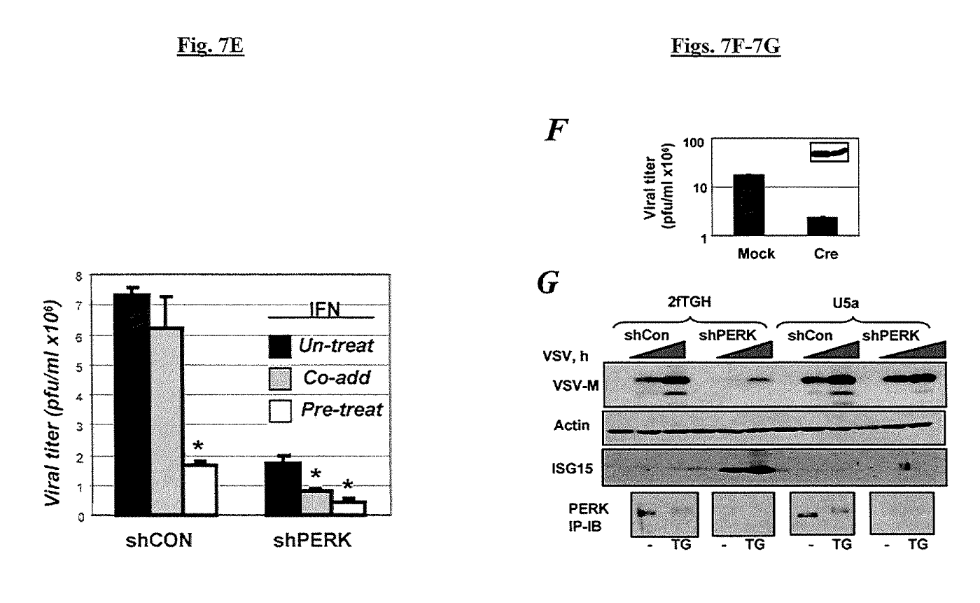

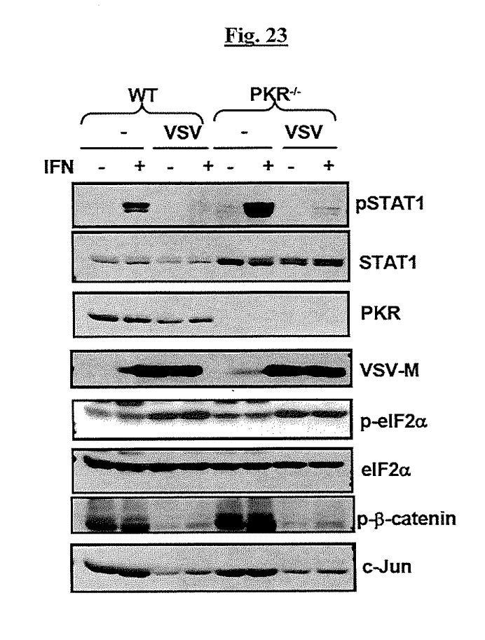

This hypothesis was further corroborated when the role of PERK in Type I IFN-induced signaling and anti-viral defense was investigated. Either knockdown of PERK in human cells (using RNAi approach) or acute genetic ablation of PERK in mouse fibroblasts (using Cre expression) led to the rescue of Stat1 phosphorylation in response to Type I IFNs (FIGS. 7A-B). Judging from expression of viral VSV-M protein, PERK-deficient cells contained fewer viruses; however, even when exposed to a five-fold higher viral titer to achieve a comparable expression of VSV-M, these cells remained competent in IFN-β-induced activation of Stat1 (FIG. 7C). Such protection was not seen in infected MEFs lacking a related kinase, PKR (FIG. 23). Specific role of PERK was further corroborated by data demonstrating that PERK knockdown in Huh7 cells expressing complete HCV genome also restored the ability of these cells to conduct Type I IFN signaling (FIG. 7D). These data suggest that PERK plays an important role in virus-mediated inhibition of cellular responses to IFN-α/β.

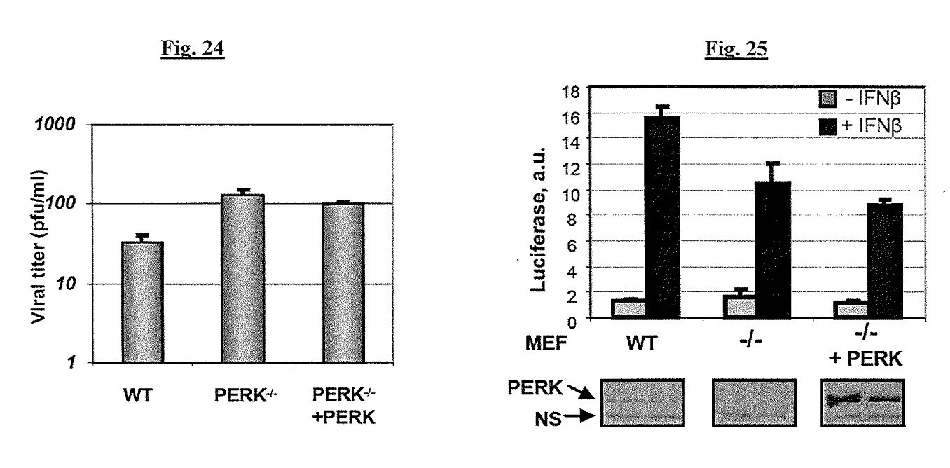

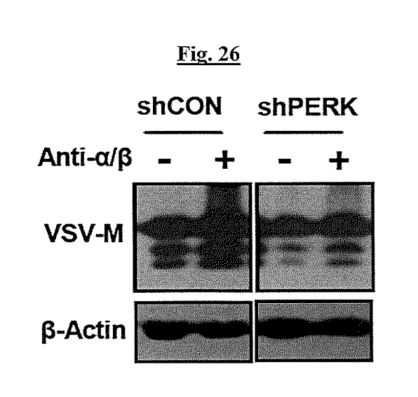

PERK knockdown in human cells increased their overall resistance to VSV and promoted the ability of cells to utilize IFN-a added after the virus to significantly suppress the replication of VSV in these cells (FIG. 7E). This finding is counterintuitive as both previous report (Baltzis, et al., 2004, J Virol 78:12747-12761) and the data relating to FIG. 24, which indicated that mouse fibroblasts derived from conventional PERK knockout embryos are somewhat more sensitive to viral infection. Accordingly, conventional PERK-null MEFs displayed a somewhat reduced response to IFN as evident from analysis of the ISRE-driven luciferase reporter (FIG. 25). However, re-expression of PERK in these cells did not stimulate either IFN responses or anti-viral defenses (FIGS. 24-25) suggesting that conventional PERK-null cells underwent an additional alteration to decrease IFN signaling. Without wishing to be bound by any particular theory, it is believed that this alteration was to compensate an impaired ability to downregulate IFNAR1 via the ligand-independent pathway that has been previously shown to impede cell growth (Liu, et al., 2008, Biochem Biophys Res Commun 367(2):388-393). Conversely, a compensatory pathway that rescued growth of cells overexpressing a dominant negative mutant of PERK has been also reported (Yamaguchi, et al., 2008, J Biol Chem 283:17020-9). Therefore, to investigate the role of PERK in murine cells, a model of Cre-mediated acute ablation of PERK in MEFs from PERKfl/fl mice that did not display defects in ISRE-dependent transcription was used.

Remarkably, expression of Cre rendered these MEFs more resistant to VSV in the absence of added IFN (as seen from a decreased viral titer and expression of VSV-M, FIG. 7F). Furthermore, PERK-deficient 2fTGH cells elevated the expression of interferon stimulated protein ISGI 5 and became more resistant to VSV infection (as judged by the lower levels of VSV-M protein, FIG. 7G). These effects of PERK loss of function appeared to largely depend on the TEN pathway as much lesser changes were observed in isogenic U5a cells lacking IFNAR2. This conclusion was also supported by the fact that expression of VSV-M in infected PERK knockdown cells was noticeably increased by treatment with anti-IFN-α/β antibodies (FIG. 26). In all, these results suggest that activation of PERK is utilized by VSV to inhibit cellular responses to IFN and anti-viral defenses; this mechanism at least partially relies on UPR-induced PERK-dependent downregulation of TEN receptor.

### Inhibition of PERK to Augment the Efficacy of Endogenous and Pharmaceutical Type I IFN

Ligand-stimulated, Jak-dependent ubiquitination and degradation of Type I IFN receptor plays a key role in the negative regulation of IFN-α/β signaling (Kumar, et al., 2003, Embo 122(20):5480-5490). However, recent evidence suggested the existence of a ligand- and Jak-independent pathway that controls stability of IFNAR1 in a phosphorylation-dependent manner. The importance of the latter pathway remained unclear as it was largely observed under the conditions of IFNAR1 overexpression (Liu, et al., 2008, Biochem Biophys Res Commun 367(2):388-393). The results presented herein demonstrate that this pathway is triggered by activation of the ER stress in a manner that requires function of PERK. Among the evidence supporting this conclusion are the following: (i) stimuli that cause UPR induce Ser phosphorylation within the IFNAR1 degron and promote IFNAR1 ubiquitination and degradation in cells that were not treated with IFN and in a Tyk2-independent manner; (ii) UPR-induced ubiquitination and degradation of IFNAR1 is inhibited in cells harboring knocked-in IFNAR1 mutant lacking phospho-acceptor site in its degron; and (iii) phosphorylation, ubiquitination and degradation of IFNAR1 induced by UPR are attenuated in PERK-deficient cells.

Furthermore, the results presented herein demonstrate that this pathway, which leads to accelerated degradation of IFNAR1, is utilized by some viruses (including VSV and HCV). Infection by VSV and expression of HCV genome led to downregulation of IFNAR1 and to inhibition of signaling and anti-viral effects induced by Type I IFN. These effects are at least partially impaired in cells that either lack PERK or contain phospho-degron mutant of IFNAR1 that is insensitive to PERK-induced phosphorylation and degradation. Given that infection with many of human and animal viruses are known to induce the UPR (He, 2006, Cell Death Differ 13(3):393-403; Schroder, et al., 2006, Curr Mol Med 6(1):5-36; Wang, et al., 2006, J Gastnienterol Hepatol 21 Stipp' 3:S34-37; Waris, et al., 2002, Biochem Pharmacol 64:1425-1430), it is believed that some rapidly propagating viruses generally employ the ligand-independent degradation of IFNAR1 to suppress anti-viral defenses in cells that have not yet been exposed to TEN. It is also believed that this mechanism plays a role in pathogenesis of some viral infectious diseases.

ER stress has evolved to help the cells to deal with protein overload, which among other scenarios also occurs during acute viral infections. According to a current paradigm, being a cellular protective mechanism, UPR as a whole helps to limit viral infection (He, 2006, Cell Death Differ 13(3):393-403). The results presented herein, however, strongly suggest that specific activation of the PERK branch of UPR instead favors viral replication via IFNAR1 degradation and suppression of IFN responses. Without wishing to be bound by any particular theory, it is believed that one major consequence of PERK activation is an inhibition of translation through eIF2a phosphorylation, which, in cells infected by viruses, can also be carried out by PKR. It is believed that this redundancy in means of translational inhibition permits a sustained stimulation of PERK to negate IFN signaling and promote the infection. Intriguingly, while viruses often impede PKR-dependent phosphorylation of eIF2α (Bergmann, et al., 2000 J Virol 74:6203-6206; Gale, et al., 1997,Virology 230(2):217227; Gil, et al., 2006, Virus Res 116(1-2):69-77; Langland, et al., 2002, Virology 299(1):133-141), the examples of perturbation of PERK activation per se are rare (He, 2006, Cell Death Differ 13(3):393-403).

During the initial rounds of infection, ligand-independent degradation of IFNAR1 could be of particular importance for a virus that has penetrated a naïve cell and started to produce massive amounts of viral proteins to prepare for replication. At this time, activation of ER-triggered IFNAR1 degron phosphorylation and ensuing degradation is expected to dramatically reduce the sensitivity of an infected cell to either exogenous or endogenously produced and secreted IFN-α/β (as seen in FIGS. 6-7). It is believed that such alterations benefit the offending virus in at least two ways. First, accelerated degradation of IFNAR1 prevents an efficient induction of expression and activities of diverse anti-viral proteins (including 2′-5′ oligoadenylate synthetases, the Mx proteins, PKR, and the double-stranded-RNA-specific adenosine deaminase) that are known to suppress various steps of viral replication (reviewed in (Guidotti et al., 2001, Annu Rev Immunol 19:65-91)). Second, downregulation of IFNAR1 protects the host cell from the proapoptotic effects of IFN (Chawla-Sarkar, et al, 2002, J Immunol 169(2):847-855; Chawla-Sarkar, et al., 2002, J Interferon Cytokine Res 22(5):603-613), and, therefore, affords the virus a sufficient time for completion of its infectious cycle. Regardless of which of these pathways are more relevant for each specific virus, the results presented herein strongly suggests that UPR-mediated downregulation of Type I IFN receptor and its signaling are important for unabated completion of initial rounds of infection when majority of target host cells are yet to encounter IFN.

Although such a mechanism is believed to briefly benefit a virus that has already entered the cell, it cannot be expected to persist for a protracted period of time or to ensure that progeny released from this infected cell will have a better chance of infecting additional host cells. In order to properly synthesize their proteins, viruses have to attenuate the UPR responses, which they are indeed known to do using a plethora of diverse mechanisms (reviewed in He, 2006, Cell Death Differ 13(3):393-403; Schroder, et al., 2006, Curr Mol Med 6(1):5-36). Once ER stress is resolved, the PERK-dependent pathway that facilitate turnover of IFNAR1 is suspended disabling a described general mechanism for impeding IFN signaling. Under these conditions, viruses have to resort to individual tricks to maintain a degree of virulence in the environment containing IFN-α/β. Such mechanisms (including prevention of microorganism-associated pattern recognition, reduced synthesis and secretion of IFN, inhibition of the activity of regulatory kinases, etc) have been indeed widely reported (reviewed in Katze, et al., 2002, Nat Rev Immunol 2(9):675-687). These mechanisms are of importance for viral replication and subsequent transmission; they contribute to the forces that drive co-evolution of the pathogen and the mammalian host.

However, from the practical point of view of the host, interfering with a non-specific yet important mechanism enabling initial steps of infection represents an attractive strategy toward either preventing viral infectious diseases or directing the development of these diseases toward an abortive course. Based on data presented herein, inhibitors of PERK-dependent phosphorylation of IFNAR1 represent a potent anti-viral activity. As Type I IFN also plays an important immunomodulatory role (Tompkins, 1999, J Interferon Cytokine Res 19(8):817-828), it is believed that the effects of inhibitors of PERK-dependent phosphorylation of IFNAR1 is even more pronounced in vivo. The inhibitors can be useful in treatment of patients with chronic viral infections (e.g., hepatitis C), multiple sclerosis and some malignancies. In cancer patients, the rationale for combining IFN with other anti-tumor agents that cause UPR (for example, proteasome inhibitors (Fribley, et al., 2004, Mol Cell Biol 24(22):9695-9704; Nawrocki, et al., 2005, Cancer Res 65(24):11510-11519; Obeng, et al., 2006, Blood 107(12):4907-4916)) might be re-evaluated, design of the means that would impede HCV-mediated ER stress and ensuing degradation of IFNAR1 (e.g., inhibitors of PERK-dependent pathway) is expected to benefit the patients whose therapeutic regiment includes IFN-α.

### Example 2

### Specific Inhibition of PTPIB to Augment the Efficacy of Endogenous and Pharmaceutical Type I IFN

Type I interferons (IFN) including diverse types of IFNα and IFNI3 are endogenously produced cytokine proteins that possess potent anti-tumor, anti-viral and immunomodulatory activities. IFNs are being produced industrially; currently, several formulations have been developed and approved by FDA including Roferon-A (Roche US Pharmaceutical), Pegasys (Hoffmann-La Roche Inc.); Intron-A, Rebetron, Peg-Intron (Schering Plough Corporation), Alferon-N (Hemispherx Biopharma, Inc), Avonex (Biogen IDEC), Betaseron (Bayer Healthcare Pharmaceuticals Inc), and Infergen (Amgen, Inc). These modalities are often used in treatment of various cancers (e.g., leukemias and malignant melanoma), viral infections (e.g., hepatitis C) and autoimmune diseases (e.g., multiple sclerosis).

All effects of IFNs in cells are mediated through a single Type I IFN receptor that consists of IFNAR1 and IFNAR2 chains. Similar to other cytokines, interaction of IFNs with their receptor leads to activation of Janus kinases (JAKS), JAKs mediate tyrosine phosphorylation of signal transducers and activators of transcription (STATs) who then form a potent transcription factor and transactivate a number of IFN-stimulated genes (ISGs) that confer the functions of IFNs. This signal transduction cascade is under tight control of several layers of negative regulation that could be ligand specific (i.e., leads to suppression of only Type I IFNs signaling—that is receptor downregulation) or shared with other cytokines (for example, interferon gamma, growth hormone, interleukin 6, etc). Nonspecific mechanisms include: (i) induction of Shp1/2 protein tyrosine phosphatases (PTP) that remove phospho-groups from JAKs and STATs, (ii) induction of SOCS proteins that inhibit and degrade JAKs; and (iii) induction of PIAS proteins that inhibit STAT transcriptional activities. Inhibitors of non-specific regulators (e.g., inhibitors of phosphatases Shp1/2) are therefore expected to display substantial toxicity because they would be affecting numerous physiologic processes (e.g., hematopoiesis).

Although potent, IFNs as drugs pose a number of problems. One is the cost of treatment (that goes beyond $30,000 per course per melanoma patient) and lack of orally available agents as it is generally more expensive to produce and more difficult to deliver protein than a small molecule. Another is development of anti-IFN antibodies that, in addition to many other mechanisms contribute to limiting the efficacy of IFN therapy. Down regulation of IFNAR1 (its disappearance from cell surface) was shown to be a pivotal regulator of the extent of cellular responses to IFN.

It has been observed that inhibition of PTP1B can be accomplished using, but not limited to, genetic (e.g., RNAi) and pharmacologic approaches (known inhibitors of PTP1B). The inhibitors of PTP1B is believed to be useful as anti-viral agents per se or used in the context of augmenting the effect of administered IFN-based drug.

It has been demonstrated that lysosomal degradation of IFNAR1 requires its ubiquitination (Kumar et al., 2003 Embo J 22:5480-90). Further studies delineated the mechanisms by which IFNAR1 ubiquitination promotes endocytosis of this receptor. These mechanisms involve the unmasking of the Tyr466-based linear endocytic motif within the intracellular domain of IFNAR1 (Kumar et al., 2007 J Cell Biol 179:935-50). In human cells that are not treated with IFN-a/13, this motif is masked by interaction with Tyk2 kinase that prevents ligand-independent endocytosis of IFNAR1 (Kumar et al., 2008 J Biol Chem 283:18566-72). The importance of this Tyr-based motif is underscored by observations that Tyk2 deficiency in human cells results in almost complete loss of cell surface IFNAR1 (Ragimbeau et al., 2003 Embo J 22:537-47) while this phenomenon is not observed in Tyk2-deficient mouse cells (Karaghiosoff et al., 2000, Immunity 13: 549-60) whose IFNAR1 lacks Tyr-based endocytic motif (Table 1).

TABLE 1
Conservation of Tyr-based endocytic motif
(YXXφ bold font) in the proximal parts of the
intracellular domains of IFNAR1
proteins from different species

| | |
|---|---|
| H. Sapiens: | FLRCINYVFFPSLKPSSSIDEYFSEQPLK |
| | NLLLSTSEEQIEKCFII |
| | (SEQ ID NO: 4) |
| P. troglodyte: | FLRCINYVFFPSLKPSSNIDEYFSEQPLK |
| | NLLLSTSEEQIEKCFII |
| | (SEQ ID NO: 5) |
| P. anubus: | LLRCINYVFFPSLKPSSNIDEYFSEQSLK |
| | NLLLSTSEEQIEKCLII |
| | (SEQ ID NO: 6) |
| M. fascicularis: | LLRCINYVFFPSLKPSSNIDEYFSEQSLK |
| | NLLLSTSEEQIEKCLII |
| | (SEQ ID NO: 7) |
| B. taurus: | FLRCVKYVFFPSSKPPSSVDEYFSDQPLP |
| | NLLLSTSEEQTERCFII |
| | (SEQ ID NO: 8) |
| E. caballus | LWRCINYVFFPSSKPPSAVDEYFSEHLLK |
| | NLLLSTSAEQIERCEVI |
| | (SEQ ID NO: 9) |
| O. aries | FLRCVKYVFFPSSKPPSSVDQYFSDQPLP |
| | NLLLSTSEEQTERCFII |
| | (SEQ ID NO: 10) |
| S. scrofa | VSRCINYVFFPSSKPPSTIDEYFAEQPLK |
| | NLLLSTSEEQTEICFIV |
| | (SEQ ID NO: 11) |
| C. familiaris | LLRCISYVFFPSSKPPSTIDEYFSEPPLW |
| | NLLLLTSEEQTERCFII |
| | (SEQ ID NO: 12) |
| R. norvegicus | SLQKYLYYVFSPPLKPPCSIDEFFSELPS |
| | KSLLLLTAEEHTERCFV |
| | (SEQ ID NO: 13) |
| M. musculus | VWKYLGHVCFPPLKPPRSIDEFFSEPPSK |
| | NLVLLTAEEHTERCFII |
| | (SEQ ID NO: 14) |
| G. gallus | VYNKIKYMFFPSCQTPLNIEGFGAQLFSS |
| | PFVPTVFFPVEICYIIE |
| | (SEQ ID NO: 15) |

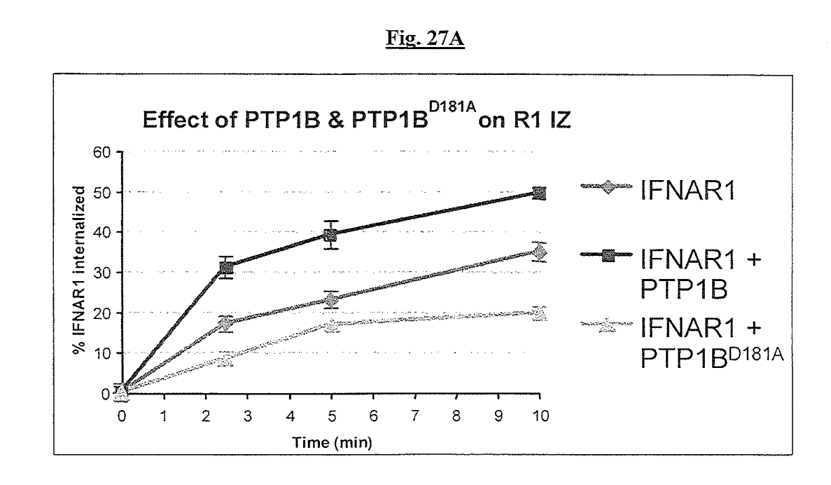

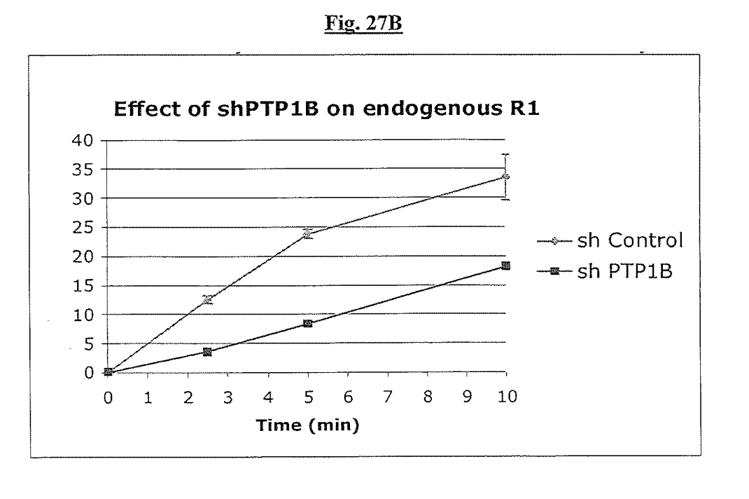

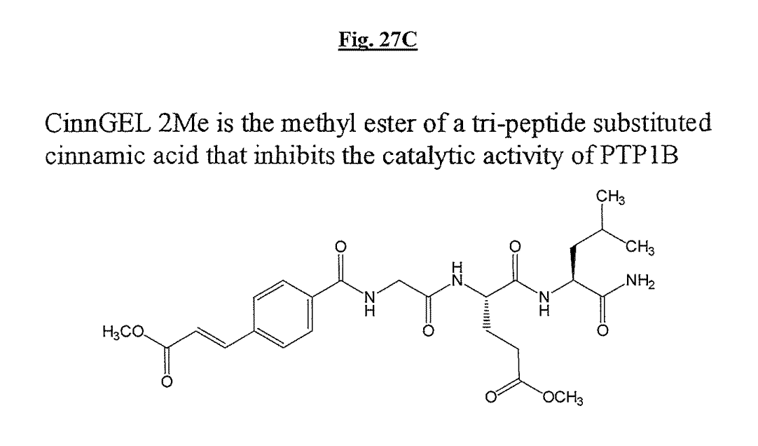

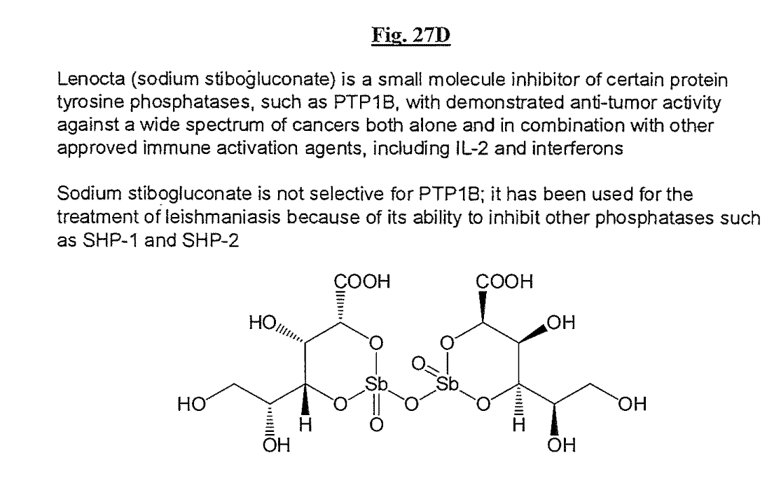

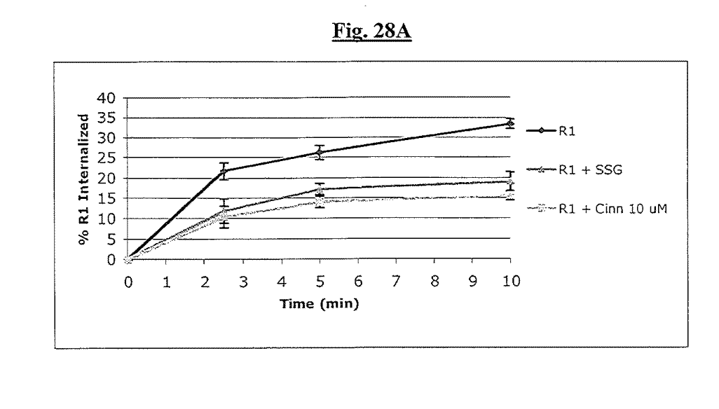

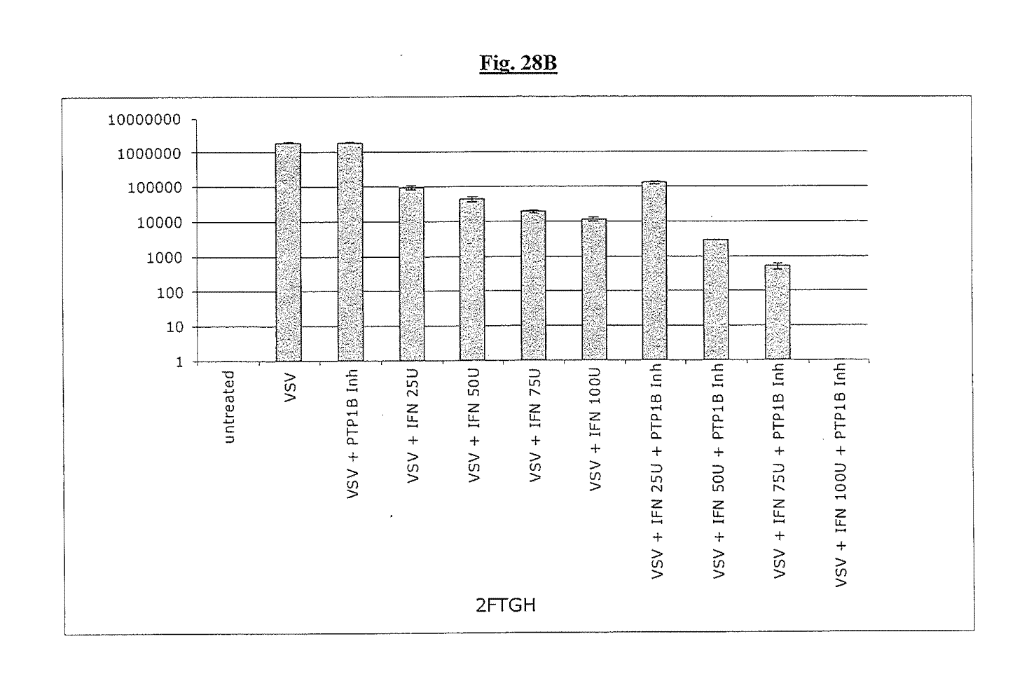

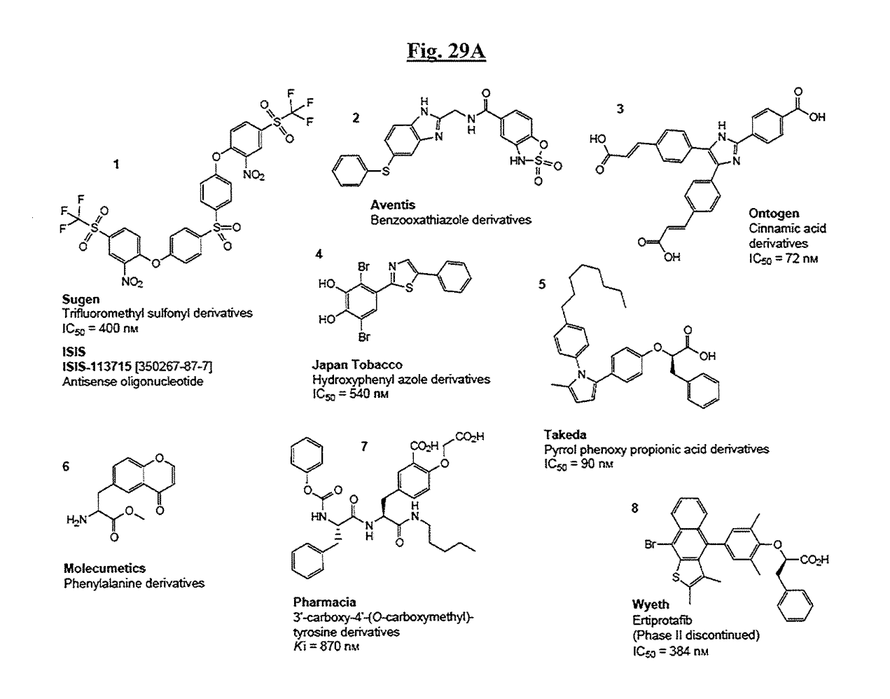

Upon IFN-α/β treatment, IFNAR1 undergoes ubiquitination that unmasks Tyr466 and allows it to interact with AP2 endocytic machinery complex leading to IFNAR1 endocytosis and subsequent lysosomal degradation of this receptor (Kumar et al., 2008 J Biol Chem 283:18566-72). Tyr-based linear endocytic motifs are known to serve as a recognition site for the AP50 subunit of the AP2 complex (Bonifacino et al., 2003 Annu Rev Biochem 72:395-447). Intriguingly, the fact that, in response to IFN-α, this Tyr466 also undergoes phosphorylation by Janus kinases (Yan et al., 1996 Embo J 15:1064-74) may add another level of regulation complexity. Phosphorylation of Y466 is expected to reduce affinity of AP50 for the Tyr-based endocytic motifs described for CTLA-4 (Chuang et al., 1997 J Immunol 159:144-51; Shiratori et al., 1997 Immunity 6: 583-9; Zhang et al., 1997 Proc Natl Acad Sci USA 94:9273-8). Thus, it is possible that tyrosine phosphatase activities play a role in regulating IFNAR1 endocytosis. Data presented herein demonstrate that protein tyrosine phosphatase 1B (known to interact with Tyk2, (Myers et al., 2001 J Biol Chem 276:47771-4)) plays an important role in regulating the endocytosis of IFNAR1 and its ability to mediate anti-viral effects of IFN-α/β (FIG. 27).

The data presented herein demonstrates a novel endocytic mechanism that governs the downregulation of the IFNAR1 chain of IFN receptor. Remarkably, this downregulation depends on specific de-phosphorylation of a specific Tyr residue within IFNAR1. This residue is present in most of known mammalian IFNAR1 sequences except in mouse IFNAR1. Dephosphorylation of this Tyr is mediated by PTP1B-a tyrosine phosphatase that is an attractive target for treatment of diabetes and obesity against which selective inhibitors are being developed. The results presented herein demonstrate that selective inhibition of PTP1B prevents IFNAR1 endocytosis and augments IFN responses (including anti-viral defense) in human but not mouse cells.

Selective inhibitors of PTP1B (that do not affect other phosphatases such as Shp) in order to stabilize IFNAR1 and the entire receptor on cell surface is useful in IFN therapy. This stabilization leads to an augmented response to IFN, which, in turn, translates into either a higher efficacy of endogenous IFN or into an enhancement of efficacy of an IFN-based in treatment the diseases including, but not limited to, cancer, multiple sclerosis and viral infections.

Use of existing and novel inhibitors of PTP1B as sole agents in treatment of diseases that are treated by Type I IFN is envisioned. In addition, these agents can be used in treatment of such diseases in combination with Type I IFN in order to (i) increase its efficacy (ii) decrease the dose and related development of anti-IFN antibody-dependent resistance and side effects, as well as decrease the costs and duration of treatment.

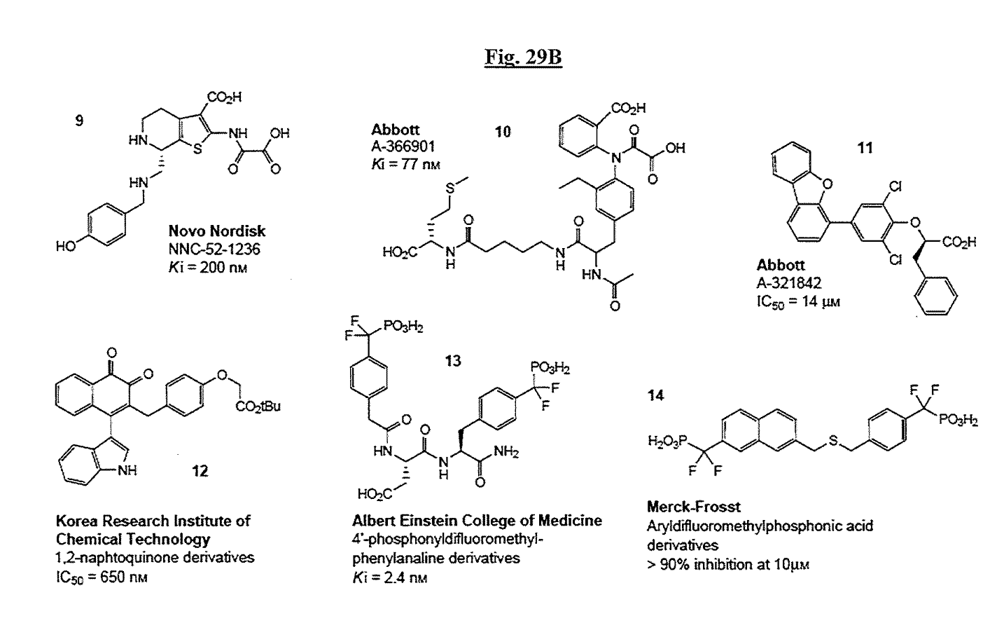

Use of selective PTP1B inhibitors will allow to augment the efficacy of Type I IFN and to decrease toxicity that is posed by less specific inhibitors (such as Sodium stibogluconate). FIG. 29 depicts a list of PTP1B inhibitors being developed by pharmaceutical companies for treatment of diabetes and obesity (as reviewed in (Hooft van Huijsduijnen et al., 2004 J. Med Chem 47:4142-6; Hooft van Huijsduijnen et al., 2002 Drug Discov Today 7:1013-9)). These and additional inhibitors such as Trodusquemine (Geneara) and [(3-bromo-7-cyano-2-naphthyl)(difluoro)-methyl]phosphonic acid Merck) could be further used.

### Example 3

### Specific Inhibition of Protein Kinase D2 (PKD2) to Augment the Efficacy of Endogenous and Pharmaceutical Type I IFN

Type I interferons (IFN) including diverse types of IFNα and IFNβ are endogenously produced cytokine proteins that possess potent anti-tumor, anti-viral and immunomodulatory activities. This is why IFNs are being produced industrially; currently, several formulations have been developed and approved by FDA including Roferon-A (Roche US Pharmaceutical), Pegasys (Hoffmann-La Roche Inc.); Intron-A, Rebetron, Peg-Intron (Schering Plough Corporation), Alferon-N (Hemispherx Biopharma, Inc), Avonex (Biogen IDEC), Betaseron (Bayer Healthcare Pharmaceuticals Inc), and Infergen (Amgen, Inc). These modalities are often used in treatment of various cancers (e.g., leukemias and malignant melanoma), viral infections (e.g., hepatitis C) and autoimmune diseases (e.g., multiple sclerosis).

All effects of IFNs in cells are mediated through a single Type I IFN receptor that consists of IFNAR1 and IFNAR2 chains. Similar to other cytokines, interaction of IFNs with their receptor leads to activation of Janus kinases (JAKs). JAKs mediate tyrosine phosphorylation of signal transducers and activators of transcription (STATs) who then form a potent transcription factor and transactivate a number of IFN-stimulated genes (ISGs) that confer the functions of IFNs. This signal transduction cascade is under tight control of several layers of negative regulation that could be ligand specific (i.e., leads to suppression of only Type I IFNs signaling—that is receptor downregulation) or shared with other cytokines (for example, interferon gamma, growth hormone, interleukin 6, etc). Nonspecific mechanisms include: (i) induction of Shp1/2 protein tyrosine phosphatases (PTP) that remove phospho-groups from JAKs and STATs, (ii) induction of SOCS proteins that inhibit and degrade JAKs; and (iii) induction of PIAS proteins that inhibit STAT transcriptional activities. Although potent, IFNs as drugs pose a number of problems. One is the cost of treatment (that goes beyond $30,000 per course per melanoma patient) and lack of orally available agents as it is generally more expensive to produce and more difficult to deliver protein than a small molecule. Another is development of anti-IFN antibodies that, in addition to many other mechanisms contribute to limiting the efficacy of IFN therapy.

Downregulation of IFNAR1 (its disappearance from cell surface) was shown to be a pivotal regulator of the extent of cellular responses to IFN. Research conducted in my lab and other groups over several years delineated the mechanisms of IFNAR1 downregulation, IFN stimulates a protein kinase that phosphorylates IFNAR1 on specific serine residues (Ser535 and Ser539 in human IFNAR1). This phosphorylation enables the recruitment of the beta-TrCP E3 ubiquitin ligase that ubiquitinates IFNAR1 (Kumar, et al., 2003, Embo J 22:5480-5490). This ubiquitination promotes exposure of a linear Tyr based endocytic motif within IFNAR1 that mediates internalization of this receptor followed by its lysosomal degradation (Kumar, et al., 2007, J Cell Biol 179:935-950).

Phosphorylation of IFNAR1 by an IFN-inducible kinase plays a critical role in modulating cellular responses to IFN. Studies using IFNAR1 mutant that cannot be phosphorylated demonstrated that this phosphorylation is critical for IFN signaling in general and efficacy of IFN in growth inhibition of human cancer cells in particular (Kumar, et al., 2003, Embo J 22:5480-5490). Human melanoma cells harboring this mutant exhibit a substantial delay in growth in vivo in xenograft mouse model (Kumar, et al., 2007, Cancer Biol Ther 6:1437-1441). These studies demonstrate that inhibition of IFNAR1 kinase is a way to increase the efficacy of Type I IFN.

Protein kinase D2 (PKD2), a member of PKD family (consisting of PKID1, PKD2 and PKD3), is an IFNAR1 kinase that regulates IFNAR1 phosphorylation, ubiquitination, abundance and signaling. It has been observed that PKD2 can be inhibited using genetic (RNAi) and pharmacologic approaches (known inhibitors of PKD that are non-specific). Specific PKD2 inhibitors are believed to have anti-viral/anti-tumor/immunomodulatory effects per se or ability to augment the effect of administered IFN-based drug. Accordingly, the invention encompasses compositions and methods relating to the use of selective inhibitors of PKD2 in order to stabilize IFNAR1 and the entire Type I IFN receptor. This stabilization leads to an augmented response to IFN, which, in turn, translates into either a higher efficacy of endogenous IFN or into an enhancement of efficacy of an IFN-based in treatment the diseases including (but not limited to) cancer, multiple sclerosis and viral infections.

It has been demonstrated that IFN-α/β stimulate phosphorylation of Ser535 within human IFNAR1 (Ser526 in mouse receptor) to promote its interaction with the βTrcp E3 ubiquitin ligase. This ligase stimulates IFNAR1 ubiquitination that leads to endocytosis of IFNAR1 and subsequent lysosomal degradation of IFNAR1. The latter decreases the sensitivity of cells to IFN-α/β (Kumar et al., 2003 Embo J, 22:5480-90). The data presented herein identify protein kinase D2 (PKD2, a.k.a. Prkd2) as a key kinase activated by IFN-α/β and promoting IFNAR1 phosphorylation. These data also show that inhibition of PKD2 promotes the signaling and anti-viral effects of IFN-α/β.

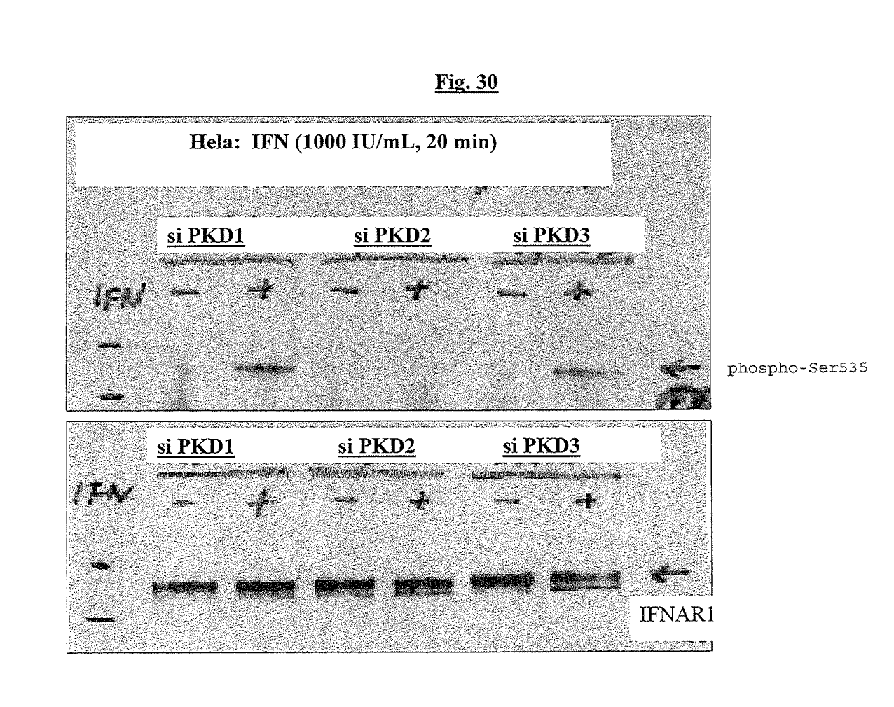

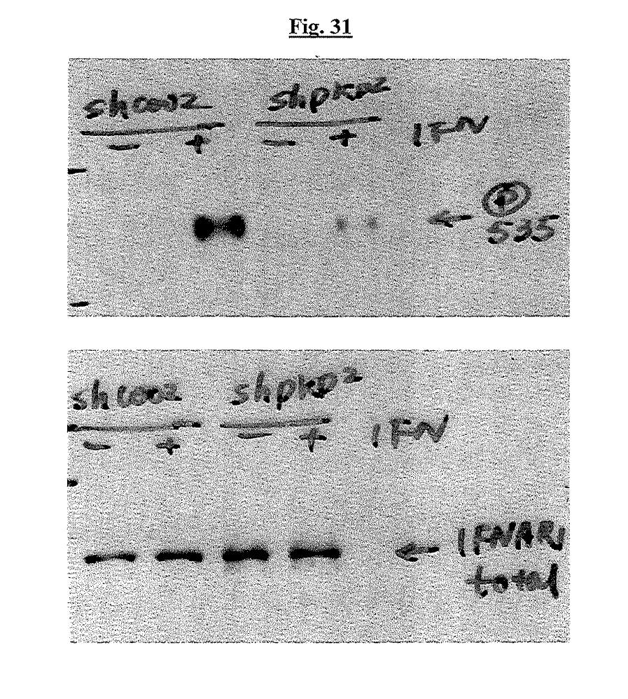

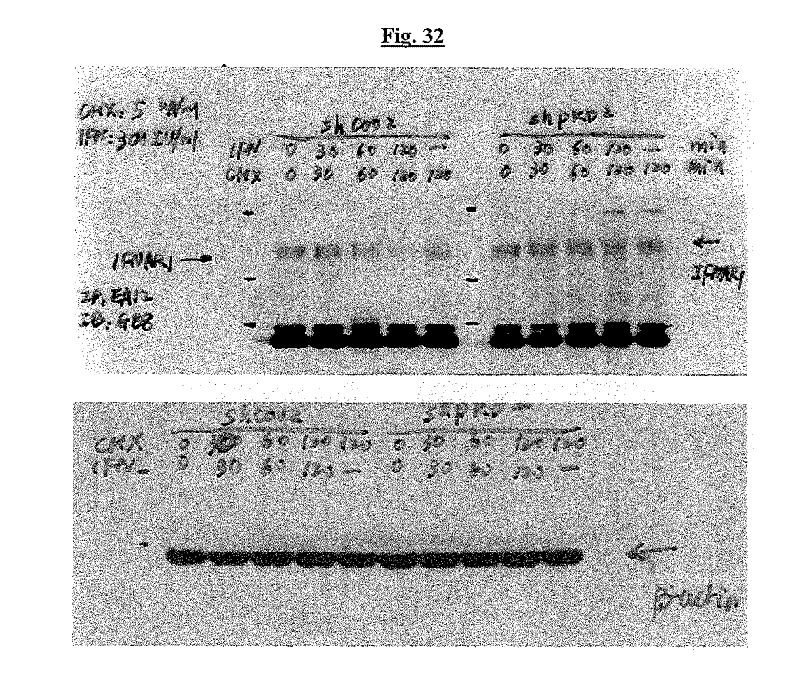

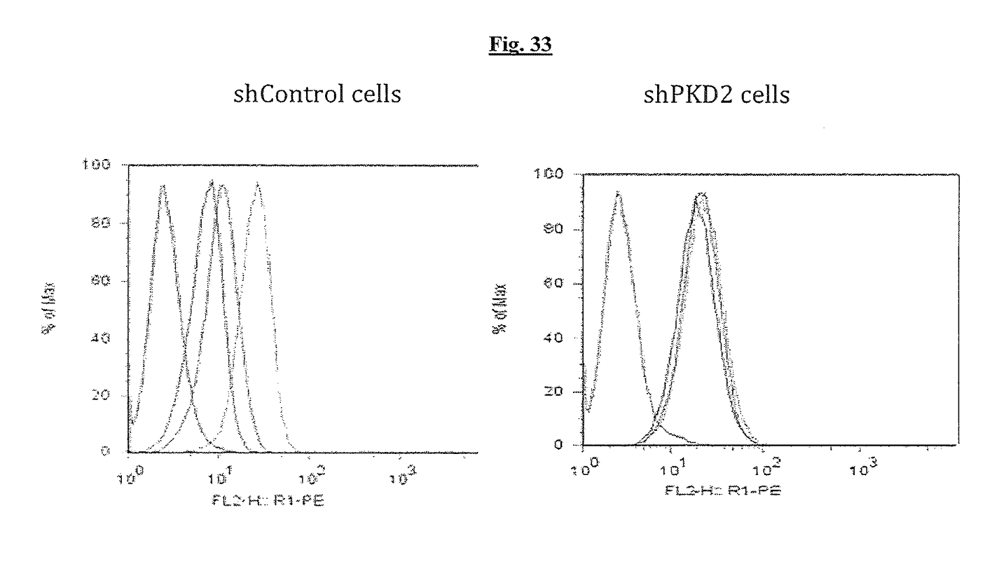

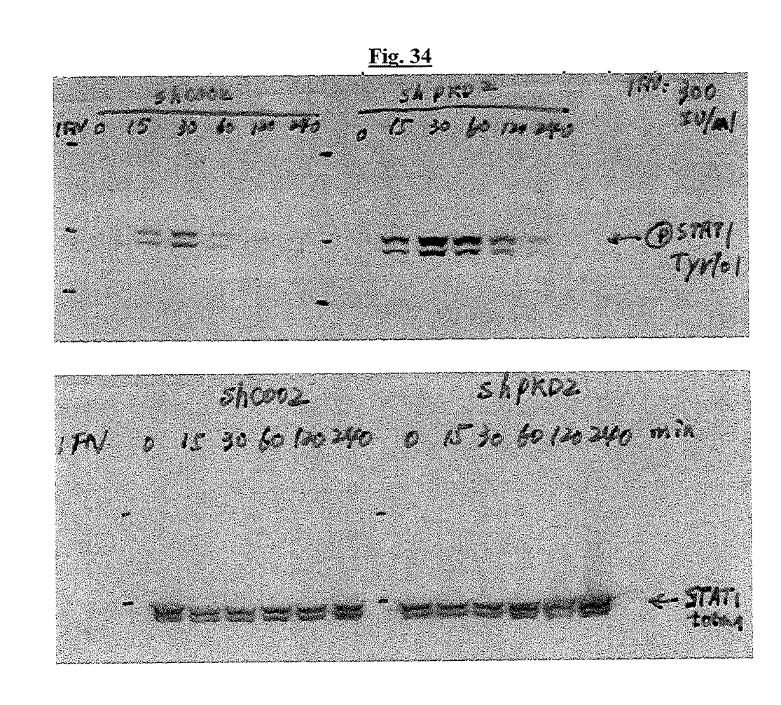

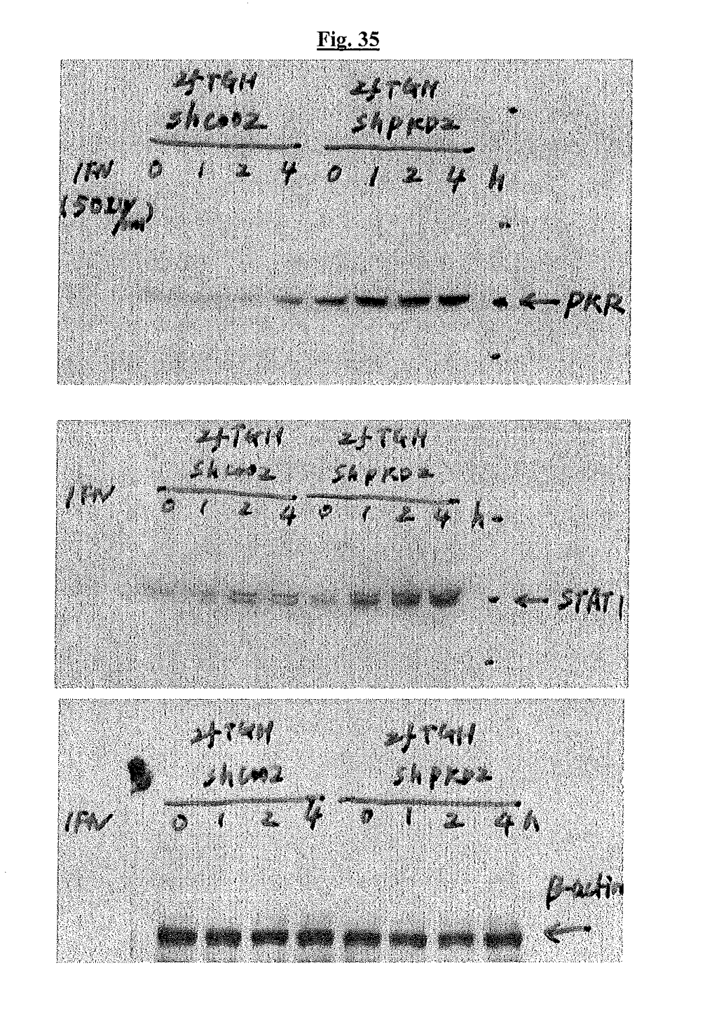

It was observed that inhibition of PKD2 (but not related kinases PKD1 or PKD3) by siRNA prevented phosphorylation of IFNAR1 on Ser535 in HeLa cells treated with IFN-α. Phosphorylation was measured by immunoblot using phospho-specific anti-Ser535 antibody (described in Kumar et al., 2004 J Biol Chem 279:46614-20). It was also observed that PKD2 is required for ligand-induced phosphorylation of IFNAR1 degron (FIG. 30). Stable knockdown of PKD2 in HeLa cells inhibited IFNAR1Ser535 phosphorylation in response to IFN-α (FIG. 31). It was observed that knockdown of PKD2 prevents IFN-α-stimulated degradation of IFNAR1 (FIG. 32). It was observed that knockdown of PKD2 prevented downregulation of cell surface levels of IFNAR1. In addition, it was observed that knockdown of PKD2 in HeLa cells stimulated IFN-a signaling measured by activating tyrosine phosphorylation of Stat1 protein (FIG. 34).

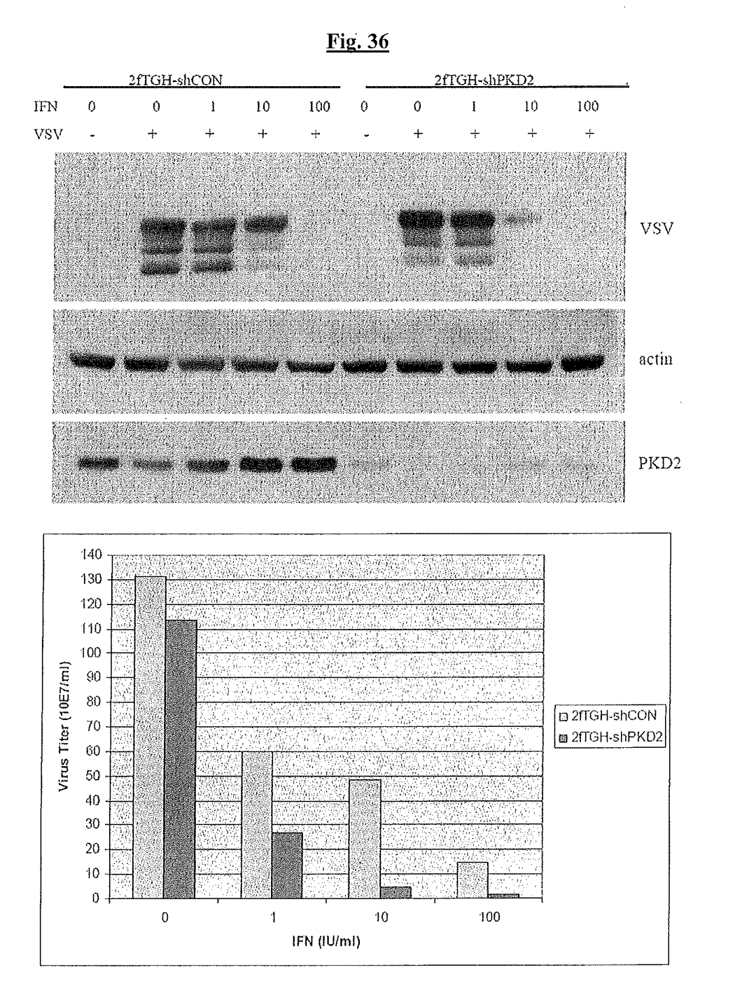

The next set of experiments were designed to study the downstream signaling mediated by interferon alpha and IFNAR1 in 2fTGH-shC0002 and 2fTGH-shPKD2 cells. It was observed that knockdown of PKD2 in human 2fTGH cells stimulated expression of interferon-inducible genes such as PKR and Stat1 (FIG. 35). It was also observed that knockdown of PKD2 in human 2fTGH cells increased the efficacy of IFN-α against infection with vesicular stomatitis virus (VSV) (FIG. 36).

Use of PKD2 inhibitors as sole agents in treatment of diseases that are treated by Type I IFN is envisioned. In addition, these agents can be used in treatment of such diseases in combination with Type I IFN in order to (i) increase its efficacy (ii) decrease the dose and related development of anti-IFN antibody-dependent resistance and side effects, as well as decrease the costs and duration of treatment. Use of selective PKD2 inhibitors allows for the specific augmentation of the efficacy of Type I IFN and to be used not only against cancers but also against viral infections and autoimmune diseases.

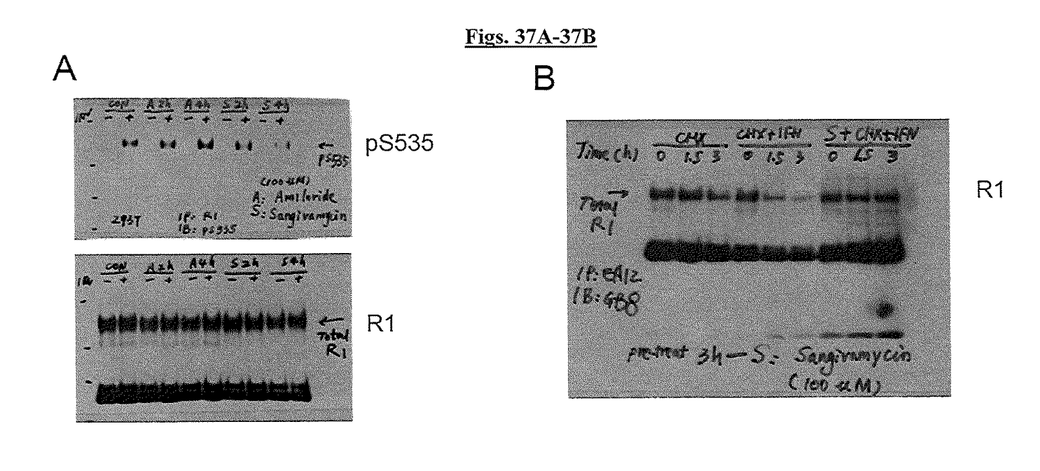

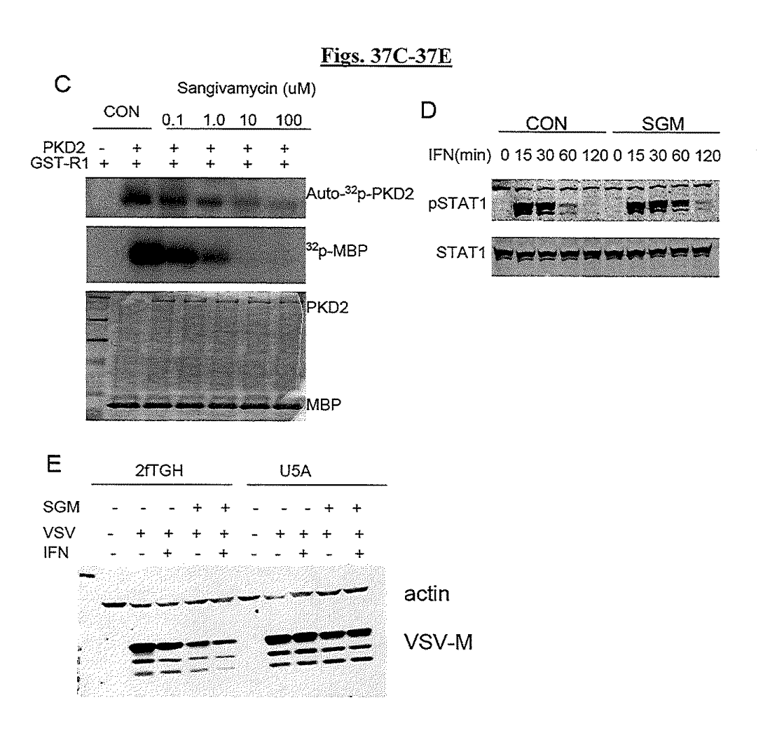

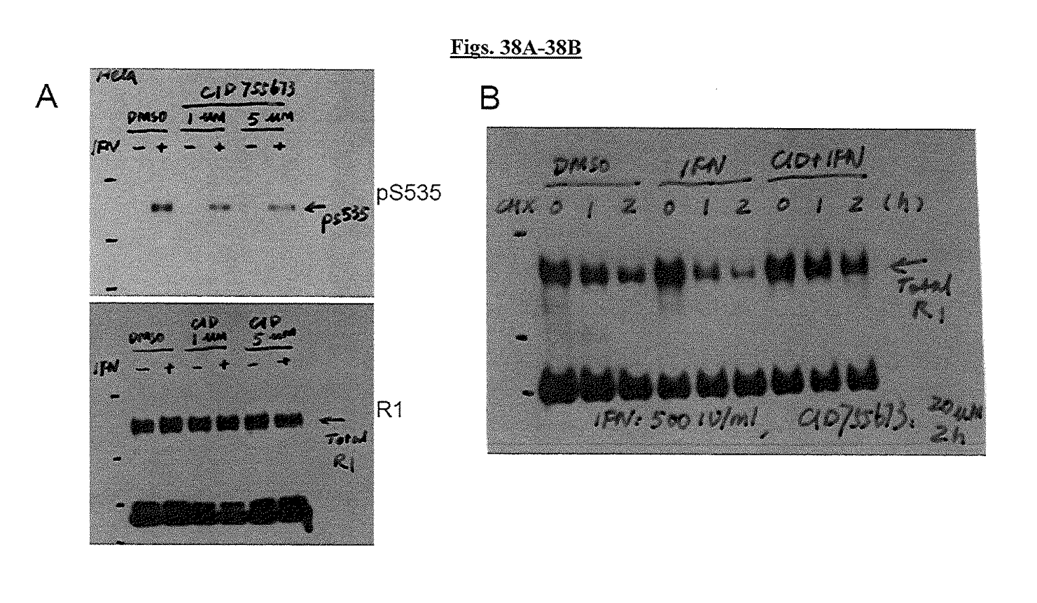

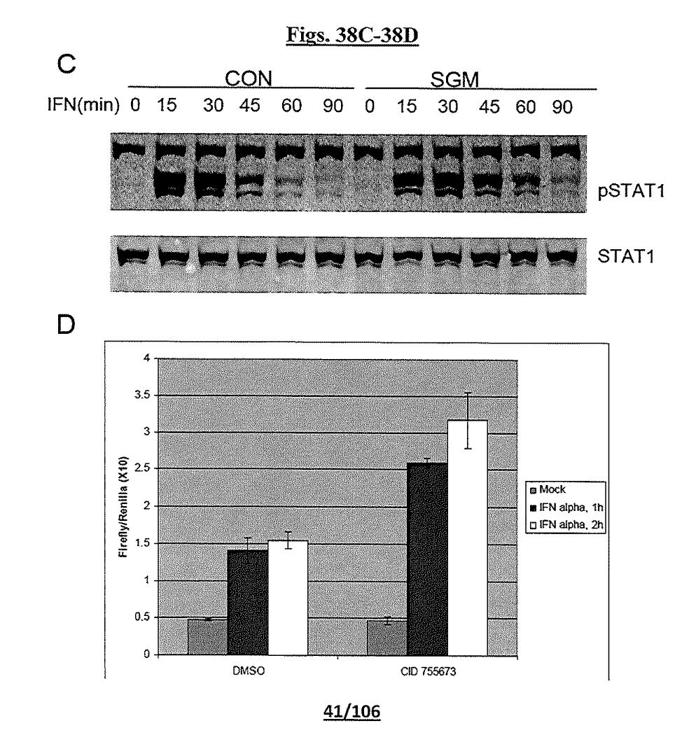

One example is depicted in FIGS. 37 and 38, FIGS. 37 and 38 depict the results of example experiments demonstrating that sangivamycin inhibits PKD2 and increases the efficiency of IFN signaling.

### Example 4

### Role of Protein Kinase D2 Proteolytic Elimination of Interferon Alpha Receptor

Extracellular ligands induce signaling pathways that mediate their functions but also limit them by the proteolytic elimination of cognate receptors. As disclosed herein, protein kinase D2 (PKD2) controls the ligand-inducible phosphorylation-dependent ubiquitination and degradation of the IFNAR1 chain of the Type I interferon (IFN) receptor. IFN-a induces PKD2 in a Tyk2-activity- and tyrosine phosphorylation-dependent manner. Activated PKD2 directly phosphorylates key serine residues within the degron of IFNARIleading to recruitment of the 13-Trcp-based E3 ubiquitin ligase, and ubiquitination and degradation of IFNAR1. Inhibition or knockdown of PKD2 augments IFN-a signaling and anti-viral defenses. PKD2-mediated phosphorylation and ubiquitination of IFNAR1 is also induced by vascular endothelial growth factor (VEGF); the ability of VEGF to induce efficient angiogenesis depends on IFNAR1 degradation. The mechanisms of ligand-inducible elimination of IFNAR1 and utilization of these mechanisms by other stimuli to counteract the biological functions of Type I IFN are disclosed along with potential medical significance of this regulation.

As disclosed herein, protein kinase D2 (PKD2) is a Type I IFN-inducible kinase that can be activated via tyrosine phosphorylation and, in turn, is capable of phosphorylating the serines within the degron of IFNAR1. PKD2 expression and activity are important for regulating ubiquitination and degradation of IFNAR1 and for control of the extent of IFN-α signaling and anti-viral defenses. The data suggest that this mode of regulation could be commandeered by some inducers of unrelated signaling pathways capable of activating PKD2 such as vascular endothelial growth factor (VEGF). VEGF promotes IFNAR1 phosphorylation and accelerates IFNAR1 proteolytic turnover, which is required for efficient angiogenesis.

The Materials and Methods used in the experiments presented in this Example are now described.

### Plasmids, Cells and Viruses

Vectors for mammalian expression of Flag-IFNAR1 and bacterial expression of GST-IFNAR1 (Kumar et al., 2003, Embo J 22:5480-5490), J3-Trcp2/HOS (Fuchs et al., 1999, Oncogene 18:2039-2046), and HA-tagged Tyk2 (Yan et al., 1996, Mol Cell Biol 16:2074-2082), as well as the 5xISRE-luciferase reporter (Parisien et al., 2002, J Virol 76:4190-4198)) have been described elsewhere. Vectors for mammalian expression of human GST-tagged PKD1-3 species (wild type or kinase-dead mutants) have been described elsewhere (Yeaman et al., 2004, Nat Cell Biol 6:106-112). Silent mutations, as well as replacement of Y438 with tyrosine were generated by site-directed mutagenesis. SiRNA and shRNA reagents were purchased from Sigma and Qiagen.

Human embryo kidney 293T cells and epithelial HeLa cells were maintained and transfected as described elsewhere (Liu et al., 2009, Cell Host Microbe 5(1):72-83). Human fibrosarcoma 2fTGH cells and their Stat1-deficient U3A derivatives (McKendry et al., 1991, Proc Natl Acad Sci USA 88:11455-11459) or Tyk2-deficient 11.1 derivatives (reconstituted with wild type or kinases dead Tyk2 have been described elsewhere (Gauzzi et al., 1997, Proc Natl Acad Sci USA 94:11839-11844). The anti-viral effect of IFN-a against VSV was determined as described elsewhere (Sharma et al., 2003, Science 300:1148-1151).

### Phosphorylation-Binding and Kinase Assays

For some binding assays, the lysates from 293T cells (treated for various tunes with IFN-α, 2000 IU/mL) were depleted of CK1α (as described previously (Liu, et al., 2009, Mal Cell Biol 29(24):6401-12) were incubated with GST-IFNAR1 proteins (wild type or S535,539A mutant that migrates slower on SDS-PAGE due to the presence of four additional amino acids in the linker), ATP and kinase inhibitors (as indicated) for 30 min at 30° C. After that, GST-IFNAR1 were purified and incubated with in vitro translated and 35S-methionine-labeled 3-Trcp2 for 1 h at 4° C.; this binding was later analyzed by autoradiography and Coomassie staining. In vitro serine phosphorylation of GST-IFNAR1 (analyzed by autoradiography or IB with pS535 antibody) by cell extracts or PKD preparations and tyrosine phosphorylation of GST-PKD2 by Tyk2 was carried out and analyzed as described elsewhere herein.

For other binding assays, the lysates from 293T cells (treated for various times with IFN-α, 2000 IU/mL) underwent two rounds of immunodepletion of CK1α as described elsewhere (Liu, et al., 2009, Mol Cell Biol 29(24):6401-12). GST-IFNAR1 proteins (wild type or S535,539A mutant that migrates slower on SDS-PAGE due to the presence of four additional amino acids in the linker (Liu et al., 2009, Cell Host Microbe 5(1):72-83)) were expressed and purified from bacterial cells using glutathione Sepharose. Purified bacterial proteins (2 μg) were incubated in the presence of CK1α-depleted cell lysates (5 μg), unlabeled ATP (0.5 mM) in total volume of 20 μL (containing 25 mM Tris-HCL pH 7.5, 5 mM MgCl2, 100 mM KCl, 1 mM EGTA, 1 mM Na3VO4, 0.1 mM DTT and 3.5% glycerol) for 30 min at 30° C. When indicated, the following kinase inhibitors were added to the reaction in I pi, of DMSO to the final concentration indicated: H89 (200 nM), BAY43-9006 (50 nM), Bis-I (25 nM), Gö6976 (25 nM), LY294002 (1.5 μM), SP600125 (100 nM), D4476 (400 nM), SB203580 (100 nM). The reactions were stopped by placing the tubes on ice and adding 150 μL of ice-cold binding buffer (PBS supplemented with 0.1% NP40 and 50 mM NaF) and incubated with glutathione Sepharose beads (20 μL) for 2 h at 4° C. The beads were then washed three times with 0.5 mL of binding buffer and incubated with 2 μL of in vitro translated and 35S-methionine-labeled13-Trcp2 for 1 h at 4° C. Beads were then washed three times with 1 mL of binding buffer, and the proteins were eluted using Laemmli buffer, resolved by SDS-PAGE, and analyzed by autoradiography and Coomassie staining.

In vitro phosphorylation of GST-IFNAR1 by cell extracts or PKD preparations (via immunopurification or GST pulldown) was carried out in kinase buffer (50 mM Tris-HCl pH 7.4, 10 mM MgC12, and 2 mM DTT) with either 0.2 mM of unlabeled ATP or 10 μCi of γ-32P ATP for 20 min at 30° C. The products of this reaction were separated by SDS-PAGE and analyzed either by immunoblotting using anti-pS535 antibody or by autoradiography.

In vitro phosphorylation of PKD2 (purified from HeLa cells) by HA-tagged Tyk2 immunopurified from 293T cells (untreated or treated with IFN-a) or by recombinant Src (purchased from Cell Signaling) was carried out in the total volume of 20 μL in 50 mM MOPS pH 7.4, 10 mM MgCl2, 5 mM MnCl2, 2 mM DTT, and 0.2 mM ATP at 30° C. for 20 min. The samples were resolved by SDS-PAGE and analyzed by immunoblotting with an anti-phospho-tyrosine antibody (4G10).

### Animals

Mice with a S526A substitution within IFNAR1 were generated using previously characterized ES cells carrying the mutation (Liu et al., 2009, Cell Host Microbe 5(0:72-83), ES cells were transduced with Cre-expressing vector to excise the Neo cassette, re-selected and used to generate germline chimeras, which were then crossed with C57/BL6 females to obtain heterozygotes. These clones were microinjected into albino C57/BL6 blastocysts to generate germline chimeras, which were then crossed with C57/BL6 females to obtain heterozygotes. Genotypes of the mice were determined by analyzing DNA with primers annealing to IFNAR1 sequences that would flank the removed neomycin cassette of the mutant allele and that of mutation site. The latter were sequenced to confirm a Ser526 to Ala substitution. Animals were maintained in a specific pathogen-free environment and tested negative for pathogens in routine screening. Matrigel Plug assay (Medhora et al., 2003, Am J Physiol Heart Circ Physiol 284:H215-224) and CD31 immunohistochemistry (Chiodoni et al., 2006, J Exp Med 203:2441-2450) was carried out as described elsewhere.

### Plasmids, Oligos, Cells and Gene Transfer

Vectors for mammalian expression of Flag-IFNAR1 and bacterial expression of GST-IFNAR1 (Kumar et al., 2003, Embo J 22:5480-5490), 3-Trcp2/HOS (Fuchs et al., 1999, Oncogene 18:2039-2046), and HA-tagged Tyk2 (Yan et al., 1996, Mol Cell Biol 16:2074-2082), as well as the 5xISRE-luciferase reporter (Parisien et al., 2002, J Virol 76:4190-4198) have been described elsewhere. Vectors for mammalian expression of human GST-tagged PKD1-3 species (wild type or kinase-dead mutants) have been described elsewhere (Yeaman et al., 2004, Nat Cell Biol 6:106-112). Silent mutations, as well as replacement of Y438 with tyrosine were generated by site-directed mutagenesis. All resulting mutants were verified by dideoxy sequencing. ShRNA against PKD2 constructs based on pLK0.1-puto were purchased from Sigma (MISSION shRNA, SHGLY-NM_{—}016457). Control shRNA and siRNA were targeted against GFP (Jin et al., 2003, Genes Dev 17:3062-3074) and luciferase (Kumar et al., 2003, Embo J 22:5480-5490), respectively. SiRNA oligos including siPKD1 (Hs_PRKCM_{—}2_HP Validated siRNA, SI00301350), siPKD2 (Hs_PRKD2_{—}5_HP Validated siRNA, SIO2224768), siPKD3 (Hs_PRKCN_{—}1_HP Validated siRNA, SI00301357) were purchased from QIAGEN and transfected into cells using the HiPerFect transfection reagent (QIAGEN).

Human embryo kidney 293T cells and epithelial HeLa cells were maintained and transfected as described elsewhere (Liu et al., 2009, Cell Host Microbe 5:72-83). Human fibrosarcoma 2fTGH cells and their Stat1-deficient U3A derivatives (McKendry et al., 1991, Proc Natl Acad Sci USA 88:11455-11459) or Tyk2-deficient 11.1 derivatives (reconstituted with wild type or kinases dead Tyk2 have been described elsewhere (Gauzzi et al., 1997, Proc Natl Acad Sci USA 94:11839-11844). All these cells were maintained in Dulbecco's modified Eagle's medium (DMEM) supplemented with 10% (v/v) fetal bovine serum (Hyclone). 11. 1-derivatives also received G418 (400 μg/ml). Human umbilical vein endothelial cells were a gift. These cells were maintained in Vasculife endothelial cell culture medium (LIFELINE Cell Technology, Inc). Transient transfections of 293T cells or 2fTGH and their derivatives using LIPOfectamine Plus (Invitrogen) and of HeLa cells using LIPOfectamine-2000 (Invitrogen) were carried out according to manufacturer's recommendations. For stable transfection, replication-deficient lentiviral particles encoding shRNA against PKD2 or vector control were prepared via co-transfecting 293T cells with three other helper vectors as described previously (Dull et al., 1998, J Virol 72:8463-8471). Viral supernatants were concentrated by PEG8000 precipitation and used to infect HeLa cells or 2fTGH cells in the presence of polybrene (3 μg/mL, Sigma). Cells were selected and maintained in the presence of puromycin (2 μg/mL).

### Chemicals, Antibodies and Immunotechniques

Immunoprecipitation and immunoblotting procedures are described elsewhere (Fuchs et al, 1999, Oncogene 18:2039-2046). Protein degradation was carried out by cycloheximide (CHX, used at 20 μg/ml) chase. IFNAR1 internalization assay was carried out using the Fluorescence-based assay as described elsewhere (Kumar et al., 2007, J Cell Biol 179:935-950). BAY 43-9006 was obtained as a gift. Recombinant human IFN-a2 (Roferon) was purchased from Roche. Other reagents or inhibitors were purchased from commercial vendors.

Antibodies against Flag, GST and j3-actin (Sigma), HA (12CA, Roche), CK1α, PICD1/2 (PKCμ), PKD3 (PKCν), Tyk2, intracellular domain of hIFNAR1 and PKR, (Santa Cruz), anti-pan-phospho-tyrosine (4G10), phospho-Stat1 and Stat1 (Cell signaling), phospho-S710 of PKD2 (Biosource), PKD2 (Bethyl Laboratories), mIFNAR1 (Leinco) and ubiquitin (FK2, Biomol) were purchased. AA3, GB8 and EAl2 antibodies which recognize endogenous IFNAR1 (Goldman et al., 1999) and antibodies against IFNAR1 phosphorylated on Ser535 (pS535; (Kumar et al., 2004, J Biol Chem 279:46614-46620)) were described previously. Secondary antibodies conjugated to horseradish peroxidase were purchased from Chemicon and LI-COR. Immunoprecipitation and immunoblotting procedures are described elsewhere (Fuchs et al, 1999, Oncogene 18:2039-2046). Protein degradation was carried out by cycloheximide (CHX) chase in the presence of CHX (20 μg/ml). Immunoblot detection and quantification were carried out using the LI-COR's Odyssey Infrared Imaging System.

IFNAR1 internalization assay was carried out using the fluorescence-based assay that determines the internalization of IFNAR1 by measuring the loss of cell-surface immunoreactivity of endogenous receptor using AA3 antibody as described elsewhere (Kumar et al., 2007, J Cell Biol 179:935-950). The same antibody in combination with anti-mouse-biotin (Jackson Laboratory) and streptavidin-PE (e-Bioscience) was used for analysis of cell surface human IFNAR1 levels using the FACSCalibur flow cytometer (BD Phanningen). Levels of mIFNAR1 were determined using an anti-mIFNAR1 antibody (Leinco).

Recombinant human IFN-a2 (Roferon) was purchased from Roche. Thapsigargin, cycloheximide, TPA and methylamine HCL were purchased from Sigma. H89, Bisindolylmaleimide (Bis-I), Gö6976, SP600125, SB203580, LY294002 and D4476 were from Calbiochem. BAY 43-9006 was a kind gift of M. Herlyn, Recombinant human VEGF (293-VE) and mouse VEGF (493-MV) used at 100 ng/ml were purchased from R&D Systems. CID755673 was purchased from TOCRIS Bioscience.

### Virus and Viral Infection

The anti-viral effect of IFN-a was assessed by pre-treating cells overnight prior to infection with VSV (Indiana serotype, propagated in HeLa cells) at a MOI of 0.1 for 1 h. After removing the virus inoculums, cells were then fed with fresh medium and incubated for 20 h. Culture supernatant was harvested and viral titer was determined in HeLa cells overlaid with methylcellulose as described elsewhere (Sharma et al., 2003, Science 300:1148-1151) and plaque-forming units (pfu/mL) calculated. Cells were observed to determine the cytopathic effect, and expression of VSV-M protein was analyzed by immunoblotting.

### In Vivo Matrigel Plug Assay

Matrigel Plug assay was carried out essentially as described (Medhora et al., 2003, Am J Physiol Heart Circ Physiol 284:H215-224). Briefly, WT/WT and heterozygous S526A WT/SA mice (6-9 weeks old, n=5 per group) were injected subcutaneously with 0.6 mL of Matrigel that was premixed with vehicle or 100 ng of mouse VEGF. After 7 days, Matrigel plugs were harvested from underneath the skin. The plugs were homogenized in 1 ml deionized water on ice and hemoglobin was measured by the Drabkin method with Drabkin's reagent kit 525 (Sigma) to quantify blood vessel formation according to the manufacturer's protocol. The absorbance was read at 540 nm. To identify of infiltrating endothelial cells, immunohistochemistry was carried out using an anti-CD31 antibody as described elsewhere (Chiodoni, et al., 2006, J Exp Med 203:2441-2450).

The results of the experiments presented in this Example are now described.

### PKD2 Mediates Ligand-Inducible Phosphorylation, Ubiquitination and Degradation of IFNAR1.

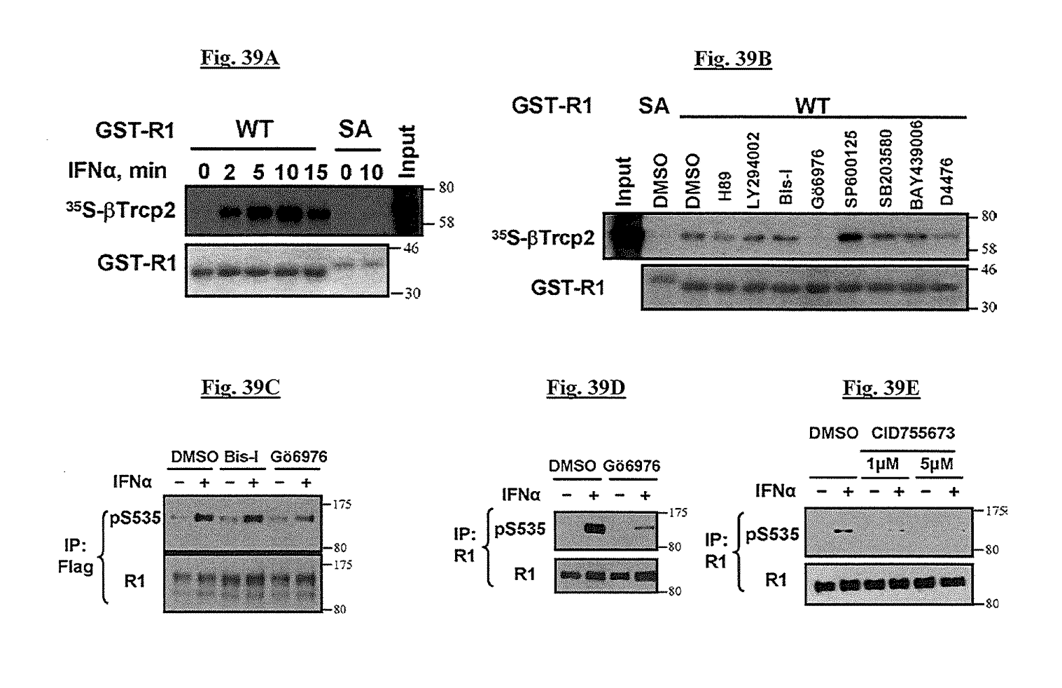

Recognition of IFNAR1 or the prolactin receptor by β-Trcp2 was dependent on the phosphorylation of specific receptor serine residues. This phosphorylation could be indirectly assessed in vitro by using cell lysates as a source of protein kinase, receptor-derived synthetic peptide or bacterially-expressed intracellular domain of a receptor as a substrate, and subsequent interaction with radio-labeled β-Trcp2 as the mode of detection (Kumar et al., 2003, Embo J 22:5480-5490; Li et al., 2004, Mol Cell Biol 24, 4038-4048). Whereas somewhat stronger binding of β-Trcp2 to IFNAR1 was seen when the lysates were prepared from IFN-α-treated cells, preparations from untreated cells also contained a robust basal kinase activity (Kumar et al., 2003, Embo J 22:5480-5490). This activity that often masked the effects of the ligand treatment was linked to the ligand-independent pathway of IFNAR1 degradation (Liu et al., 2008, Biochem Biophys Res Commun 367:388-393). Follow up studies have identified CKlu as a major source of this activity; knockdown of CK1α in cells or immunodepletion of CK1α, from the lysates abolished basal phosphorylation of the IFNAR1 degron (Liu, et al., 2009, Mol Cell Biol 29(24):6401-12). Using CK1α-immunodepleted lysates in the phosphorylation-binding assay, it was observed that pre-treatment of cells with IFN-a increased the efficacy of binding of p-Trcp2 to GST-IFNARIWT but not to its S535, 539A mutant (FIG. 39A). This result is suggestive of the existence of a ligand-inducible serine kinase activity that mediates phosphorylation-dependent recruitment of 13-Trcp2 to IFNAR1.

Known serine kinases that could be activated by IFN includes the members of the protein kinase C family, PKA, PI-3K-Akt-IKK, and MAPK (JNK, p38, Erk and their downstream kinases; reviewed in (Du et al., 2007, J Cell Biochem 102:1087-1094; Lamer et al., 1996, Biotherapy 8:175-181; Platanias, 2005, Nat Rev Immunol 5:375-386). Various pharmacologic kinase inhibitors (whose activity was verified in kinase-specific assays) were added in vitro to the phosphorylation-binding assay. As seen from FIG. 39B, the recruitment of 3-Trcp2 was not inhibited by inhibitors of PI-3K (LY294002) or JNK (SP600125) or c-Raf (BAY 43-9006) or p38 (SB203580). In contrast, a noticeable inhibition was observed when inhibitors of PKA (H89) or CKI (D4476) were used. Intriguingly, two inhibitors of PKC produced the opposite results. Whereas a dramatic inhibition was achieved using Gö6976 composition, a pan-PKC inhibitor bisindolylmaleimide I(Bis I) did not affect β-Trcp2 recruitment (FIG. 39B).

These latter two inhibitors were used to pre-treat human U3A cells stably expressing Flag-IFNAR1 and Ser535 phosphorylation of immunopurified receptor was assessed. Neither composition impaired the basal degron phosphorylation, which occurs independently of ligand presence and Jak activity (Liu et al., 2008, Biochem Biophys Res Commun 367:388-393). However, pre-treatment with Gö6976 (but not with Bis I) noticeably decreased the induction of Ser535 phosphorylation by IFN-a (FIG. 39C). Similar effects of Gö6976 were seen when ligand-induced Ser535 phosphorylation of endogenous IFNAR1 in 293T cells was examined (FIG. 39D). Given the lack of effect of Bis I, it is plausible that Gö6976 interferes with ligand-inducible IFNAR1 degron kinase activity that might be different from PKC.

Gö6976 was indeed shown to inhibit other kinases including protein kinase D (PKD, (Gschwendt et al., 1996)). PKD represents a family of serine/threonine protein kinases that is comprised of three members (PKD1/PKCμ, PKD2 and PKD3/PKCν). These kinases are responsive to activation by numerous stimuli including the phorbol esters and oxidative radicals (reviewed in (Rozengurt et al., 2005, J. Biol Chem 280:13205-13208; Rykx et al., 2003, FEBS Lett 546:81-86; Wang, 2006, Trends Pharmacol Sci 27:317-323)). It was then investigated whether PKD may play a role in ligand-inducible degron phosphorylation of IFNAR1. To this end, a recently identified benzoxoloazepinolone (CID755673) shown to exhibit high specificity against PKD in vitro and in cells (Sharlow et al., 2008, J Biol Chem 283:33516-33526) was used. Pre-treatment of 293T cells with this composition led to a significant inhibition of Ser535 phosphorylation of endogenous IFNAR1 in response to IFN-α (FIG. 39E). These data implicate PKD in regulating the ligand-stimulated phosphorylation of IFNAR1 's degron.

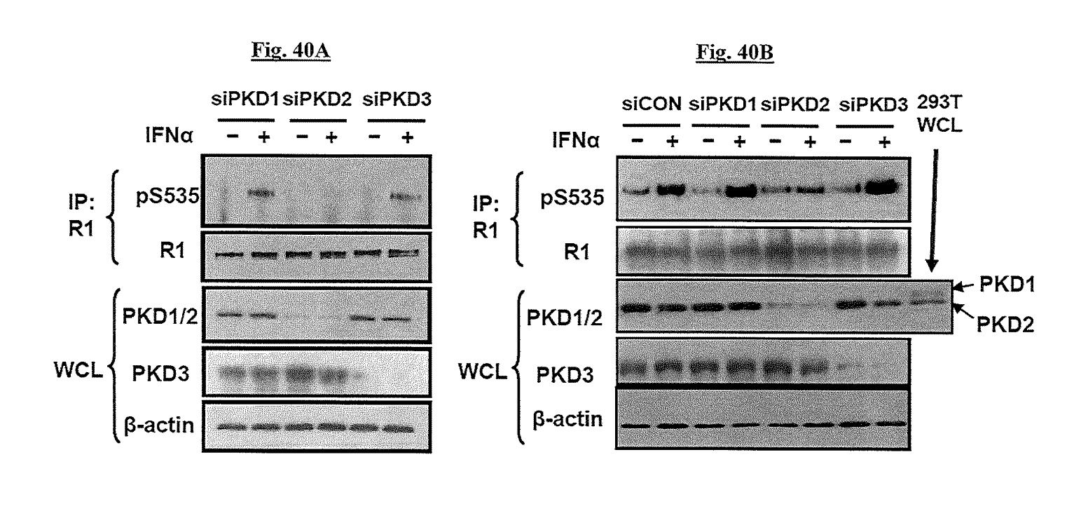

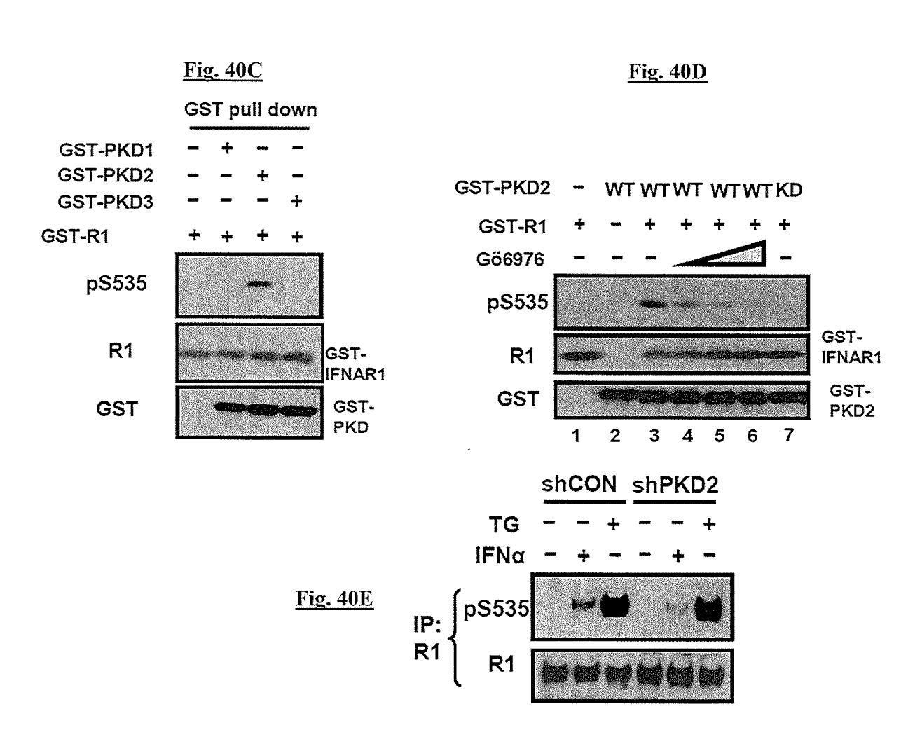

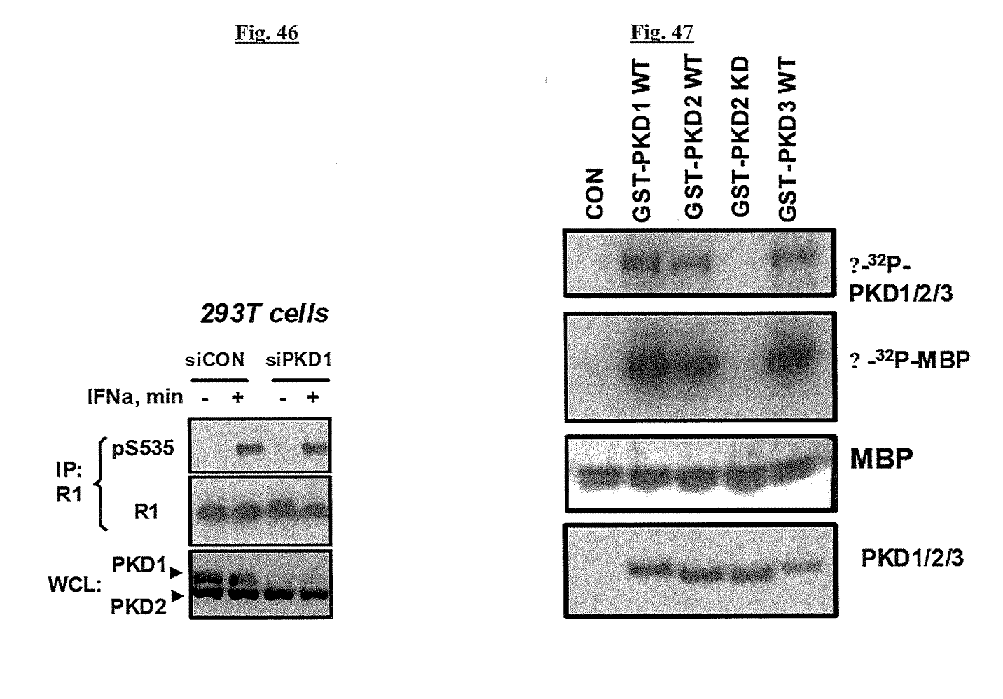

Next an RNAi approach was used to determine the putative role of various PKD isoforms. In human epithelial HeLa cells that do not express PKD1 (Bossard et al., 2007, J Cell Biol 179:1123-1131), siRNA specific against PKD2 (but not PKD1 or PKD3) markedly inhibited IFN-a-stimulated IFNAR1 phosphorylation on Ser535 (FIG. 40A). Similar data were obtained on exogenous Flag-IFNAR1 stably expressed in U3A human fibrosarcoma cells (FIG. 40B). In 293T cells that express both PKD1 and PKD2, a specific and efficient knock-down of PKD1 did not influence IFNAR1 degron phosphorylation (FIG. 46). These results strongly suggest that PKD2 is important for the ligand-inducible phosphorylation of the IFNAR1 degron, whereas the role of other PKD forms remain to be proven.

GST-tagged PKD2 expressed in 293T cells and purified by pull down with glutathione beads was capable of directly phosphorylating bacterially produced GST-IFNAR1 on Ser535 in vitro. Intriguingly, this outcome was not seen when either PKD1 or PKD3 were used as a source of kinase (FIG. 40C) despite comparable activity of all three kinases in an autophosphorylation assay or phosphorylation of an artificial substrate, myelinic basic protein (FIG. 47). Ser535 phosphorylation on GST-IFNAR1 incubated with ATP and GST-PKD2 was not evident when a catalytically deficient (kinase-dead, KD) K580N mutant was used in the reaction (FIG. 40D, lane 3 vs 7). Furthermore, adding the PKD inhibitor Gö6976 to this reaction decreased the efficacy of IFNAR1 phosphorylation in a dose dependent manner (FIG. 40D, lanes 3-6). These results collectively suggest that PKD2 is a direct Ser535 kinase of IFNAR1.

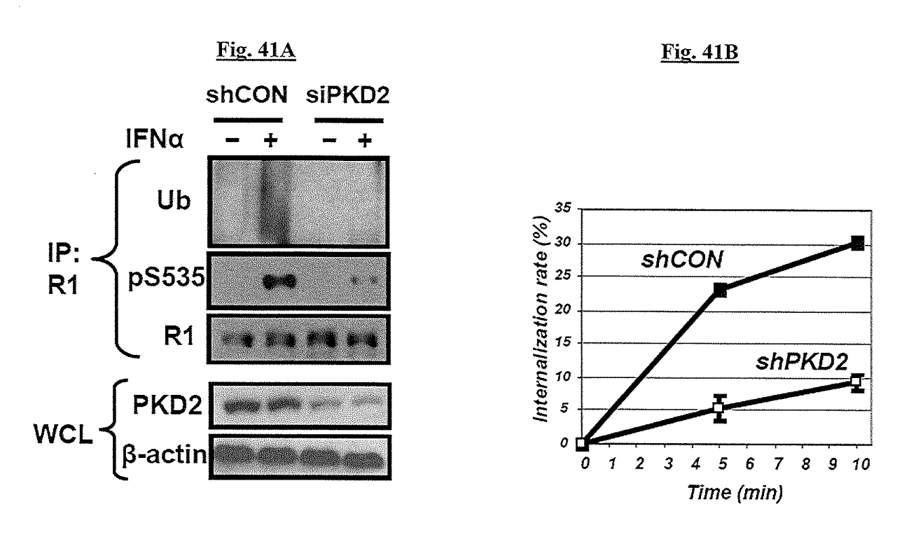

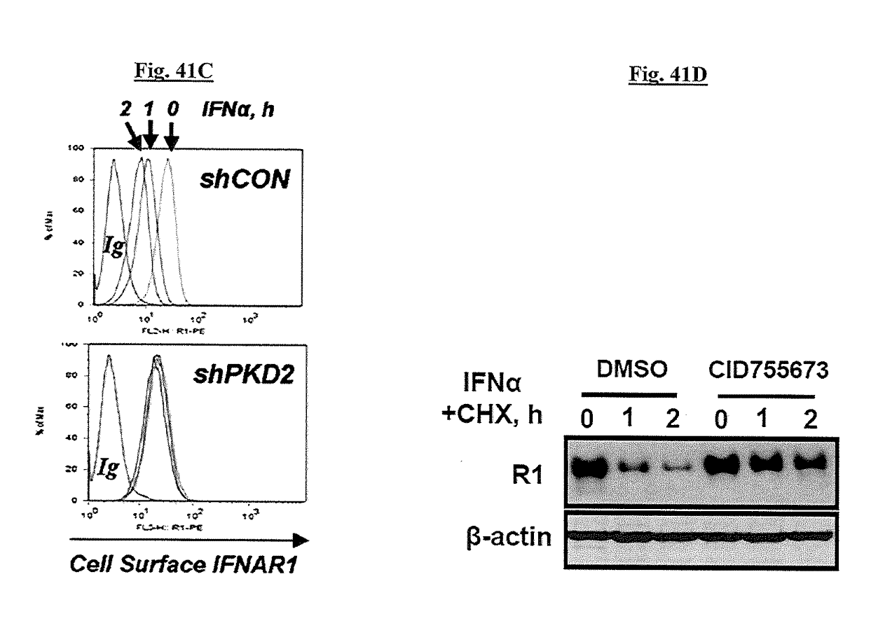

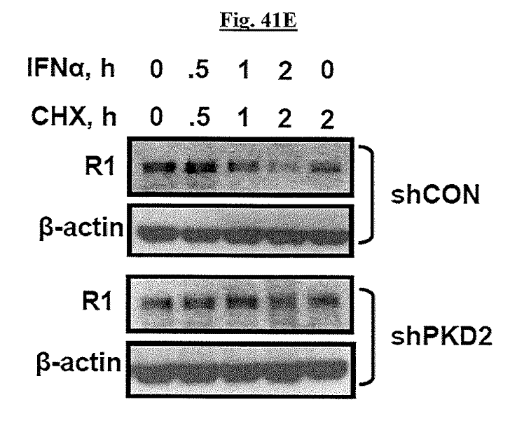

Knockdown of PKD2 inhibited phosphorylation of the IFNAR1 degron in response to IFN-α, but not to thapsigargin, and inducer of the unfolded protein response (UPR) (FIG. 40E). Thapsigargin and other inducers of UPR, including forced expression of IFNAR1-stimulated Ser535 phosphorylation and IFNAR1 ubiquitination, act in a ligand- and Jak-independent manner (Liu et al., 2009, Cell Host Microbe 5(1):72-83; Liu et al., 2008, Biochem Biophys Res Commun 367:388-393). Accordingly, careful examination of FIGS. 39C and 40B also reveals that neither the PKD inhibitor Gö6976 nor siRNA against PKD2 decreases the levels of basal Ser535 phosphorylation of overexpressed IFNAR1. These data suggest that the role of PKD2 in regulating phosphorylation of IFNAR1 's degron might be limited to the ligand-inducible pathway. Given that IFN-a-induced Ser535 phosphorylation is required for ubiquitination, endocytosis, down regulation and degradation of IFNAR1, it was investigated whether these events are mediated by PKD2. Knockdown of PKD2 in HeLa cells robustly inhibited IFN-a-induced ubiquitination of IFNAR1 (FIG. 41A) and decreased the rate of internalization of this receptor (FIG. 41B). Consistent with these data, ligand-stimulated downregulation of IFNAR1 on the cell surface (measured by FACS analysis) was noticeably impeded in cells expressing shRNA against PKD2 (FIG. 41C). Treatment of HeLa cells with the PKD inhibitor CID755673 resulted in an appreciable decrease in the rate of proteolytic turnover of IFNAR1 (FIG. 41D). Furthermore, ligand-stimulated degradation of IFNAR1 was noticeably impaired in cells that received shRNA against PKD2 (FIG. 41E). In all, these results suggest that PKD2 plays an important role in the ligand-inducible ubiquitination, endocytosis, and degradation of IFNAR1.

### PKD2 is Activated in Response to IFNa in a Tyk2-Dependent Manner

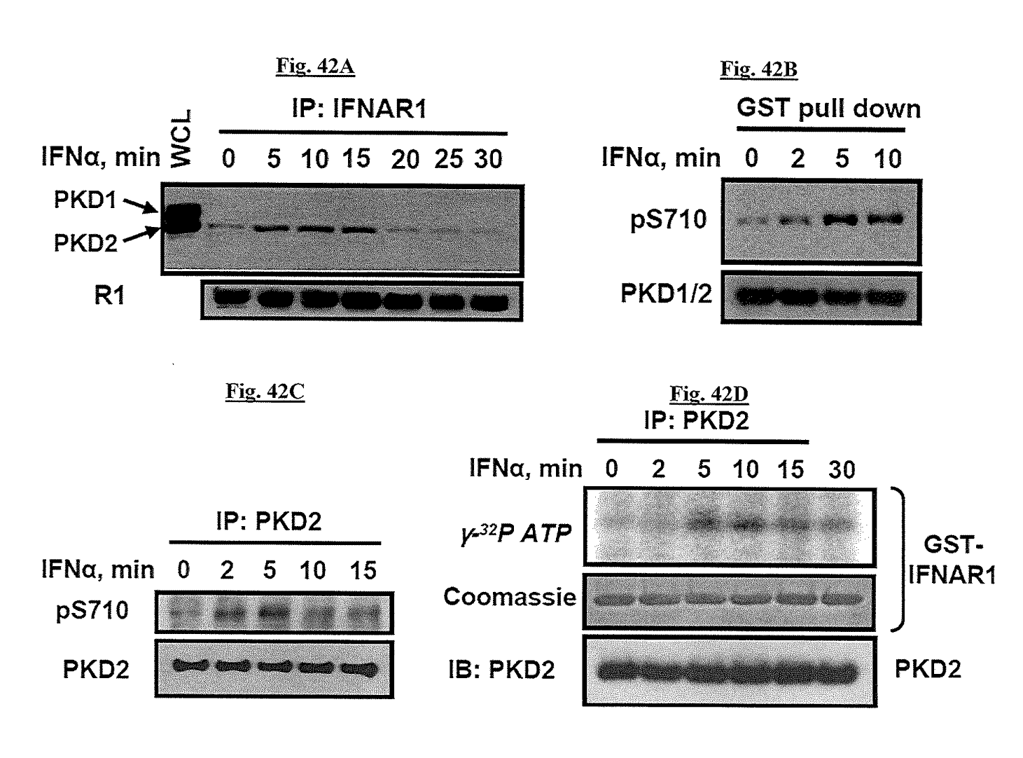

It was next assessed whether IFN-α-induced signaling stimulates phosphorylation of IFNAR1 on Ser535 by PKD2. Interaction between endogenous IFNAR1 and PKD2 in untreated 293T cells was demonstrated using co-immunoprecipitation reactions (FIG. 48). An increase in this interaction upon treating cells with IFN-α was also observed (FIG. 42A). Intriguingly, neither basal nor IFN-inducible interaction between the receptor and PKD 1 was detected (FIGS. 48 and 42A). These data suggest that PKD2 is specifically recruited to IFNAR1 and that this recruitment is stimulated by ligand treatment.

Phosphorylation of GST-PKD2 expressed in HeLa cells on Ser710 (indicative of PKD2 activation (Sturany et al., 2002, J Biol Chem 277:29431-29436)) was stimulated upon treating the cells with IFN-a (FIG. 42B). Comparable results were obtained when endogenous PKD2 was analyzed (FIG. 42C). Furthermore, treatment of cells with IFN-α increased activity of endogenous PKD2 assessed by an immunokinase assay with GST-IFNAR1 as a substrate (FIG. 42D), These results collectively suggest that catalytic activity of PKD2 is stimulated as a result of signaling events triggered by the ligand.

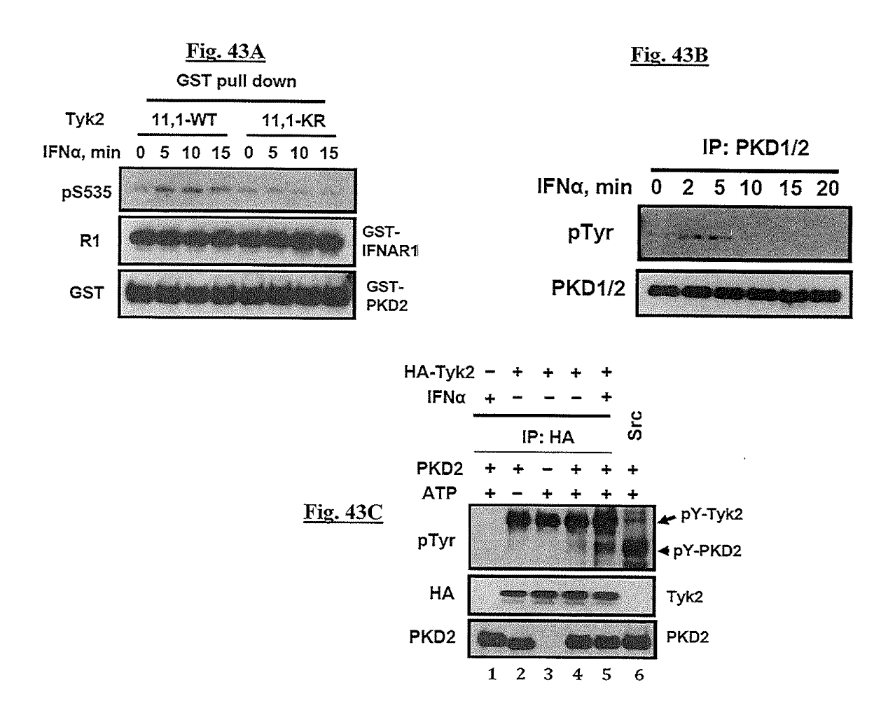

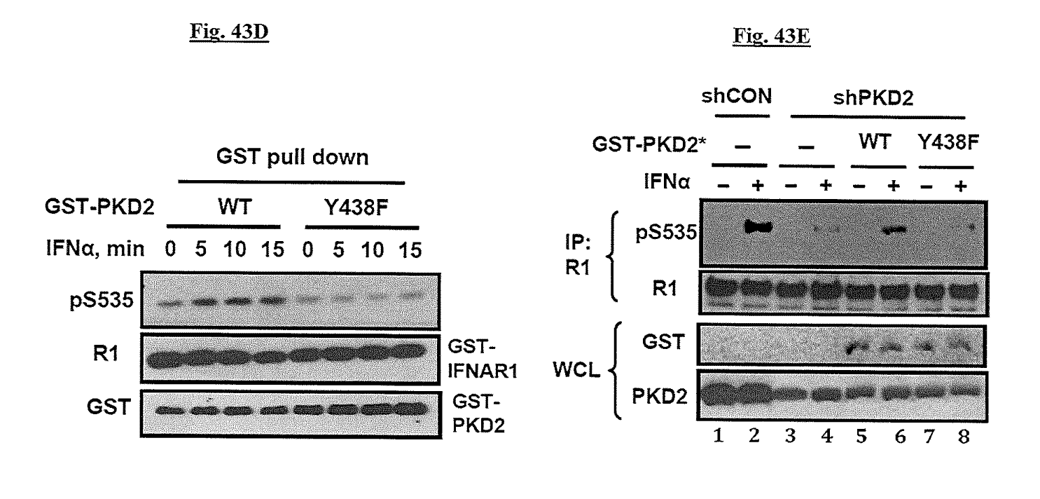

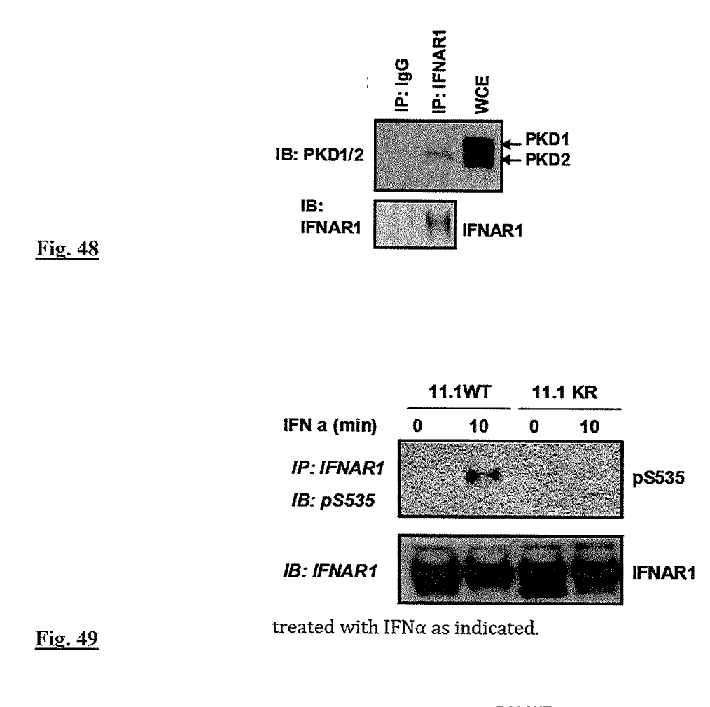

Ligand-inducible phosphorylation of the IFNAR1 degron depends on the kinase activity of Tyk2. Indeed, this phosphorylation was observed in 1L1-Tyk2-null human cells reconstituted with wild type Tyk2 but not with catalytically inactive Tyk2KR mutant (Marijanovic et al., 2006, Biochem J 397:31-38) and FIG. 49). Remarkably, IFN-α treatment activated GST-PKD2 (as assessed by its ability to phosphorylate GST-IFNAR1 on Ser535 in vitro) in 11.1-Tyk2WT but not in 11.1-Tyk2KR cells (FIG. 43A). Importantly, status of Tyk2 had no bearing on the basal activity of expressed PKD2. This result suggests that activation of PKD2 in ligand-treated cells requires the tyrosine kinase activity of Tyk2. Furthermore, treatment of HeLa cells with IFN-a noticeably stimulated phosphorylation of endogenous PKD2 on Tyr residues (FIG. 43B). Considering that Tyk2 is tightly associated with IFNAR1 (Platanias, 2005, Nat Rev Immunol 5:375-386) and increasing amounts of PKD2 are recruited to the vicinity of IFNAR1 (FIG. 42A), it was proposed that Tyk2 induced by IFN-a might activate PKD2 via direct phosphorylation on tyrosines.

To test this hypothesis, HA-tagged Tyk2 was expressed and immunopurified from cells (treated or not with IFN-a) and then incubated with recombinant bacterially produced PKD2 in the presence of ATP. The reaction was analyzed by immunoblotting using an anti-phospho-Tyr antibody. In this assay, tyrosine phosphorylation of PKD2 was easily detected when recombinant Sre protein was used as positive control (FIG. 43C, lane 6). In the presence of HA-Tyk2, a noticeable tyrosine phosphorylation signal on PKD2 was also observed in an ATP-dependent manner (FIG. 43C, compare lanes 2 and 4). A robust increase in Tyr phosphorylation of PKD2 was seen when HA-Tyk2 was purified from IFN-a-treated cells (FIG. 43C, lanes 4-5) indicating that ligand-stimulated Tyk2 is capable of directly phosphorylating PKD2.

Two non-exclusive mechanisms have been proposed for activation of PKD1 via relieving an autoinhibitory effect of its PH domain. One is a phosphorylation of the activation loop of PKD on S744/S748 (Sinnett-Smith et al., 2009, J Biol Chem 284:13434-13445; Waldron et al., 2003, J Biol Chem 278:154-163); another is a tyrosine phosphorylation of Y463 stimulated by Src (Storz et al., 2003, Embo J 22:109-120). A homologous tyrosine residue, Y438 was found on PKD2 and proposed to play a role in modulating its activity (Mihailovic et al., 2004, Cancer Res 64:8939-8944). It was next investigated whether the role of this site in IFN-α-induced PKD2 activation and IFNAR1 phosphorylation. Basal Ser535-phosphorylating activity of GST-PKD2Y438F mutant expressed in HeLa cells was similar to that of wild type enzyme. However, activation of this mutant kinase in response to IFN-a treatment was visibly impaired (FIG. 43D).

This result suggests that phosphorylation of Y438 might be required for stimulation of PKD2 activity by IFN-α. To further test this possibility, GST-PKD2 expression constructs (depicted as GST-PKD2*) were generated that contained silent mutations making them insensitive to shPKD2 that were used for kinase knockdown. HeLa cells were stably transduced with control shRNA or shPKD2 and with empty vector or GST-PKD2*constructs (wild type or Y438 mutant). Consistent with data shown in FIG. 40B, knockdown of endogenous PKD2 impeded IFN-a-induced phosphorylation of IFNAR1 on Ser535 (FIG. 43E, lane 2 vs. lane 4). Whereas re-expression of wild type PKD2 distinctly restored this phosphorylation to some extent, such an effect was not observed when PKD2 was mutated at Y438 (FIG. 43E, lane 6 vs. 8). These results collectively suggest that ligand-induced phosphorylation of this Tyr residue within PKD2 is required for PKD2-mediated phosphorylation of the IFNAR1 degron.

Ligand-Induced PKD2-Mediated Down Regulation of Ifnar1 Restricts the Extent of cellular Responses to IFN-α

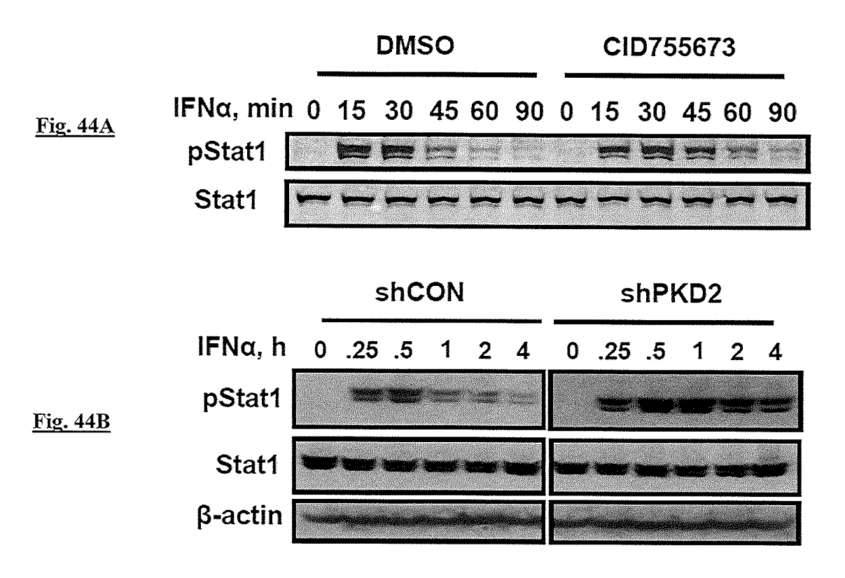

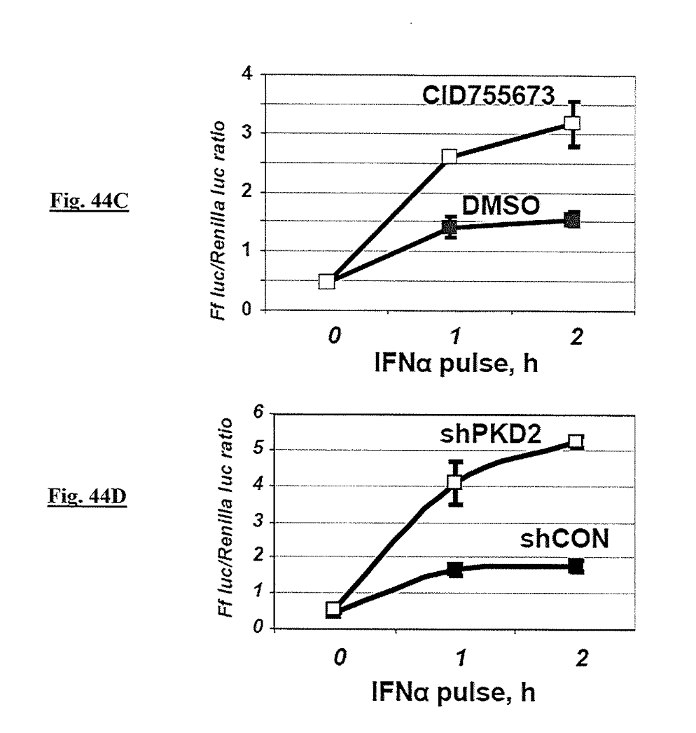

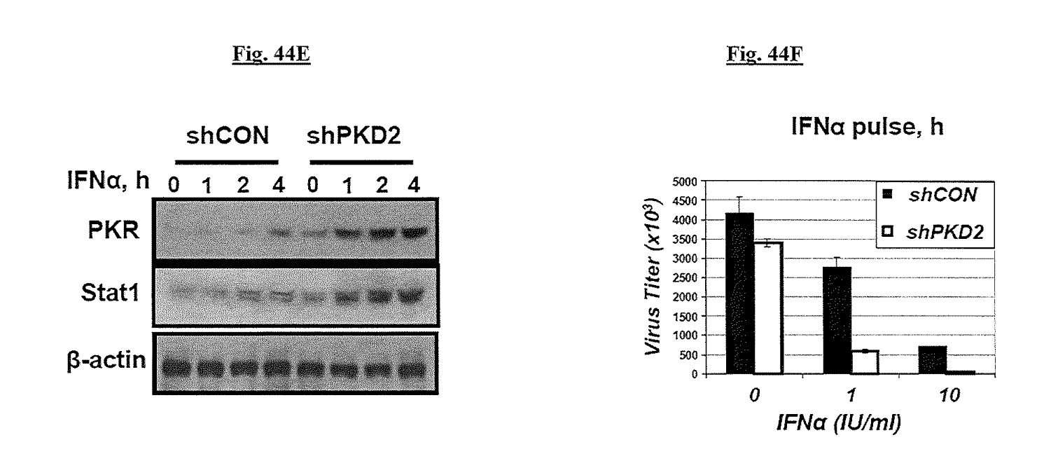

It was next investigated whether PKD2-mediated ubiquitination and degradation of IFNAR1 contributes to the regulation of cellular responses to Type I IFN. Pulse treatment of HeLa cells with IFN-α led to a robust activation of Stat1 (assessed by its tyrosine phosphorylation) that peaked at 15-30 min and declined during the next hour. A brief pre-treatment of cells with PKD inhibitor CID755673 noticeably prolonged ligand-induced phosphorylation of Stat1 (FIG. 44A). Similarly, knocking down PKD2 in HeLa cells resulted in a more robust and prolonged Stat1 activation (FIG. 44B). These results suggest that PKD2 contributes to the regulation of the magnitude and duration of IFN-α signaling.

To evaluate the role of PKD2 in Type I IFN-induced transcription and anti-viral effects human fibrosarcoma 2fTGH cells were used that are highly sensitive to IFN-α/β (McKendry et al., 1991, Proc Natl Acad Sei USA 88:11455-11459) and, unlike HeLa cells, do not harbor human papillomavirus genes. Upon pulse treatment of these cells with IFN-α for 1-2 h, the ratio in activity of the firefly luciferase reporter driven by an IFN-stimulated response element (ISRE) to CMV-driven renilla luciferase was noticeably increased. Pre-treatment of cells with the PKD inhibitor C1D755673 robustly augmented this transcriptional activity (FIG. 44C). Consistent with this result, ligand-induced ISRE-driven luciferase activity was much higher in cells where PKD2 was knocked out (FIG. 44D). Furthermore, cells that harbored shRNA against PKD2 exhibited a noticeably higher expression of the products of interferon-stimulated genes such as PKR and Stat1 (FIG. 44E).

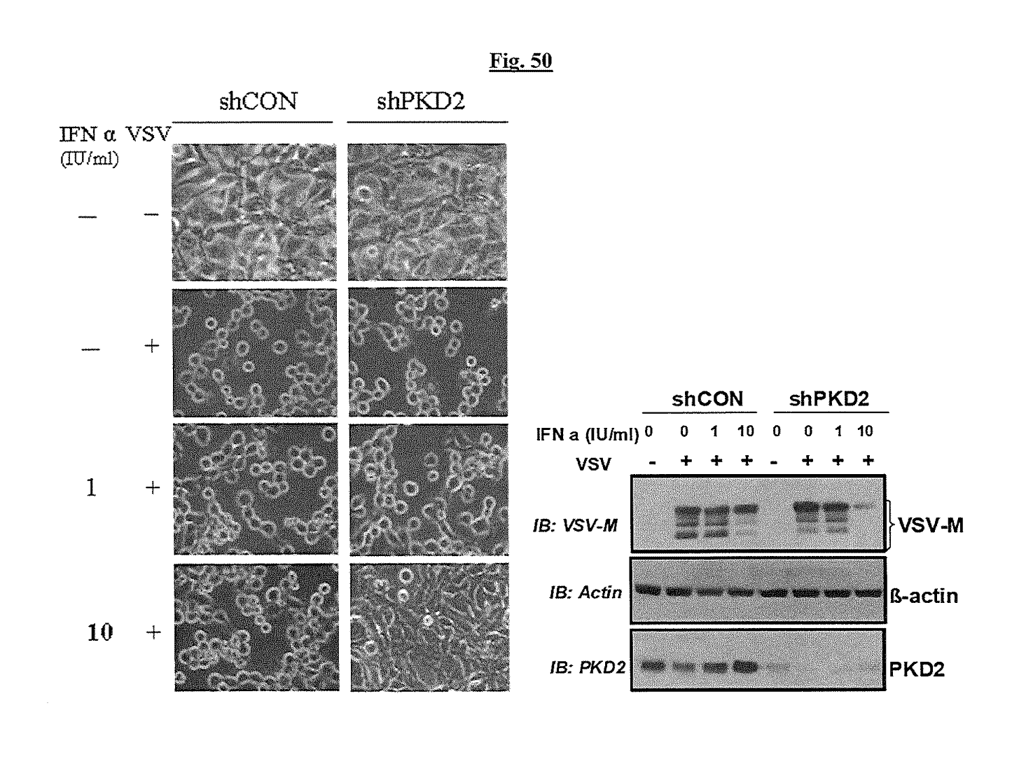

Knock down of PKD2 in 2fTGH cells resulted in an approximately 20-25% lesser viral titer upon infection with vesicular stomatitis virus (VSV) (FIG. 44F); most likely in lieu of a well characterized role of PKD in viral protein transport along the secretory pathway (Jamora et al., 1999, Cell 98:59-68). Remarkably, pre-treatment of PKD2 knockdown cells with low doses of IFN-α led to a much more robust protection against infection with VSV as evident from the assessment of viral titer (FIG. 44F), or cytopathogenic effects of VSV (FIG. 50). These data together suggest that PKD2 plays an important role in regulating the cellular responses to Type I IFN including the induction of anti-viral defenses.

### Phosphorylation-Dependent Downregulation of IFNAR1 by VEGF is Required for Efficient Angiogenesis.

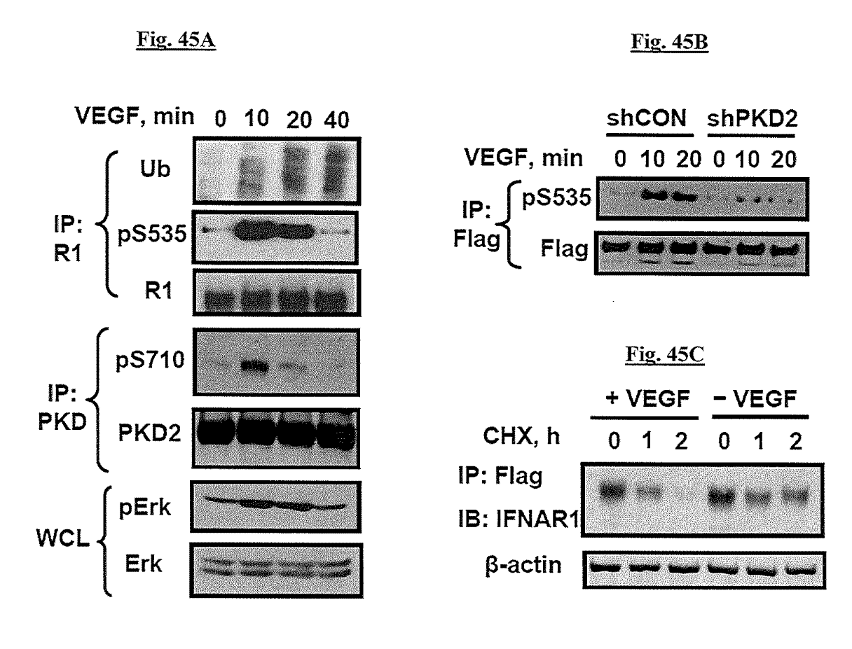

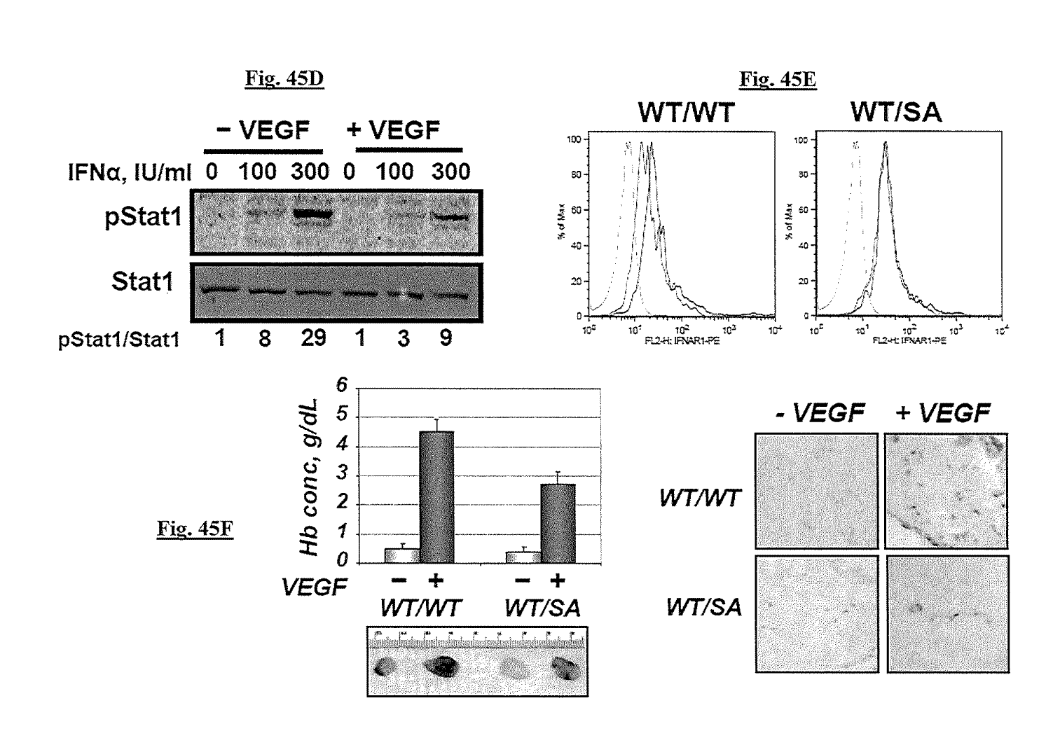

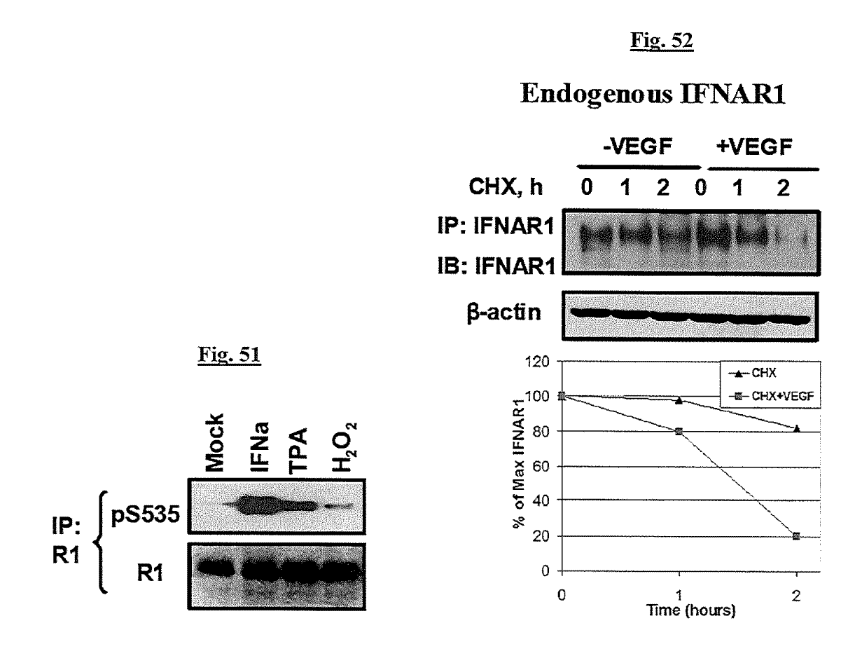

Numerous extracellular stimuli have been reported to activate PKD family kinases (reviewed in (Rozengurt et al., 2005, J Biol Chem 280:13205-13208; Rykx et al., 2003, FEBS Lett 546:81-86; Wang, 2006, Trends Pharmacol Sci 27:317-323)). It was investigated whether other signaling pathways capable of activating PKD2 may affect IFNAR1 stability and signaling. Treatment of HeLa cells with known PKD inducers such as phorbol esters or hydrogen peroxide noticeably stimulated phosphorylation of endogenous IFNAR1 on Ser535 even in the absence of IFN-α (FIG. 51). It was next tested the effects of VEGF (shown to induce PKD via a tyrosine phosphorylation-dependent mechanism (Qin et al., 2006, J Biol Chem 281:32550-32558; Wong et al., 2005, J Biol Chem 280:33262-33269)) on recombinant Flag-IFNAR1 stably expressed 2fTGH-derived U3A cells that do not express PKD1 (FIG. 40B). These cells responded to VEGF treatment by activation of both Erk and PKD2 and by an increased phosphorylation of Flag-IFNAR1 on Ser535 (FIG. 45A), The latter event was apparently PKD2-dependent as evident from analysis of cells that received shRNA against PKD2 (FIG. 45B). These results suggest that other signaling pathways capable of activating PKD2 may appropriate this kinase to target IFNAR1 for phosphorylation of its degron. Given that Type I IFNs are known to inhibit angiogenesis (Sidky et al., 1987, Cancer Res 47:5155-5161), a process that involves proliferation and migration of endothelial cells induced by VEGF (reviewed in (Ferrara, 2004, Endocr Rev 25:581-611; Ho et al., 2007, Int J Biochem Cell Biol 39:1349-1357)), it was hypothesized that VEGF-stimulated IFNAR1Ser535 phosphorylation might be of functional importance.

Immunoblotting analysis of Flag-IFNAR1 immunopurified from U3A-Flag-IFNAR1 cells using an anti-ubiquitin antibody revealed that ubiquitination of IFNAR1 was stimulated by VEGF treatment (FIG. 45A). Consistently, VEGF accelerated the proteolytic turnover of Flag-IFNAR1 in U3A cells (FIG. 45C). A similar outcome was observed for the endogenous IFNAR1 in human umbilical vein endothelial cells (FIG. 52). Furthermore, VEGF treatment of these cells noticeably inhibits Stat1 phosphorylation in response to IFN-a (FIG. 45D), These results suggest that VEGF promotes IFNAR1 degradation and limits the extent of IFN-a signaling.

VEGF stimulates numerous signaling pathways that confer its ability to promote angiogenesis (Ferrara, 2004, Endocr Rev 25:581-611; Kowanetz et al., 2006, Clin Cancer Res 12:5018-5022). It was investigated whether phosphorylation-dependent degradation of IFNAR1 may contribute to this function of VEGF. In order to test this possibility one may either modulate PKD2 kinase activity/expression or alter the availability of the degron's phospho-acceptor site within IFNAR1, Remarkably, a recently published report has already demonstrated that PKD2 activation is crucial for VEGF-stimulated growth and migration of endothelial cell, as well as angiogenesis per se (Hao et al., 2009, J Biol Chem 284:799-806). Given that VEGF-activated PKD2 may mediate these biological outcomes via phosphorylating diverse substrates, another approach that focuses on altering IFNAR1 itself was warranted to determine the role of PKD2-mediated IFNAR1 phosphorylation.

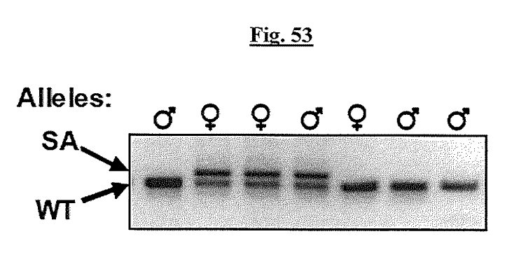

Mouse ES cells, in which one wild Type Ifnar1 allele has been replaced with the mutant that lacks Ser526, a serine residue homologous to Ser535 within human IFNAR1 (described in (Liu et al., 2009, Cell Host Microbe 5(1):72-83)) were subjected to expression of Cre recombinase to get rid of the Neo cassette, and then used to generate knock-in mice that express the mIFNAR1S526A mutant (“SA”, FIG. 53). Bone marrow-derived CD31-positive cells from wild type mice displayed a noticeable decrease in the cell surface levels of IFNAR1 upon treatment with murine VEGF (FIG. 45E). This effect was much less pronounced in the heterozygous mice (“WT/SA”) indicating that phosphorylation of the degron of IFNAR1 is required for downregulation of IFNAR1 in response to VEGF.

The formation of new vessels stimulated by VEGF in vivo was assessed using a matrigel plug assay. Visual examination of retrieved plugs revealed that mice that carry one allele of the S526A mutant of mIFNAR1 were less responsive to VEGF-stimulated angiogenesis (FIG. 45F). This observation was independently supported by immunohistochemicala analysis of CD31-positive cells within the paraffin-embedded plugs as well as by a quantitative analysis of the hemoglobin content in the extracts from the matrigel plugs (FIG. 45F). These data suggest that phosphorylation-dependent downregulation of IFNAR1 plays an important role in VEGF-stimulated angiogenesis.

### PKD2 is a Ligand-Inducible Regulator of IFNAR1 Stability

Ligand-stimulated lysosomal degradation of IFNAR1 plays an important role in limiting the magnitude and duration of Type I IFN signaling. Previous studies demonstrated that this degradation is mediated by IFNAR1 ubiquitination (Kumar et al., 2007, J Cell Biol 179:935-950), which is facilitated by the SCFOTrcp E3 ubiquitin ligase. This ligase is recruited to the receptor upon phosphorylation of its degron (Kumar et al., 2004, J Biol Chem 279:46614-46620; Kumar et al., 2003, Embo J 22:5480-5490). Unlike ligand-independent phosphorylation, ubiquitination and degradation of IFNAR1 (Liu et al., 2008, Biochem Biophys Res Commun 367:388-393), the IFN-inducible events rely on Tyk2 kinase activity (Marijanovic et al., 2006, Biochem J 397:31-38). Given that Tyk2 is a tyrosine protein kinase that displays poor (if any) ability to phosphorylate serine residues (Barbieri et al., 1994, Eur J Biochem 223:427-435), the existence of a ligand-activated Tyk2-dependent serine kinase has been proposed (Kumar et al., 2004, J Biol Chem 279:46614-46620; Marijanovic et al., 2006, Biochem J397:31-38).

As disclosed herein, (i) treatment of cells with inhibitors of PKD or specific knockdown of PKD2 attenuated phosphorylation of Ser535 of IFNAR1 in response to IFN-a but not to thapsigargin or IFNAR1 overexpression (FIGS. 39-40), (ii) purified PKD2 is capable of directly phosphorylating Ser535 (FIG. 40), (iii) other known inducers of PKD such as phorbol esters or VEGF are capable of stimulating IFNAR1 phosphorylation (FIGS. 51 and 45A); and (iv) PKD2 expression is important for regulating IFNAR1 ubiquitination, endocytosis and degradation (FIG. 41). Collectively, these data of pharmacologic, genetic and biochemical analyses indicate a novel and important function of previously discovered kinase, PKD2, as a bona fide IFNAR1's degron kinase that functions within the ligand-inducible pathway to enable the recruitment of βTrcp and mediate the proteolytic elimination of IFNAR1.

Whereas there experiments disclosed here focused on PKD2, they do not entirely rule out a role of other members of the PKD family (e.g., PKD1 and PKD3) in regulation of IFNAR1 phosphorylation and stability. However, it appears that some unique attributes of PKD2 enable its preferential recruitment to the vicinity of IFNAR1 and its ability to phosphorylate IFNAR1 on Ser535 (FIGS. 42A and 40C).

### Example 5

### Mammalian Casein Kinase 1a and its Leishmanial Ortholog Regulate Stability of IFNAR1 and Type I Interferon Signaling

Phosphorylation of the degron of the IFNAR1 chain of the Type I interferon (IFN) receptor triggers ubiquitination and degradation of this receptor and, therefore, plays a crucial role in negative regulation of IFN-α/β signaling. Besides the IFN-stimulated and Jak activity-dependent pathways, a basal ligand-independent phosphorylation of IFNAR1 has been described and implicated in down-regulating IFNAR1 in response to virus-induced endoplasmic reticulum (ER) stress. Disclosed herein is the purification and characterization of casein kinase 1a (CK1α) as a bona fide major IFNAR1 kinase that confers basal turnover of IFNAR1 and cooperates with ER stress stimuli to mediate phosphorylation-dependent degradation of IFNAR1. Activity of CK1α was required for phosphorylation and downregulation of IFNAR1 in response to ER stress and viral infection. While many forms of CK1 were capable of phosphorylating IFNAR1 in vitro, human and L-CK1 produced by the protozoan Leishmania major were also capable of increasing IFNAR1 degron phosphorylation in cells. Expression of leishmania CK1 in mammalian cells stimulated the phosphorylation-dependent downregulation of IFNAR1 and attenuated its signaling. Infection of mammalian cells with L. major modestly decreased IFNAR1 levels and attenuated cellular responses to IFN-α in vitro.

Disclosed herein is the identification and characterization of casein kinase 1a (CK1α) as a major bona fide kinase of IFNAR1 that mediates basal phosphorylation, ubiquitination, and turnover of IFNAR1. Experiments using genetic and pharmacological approaches further demonstrate the involvement of CK1α, in ligand-independent degron phosphorylation and degradation of IFNAR1 stimulated by ER stress inducers, including VSV. Intriguingly, CK1 activity secreted by Leishmania is also capable of phosphorylating the IFNAR1 degron. Expression of leishmanial CK1 (LCK1) in mammalian cells downregulates IFNAR1 and attenuates IFN-α/β signaling in a phosphorylation-dependent manner. Together with previous observations with viral pathogens, these results highlight the involvement of members of the CK1 family of kinases in the ligand-independent IFNAR1 degradation pathway, which plays a role in shaping the interaction between a mammalian host and infectious agents.

The Materials and Methods used in this Example are now described.

### Purification of Basal IFNAR1 Kinase Activity.

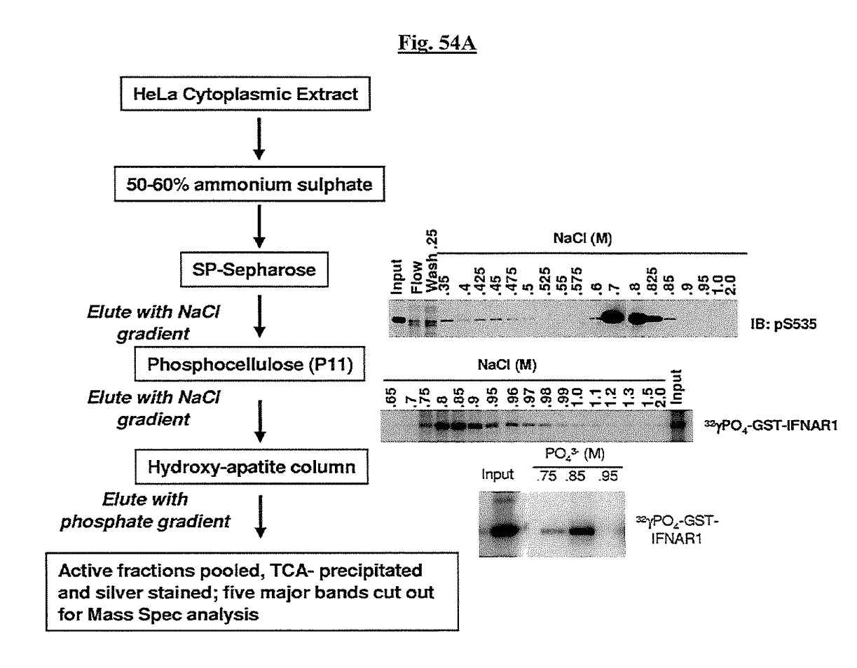

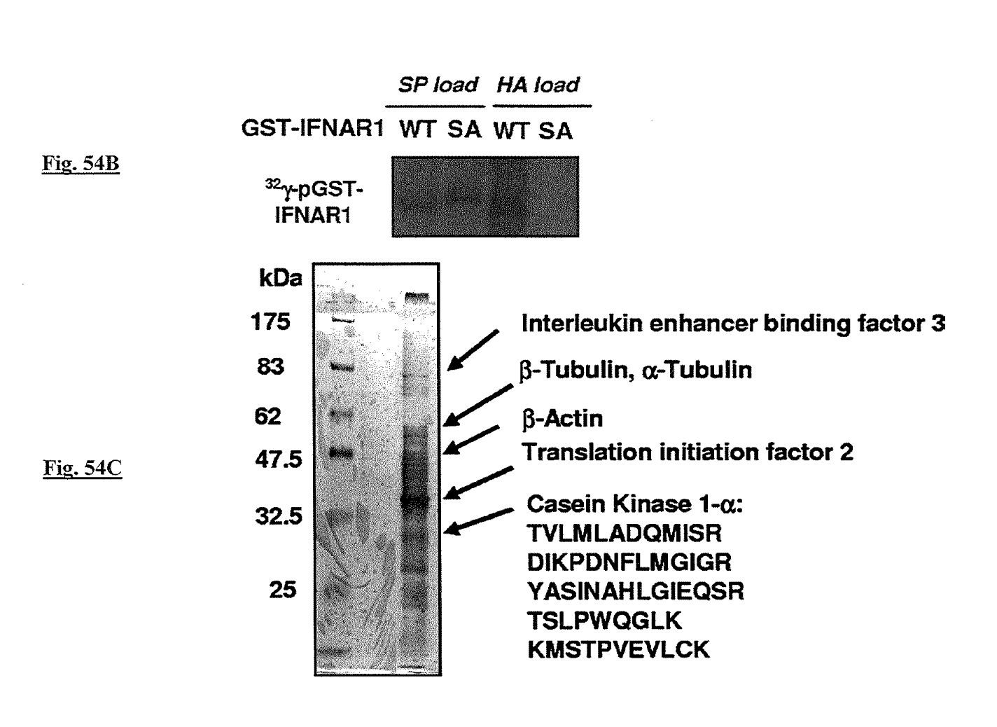

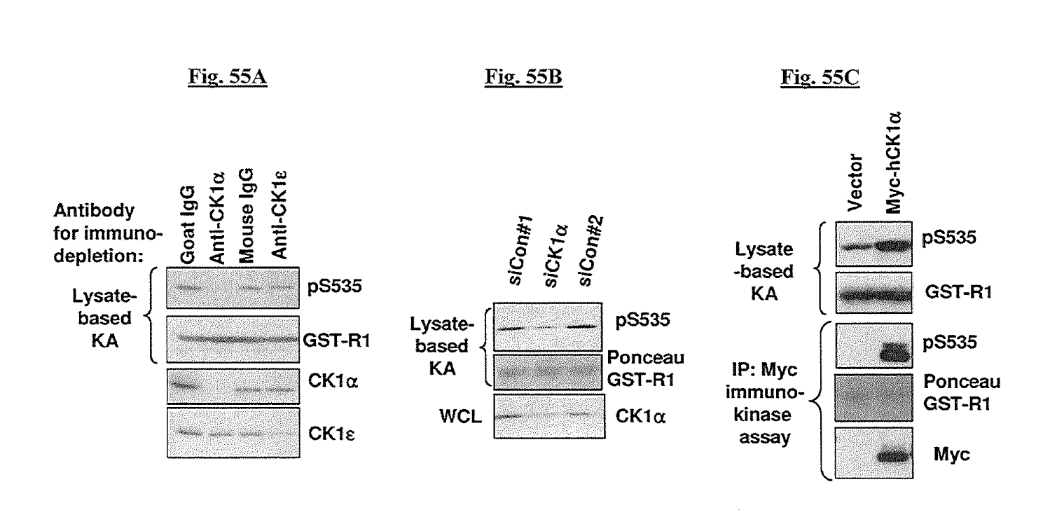

Basal IFNAR1Ser535 kinase activity was measured in vitro using bacterially expressed glutathione S-transferase (GST)-IFNAR1 (1 μg) as a substrate, lysates from indicated cells (1 μg intact or 4 μg immunodepleted) as a source of kinase, and immunoblotting (IB) with anti-pS535 antibody as a method of detection, as described in detail elsewhere (Liu et al., 2009, Cell Host. Microbe 5:72-83; Liu et al., 2008, Biochem. Biophys. Res. Commun. 367:388-393). Untreated HeLa cells were harvested, suspended in 10 mM Tris-HCl (pH 8.0), 10 mM KCl, 1.5 mM MgCl2, 0.5 mM dithiothreitol, 0.2 mM EDTA, and a cocktail of protease inhibitors (suspension buffer), and lysed by passing through a 23-gauge needle. After centrifugation, the nuclear pellet was discarded and the supernatant was ultracentrifuged at 100,000×g for 60 min. Following centrifugation, the supernatant was kept at 4° C. in buffers containing a cocktail of protease inhibitors. Approximately 90 ml of HeLa cell 5100 extract (−10 mg/ml) was precipitated with ammonium sulfate (50% to 60% saturation), and the pellet was redissolved, dialyzed, and applied onto a SP Sepharose (Amersham-Pharmacia) column and eluted with a linear gradient (100 to 2,000 mM NaCl) in buffer A containing 100 mM phosphate buffer, 50 mM KCl, 0.1 mM EDTA, and 10% glycerol. Fractions that contained Ser535 IFNAR1 kinase activity were pooled, concentrated, and further characterized by their ability to facilitate the incorporation of radioactive phosphate from 32P-labeled -y-ATP into the wild-type GST-IFNAR1 (GST-IFNARIWT) but not the GST-IFNAR1S535A mutant. Active fractions were applied onto a phosphocellulose column (P11; Whatman) and eluted with a linear gradient (500 to 2,000 mM NaCl) in buffer B containing 20 mM Tris-HCl (pH 7.6), 100 mM KCl, 0.1 mM EDTA, and 10% glycerol. Active fractions were concentrated on a hydroxyappatite column (Bio-Rad), eluted stepwise using orthophosphate buffer, concentrated, and separated by sodium dodecyl sulfate-polyacrylamide gel electrophoresis (SDS-PAGE). Five major bands (see FIG. 54B, below) were excised and subjected to in-gel tryptic digestion followed by nano-electrospray ionization (ESI) liquid chromatography-mass spectrometry (MS) by using a Waters Q-ToF mass spectrometer with a Waters nanoAcquity HPLC apparatus. The resulting tandem MS (MS/MS) spectra of the peptides derived from one of the bands contained several peptides (including DIKPDNFLMGIGR (SEQ ID NO:16), YASINAHLGIEQSR (SEQ ID NO:17), TSLPWQGLK (SEQ ID NO:18), KMSTPVEVLCK (SEQ ID NO:19), and FEEAPDYMYLR (SEQ ID NO:20)) that were identified as derivatives of human CSNK1A1 (CK1a).

Constructs for mammalian expression of IFNAR1 and bacterial expression of GST-IFNAR1 were previously described (Kumar et al., 2007, J. Cell Biol. 179:935-950; Kumar et al., 2003, EMBO J. 22:5480-5490). The construct for bacterial expression of GST-CK1α (described in Chen et al., 2005, Mol. Cell. Biol. 25:6509-6520). Constructs for expression of human Myc-tagged CK1 and shRNA vectors against CKla or green fluorescent protein (GFP) were previously described (Shirogane et al., 2005, J. Biol. Chem. 280:26863-26872). Human CK1α and L-CK1 cDNA (described in Allocco et al., 2006, Int. J. Parasitol. 36:1249-1259) were subcloned into a pEF-BOS vector with a hemagglutinin (HA) tag. A point mutation of K40R in L-CK1 was introduced via site-directed mutagenesis. Vaccinia virus B1 kinase and its kinase-dead mutant form (K149Q [KD]) expression constructs were previously described (Santos et al., 2004, Virology 328:254-265). Recombinant human IFN-a2a was purchased from Roche. Thapsigargin, cycloheximide, and D4476 were from Sigma. Murine IFN-(3 and human IFN—y were purchased from PBL. Sinall interfering RNA (siRNA) oligos against the luciferase gene (5′-CUUACGCU GAGUACUUCGAdTdT-3′ (SEQ ID NO:21)) or hCK1α (5′-CCAGGCAUCCCCAGUUGCUd TdT-3′ (SEQ ID NO:22)) were purchased from Dharmacon Inc. In some experiments, the siRNA oligos that contained several substitutions (underlined) of correct bases in siCK1α were used as another control (siCon#2,5′-CCAGGCUAGGCCAGU UGCUdTdT-3′ (SEQ ID NO:23)),

### Cell Culture, Transfections, Virus, and Parasites.

All cells were maintained in Dulbecco's modified Eagle's medium supplemented with 10% (vol/vol) fetal bovine serum (FBS; HyClone) unless otherwise specified. Mouse bone marrow-derived macrophages from the C57/BL6 mice were obtained by cultivating bone marrow cell isolates in RPMI medium containing 10% FBS and 30% of the L929 cell supernatant (a source of macrophage colony-stimulating factor) for 7 days according to a standard protocol. Human peripheral blood monocytes were obtained from University of Pennsylvania Human Immunology Core, and derivation of dendritic cells was done according to a standard protocol (Sallusto et al., 1994, J. Exp. Med. 179:1109-1118). A cell proliferation assay was carried out using the CellTiter 96 nonradioactive cell proliferation assay kit (catalog number G4001; Promega) according to the manufacturer's recommendations.

293T cells and HeLa cells were transfected with Lipofectamine Plus reagent and Lipofectamine 2000 reagent, respectively. VSV (Indiana serotype) was propagated in HeLa cells. L major (WHO MHOM/IL-1/80 Freidlin clone) was maintained in a log phase of growth in Schneider's growth medium containing 20% FBS.

### Viral and Parasite Infection of Cultured Cells.

HeLa or 2fTGH cells were inoculated with a multiplicity (MOI) of 0.1 of VSV for 1 h, washed, and added with fresh medium. At 12.5 h later, uninfected or infected cells were treated with D4476 or vehicle (dimethyl sulfoxide [DMSO]). Total cell lysates were harvested at different ensuing time points. For Leishmania infections, the macrophages were resuspended in 106 cells/ml and were infected with a 10-fold excess of L. major (50%) metacyclic in suspension culture for 4 h. Cells were subsequently washed two times to remove free parasites and further incubated as indicated.

Measurement of L. major-Secreted Kinase Activity.

A total of 50×106 confluent L. major promastigotes were washed with buffer A (20 mM Tris-HCl, pH 7.4, 150 mM NaCl, 10 mM MgCl2, 1 mM glucose, and 10 mM NaF). Cells were then resuspended in buffer A containing 50 μg/ml of GST-IFNAR1 at 30° C. for 20 min as described previously (Sacerdoti-Sierra et al., 1997, J. Biol. Chem. 272:30760-30765). The supernatant was collected, supplemented with 2 mM of ATP, and further incubated at 30° C. for 15 min. The substrate was captured by glutathione beads and analyzed in Western blot assay for phosphorylation at site Ser535.

### Immunotechniques.

Antibodies against pSTATI and p-eIF2α (Cell Signaling), eIF2a (Biosources), CK1å (BD Pharmingen), STAT1, Myc tag, HA tag, GST, CK1α (Santa Cruz), Flag tag, β-actin (Sigma), and ubiquitin (clone FK2; Biomol) were used for immunoprecipitation and immunoblotting. Monoclonal antibody 23H12, specific for the M protein of VSV (VSV-M) was used. Antibodies which recognize endogenous IFNAR1 (Goldman et al., 1999, J. Interferon Cytokine Res. 19:15-26) and IFNAR1 phosphorylated on Ser535 (or Ser526 in mouse IFNAR1 [29]) were described previously. Cell lysis, immunoprecipitation, and immunoblotting procedures as well as the kinase assay using cell lysates and GST-IFNAR1 as a substrate were previously described (Liu et al., 2009, Cell Host Microbe 5:72-83; Liu et al., 2008, Biochem. Biophys. Res. Commun. 367:388-393). Quantification of IB analyses was done using Li-Cor's Odyssey infrared imaging system.

### Flow Cytometry

Cell surface levels of IFNAR1 in human and mouse cells were determined by staining cells with anti-hIFNAR1 (AA3 [20]) or anti-mIFNAR1 (Leinco) in combination with anti-mouse-biotin (Jackson Laboratory) and streptavidin-phycoerythrin (e-Bioscience). Cell surface antigen levels were examined by using a FACSCalibur flow cytometer (BD Pharmingen). The data were analyzed with the FlowJo program (Tree Star).

The results of this experimental example are now described.

CK1α is a Kinase that Directly Phosphorylates the IFNAR1 Degron

The detection of a major ligand and JAK-independent Ser535 kinase activity in lysates from human cells was previously reported. Such activity could be monitored by an in vitro kinase assay using the bacterially expressed cytoplasmic domain of IFNAR1 fused with GST (GST-IFNAR1) as a substrate, the cell lysates as the source of kinase, and anti-phospho-Ser535 immunoblotting as a mode of detection (Liu et al., 2008, Biochem. Biophys. Res. Commun. 367:388-393). Purification of basal IFNAR1 kinase activity was carried out as outlined in FIG. 54A. Cytoplasmic lysates from untreated HeLa cells were fractionated by ammonium sulfate precipitation followed by purification on a cation exchange SP Sepharose column (as described in detail in Materials and Methods) to identify fractions that were active in this kinase assay and that could discriminate between wild type GST-IFNAR1 and the mutant GST-IFNAR1S535A counterpart in a modified assay that used radioactive ATP for detection (FIG. 54B). This modified assay was used for further purification of enriched IFNAR1 kinase activity through additional steps (FIG. 54A). Mass spectrometry analysis of five major bands obtained from pooled active fractions resolved on SDS-PAGE (FIG. 54C) revealed the presence of several peptides derived from human CK1α (see Materials and Methods).

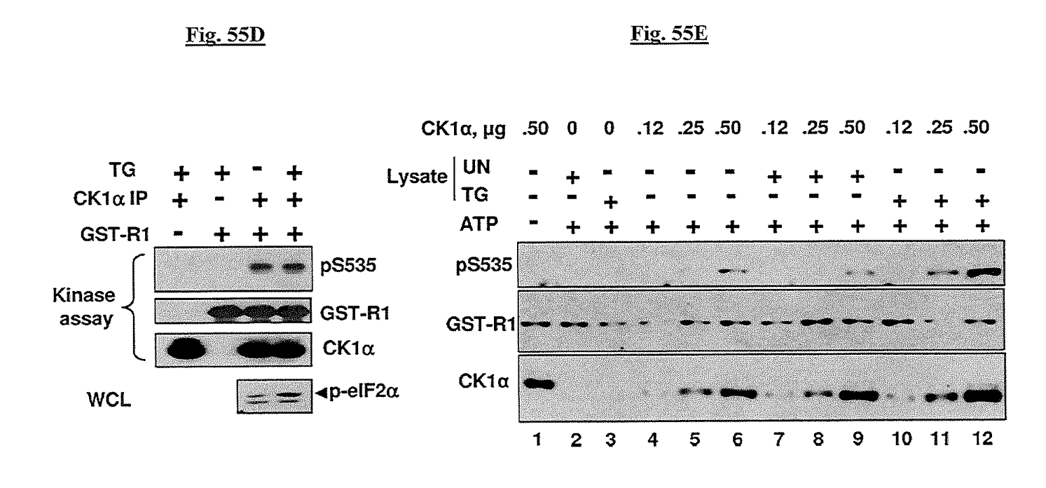

CK1α and six other members of the human CK1 family of ubiquitous pleiotropic kinases phosphorylate numerous substrates (Knippschild et al., 2005, Cell Signal. 17:675-689), some of which share the presence of a potentially phosphorylated serine or threonine residue at position n3 to enable hierarchical mechanism of primed subsequent phosphorylation (Bustos et al., 2006, Proc. Nati, Acad. Sci. USA 103:19725-19730; Bustos et al., 2005, Biochem. J. 391:417-424; Donella-Deana et al., 1985, Biochim. Biophys. Acta 829:180-187; Flotow et al., 1990, J. Biol. Chem. 265:14264-14269; Meggio et al., 1979, FEBS Lett, 106:76-80; Roach, 1991, J. Biol. Chem. 266:14139-14142; Umphress et al., 1992, Eur. J. Biochem, 203:239-243), Intriguingly, mouse and human IFNAR1 harbor similar residues (underlined), Ser529 and Ser532, in the sequence that directly precedes the degron (529SQTSQDSGNYS). Consistent with a possibility that CK1αmight function as a direct basal Ser535 IFNAR1 kinase in human cells, immunodepletion of HeLa cell lysate using the antibody against CK1α(but using neither control irrelevant monoclonal or polyclonal antibodies nor antibody against CK1 E) indeed decreased the efficacy of GST-IFNAR1 phosphorylation in vitro by this lysate (FIG. 55A). Furthermore, while RNA interference (RNAi)-mediated knockdown of CK1α in

HeLa cells decreased the ability of lysates from these cells to mediate Ser535 phosphorylation in vitro (FIG. 55B), a reverse effect was obtained upon overexpression of CK1α in 293T human embryo kidney cells (FIG. 55C). In addition, both immunopurified (FIG. 55C) and bacterially produced CK1α (FIG. 55E, lane 6) also phosphorylated GST4FNAR1 on Ser535 in vitro. Collectively, these data validate the biochemical purification strategy and indicate that CK1α is a bona fide direct kinase of Ser535 of IFNAR1.

A substantial body of literature indicates that members of the CK1 family are constitutively active kinases (Knippschild et al., 2005, Cell Signal. 17:675-689). However, given that ligand-independent phosphorylation of IFNAR1 can be further stimulated in cells treated with the inducers of ER stress, such as TG or viruses (Liu et al., 2009, Cell Host Microbe 5:72-83), it was investigated whether TG treatment activates CKla. As expected, treatment of cells with TG caused activation of PERK as assessed via phosphorylation of its substrate, eIF2α (FIG. 55D). Remarkably, CK1αpurified from the lysates from these cells (or cells treated with IFN-a) did not display a higher activity in an in vitro kinase reaction with GST-IFNAR1 as a substrate (FIG. 55D).

To examine whether a CK1α-independent factor may facilitate this kinase's actions in cells undergoing ER stress, CK1α was immunodepleted from the lysates of cells treated or not with TG. In line with the results shown in FIG. 55A, the supernatants of these reaction mixtures were not efficient in mediating phosphorylation of GST-IFNAR1 on Ser535 (FIG. 55E, lanes 2 and 3). However, when combined with bacterially expressed CK1α, the depleted lysates from TG-treated cells noticeably increased the efficacy of IFNAR1 phosphorylation (FIG. 55E, lanes 11 and 12). These results indicate that ER stress induces yet-to-be-identified cellular factors that cooperate with CK1α to increase the phosphorylation of the IFNAR1 degron.

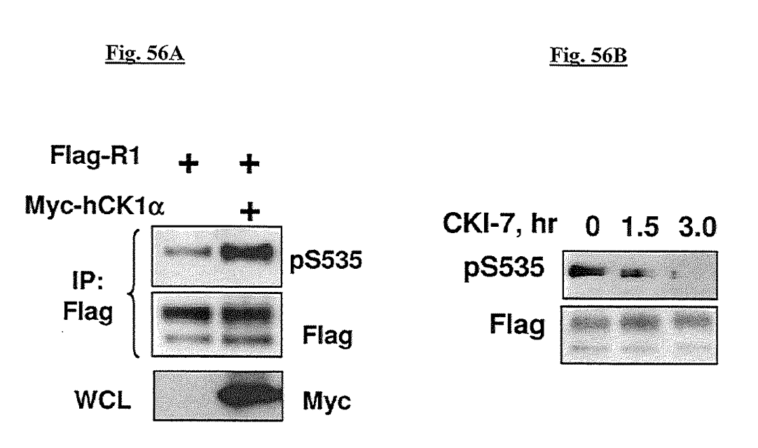

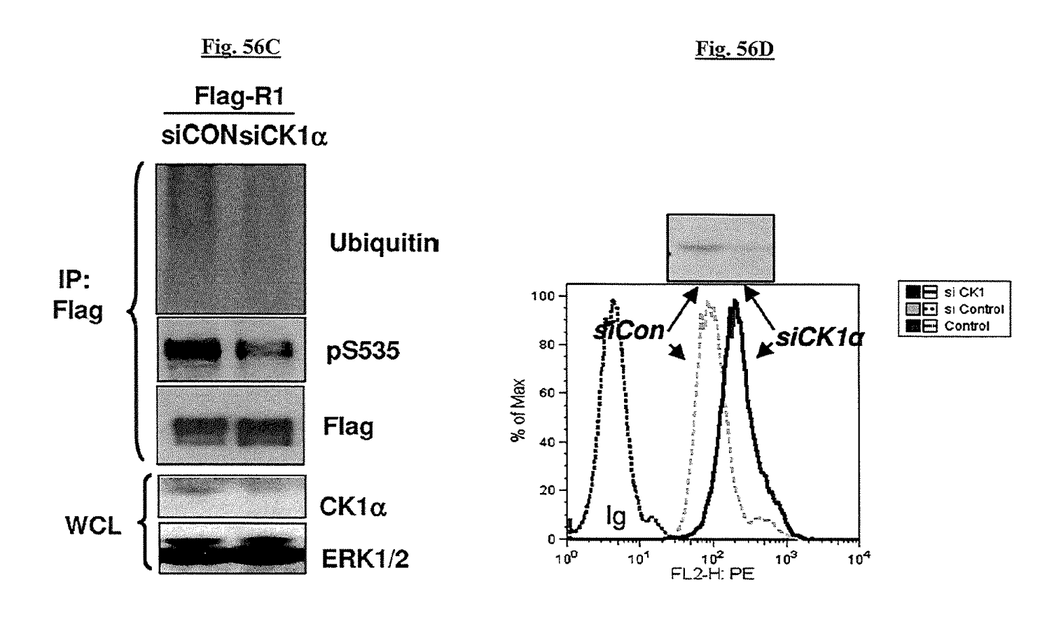

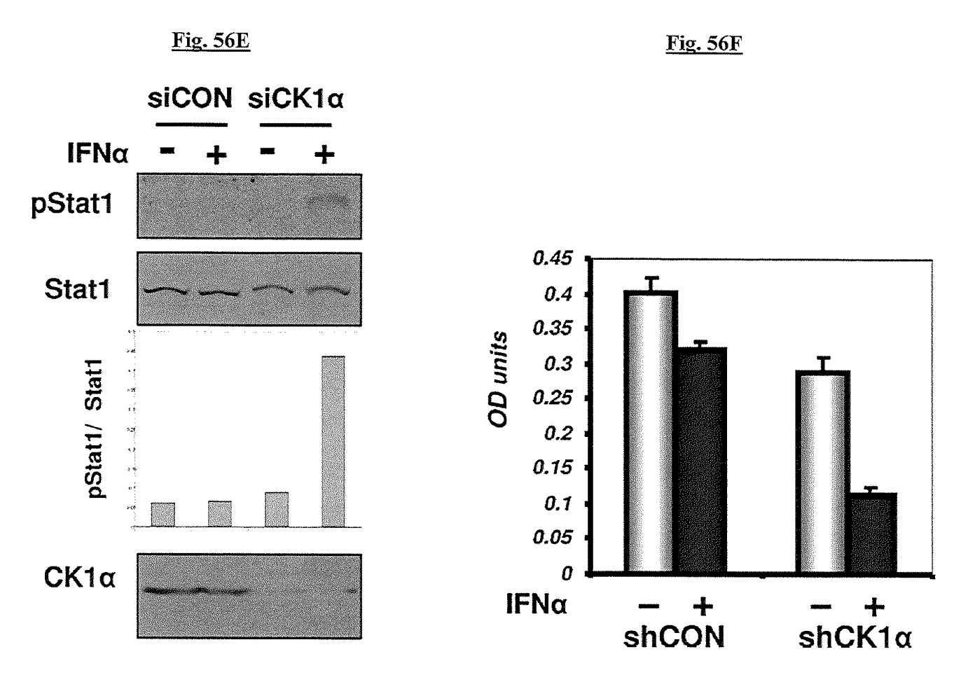

It was next examined whether CK1α mediates ligand-independent IFNAR1 phosphorylation at Ser535 in the cells. Consistent with previously published observations (Liu et al., 2008, Biochem. Biophys. Res. Commun. 367:388-393), this phosphorylation was easily detectable on Flag-tagged IFNAR1 expressed and immunopurified from human cells. Under these conditions, coexpression of human CK1α further promoted phosphorylation of the IFNAR1 degron (FIG. 56A). In addition, this phosphorylation was decreased in 293T cells treated with a CK1 inhibitor, CKI-7 (FIG. 56B). Importantly, knockdown of CK1α decreased basal Ser535 phosphorylation of coexpressed Flag-IFNAR1 (FIG. 56C).

In line with previous reports that basal phosphorylation of IFNAR1 mediates its ubiquitination in cells not exposed to IFN (32), it was also observed that knockdown of endogenous CK1α decreased the extent of IFNAR1 ubiquitination in untreated HeLa cells (FIG. 56C). Consistent with the role of IFNAR1 ubiquitination in endocytosis of this receptor (Kumar et al., 2007, J. Cell Biol. 179:935-950; Kumar et al., 2003, EMBO J. 22:5480-5490), the cell surface levels of IFNAR1 measured by fluorescence-activated cell sorting (FACS) analyses were noticeably higher in the cells transfected with siRNA against CK1α (FIG. 56D). Given that IFNAR1 levels are important for IFN-α/β signaling (Hwang et al., 1995, Proc. Natl. Acad. Sci. USA 92:11284-11288), it was tested whether modulation of CK1α expression affects the extent of cellular responses to IFN-α. A brief treatment of HeLa cells that received control siRNA by a low dose of IFN-α caused a negligible level of Stat1 phosphorylation. Under these conditions, a noticeably more pronounced activation of Stat I was observed in cells where CK1α was knocked down (FIG. 56E). Furthermore, stable downregulation of CK1α expression by shRNA constructs against CK1α augmented the antiproliferative effect of IFN-α in 2fTGH human cells (FIG. 56F). Given that CK1α is an abundant protein and its knockdown was incomplete in all these experiments, the extent of CK1α-mediated effects on IFNAR1 phosphorylation, ubiquitination, cell surface levels, and signaling are likely to be underestimated. Collectively, these data suggest that CK1α contributes to the control of IFNAR1 ubiquitination and cell surface levels of IFNAR1 as well as the sensitivity of cells to IFN-α.

### CK1α is Required for Efficient Phosphorylation and Down-Regulation of IFNAR1 Via the Ligand-Independent Pathway.

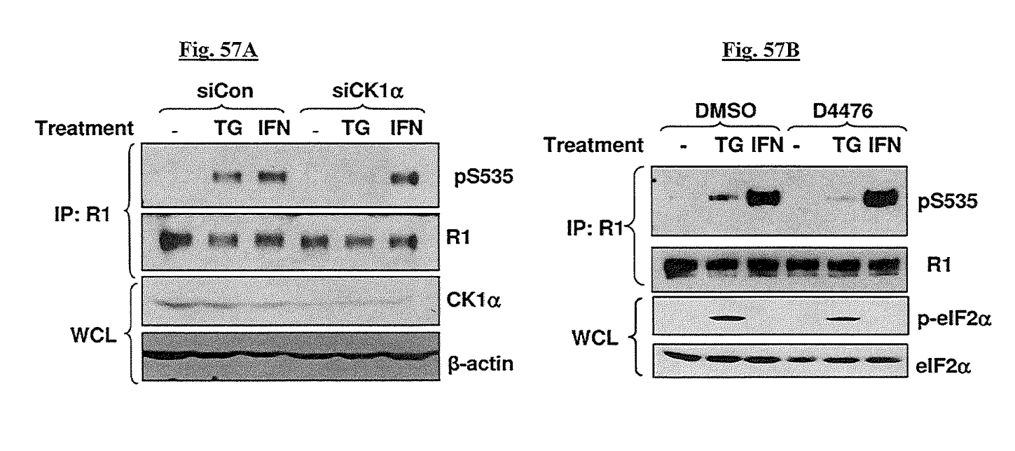

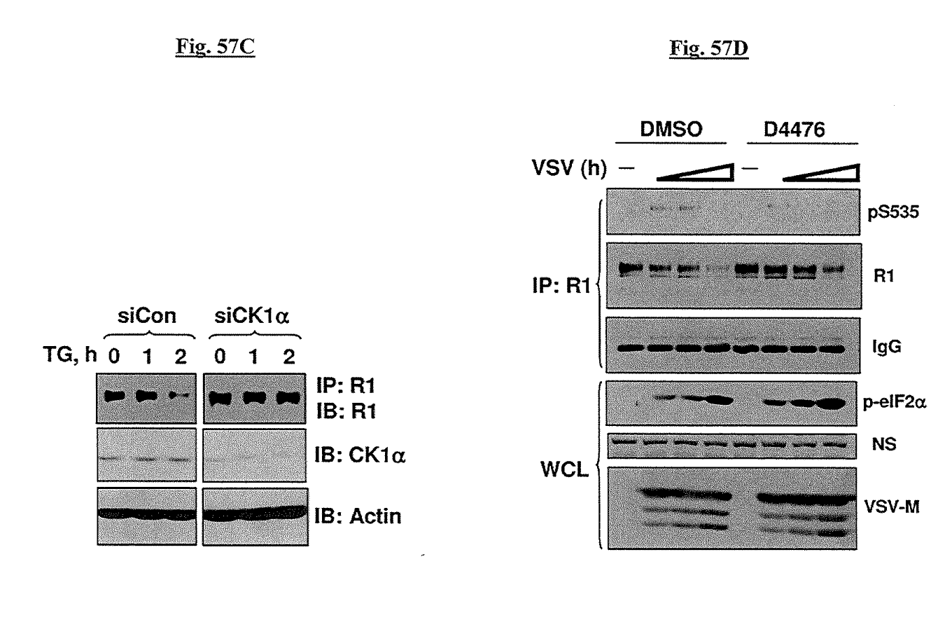

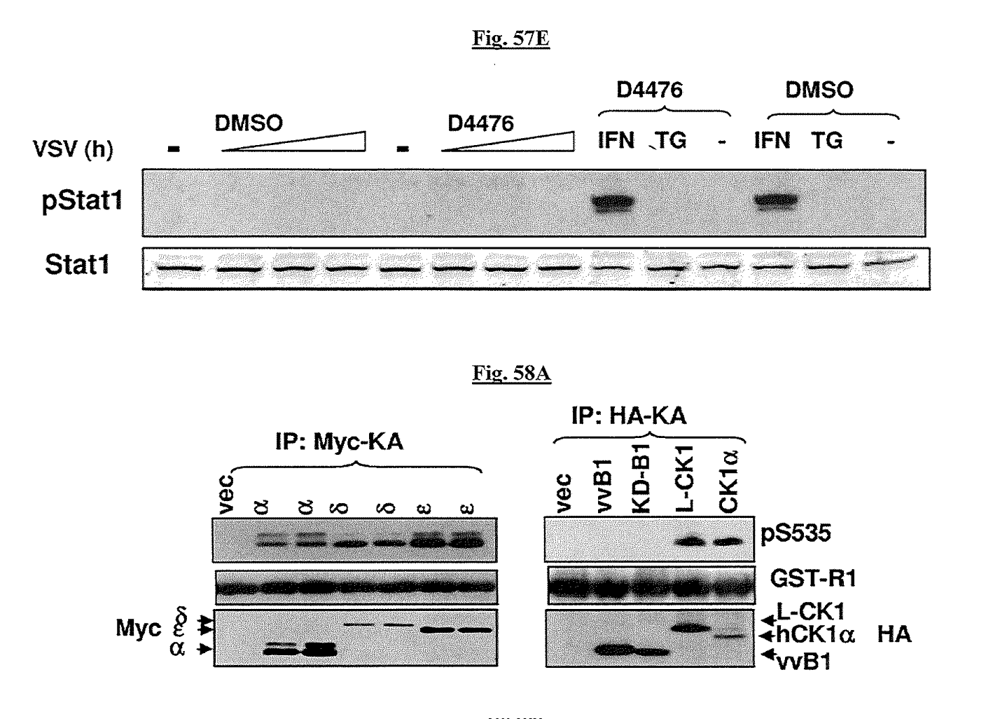

Ligand-independent phosphorylation and degradation of IFNAR1 could be further stimulated by inducers of ER stress, such as TG and infection with VSV (Liu et al., 2009, Cell Host Microbe 5:72-83). Knockdown of endogenous CK1α by RNAi noticeably decreased the extent of Ser535 phosphorylation in the cells treated with TG. Importantly, phosphorylation of IFNAR1 in response to IFN-α was not affected by siRNA against CK1α (FIG. 57A). These results indicate that CK1α is dispensable for the ligand-inducible phosphorylation of IFNAR1 but might be required for the ligand-independent pathway.

The latter possibility was further tested by a pharmacologic approach using a cell-permeable and selective CK1 inhibitor, D4476 (Bain et al., 2007, Biochem. J. 408:297-315; Rena et al., 2004, EMBO Rep. 5:60-65). Although TG caused a comparable induction of phosphorylation of eIF2α (a canonical substrate of TG-inducible PERK [He, 2006, Cell Death Differ. 13:393-403, Umphress et al., 1992, Eur. J. Biochem. 203:239-243]) regardless of pretreatment with D4476, this inhibitor noticeably attenuated the Ser535 phosphorylation of IFNAR1 in response to TG but not to IFN-α in 2fTGH cells (FIG. 57B). These data together suggest that CK1 activity is required for ligand-independent phosphorylation of the degron of IFNAR1.

ER stress induces S535 phosphorylation of IFNAR1 and accelerates its phosphorylation-dependent endocytosis and subsequent degradation (Liu et al., 2009, Cell Host Microbe 5:72-83). Consistently, in cells transfected with siRNA against CK1α, thapsigargin-induced dowmegulation of IFNAR1 was noticeably attenuated (FIG. 57C). Collectively, these results demonstrate that CK1α phosphorylates S535 to accelerate subsequent downregulation of IFNAR1, therefore controlling the levels of IFNAR1 in cells that undergo ER stress.

To further test this possibility the role of CK1 in phosphorylation and downregulation of IFNAR1 in 2fTGH cells infected with VSV, which was previously shown to induce IFNAR 1 phosphorylation and degradation in a ligand- and JAK-independent manner (Liu et al., 2009, Cell Host Microbe 5:72-83), was investigated. RNAi was not used because of the potential pleiotropic effects of loss of CK1α on viral replication and expression of viral proteins reported in literature (Bhattacharya et al., 2009, Virus Res. 141:101-104; Boyle et al., 2004, J. Virol. 78:1992-2005; Campagna et al., 2007, J. Gen. Virol. 88:2800-2810; Eichwald et al., 2004, Proc. Natl. Acad. Sci. USA 101:16304-16309; Huber et al., 2004, J. Virol. 78:7478-7489; MacLaine et al., 2008, J. Biol. Chem., 283:28563-28573; Quintavalle et al., 2006, J. Viral. 80:11305-11312; Quintavalle et al., 2007, J. Biol. Chem., 282:5536-5544). Instead, a pharmacological approach was used to acutely inhibit CK1 activity by treatment with D4476. Previous reports demonstrated that VSV infection promoted ER stress (He, 2006, Cell Death Differ. 13; 393-403) and phosphorylation-dependent ubiquitination and degradation of IFNAR1 (Liu et al., 2009, Cell Host Microbe 5:72-83). When D4476 was added to the VSV-infected cells shortly before a point where significant accumulation of a viral protein (VSV-M) can be seen, this inhibitor markedly attenuated virus-induced S535 phosphorylation of IFNAR1 and downregulation of IFNAR1 without affecting eIF2a phosphorylation (FIG. 57D). Under these conditions, it is unlikely that IFNAR1 ownregulation is driven by signaling initiated by endogenous IFN-α/β because of the lack of basal Stat1 phosphorylation in these lysates (FIG. 57E), although a possibility that Type I IFN might be produced and act at other time points of infection cannot be ruled out. In all, these results indicate the involvement of CK1α in VSV-induced S535 phosphorylation and ensuing degradation of IFNAR1.

### Leishmanial Casein Kinase Regulates IFNAR1 Levels and IFN-α/β Signaling.

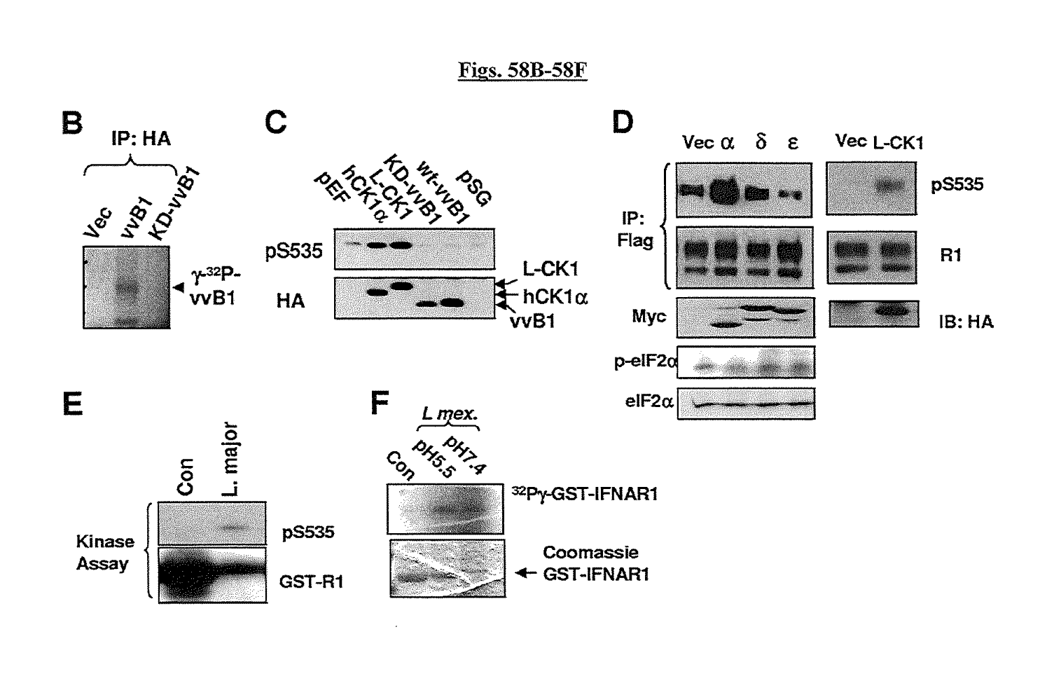

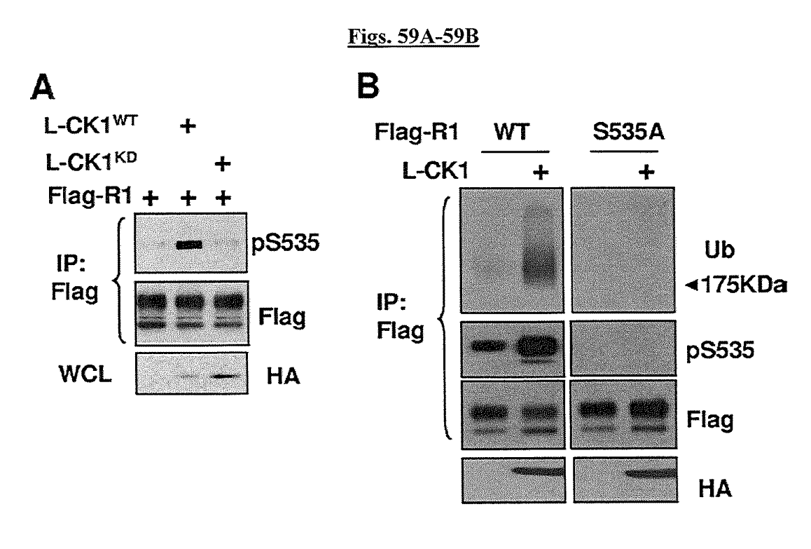

Casein kinase 1 comprises a large family of evolutionarily conserved kinases that include numerous isoforms in mammalian cells as well as CK1 orthologs and CK1-like proteins expressed in some lower organisms. It was next examined whether different members in the CK1 superfamily are capable of phosphorylating S535 of IFNAR1 in vitro and in the cells. Vaccinia virus is known to express a CK1-like kinase B1 (vvB1) that plays an important role in its replication (Rempel et al., 1992, J. Virol. 66:4413-4426). When expressed and immunopurified from 293T cells, this kinase was not capable of direct phosphorylation of IFNAR1 on Ser535 (FIG. 58A, right panel) despite being active in autophosphorylation (FIG. 58B) and against other substrates, including casein (Santos et al., 2004, Virology 328:254-265). On the contrary, immunopurified human CK18, CK1E, and protozoan parasite L-CK1 were active against IFNAR1S535 in the immunokinase assay in vitro (FIG. 58A). Accordingly, lysates from cells overexpressing hCK1α and L-CK1, but not vvB1, exhibited elevated levels of S535 kinase activity (FIG. 58C). Interestingly, although all tested human CK1 isoforms were capable of phosphorylating GST-IFNAR1 in vitro, only expression of hCKla increased the phosphorylation of Flag-IFNAR1 in the cells (FIG. 58D, left panel). Such an effect of hCK1α was unlikely to represent an artifact of specific induction of ER stress, since levels of phosphorylated eIF2a were similar in cells overexpressing all tested human CK1 forms. Similar to hCK1α, expression of L-CK1 also sufficed to promote phosphorylation of the IFNAR1 degron in the cells (FIG. 58D, right panel). These results together suggest that there is a specificity in the ability of diverse CK1 species to phosphorylate Ser535 of IFNAR1 and that there are certain structural determinants present in hCK1α and L-CK1 that enable this function in cells.

It is plausible that mammalian IFNAR1 encounters L-CK1 when the cells are infected with Leishmania parasites that shuffle between sandflies and mammalian hosts during the infectious life cycle. Within this cycle, Leishmania promastigotes are released from the insect gut to invade macrophages and dendritic cells in the mammalian hosts via phagocytosis to become mammal-parasitizing amastigotes (reviewed in Polonio et al., 2008, Int. J. Mal. Med. 22:277-286). Intriguingly, there are reports that various species of Leishmania are capable of secreting the CK1-like kinase that is active against several host mammalian substrates, including membrane proteins (Sacerdoti-Sierra et al., 1997, J. Biol. Chem. 272:30760-30765; Vieira et al., 2002, Int. 3. Parasitol. 32:1085-1093). The reported experimental conditions were used to test whether such activity is capable of phosphorylating IFNAR1. Incubation of concentrated medium obtained from L. major promastigotes with ATP and GST-IFNAR1 led to a noticeable phosphorylation of this substrate on Ser535 (FIG. 58E). In addition, kinase activity secreted by amastigotes from another Leishmania species (L. mexicana) under two different acidity conditions resulted in phosphorylation of IFNAR1 detected via incorporation of radiolabeled ATP into this substrate (FIG. 58F). These results suggest that different forms of Leishmania secrete a kinase activity that is capable of directly phosphorylating IFNAR1 within its degron.

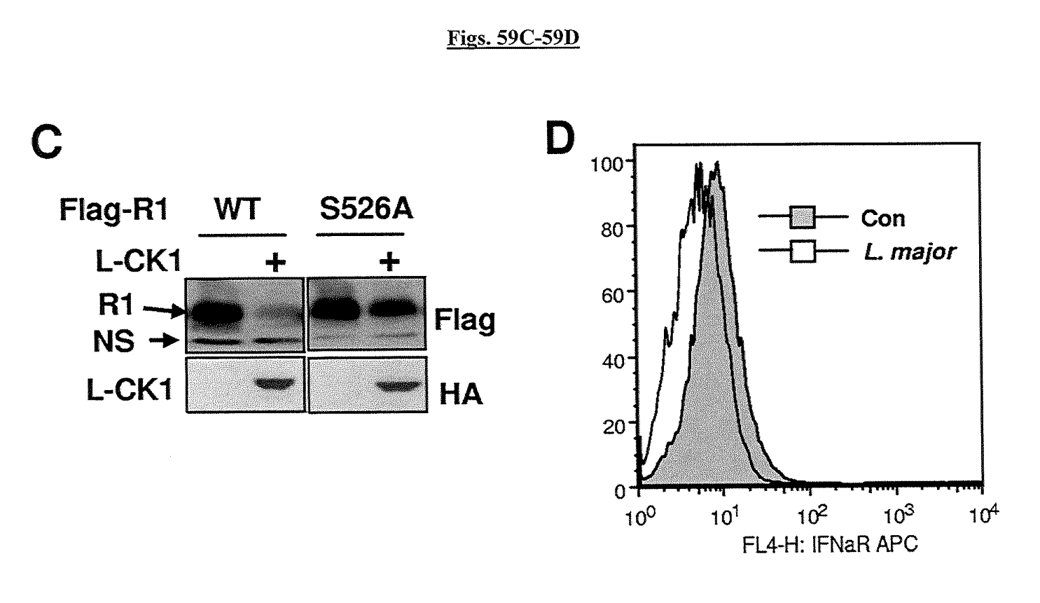

L-CK1 has been cloned and, based on studies that used inhibitors of this kinase, is implicated in controlling the growth of Leishmania (Allocco et al., 2006, Int, J. Parasitol. 36:1249-1259; Donald et al., 2005, Mol. Biochem. Parasitol. 141:15-27; Knockaert et al., 2000, Chem. Biol. 7:411-422). It was further investigated whether this kinase might regulate phosphorylation-dependent ubiquitination and degradation of IFNAR1. Expression of wild-type L-CK1 but not of its catalytically inactive mutant promoted phosphorylation of coexpressed Flag-tagged IFNAR1 on Ser535 (FIG. 59A). Furthermore, expression of L-CK1 increased ubiquitination of wild-type Flag-IFNAR1 but not of its S535A mutant, which was insensitive to the phosphorylating effects of L-CK1 (FIG. 59B), In some of these experiments, a slight decrease in the levels of wild-type Flag-IFNAR1 was observed in the cells where L-CK1 was coexpressed; however, these changes were difficult to interpret because of the presence of endogenous IFNAR1, To test whether the presence of leishmanial kinase might affect the levels of IFNAR1, mouse embryo fibroblasts obtained from IFNAR1 knockout animals were used. These fibroblasts were reconstituted with either wild-type mouse Flag-IFNAR1 or its mutant that harbors the S526A mutation (analogous to the human S535A substitution). Given that coexpression of L-CK1 decreased the levels of wild-type Flag-IFNAR1 much more dramatically than that of the phosphorylation-insensitive receptor mutant (FIGS. 59C and 59B, lower panel), it is likely that L-CK1 down regulates IFNAR1 at least in part through a phosphorylation-dependent mechanism.

Furthermore, infection of human dendritic cells with L. major led to a modest but reproducible decrease in the cell surface levels of endogenous IFNAR1 assessed by FACS (FIG. 59D). Similar results were obtained when mouse bone marrow macrophages were used for infection. Collectively these data suggest that the presence of L-CK1 in mammalian cells leads to phosphorylation of the IFNAR1 degron and ensuing phosphorylation-dependent downregulation of IFNAR1.

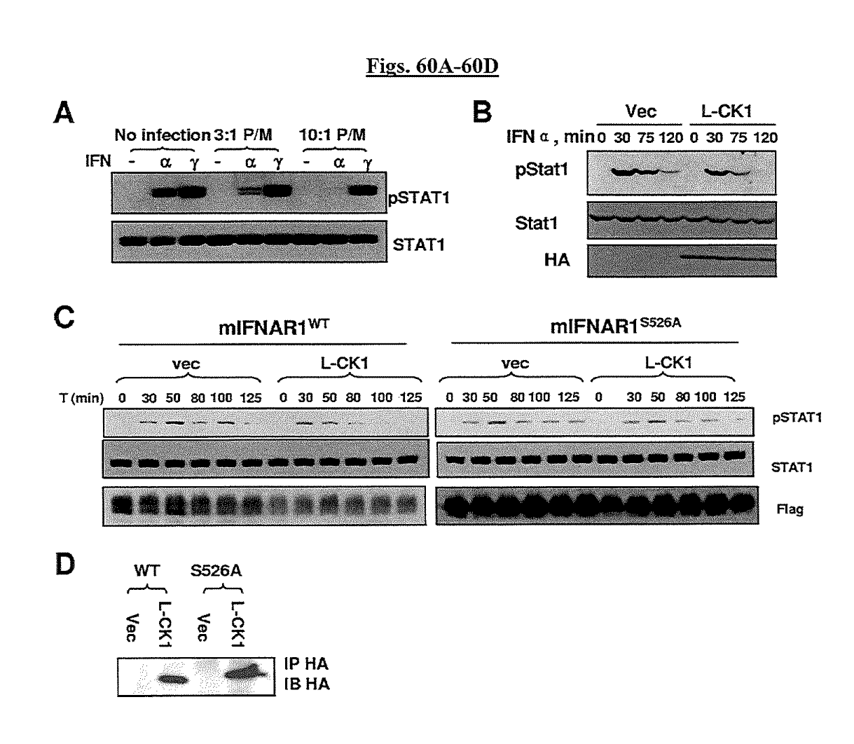

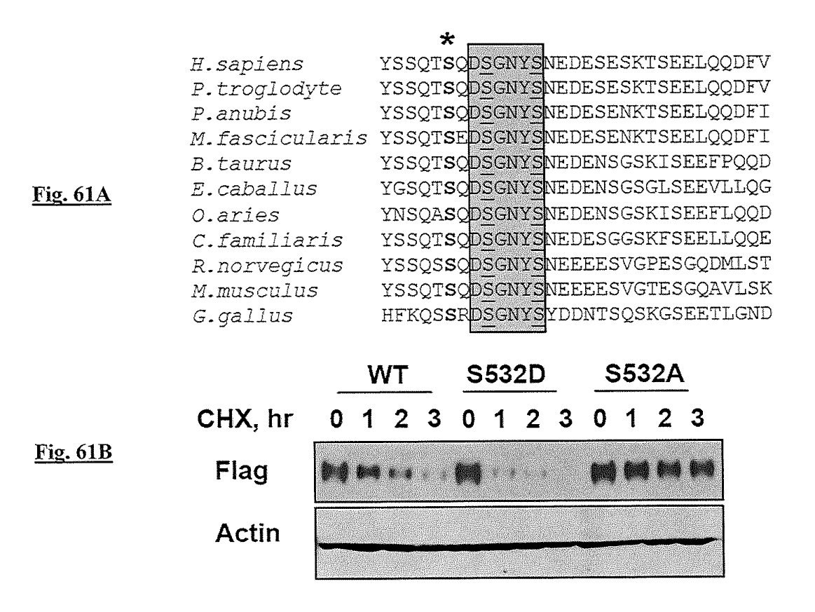

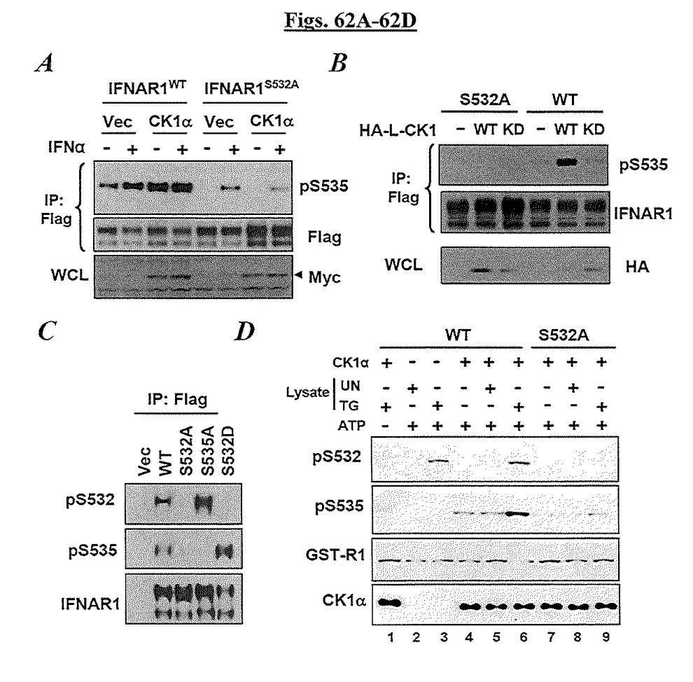

Maintenance of IFNAR1 levels plays an important role in regulation of the duration and magnitude of Type I IFN signaling (Huang-Fu et al., 2008, FEBS Lett. 582:3206-3210; Hwang et al., 1995, Proc. Natl. Acad. Sci. USA 92:11284-11288; Kumar et al., 2003, EMBO J. 22:5480-5490). The results that L. major secretes an S535 kinase activity and that L-CK1 is sufficient to cause S535-dependent IFNAR1 loss suggested that Leishmania may attenuate the extent of IFN signaling. Infection of mouse bone marrow macrophages with L. major indeed led to a dose-dependent inhibition of Stat1 phosphorylation in response to IFN-α (FIG. 60A). Remarkably, this suppression was specific, as Leishmania infection did not affect Stat1 phosphorylation induced by Type II IFN (IFN—y). Since Type I and II IFNs utilize different receptors, yet similar intracellular kinases, to activate Stat1, the latter data suggest that L. major inhibits cellular responses to Type I IFN via targeting its receptor.

To directly test the role of L-CK1 in the inhibition of Type I IFN signaling plasmid for expression of L-CK1 or empty vector were transfected in human 293T cells and followed up activation of Stat1 after pulse treatment with human IFN-α. Cellular responses to this cytokine were noticeably attenuated in cells that received L-CK1 (FIG. 60B). A similar experiment was performed on IFNAR1-null mouse embryo fibroblasts that were reconstituted with either wild-type IFNAR1 or its L-CK1-insensitive IFNAR1S526A mutant. A pulse treatment of cells with mouse IFN-α led to a temporal induction of Stat1 phosphorylation, the extent of which was reduced over time (FIG. 60C). Expression of L-CK1 in cells that harbor wild-type IFNAR1 led to a noticeable signaling inhibition that manifested itself in both a lesser magnitude and a shorter course of Stat1 phosphorylation. Importantly, these changes were much less prominent when L-CK1 was expressed in cells that harbor the IFNAR1S526A mutant (FIG. 60C), despite similar levels of LCK1 achieved in these cells (FIG. 60D). These results collectively indicate that the presence of the leishmanial CK1 in the host cells suppresses the cellular responses to IFN-α in a manner that at least in part depends on phosphorylation of the IFNAR1 degron.

### Example 6

### Inducible Priming Phosphorylation Promotes Ligand-Independent Degradation of the IFNAR1 Chain of Type I Interferon Receptor

Phosphorylation-dependent ubiquitination and ensuing downregulation and lysosomal degradation of the IFNAR1 chain of the receptor for Type I interferons (IFNs) plays an important role in limiting the cellular responses to these cytokines. These events could be stimulated either by the ligands (in a Janus kinase (JAK)-dependent manner) or by unfolded protein response (UPR) inducers including viral infection (in a manner dependent on the activity of pancreatic ER kinase, PERK). Both ligand-dependent and -independent pathways converge on phosphorylation of Ser535 within the IFNAR1 degron leading to recruitment of P-Trcp E3 ubiquitin ligase, and concomitant ubiquitination and degradation. Casein kinase I (CK1a) was shown to directly phosphorylate Ser535 within the ligand-independent pathway. Yet, given the constitutive activity of CK1α, it remained unclear how this pathway is stimulated by UPR. It is disclosed herein that induction of UPR promotes the phosphorylation of a proximal residue, Ser532, in a PERK-dependent manner. This serine serves as a priming site that promotes subsequent phosphorylation of IFNAR1 within its degron by CK1α. These events play an important role in regulating ubiquitination and degradation of IFNAR1 as well as the extent of Type I IFN signaling.

The Materials and Methods used in this Example are now described.

### Plasmids and Reagents

Thapsigargin (TG), cycloheximide (CHX), and methylamine HCl were purchased from Sigma. Human pcDNA3-Flag-IFNAR1 mammalian expression construct and retroviral pBABE-puro-based construct for expression of Flag-tagged mouse IFNAR1 as well as GST-IFNAR1 bacterial expression vector were described previously (Aaronson et al., 2002, Science 296:1653-1655). Mutants lacking the priming sites (Ser532 in human IFNAR1 and Ser523 in mouse IFNAR1) were generated by site directed mutagenesis. Sequence of mutants was confirmed by dideoxy sequencing. Constructs for expression of human Myc-tagged CK1α was described previously (Shirogane et al., 2005, J Biol Chem 280:26863-26872). HA-tagged leishmania CK1 (L-CK1) pEF-BOS-based expression vector (wild type or kinase dead K40R mutant) was described elsewhere(Liu et al., 2009, Mol Cell Biol 29: 6401-6412). pLK0.1-puro (Sigma) vector-based shRNA constructs targeted against PERK or irrelevant control were described previously (Liu et al., 2009, Cell Host Microbe 5:72-83). Construct for bacterial expression of GST-CK1α was described in (Chen et al., 2005, Mol Cell Biol 25:6509-6520). Construct for bacterial expression of constitutively active PERK (ΔN-PERK described in (Cullina et al., 2003, Mol Cell Biol 23:7198-7209)) as well as for mammalian expression of wild type or catalytically inactive PERK (K618R, (Cullina et al., 2003, Mol Cell Biol 23:7198-7209)) were previously described. Human IFN-α (Roche) and murine IFN(3 (PBL) were purchased.

### Cell Culture, Treatment, and Viral Infection

All cell lines were maintained in Dulbecco's modified Eagle's medium (DMEM) supplemented with 10% (v/v) fetal bovine serum (Hyclone) and various selection antibiotics when indicated. Human HeLa and 293T cells were obtained from ATCC. Mouse embryo fibroblasts (MEFs) from IFNAR1−/−mice and their wild type counterparts were gifted. To obtain reconstituted cells expressing wild type or mutant IFNAR1, these cells were transduced by pBabe-Puro-based InIFNAR1 constructs and selected in puromycin for two weeks before analysis. 11,1-Tyk2-null cells reconstituted with catalytically inactive Tyk2 (KR cells) were previously described (Marijanovic et al., 2006, Biochem J 397:31-38). Huh7 and derivative cells that express a complete HCV genome were described previously (Liu et al., 2009, Cell Host Microbe 5:72-83; Luquin et al., 2007, Antiviral Res 76:194-197). These cells were cultured in the presence of 500 μg/mL of G418. Transfection of 293T cells, Hela cells, and Huh7 cells was carried out with Lipofectamine Plus reagent (Invitrogen) according to manufacturer's recommendations. VSV (Indiana serotype) was propagated in HeLa cells. For infection, cells were inoculated with MOI 0.1-0.2 of VSV for 1 h, washed, and incubated with fresh medium as indicated.

### Antibodies and Immunotechniques

Commercially available antibodies against pSTAT1, p-eIF2a, STAT1 (Cell Signaling), eIF2a (Biosources), hIFNAR1, (Santa Cruz), Flag (M2), β-actin (Sigma), mouse IFNAR1 (Leinco), and ubiquitin (clone FK2, Biomol) were purchased. Monoclonal antibodies against human IFNAR1 that were used for immunoprecipitation (EAl2) or immunoblotting (GB8) were described in detail elsewhere (Goldman et al., 1999, J Interferon Cytokine Res 19:15-26). Monoclonal 23H12 antibody against the M protein of VSV (VSV-M) was a generous gift. Antibodies against IFNAR1 phosphorylated on Ser535 (Kumar et al., 2004, J Biol Chem 279:46614-46620) and against PERK (Liu et al., 2009, Cell Host Microbe 5:72-83) were described previously. Polyclonal antibody against IFNAR1 phosphorylated on Ser532 (Ser523 in the mouse receptor) was raised in rabbits using synthetic mono phosphopeptide EDHKKYSSQTpSQDSGNYSNEDE (SEQ ID NO:24) in collaboration with PhosphoSolutions Inc. (Golden, Colo.). Antibody was further affinity purified using mono-phosphopeptide affinity columns and tested for specificity by immunoblotting, Immunoprecipitations, immunoblotting, in vivo ubiquitination assay using denaturing immunoprecipitation, and assessment of the kinetics of degradation of IFNAR1 by cycloheximide chase were carried out as described previously (Kumar et al., 2003, Embo J 22:5480-5490; Kumar et al., 2004, J Biol Chem 279:46614-46620; Kumar et al., 2007, Cell Biol 179:935-950; Liu et al., 2008, Biochem Biophys Res Common 367:388-393; Liu et al., 2009, Cell Host Microbe 5:72-83).

### In-Vitro Kinase Assay

Kinase assays were carried out as described in detail elsewhere (Liu et al., 2009, Mol Cell Biol 29: 6401-6412), Briefly, 2 μg of substrates (bacterially expressed and purified GST-IFNAR1, wild type, or S532A mutant) were incubated with 4 μg of lysate (from untreated or thapsigargin treated cells) that were cleared of CK1α (by immunodepletion) and 0.25 μg of bacterially produced GST-CK1α (where indicated) in kinase buffer (25 mM Tris HCl, pH 7.4, 10 mM MgCl2, 1 mM NaF, 1 mM NaVO3) and ATP (1 mM). Where indicated, 100 μg of bacterially-produced ΔN-PERK or undepleted lysates from 293T cells were used as a source of kinase activity. Radiolabel was provided as 32P-γ-ATP (1 μCi, Amersham). The reactions were carried out at 30° C. for 30 minutes shaking at 600 rpm on the tabletop incubator. Products were analyzed either by immunoblotting with phosphospecific antibodies or by autoradiography.

The results of this example are now described.

### Inducers of UPR Promote Phosphorylation-Dependent Ubiquitination and Degradation of IFNAR1

How inducers of UPR promote phosphorylation-dependent ubiquitination and degradation of IFNAR1 was investigated. Previous studies demonstrated that these signals feed into the ligand independent pathway (Liu et al., 2008, Biochem Biophys Res Commun 367:388-393; Liu et al., 2009, Cell Host Microbe 5:72-83) that utilizes CK1α, which directly phosphorylates Ser535 within the degron of IFNAR1 (24). Given that constitutively high activity of CK1α was not further stimulated in cells treated with UPR inducers yet lysates from these cells augmented the ability of CK1α to phosphorylate Ser535 in vitro (Liu et al., 2009, Mol Cell Biol 29: 6401-6412) it was proposed that UPR signaling may lead to additional post-translational modification of IFNAR1 that improves its phosphorylation by CK1α on Ser535. Indeed, a large body of literature suggests that priming phosphorylation of a substrate at a Ser/Thr residue in the n−3 position may greatly increase its phosphorylation by various casein kinase 1 species (30-37). Analysis of primary sequences of IFNAR1 showed that a highly conserved Ser residue (Ser532 in human; Ser523 in mice) is located at this position, and may act as a priming phosphorylation site (FIG. 61A).

Ligand-independent IFNAR1 phosphorylation, ubiquitination, and degradation is readily observed in cells that overexpress this receptor (Liu et al., 2008, Biochem Biophys Res Commun 367:388-393; Liu et al., 2009, Cell Host Microbe 5:72-83). The stability of wild type Flag-IFNAR1 expressed in 293T cells with its mutant counterpart that lacks Ser532 was compared using a cycloheximide (CHX) chase assay. In this assay, levels of protein become indicative of its proteolytic turnover because they are assessed under conditions when protein synthesis in cells is inhibited for various times. Replacement of Ser at the putative priming site within IFNAR1 with Ala yielded a receptor chain that displayed a noticeably longer half life (FIG. 61B). Furthermore, substation of this serine residue with a phospho-mimicking Asp produced IFNAR1 mutant protein that underwent a more robust turnover that its wild type counterpart (FIG. 61B). This result indicates that priming phosphorylation might be important for regulating the rate of IFNAR1 proteolytic turnover.

Next it was assessed whether the priming site contributes to CK1-mediated phosphorylation of the IFNAR1 degron on Ser535. In line with previous observations (Liu et al., 2008, Biochem Biophys Res Commun 367:388-393; Liu et al., 2009, Cell Host Microbe 5:72-83; Liu et al., 2009, Mol Cell Biol 29: 6401-6412), forced expression of wild type Flag-IFNAR1 in 293T cells allowed the observation of the basal level of Ser535 phosphorylation, and co-expression of Myc-tagged CK1α further increased this phosphorylation. Under these conditions, Ser535 phosphorylation was not found in mutant IFNAR1S532A (FIG. 62A) although phosphorylation of another proximal serine mutant, Ser529A, remained unaffected. This result could be explained neither by differences in the levels of Myc-CK1α expression (FIG. 62A, lower panel) nor by the possibility that mutation in Ser532 might alter the recognition of the phospho-Ser535 specific epitope by the antibody, since Ser535 phosphorylation of IFNAR1S532A mutant was still observed in the cells treated with IFN-α. These observations suggest that the priming site is indispensable for ligand-independent IFNAR1 degron phosphorylation but not when phosphorylation is induced by IFN-α.

Similar to human CK1α, the leishmanial L-CK1 was also shown to promote phosphorylation of IFNAR1 on Ser535 upon expression in human or mouse cells (Liu et al., 2009, Mol Cell Biol 29: 6401⁻⁶⁴¹²). In line with this report, expression of wild type HA-tagged L-CK1, but not of a kinase dead mutant of L-CK1, stimulated Ser535 phosphorylation of co-expressed Flag-IFNAR1WT (FIG. 62B). However, phosphorylation of Ser535 in the S532A mutant was not observed under these conditions (FIG. 62B). Together these data further indicate that a priming phosphorylation might be required for ligand-independent CK1-mediated phosphorylation of the IFNAR1 degron. To determine whether the putative priming site is phosphorylated in cells, a polyclonal anti-pSer532 antibody was generated. Flag-IFNAR1 proteins expressed in 293T cells were immunopurified and analyzed by immunoblotting using this antibody as well as the previously characterized anti-pS535 antibody (Kumar et al., 2004, J Biol Chem 279:46614-46620). The latter antibody recognized wild type receptor but neither the S535A mutant nor the S532A mutant, whereas S532D mutant exhibited an increased phosphorylation on Ser535 (FIG. 62C). This result is consistent with data shown in FIG. 62A. Importantly, the anti-pS532 antibody recognized both wild type and the S535A mutant (but not the S532A or S532D priming site mutants; FIG. 62C), indicating that overexpressed IFNAR1 undergoes phosphorylation on the putative priming site in cells.

Whether this priming phosphorylation is directly mediated by CK1α or by another kinase that is induced by UPR was assessed. Incubation of recombinant CKla with wild type GST-IFNAR1 substrate and ATP in vitro resulted in phosphorylation of Ser535 but not of Ser532 was next evaluated (FIG. 62D, lane 4). This result confirms the previously published suggestion that CK1α is a direct kinase for the IFNAR1 degron residue, Ser535 (Liu et al., 2009, Mol Cell Biol 29: 6401-6412) but also indicates that phosphorylation of the putative priming site might be mediated by another kinase. Indeed phosphorylation of Ser532 was detected in CK1α-depleted lysates from cells treated thapsigargin (TG), an inducer of UPR. Moreover, the extent of this phosphorylation was not changed when recombinant CK1α was added to this reaction (FIG. 62D, compare lanes 3 vs. 6).

Importantly, a combination of CK1α and lysates from TO-treated cells increased the efficacy of phosphorylation of Ser535 in a manner that depended on the integrity of Ser532 as seen from the reaction using the GST-IFNAR1S532A mutant (lane 6 vs. 9). These results suggest that TO treatment induces activity of an unknown (yet different from CK1α) protein kinase that phosphorylates IFNAR1 on Ser532. Furthermore, this phosphorylation increases the efficacy of CK1a-mediated phosphorylation of Ser535 within the degron of IFNAR1, suggesting that Ser532 represents a bona fide priming site.

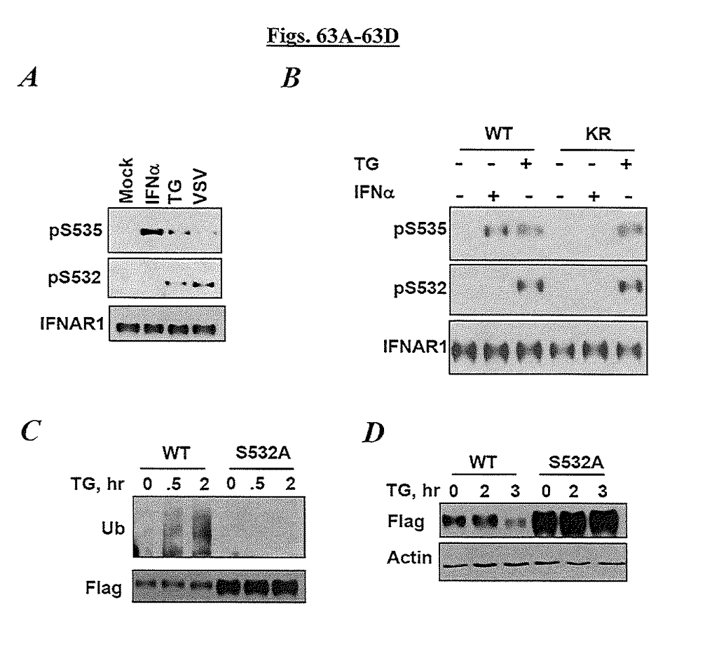

It was next investigated whether phosphorylation of the priming site may occur within the context of endogenous IFNAR1 in cells where UPR is induced. Treatment of HeLa cells with TG or infection of these cells with VSV led to phosphorylation of endogenous IFNAR1 on both Ser532 and Ser535 (FIG. 63A). In line with previously published results (Kumar et al., 2004, J Biol Chem 279:46614-46620; Marijanovic et al., 2006, Biochem J 397:31-38; Kumar et al., 2007, J Cell Biol 179:935-950), treatment of cells with IFN-α stimulated Ser535 phosphorylation. However, priming phosphorylation on Ser532 in response to the ligand was not efficient (FIG. 63A). This result, together with ligand-induced Ser535 phosphorylation of the IFNAR1S532A mutant (FIG. 62A), suggests that IFN-a-induced signaling is capable of promoting IFNAR1 degron phosphorylation in a manner that does not require priming phosphorylation. Furthermore, in human KR cells (which harbor catalytically inactive Tyk2 and were shown not to support IFN-α-induced IFNAR1 phosphorylation, ubiquitination, and degradation (Marijanovic et al., 2006, Biochem J 397:31-38; Liu et al., 2008, Biochem Biophys Res Commun 367:388-393)), the phosphorylation of the priming Ser532 site and of the degron Ser535 in response to TG was also detected (FIG. 63B). Collectively, these results suggest that phosphorylation of the priming site occurs in a ligand- and Tyk2-independent manner and is dispensable for the ligand-induced pathway.

It was previously reported that induction of UPR promotes ubiquitination and degradation of endogenous or exogenously expressed wild type IFNAR1 in human cells (Liu et al., 2009, Cell Host Microbe 5:72-83). Here the role of phosphorylation of the priming site in UPR-induced ubiquitination of IFNAR1 was investigated. Treatment of cells with TG noticeably increased the extent of ubiquitination of wild type IFNAR1 but not of the S532A mutant (FIG. 63C). Furthermore, this mutant was less sensitive to a decrease in the levels of IFNAR1 induced by TG (FIG. 63D). These data suggest that priming phosphorylation of IFNAR1 plays an important role in its ubiquitination and downregulation of IFNAR1 in response to UPR induction.

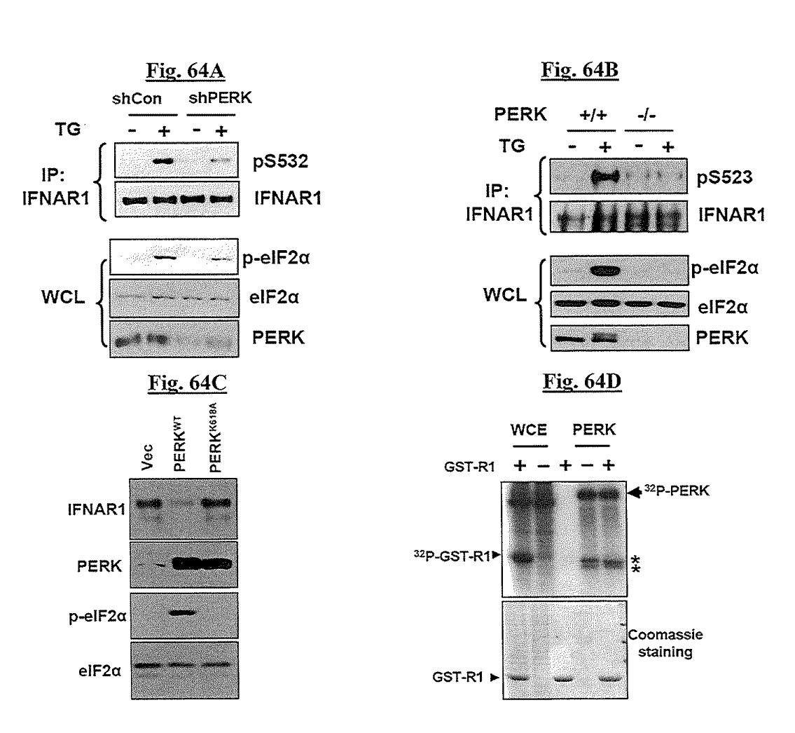

UPR stimulates Ser535 phosphorylation of IFNAR1 and accelerates ubiquitination and degradation of this receptor in a manner that relies on PERK activity (Liu et al., 2009, Cell Host Microbe 5:72-83). Whether PERK is required for phosphorylation of the priming site within IFNAR1 was next investigated. Transfection of HeLa cells with shRNA targeted against PERK led to a partial knockdown of this kinase as evident from its decreased level and a decreased phosphorylation of its known substrate eIF2α in cells treated with TG (FIG. 64A, lower panels). Under these conditions, the efficacy of TG-induced phosphorylation of IFNAR1 on the priming Ser532 was also decreased (FIG. 64A, upper panel). This result suggests that PERK is required for UPR-induced priming phosphorylation. Consistent with this suggestion, TG-induced phosphorylation of mouse IFNAR1 on Ser523 (analogous to Ser532 in human receptor) was not observed in mouse embryo fibroblasts (MEFs) from PERK knockout animals (FIG. 64B). Given that PERK plays an important role in UPR-induced ubiquitination and degradation (Liu et al., 2009, Cell Host Microbe 5:72-83) and these events also depend on the priming site of IFNAR1 (FIGS. 63C-63D), the findings that PERK regulates Ser532 phosphorylation also indicate that this kinase might function upstream of the phosphorylation of the priming site.

Expression of wild type but not catalytically inactive PERK mutant led to a noticeable downregulation of endogenous IFNAR1 (FIG. 64C) suggesting that kinase activity of PERK is required for ligand-independent IFNAR1 degradation. Whether PERK may serve as a direct kinase for the priming site was next investigated. Incubation of recombinant active PERK with GST-IFNAR1 and ATP in an in vitro kinase assay similar to one shown in FIG. 62D did not yield any phosphorylation of the substrate on Ser532 that would be detectable by immunoblotting using anti-pS532 antibody. Furthermore, when this reaction was carried out in the presence of radiolabeled ATP the incorporation of phosphate into GST-IFNAR1 was not detected (FIG. 64D). Having excluded the possibilities that integrity of substrate might be somehow compromised (by analyzing protein load using Coomassie staining and demonstrating that this very substrate was efficiently phosphorylated by the whole cell lysate) or that the kinase was inactive (given efficient autophosphorylation and phosphorylation of contaminants denoted by asterisks on FIG. 64D) it was concluded that PERK is not capable of directly phosphorylating IFNAR1. This result together with data from FIG. 62D demonstrating induction of a Ser532 kinase in cells treated with TG also suggests that UPR stimulates a PERK-dependent activation of another serine kinase that function as a direct kinase for priming phosphorylation. Alternatively, PERK activity might negatively regulate a hypothetical Ser532 phosphatase.

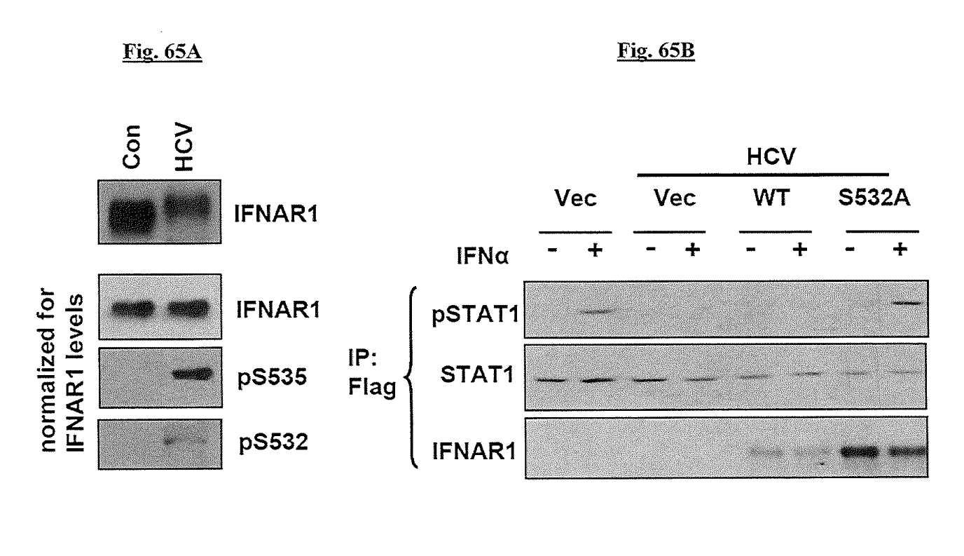

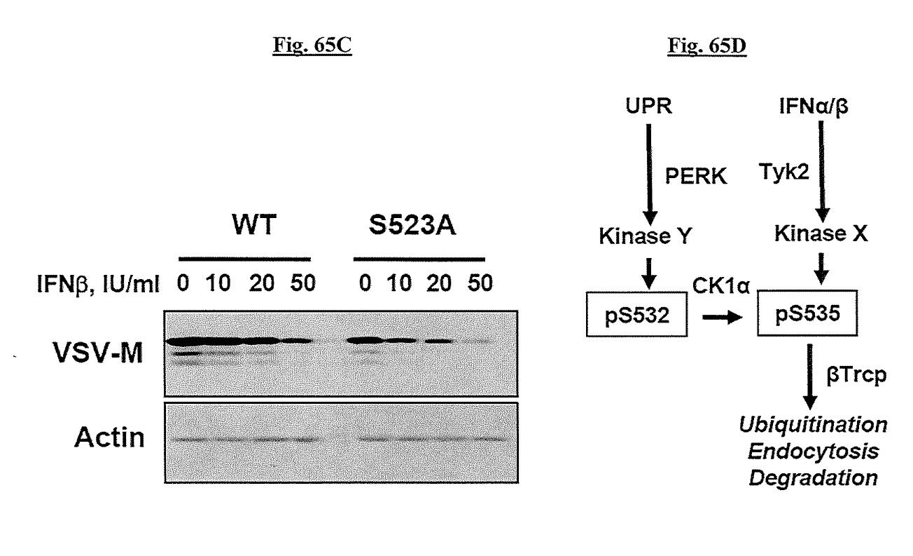

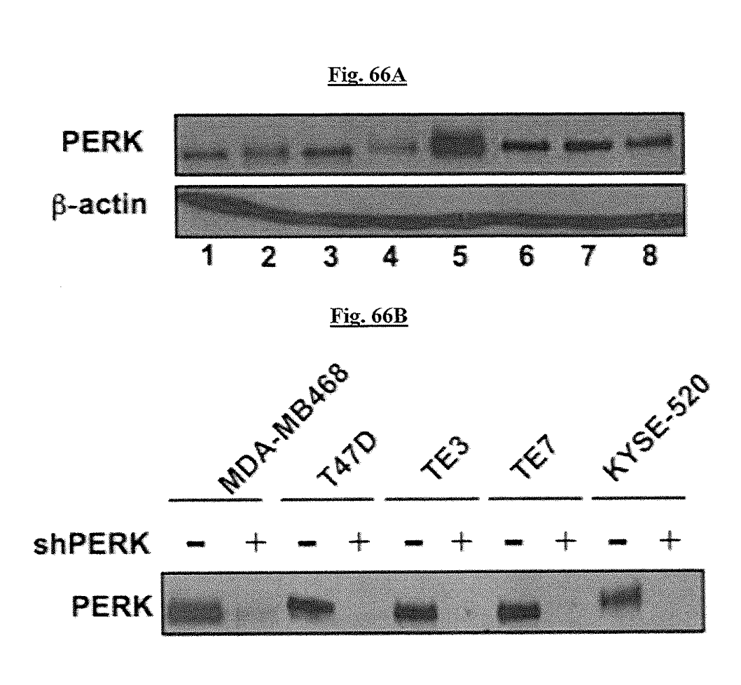

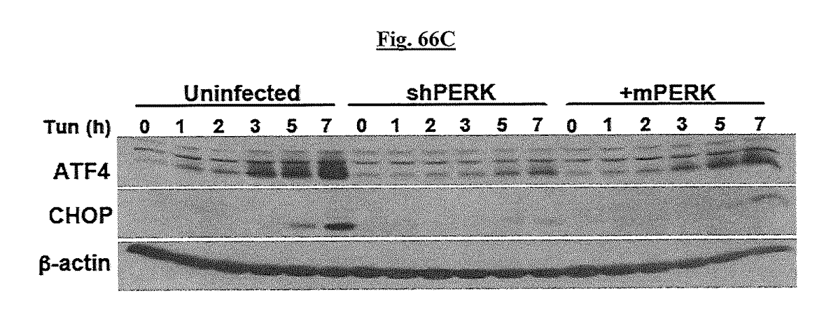

UPR induced by some viruses including VSV and HCV was shown not only to downregulate IFNAR1 but also to inhibit the extent of IFN-α/β signaling, providing these viruses with the means to evade the control from Type I IFN system (Liu et al., 2009, Cell Host Microbe 5:72-83). Whether phosphorylation of the priming site is important for attenuation of cellular responses to IFN was next investigated. In line with previously reported data (Liu et al., 2009, Cell Host Microbe 5:72-83), expression of the HCV genome in human Huh7 hepatoma cells noticeably downregulated the level of endogenous IFNAR1 (FIG. 65A, upper panel). When loading was normalized to yield comparable amounts of IFNAR1 in the immunoprecipitation reaction, HCV induced phosphorylation on both Ser535 and Ser532 was also observed (FIG. 65A, lower panel). This result suggests that cells expressing the HCV genome display an increased priming phosphorylation of IFNAR1 that may lead to downregulation of the receptor.

The response of these cells to IFN-α was markedly attenuated (Liu et al., 2009, Cell Host Microbe 5:72-83). Whether this inhibition could be rescued by expression of IFNAR1 deficient in Ser532 phosphorylation was assessed. Because of limited transfection efficacy in Huh7 cells, Flag-tagged Stat1 with Flag-tagged IFNAR1 proteins were coexpressed and then analyzed Stat1 phosphorylation and levels in Flag immunoprecipitation reactions. This analysis revealed a decreased phosphorylation of Flag-Stat1 (FIG. 65B) likely due to a decreased level of IFNAR1 (as shown in FIG. 65A). Co-expression of Flag-IFNAR1S532A mutant that is insensitive to HCV-induced priming phosphorylation restored the efficacy of IFN-α-induced Flag-Stat1 phosphorylation. However, equal amounts of vector for expression of wild type Flag-IFNAR1 failed to reverse the HCV-mediated inhibition, most likely due to the fact that wild type IFNAR1 is susceptible to ligand-independent ubiquitination and degradation (as seen from FIGS. 63C-63D) and, as a result, is expressed at levels markedly lower than that of the priming site phosphorylation-deficient mutant (FIG. 65B, lower panel).

These data indicate that priming phosphorylation of IFNAR1 may regulate IFN-α/β signaling. To further explore this possibility MEFs from IFNAR1 knockout mice weer reconstituted with either wild type murine IFNAR1 or its priming site Ser523 mutant and compared the ability of murine IFN-β to induce an anti-viral state in these cells. Cells that express the priming site mutant exhibited a noticeably higher innate resistance to VSV infection (as judged from lower levels of expression of VSV-M protein in the absence of exogenous IFN-β (FIG. 65C). Furthermore, these cells required at least five times a lower dose of exogenous IFN-β than cells expressing wild type receptor to mount a comparable defense against VSV (compare VSV-M levels at dose 50 IU/ml in WT cells versus 10 IU/ml in S523A in FIG. 65C). These data together indicate that priming phosphorylation of IFNAR1 contributes to the regulation of the cellular responses to Type I IFN.

### Example 7

### Targeted Deletion of PERK Promotes Oxidative DNA Damage Checkpoint Activation and Limits Tumor Expansion

Initial findings implicated PERK as anti-tumorigenic regulator as PERK deficiency inhibited the ability of Ras-transformed MEFs to grow as subcutaneous transplants (Bi et al., 2005, Embo J 24:3470-81; Blais et al., 2006, Mol Cell Biol 26:9517-32). However, subsequent studies revealed that overexpression of a dominant negative PERK allele in MCF10A normal mammary epithelial cells rendered neoplastic growth characteristics (Sequeira et al., 2007, PLoS ONE 2:e615). In addition, activation of Fv2E-PERK engineered to contain a drug-inducible dimerization domain reduced tumorigenic potential of squamous carcinoma T-HEp3 cells and SW620 colon carcinoma cells (Ranganathan et al., 2008, Cancer Res 68:3260-8). Finally, activation of PERK by overexpression of H-Ras in melanocytes was associated with a senescent phenotype (Denoyelle et al., 2006, Nat Cell Biol 8:1053-63) suggesting that PERK may function as a barrier to malignant growth in certain contexts.

In the experiments disclosed herein, the importance of PERK for tumorigenesis utilizing short hairpin RNA approach to reduce PERK levels in human breast and esophageal carcinoma cells was investigated. In addition, a mammary gland-specific knockout of PERK in the mammary tumor-prone MMTV-Neu mouse strain was generated. Previous results revealed that loss of PERK renders tumor cells acutely susceptible to oxidative DNA damage. The subsequent induction of the DNA damage checkpoint significantly reduces tumor cell growth in vitro and in vivo. In order to proliferate and expand in an environment with limited nutrients, cancer cells co-opt cellular regulatory pathways that facilitate adaptation and thereby maintain tumor growth and survival potential. The endoplasmic reticulum (ER) is uniquely positioned to sense nutrient deprivation stress and subsequently engage signaling pathways that promote adaptive strategies. As such, components of the ER stress-signaling pathway represent potential anti-neoplastic targets. However, recent investigations into the role of the ER resident protein kinase PERK have paradoxically suggested both pro- and anti-tumorigenic properties. Animal models of mammary carcinoma have been used to interrogate PERK contribution in the neoplastic process. The ablation of PERK in tumor cells resulted in impaired regeneration of intracellular antioxidants and accumulation of reactive oxygen species triggering oxidative DNA damage. Ultimately, PERK deficiency impeded progression through the cell cycle due to the activation of the DNA damage checkpoint. The data disclosed herein reveal that PERK-dependent signaling is utilized during both tumor initiation and expansion to maintain redox homeostasis and thereby facilitates tumor growth.

The Materials and Methods used in this Example are now described.

### Animals and Tissue

Mammary gland-specific PERK knockout animals (Bobrovnikova-Madon et al., 2008, Proc Natl Acad Sci USA 105:16314-9) were mated to mice bearing the Neu transgene under the control of MMTV-LTR promoter (Guy et al., 1992, Proc Natl Acad Sci USA 89:10578-82). The Neu and Cre transgene bearing offspring were bred to homozygocity for the LoxP allele of PERK thus generating mammary gland-specific PERK ‘null’. Littermates bearing Neu but not the Cre transgene were used as controls, The No. 4 inguinal gland was extracted and processed for whole-mount analysis as previously described (Lin et al., 2008, Oncogene 27:1231-42).

### Immunoprecipitation and Immunoblotting

Cells were lysed in EBC buffer (50 mM Tris pH 8.0; 120 mM NaCl; 0.5% NP-40) supplemented with protease and phosphatase inhibitors. Antibodies used for immunoblotting analysis, immunofluorescence and IHC included PERK (Rockland Immunochemicals); human ATF4, histone H3 (trimethyl K₉), phospho-Chk2 (Thr68) (Abeam); human CHOP (Affinity Bioreagents), R-actin (Sigma, AC-15), Nrf2, Keap 1, CDK2, and p DARE (Santa Cruz Biotechnology); y-H2AX (Ser139), phospho-eIF2, eIF4E, Cdc25A, phospho-Tyr15 CDK2, phospho-Thr160 CDK2, phospho-Thr (Cell signaling); troma-1 (Developmental Studies Hybridoma Bank, University of Iowa), ErbB2 (Calbiochem), Chk2 (BD Pharmingen), eIF2 (BioSource), phospho-ATM (Millipore).

Lentivirus, Retrovirus shRNA/siRNA

293T cells were transfected with PMDL, VSVG, REV and pLK0.1 containing shRNA against PERK (IDTRCN0000001401, Open Biosystem) or pLK0.1 empty vector as control using Lipofectamine Plus (Invitrogene) for stable knockdown or FuGene (Roche) for acute knockdown experiment. Viral supernatants were harvested 48 h after transfection and concentrated using SW-28 rotor for stable knockdown infections. Concentrated virus was used to infect human cell lines in the presence of 10 g/ml polybrene. Selection to create stably knocked down cell lines was conducted with puromycin at 5 g/ml. Retroviruses were produced as previously described (Brewer et al., 2000, Proc Natl Acad Sci USA 97:12625-30). MDA-MB468 PERK knockdown cells were transfected with siRNA by using HiPerfect (Qiagen). Scrambled (Sam) and keapl-specific siRNA Smartpool were from Dharmacon. Experiments were conducted 72 h after transfection.

### Growth Curves

3×10̂4 cells were plated in 6 cm dish. Cells were counted every 24 h for 5 days using hemocytometer. ROS scavenger N-acetylcysteine (NAC) was used at 5 mM where indicated. Culture media was changed every 3 days. Each experiment was done in triplicate.

### Quantitative and Semiquantitative RT-PCR

RNA was prepared from cultured cells or frozen tissues using TRIzol (Invitrogen), followed by isopropanol precipitation. Genomic DNA (gDNA) was isolated using Qiagen DNeasy kit. Quantitative RT-PCR reactions were performed using SYBR Green (SuperArray). All primer sequences are available upon request. Semiquantitative RT-PCR for PERK excision efficiency was performed as described (Zhang et al., 2006, J Biol Chem 281:30036-45).

### Immunofluorescence

Cells were permeabilized with ice cold MeOH:acetone (1:1) for 10 min at −20° C., allowed to air dry, and rehydrated for 10 min with PBS. Blocking was performed with 10% FBS/PBS for 40 minutes at room temperature. Primary and secondary antibodies were diluted in 10% FBS/PBS and incubated for 2 h or 30 minutes at RT, respectively.

### FISH

Fluorescent in situ hybridization for ErbB2 was performed on paraffin sections following treatment with proteinase K. Biotin-labeled probe was generated by random priming method with ErbB2 full-length cDNA (ID 5356166, Open Biosystems) and visualized with streptavidin-Texas Red.

### ROS Measurement

Cells were incubated with 3 ml PBS (with Calcium and Magnesiun) containing 5 mM 5-(and -6)-chloromethyl-2′,7′-dichlorodihydrofluorescein diacetate, acetyl ester (CM-H2DCFDA, Invitrogen) for 30 min in the dark at 37° C. Cells were washed with PBS, trypsinized, washed, resuspended in PBS and analyzed by FACS.

### 8-Oxyguanine Staining

4×10̂5 cells were plated on glass coverslips. 8-oxyguanine was detected using OxyDNA test (Biotrin). Briefly, cells were permeabilized with MeOH:Acetone (1:1), washed with wash solution, and incubated with protein-FITC for 1 h. For tumor sections, antigen retrieval was performed by heating in 50 mM Tris pH 9.5 for 12 min. Slides were immersed in ice cold MeOH for 10 min at −20° C. Sections were blocked with 10% FBS in PBS and incubated overnight at 4° C. with FITC-conjugate in wash solution.

### Comet Assay

DNA fragmentation was tested by alkaline electrophoresis comet assay (Trevigen) according to manufacturer's instructions. Data were analyzed using Comet assay IV software (Perceptive Instruments),

### Orthotopic Injections

5×10̂6 cells were mixed with 301 matrigel:media (1:1) and were injected into the mammary fat pad of 3 months old female SCID mice (Charles River). Animals were sacrificed after 37 (human cells) or 28 (mouse cells) days, tumor size was measured using a caliper, and tumor volume was calculated using the formula: Volume (cc)=p×(length)×(width)2/6 (Bruns et al., 2004, Clin Cancer Res 10:2109-19).

### Immunohistochemistry.

Antigen retrieval was performed in 10 mM citrate buffer, pH 6.0 (Biogenex). Endogenous peroxidase activity was blocked with 3% peroxide in MeOH. Sections were blocked with 1× Power Block Reagent (Biogenex) followed by incubation with primary antibody. Detection was performed with biotinylated secondary antibodies and ABC-HRP reagent followed by DAB substrate (Vector laboratories).

### In Vitro Kinase Assay

For the detection of CDK2 kinase activity, cells or tissues were solubilized in EBC buffer. Complexes were isolated by precipitation with a CDK2 reactive antibody from 200 g total protein. The kinase assay was performed using recombinant histone H1 with 10Ci of −32P-ATP for 10 min at 30° C. Reactions were resolved by SDS-PAGE, transferred to PVDF membrane, and visualized by autoradiography. Total Histone H1 was visualized by ponceau stain.

### Cell Cycle Analysis

Cells were pulsed with 10M BrdU 45 min prior to being harvested. Cells were washed with PBS, fixed with ethanol, and stained with anti-BrdU (BD Pharmingen) and FITC-conjugated secondary antibody (BD Pharmingen) and then with propidium iodide (10 g/ml) for 30 min prior to FACS analysis. Cell cycle profiles based on DNA content and BrdU incorporation were assessed using FlowJo software, and the sub-G1 population of cells served as a readout for apoptotic cells.

The Results of this Example are now described.

### PERK is Expressed in Cancer Cells Wherein it Potentiates Tumor Expansion

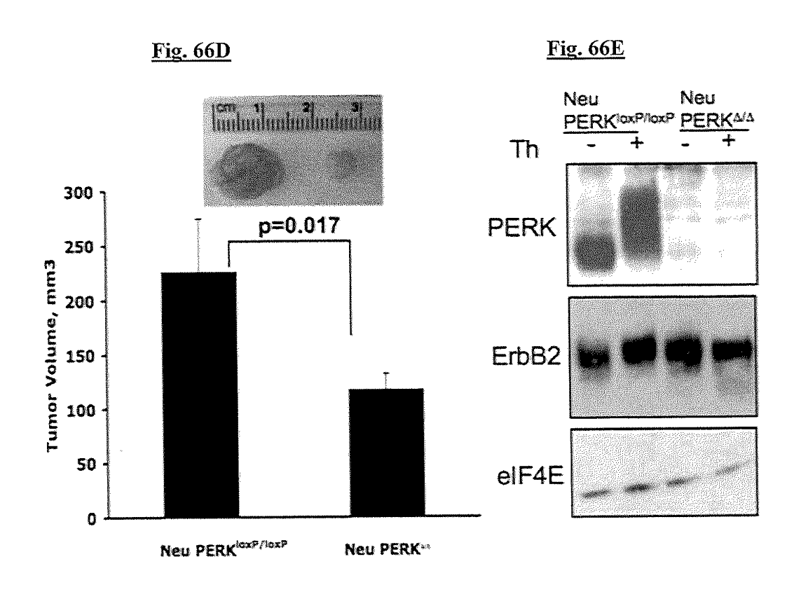

Markers of ER stress signaling, including phospho-eIF2 and GRP78 expression, are increased in a variety of tumor types (Daneshmand et al., 2007, Hum Pathol 38:1547-52; Fernandez et al., 2000, Breast Cancer Res Treat 59:15-26; Gazit et al., 1999, Breast Cancer Res Treat 54:135-46; Lee et al., 2008, Neuro Oncol 10:236-43). Because PERK mediates cell growth and survival under conditions of ER stress, it was first determined whether tumor-derived cells retain functional PERK. PERK expression was assessed in 4 breast and 3 esophageal human carcinoma-derived cell lines and compared it to the PERK levels in MCF10A cells, an immortalized, non-transformed breast epithelial cell line. PERK protein was readily detectable in all cell lines (FIG. 66A), and PERK function was preserved in cancer cells, as evidenced by their ability to activate PERK-dependent effectors (FIG. 66C).

To assess the role of PERK in human tumor cell growth and survival, lentivirus-delivered short hairpin RNAs (shRNA) were utilized to reduce endogenous levels of PERK (FIG. 66B), which also resulted in attenuated activation of PERK effectors such as ATF4/CHOP in MDA-MB468 cells challenged with tunicamycin (FIG. 66C).

To determine whether PERK deficiency affects the ability of mammary carcinoma cells to form solid tumors in vivo, tumor-prone MMTV-Neu transgenic mice bearing PERKloxP/loxP allele (MMTV-Neu/PERKloxP/loxP) were utilized. Primary tumors from MMTV-Neu/PERKloxP/loxP mice were isolated and transduced with empty vector retrovirus or retrovirus encoding Cre recombinase to excise PERK (FIG. 66E). The primary tumor cells were then transplanted into mammary fat pads of 3-week old SCID mice. During a 28-day interval, PERK-deficient tumor cells generated tumors with a significantly reduced volume relative to PERK positive cells (FIG. 66D). Similar reduction in tumor volume was observed upon PERK knockdown in human MDA-MB468 cells (FIG. 74). These data collectively demonstrate a role for PERK as a critical regulator of mammary tumor expansion.

### Loss of PERK in Human Cancer Cells Delays Cell Cycle Progression Through the G2/M Phase

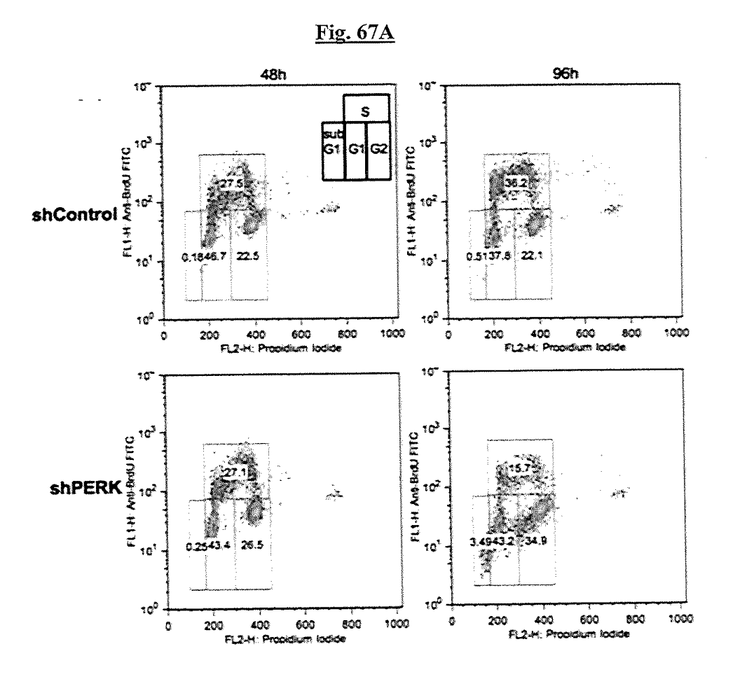

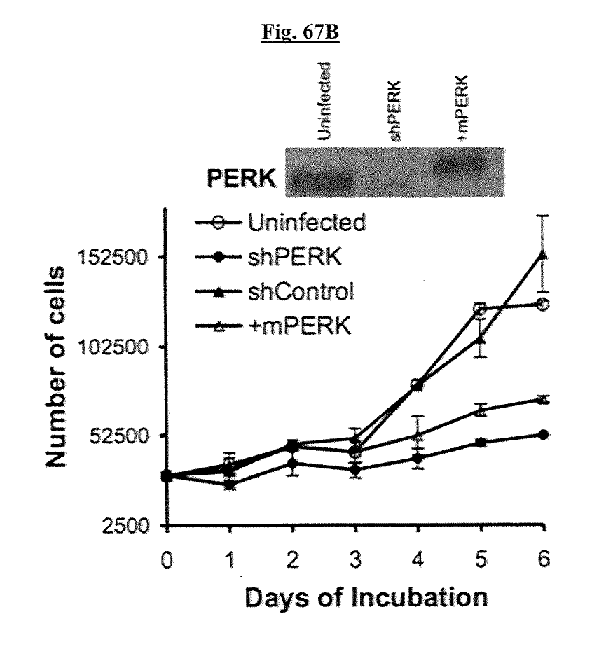

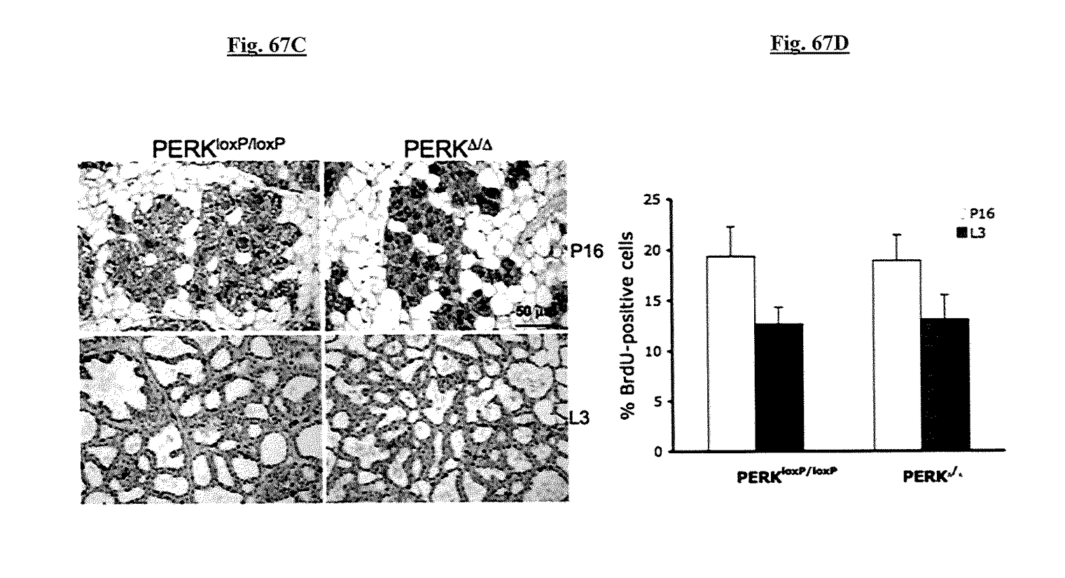

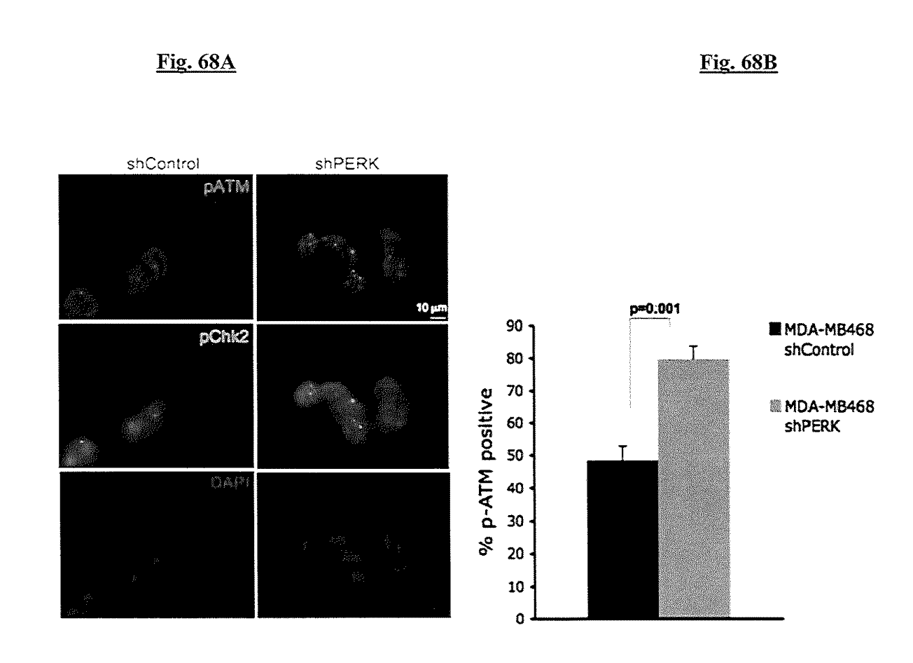

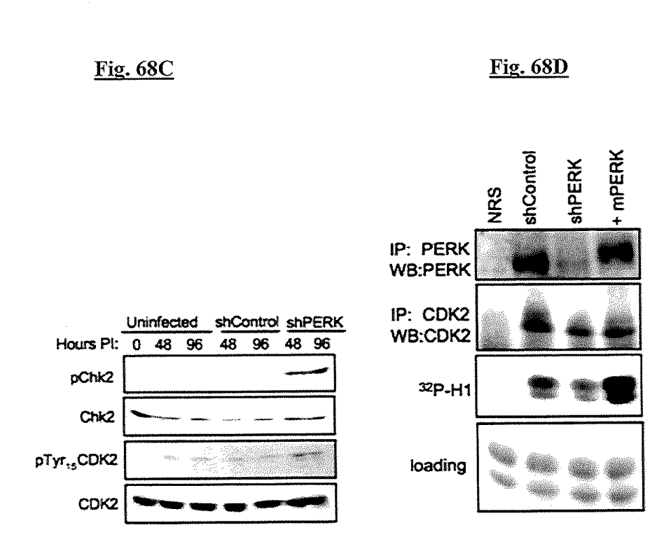

Gain and loss of PERK function can influence cell cycle progression of certain cells (Wei et al., 2008, J Cell Physiol 217:693-707; Zhang et al., 2006, Cell Metab 4:491-7). Accordingly, subsequent to acute PERK knockdown in MDA-MB468 and T47D cells, we noted a 50% reduction in BrdU-incorporating S-phase cells with a concomitant increase in G2/M phase cells and a small increase in cell death (FIGS. 67A and 75A). These data demonstrate that knockdown of PERK triggers a cell cycle delay at the G2/M boundary, resulting in a significant decrease in cancer cell proliferation in vitro. To confirm this, growth rates of parental versus stable PERK knockdown cells utilizing three different human carcinoma cell lines derived from two distinct cancer types were measured. Strikingly, PERK knockdown resulted in a significant reduction in the growth rate of human breast carcinoma MDA-MB468 (FIG. 67B) and T47D (FIG. 75B) cells, as well as esophageal carcinoma TE3 (FIG. 75C) cells compared to control cell lines. To ensure that the reduced growth kinetics specifically reflected reduced levels of PERK, PERK function was restored by transducing cells with retrovirus encoding myc-tagged murine PERK that is refractory to shRNA (FIG. 67B). Murine PERK rescued tunicamycin-dependent induction of ATF4 and CHOP (FIG. 66C) and resulted in a significant rescue of attenuated cell growth (FIG. 67B). To determine whether attenuated proliferation of tumor cells following loss of PERK function is restricted to tumors, it was examined whether excision of PERK in normal mammary epithelium inhibits proliferation. Critically, PERK excision in mammary epithelial cells did not influence their proliferative capacity (FIG. 67C-D).

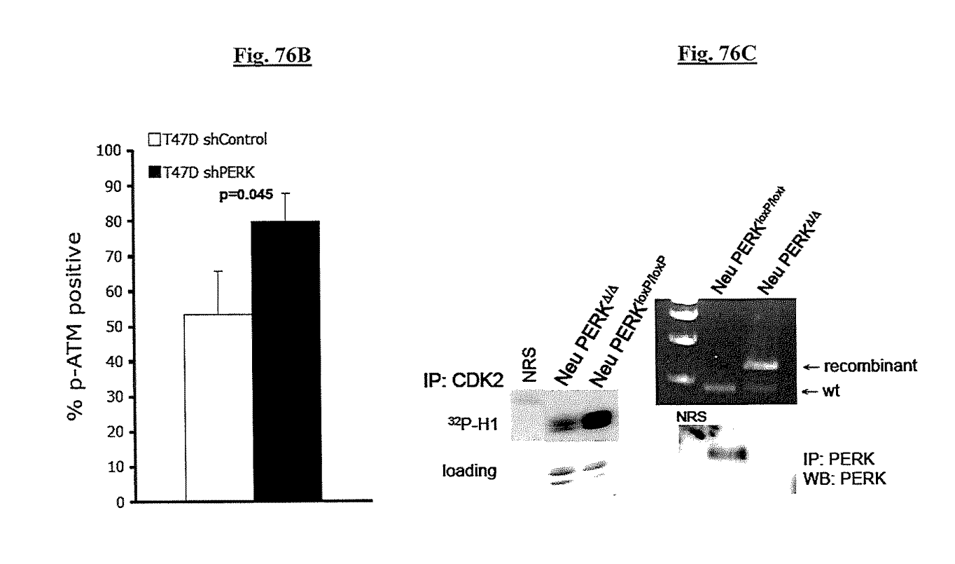

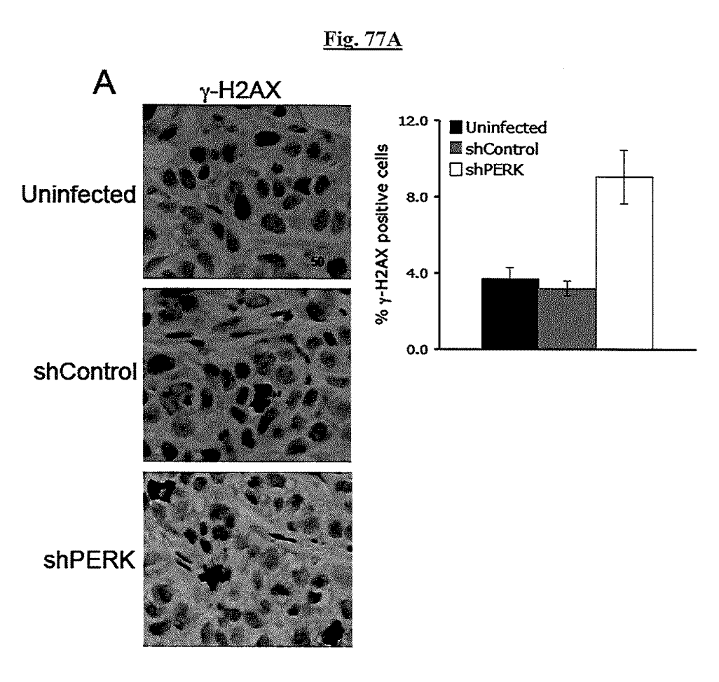

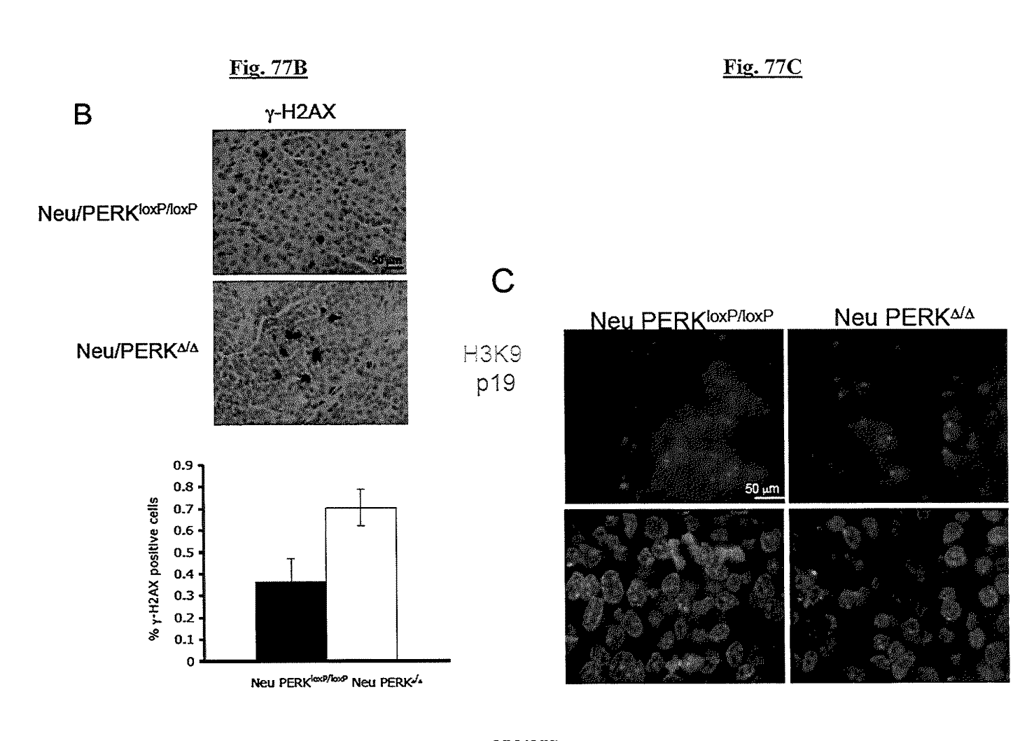

G2/M cell cycle delay/arrest is frequently associated with the activation of a double strand DNA break (DSB) checkpoint. Thus, we next tested for the evidence of DNA damage response pathway activation. Indeed, acute PERK knockdown coincided with accumulation of phospho-ATM and phospho-Chk2 positive foci in MDA-MB468 (FIG. 68A-B) and T47D cells (FIG. 76A-B). Coordinately, increased phospho-Chk2 and pTyr-15 on CDK2 (FIG. 68C) as well as reduced CDK2 kinase activity, which could be restored by introduction of murine PERK (FIG. 68D), was noted. In addition, a significant inhibition of CDK2 activity in a lysate prepared from tumor wherein PERK was excised was noted (FIG. 76C). These data demonstrate that loss of PERK delays progression through the G2/M transition due to the activation of DNA damage checkpoint.

### Reactive Oxygen Species (ROS) Accumulate in PERK Deficient Cells

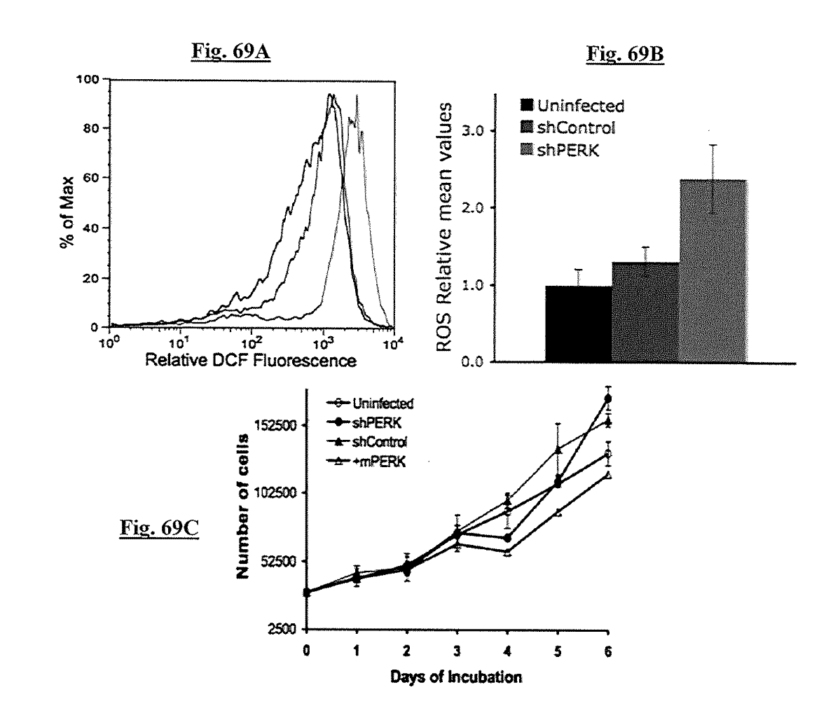

Previous work revealed a role for PERK in the regulation of cellular redox homeostasis via direct phosphorylation of Nrf2 (Cullinan et al., 2004, J Biol Chem 279(19):20108-17; Cullinan et al., 2003, Mol Cell Biol 23:7198-7209) and translational regulation of ATF4 (Harding et al., 2003, Mol Cell 11:619-33). Thus, it was determined whether PERK loss contributed to increased cellular ROS in human breast carcinoma cells. Indeed, PERK knockdown led to significantly increased levels of ROS (FIG. 69A-B). Furthermore, growth curve analysis in the presence of ROS scavenger, N-acetyl cysteine (NAC), revealed that ROS accumulation contributed to reduced cell growth in PERK knockdown cells (FIG. 69C).

### ROS Accumulation Triggers Oxidative DNA Damage

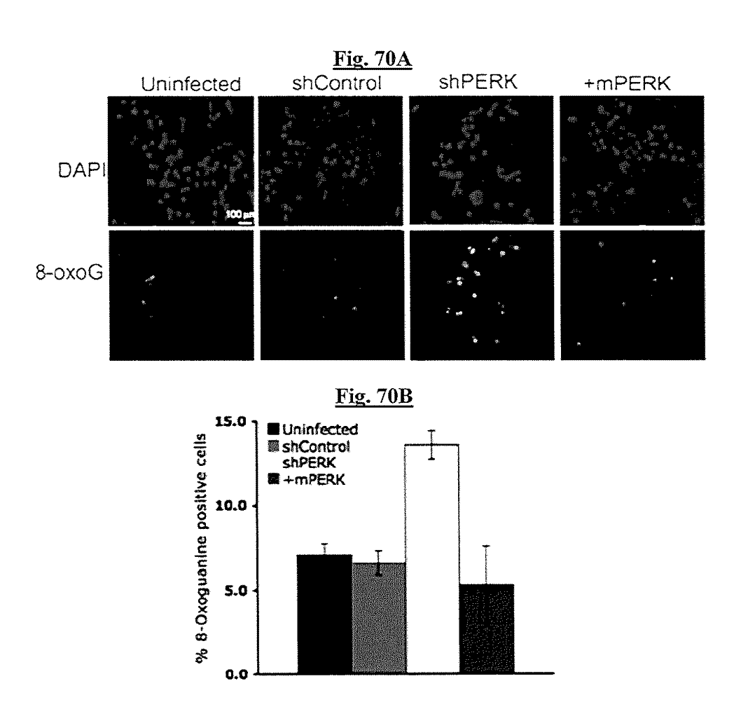

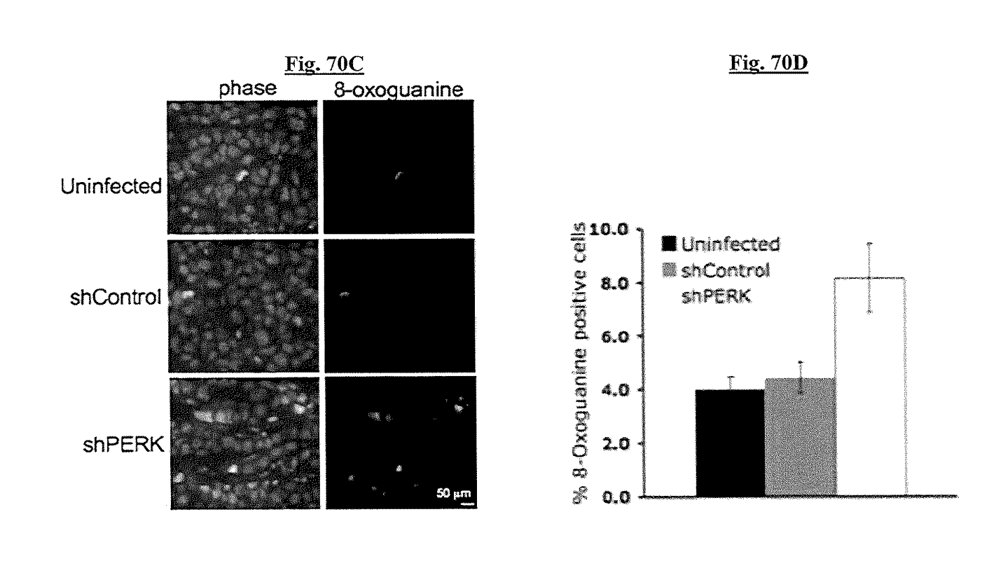

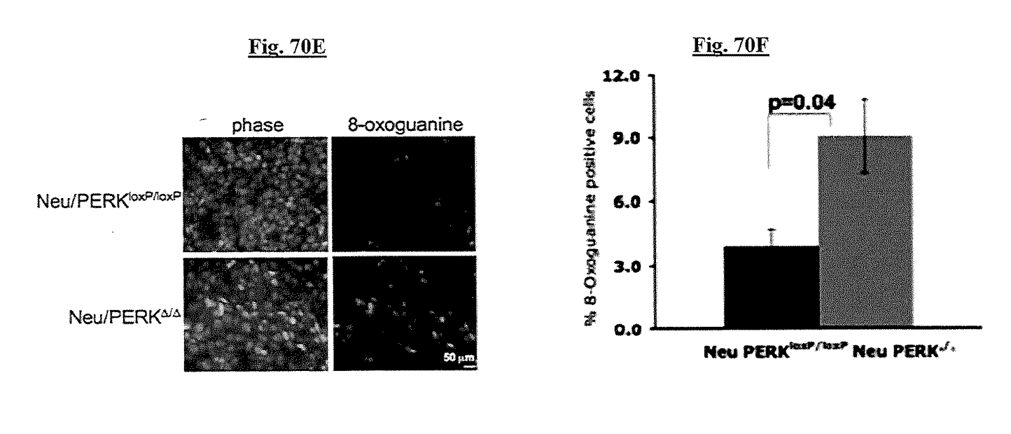

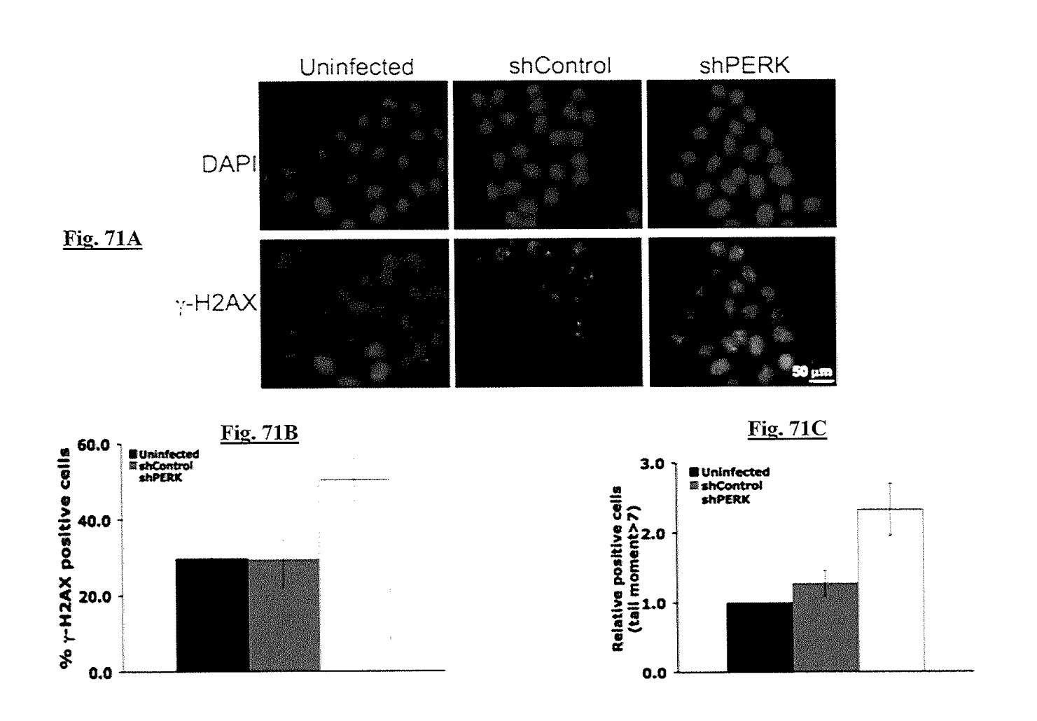

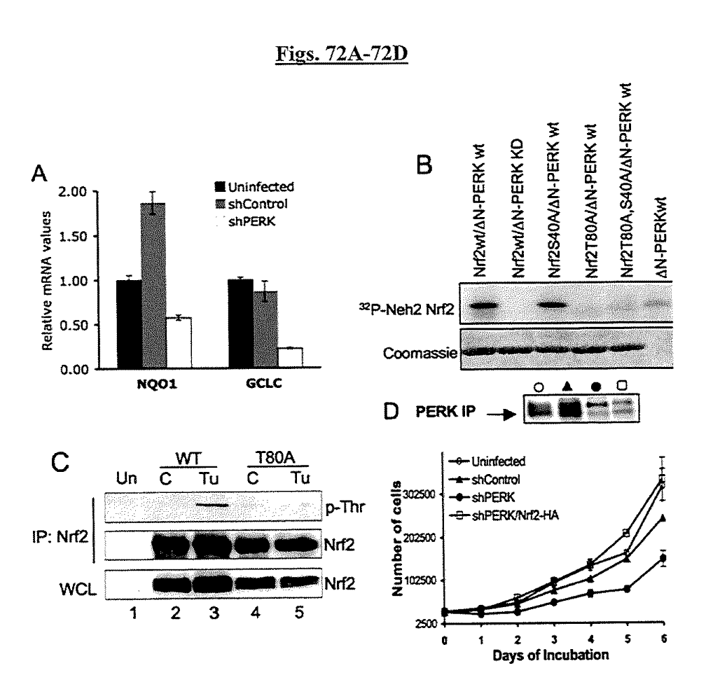

To determine whether PERK loss and subsequent accumulation of ROS triggers oxidative DNA damage, 8-oxoguanine adducts, an oxidation product of guanine, were quantified. PERK knockdown resulted in a significant increase in 8-oxoguanine adducts relative to parental cells (FIG. 70A-B). A significant increase in 8-oxoguanine adducts in both human and mouse PERK-deficient tumors was also noted (FIG. 70C-F), consistent with the idea that ROS-mediated cellular damage could also contribute to reduced growth of PERK deficient tumors in vivo. Following DNA damage, H2AX, a histone H2 variant, is phosphorylated around regions of double strand breaks (DSBs) by the PI3K family members ATM and ATR, and it accumulates in DNA repair foci wherein it contributes to the recruitment of DNA repair proteins (Basing et al., 2004, Cell Cycle 3:149-53). Thus, the accumulation of phosphorylated H2AX, -H2AX, was measured, as a surrogate marker for DSBs formation. A 2-fold increase in -H2AX. was noted in PERK knockdown cells relative to parental cells and/or cells infected with an empty vector consistent with increased DNA breaks (FIG. 71A-B). As −H2AX is also thought to accumulate under conditions not associated with DSBs, the accumulation of damaged DNA in single cells was assessed through the use of a COMET assay. Indeed, PERK knockdown triggered accumulation of cells with pronounced tail moment (FIG. 71C). Critically, increased levels of −H2AX-positive cells in PERK deficient tumors in vivo was also noted (FIG. 77A-B).

While activation of a DSB checkpoint typically results in a transient arrest and cell cycle restart following repair, it is also associated with cellular senescence when triggered by oncogene induction. However, increased accumulation of p19Arf and tri-methylated H3K9 was not observed in PERK deficient tumors suggesting that loss of PERK does not induce a senescent phenotype (FIG. 77C).

### Reduced Activity of Nrf2 Leads to Increased Oxidative Stress in Perk Knockdown Cells

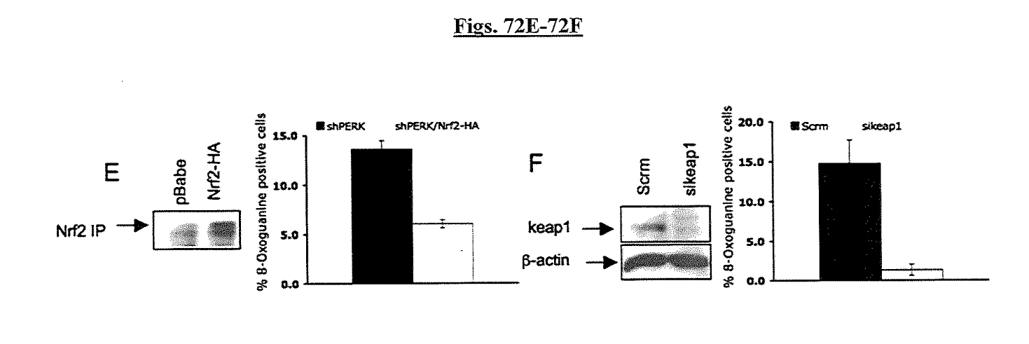

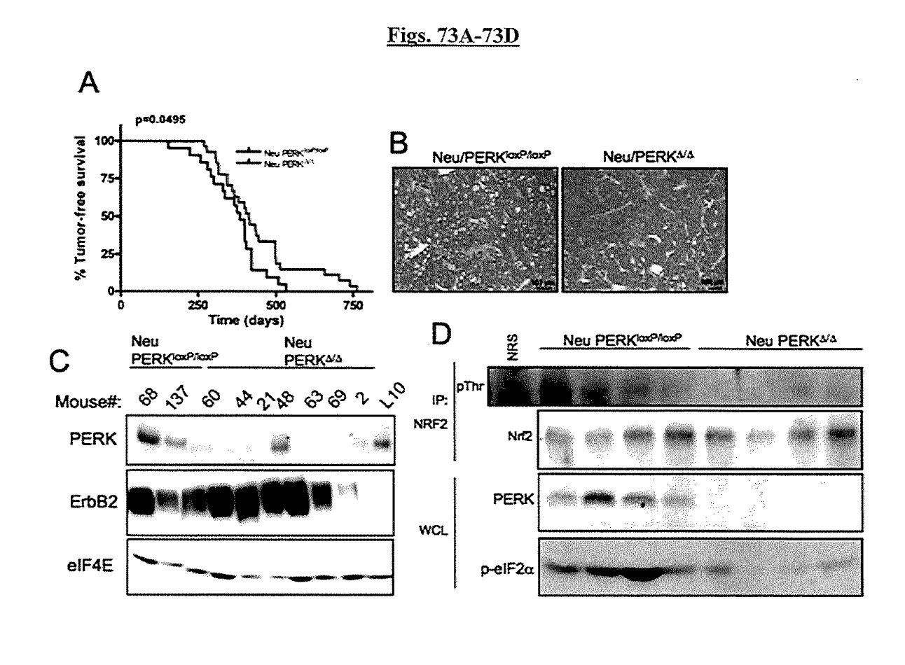

Nrf2, a direct PERK substrate (Cullinan et al., 2003, Mol Cell Biol 23:7198-7209), contributes to the transcriptional regulation of genes whose protein products mediate cellular redox homeostasis (Buetler et al., 1995, Toxicol Appl Pharmacol 135:45-57; Hayes et al., 2000, Biochem Soc Trans 28:33-41). Consistent with impaired Nrf2 activation in PERK knockdown cells, expression of two distinct Nrf2 target genes, NQO1 (Itoh et al., 1997, Biochem Biophys Res Commun 236:313-22) and GCLC (Wild et al., 1999, J Biol Chem 274:33627-36) was decreased compared to the uninfected or control cells (FIG. 72A).

To address the role of Nrf2 downstream of PERK, the site of PERK phosphorylation in Nrf2 was examined. Because PERK-dependent phosphorylation disrupts Nrf2-Keap1 binding, the Neh2 domain of Nrf2 that binds directly to Keap1 was assessed (Lo et al., 2006, Embo J 25:3605-17). Indeed, PKC can phosphorylate serine 40 in this domain (Huang et al., 2002, J Biol Chem 277:42769-74). Purified recombinant PERK phosphorylated wild type Nrf2-Neh2 and a serine 40 to alanine mutant; however, mutation of threonine 80 to alanine abrogated phosphorylation (FIG. 72B). The stress-dependent phosphorylation of this residue in Nrf2 was confirmed using a phospho-threonine reactive antibody (FIG. 72C). The threonine 80 is essential for Keap1-Nrf2 binding (Lo et al., 2006, Embo J 25:3605-17), providing a biochemical basis for phosphorylation-dependent disruption of the Keap1-Nrf2 interaction.

Subsequently, it was determined whether cellular phenotypes resulting from loss of PERK could be rescued through enforced Nrf2 function. Nrf2 activity is restricted via its association with an E3 ligase wherein Keap1 functions as Nrf2-specific adaptor thereby targeting Nrf2 to cullin 3 (Cullinan et al., 2004, Mol Cell Biol 24:8477-86; Furukawa et al., 2003, Nat Cell Biol 5:1001-7; Furukawa et al., 2005, Mol Cell Biol 25:162-71; Kobayashi et al., 2004, Mol Cell Biol 24:7130-9; Zhang et al., 2004, Mol Cell Biol 24:10941-53). It previously demonstrated that basal levels of active Nrf2 can be elevated via either overexpression of Nrf2 or knockdown of Keap1 (Cullinan et al., 2004, J Biol Chem 279(19):20108-17). Accordingly, expression of HA-Nrf2 restored normal growth to MDA-MB468 cells wherein PERK was ablated (FIG. 72D). Consistent with its role in PERK-dependent regulation of redox homeostasis, introduction of HA-Nrf2 significantly attenuated oxidative DNA damage (FIG. 72E). To independently confirm that increased Nrf2 activity can compensate for loss of PERK function and reduce oxidative DNA damage, Keap1 was knocked down in cells wherein PERK was stably reduced. This resulted in a significant reduction in oxidative DNA damage (FIG. 72F).

### Dual Role for PERK in Tumorigenesis In Vivo

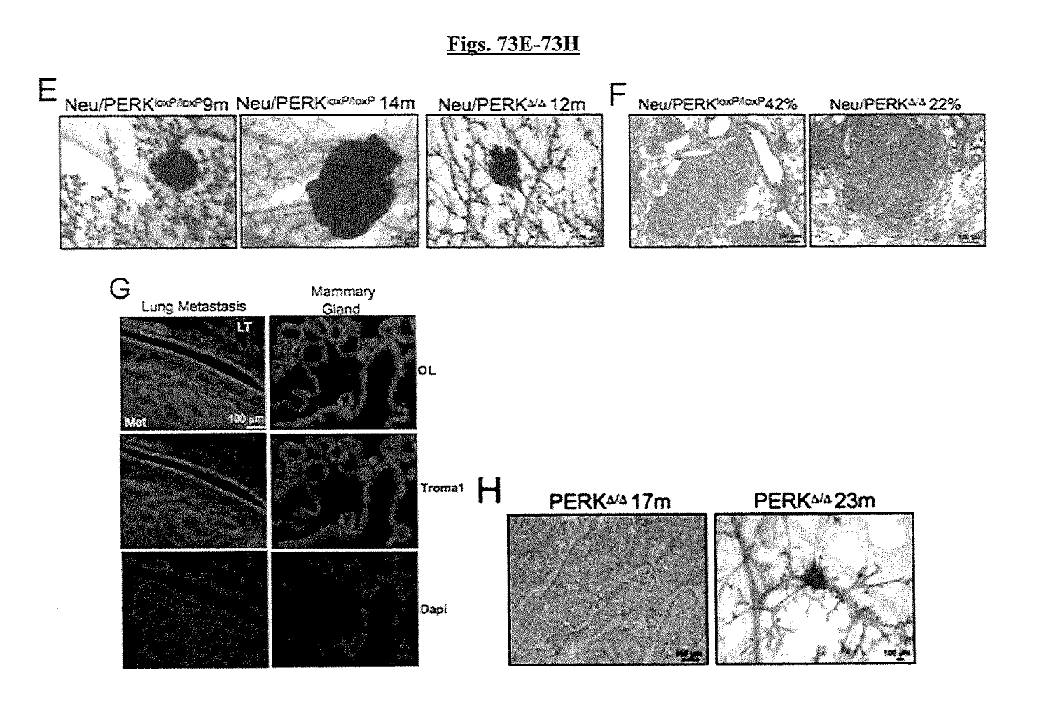

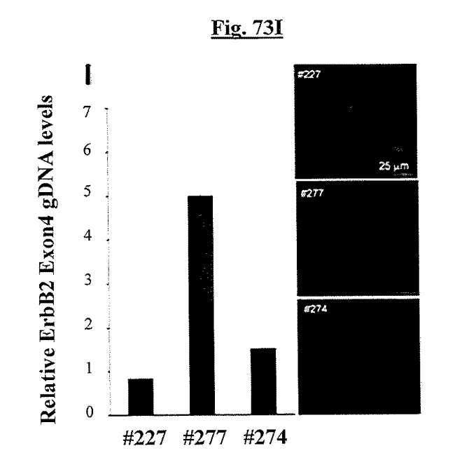

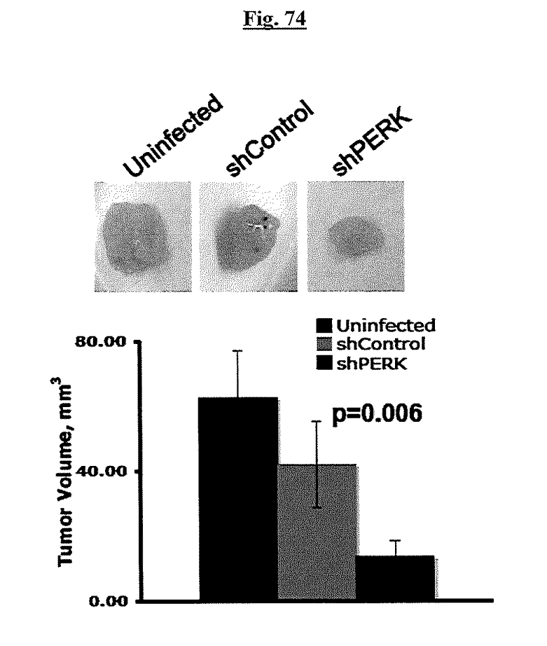

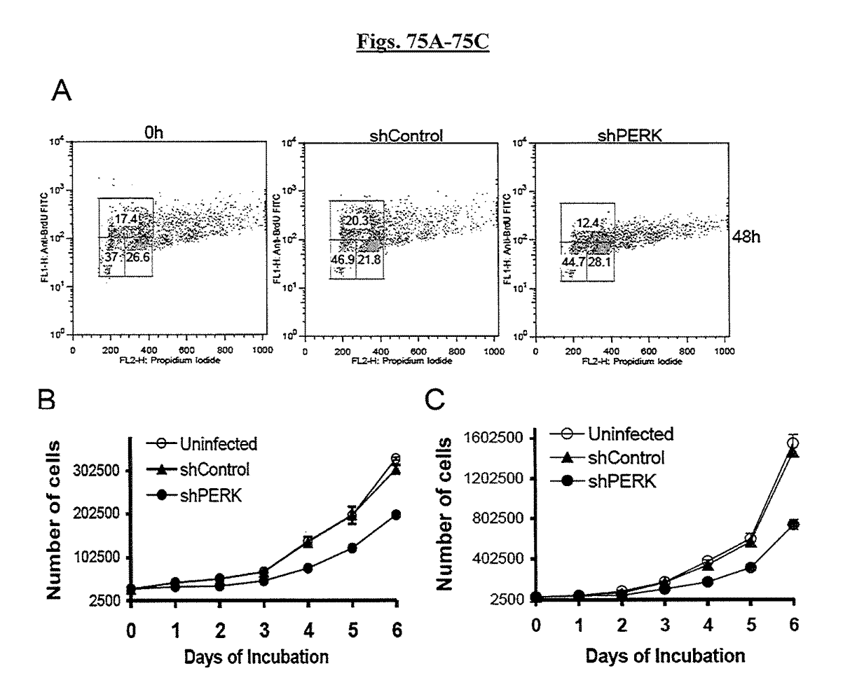

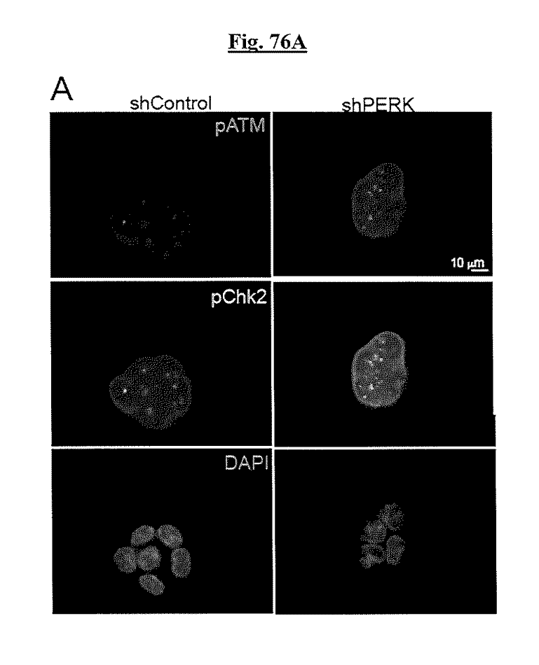

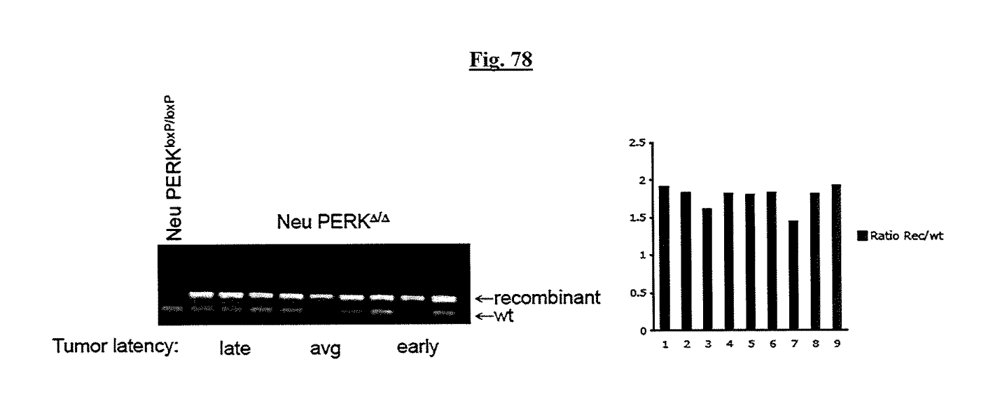

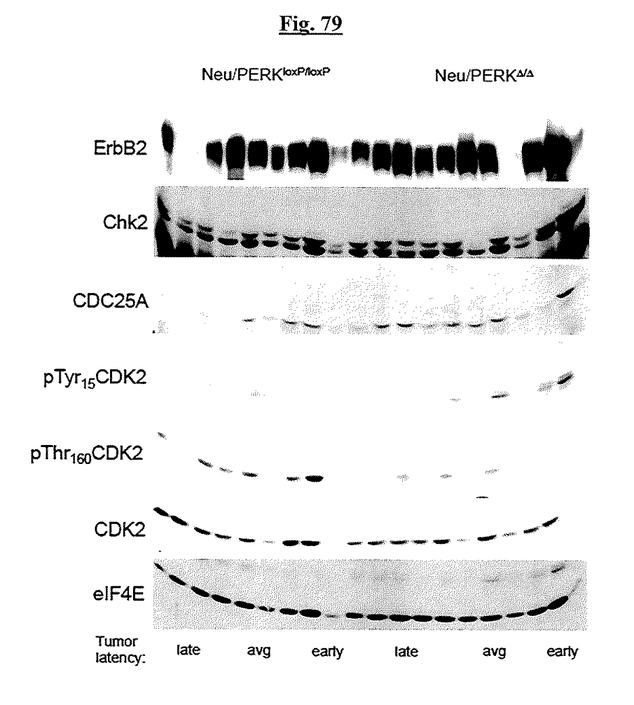

Using mouse models, additional issues were addressed. First, whether deletion of PERK attenuated MMTV-Neu initiated tumorigenesis was investigated. MMTV-Neu transgenic mice were crossed with PERKloxP/loxP/MMTV-Cre mice (Bobrovnikova-Maijon et al., 2008) generating MMTV-Neu/PERK/. Mice that did not inherit the MMTV-Cre transgene, thereby retaining PERK, were used as a control (MMTV-Neu/PERKloxP/loxP). Analysis of tumor-free survival revealed that PERK loss delayed MMTV-Neu-induced tumor formation (FIG. 73A). Histological signature of tumors was characteristic of the MMTV-Neu mouse model (FIG. 73B). PERK excision in mammary epithelium was assessed by immunoblot (FIG. 73C; Bobrovnikova-Marjon et al., 2008, Proc Natl Acad Sci USA 105:16314-9) and RT-PCR (FIG. 78).

Tumor formation was not due to outgrowth of cells exhibiting inefficient PERK excision as only two tumors retained detectable PERK protein (FIG. 73C). To determine whether Thr-80 phosphorylation of Nrf2 was dependent upon PERK in vivo, Nrf2 was immunoprecipitated from tumor lysates and p-Thr levels were assessed. Reduced levels of p-Thr were observed in immunocomplexes from all 4 MMTV-Neu/PERK/tumor lysates analyzed (FIG. 73D), suggesting that phosphorylation of Nrf2 in the tumor environment is a non-redundant function of PERK.

While use of the MMTV promoter to drive both PERK excision and oncogene expression permits targeting of the same cell population, it does not allow for the control of the timing of PERK excision with tumor onset. Previous work revealed that PERK is efficiently excised in the mammary gland of virgin mice by 4 months of age (Bobrovnikova-Marjon et al., 2008, Proc Natl Acad Sci USA 105:16314-9). Because this is substantially prior to MMTV-Neu-induced tumor onset, it was presumed that PERK excision occurs prior to tumor initiation. It was inferred from this that loss of PERK delays tumor onset. To further address this possibility, mammary glands from 9 through 14-months old MMTV-Neu/PERKloxPiloxP and MMTV-Neu/PERK/mice were collected to assess the onset of pre-malignant lesions. No pre-malignant lesions were identified in 9-months old MMTV-Neu/PERK/mice (n=4), and 1 out of 4 mice exhibited a pre-neoplastic lesion in 12-14 months-old group (FIG. 73E). In contrast, hyperplastic lesions were apparent in MMTV-Neu/PERKloxP/loxP mice at 9 and 14 months (FIG. 73E). Although tumor histology did not suggest that loss of PERK resulted in a less aggressive tumor, a 2-fold reduction in the incidence of lung metastases in PERK knockout mice was noted (FIG. 73F). Mammary origin of the metastases was consistent with positive cytokeratin-8 staining (FIG. 73G). Collectively, the data disclosed herein demonstrate at least two critical points. First, acute PERK loss of function compromises tumor growth and expansion. Second, deletion of PERK delays Neu-dependent tumor onset and significantly reduces lung metastases.

DNA damage and activation of DNA damage response may serve as a tumor barrier, while long-term genotoxic stress accompanied by mutational inactivation of DNA damage response mechanisms is pro-tumorigenic (Bartkova et al., 2005, Nature 434:864-70; Gorgoulis et al., 2005, Nature 434:907-13; Stracker et al., 2008, Mol Cell 31:21-32). Thus, it was considered whether loss of PERK might contribute to increased spontaneous mammary tumorigenesis. MMTV-Cre/PERKlo7P/lo7P mice, which do not exhibit detectable proliferative defects during postnatal mammary gland development (Bobrovnikova-Maijon et al., 2008, Proc Natl Acad Sci USA 105:16314-9) were utilized after aging these animals for up to 24 months, During this interval, 6 of 29 animals developed overt mammary adenocarcinoma; in addition, pre-malignant adenomas were observed in several aged mice analyzed (FIG. 73H), while only 2 of 19 control PERKlo7P/lo7P mice developed carcinomas over this same interval. Amplification of the ErbB2 allele in 2 of 6 MMTV-Cre/PERKlo7P/lo7 Panimals was observed using FISH and confirmed with the amplification in one of these tumors by qRT-PCR (FIG. 73I). These findings demonstrate that long-term PERK inactivation could increase susceptibility to spontaneous tumor formation due to increased genomic instability.

The disclosures of each and every patent, patent application, and publication cited herein are hereby incorporated herein by reference in their entirety.

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of modulating the stability of IFNAR1 in a cell, wherein said method comprises contacting said cell with an effective amount of a composition comprising an inhibitor of a regulator of IFNAR1.

2. The method of claim 1, wherein said regulator of IFNAR1 is at least one selected from the group consisting of PERK, PTP1B, and PKD2.

3. The method of claim 1, wherein said inhibitor is at least one selected from the group consisting of an siRNA, a mieroRNA, an antisense nucleic acid, a ribozyme, an expression vector encoding a transdominant negative mutant, an intracellular antibody, a peptide and a small molecule.

4. The method of claim 1, wherein said inhibitor is at least one selected from the group consisting of sangivamycin, a quinoline-difluoromethylphosphonate and a naphthalene-difluoromethylphosphonate.

5. The method of claim 1, wherein said composition further comprises a pharmaceutically acceptable excipient.

6. A method of treating a disease or disorder associated with a dysfunctional IFN response in a subject in need thereof, wherein said method comprises administering to said subjcctin need thereof, a therapeutically effective amount of a composition comprising an inhibitor of a regulator of IFNAR1.

7. The method of claim 6, wherein said regulator of IFNAR1 is at least one selected from the group consisting of PERK, PTP IB, and PKD2.

8. The method of claim 6, wherein said inhibitor is at least one selected from the group consisting of an siRNA, a microRNA, an antisense nucleic acid, a ribozyme, an expression vector encoding a transdominant negative mutant, an intracellular antibody, a peptide and a small molecule.

9. The method of claim 6, wherein said inhibitor is at least one selected from the group consisting of sangivamycin, a quinoline-difluoromethylphosphonate and a naphthalene-difluoromethylphosphonate.

10. The method of claim 6, wherein said composition further comprises a pharmaceutically acceptable excipient.

11. The method of claim 6, wherein said composition is administered in combination with another therapeutic agent.

12. The method of claim 11, wherein said another therapeutic agent is IFN.

13. The method of claim 6, wherein said disease is selected from the group consisting of a viral infection, cancer and an autoimmune disease.

14. (canceled)

15. (canceled)

16. A method of increasing the efficacy of endogenous IFN in a mammal in need thereof, wherein said method comprises administering to said mammal a therapeutically effective amount of a composition comprising an inhibitor of a regulator of IFNR1.

17. The method of claim 16, wherein said regulator of IFNAR1 is at least one selected from the group consisting of PERK, PTP1B, and PKD2.

18. The method of claim 16, wherein said inhibitor is at least one selected from the group consisting of an siRNA, a microRNA, an antisense nucleic acid, a ribozyme, an expression vector encoding a transdominant negative mutant, an intracellular antibody, a peptide and a small molecule.

19. The method of claim 16, wherein said inhibitor is at least one selected from the group consisting of sangivamycin, a quinoline-difluoromethylphosphonate and a naphthalene-difluoromethylphosphonate.

20. The method of claim 16, wherein said composition further comprises a pharmaceutically acceptable excipient.

21. The method of claim 16, wherein said composition is administered in combination with another therapeutic agent.

22. The method of claim 16, wherein said disease is selected from the group consisting of a viral infection, cancer and an autoimmune disease.

23. (canceled)

24. (canceled)

25. A method of increasing the efficacy of IFN-based drug treatment in a mammal in need thereof, wherein said method coniprises administering to said mammal a therapeutically effective amount of a composition comprising an inhibitor of a regulator of IFNR1.

26. The method of claim 25, wherein said regulator of IFNAR1 is at least one selected from the group consisting of PERK, PTPIB, and PKD2.

27. The method of claim 25, wherein said inhibitor is at least one selected from the group consisting of an siRNA, a microRNA, an antisense nucleic acid, a ribozyme, an expression vector encoding a transdominant negative mutant, an intracellular antibody, a peptide and a small molecule.

28. The method of claim 25, wherein said inhibitor is at least one selected from the group consisting of sangivamycin, a quinoline-difluoromethylphosphonate and a naphthalene-difluoromethylphosphonate.

29. The method of claim 25, wherein said composition further comprises a pharmaceutically acceptable excipient.

30. The method of claim 25, wherein said composition is administered in combination with another therapeutic agent.

31. The method of claim 30, wherein said another therapeutic agent is IFN.

32. The method of claim 25, wherein said disease is selected from the group consisting of a viral infection, cancer and an autoimmune disease.

33. (canceled)

34. (canceled)

35. A method of modulating the stability of IFNAR1 in a cell, wherein said method comprises contacting said cell with an effective amount of a composition comprising an activator of a regulator of IFNAR1.

36. The method of claim 35, wherein said regulator of IFNAR1 is at least one selected from the group consisting of PERK, PTP1B, and PKD2.

37. The method of claim 35, wherein said activator is at least one selected from the group consisting of an siRNA, a microRNA, an antisense nucleic acid, a ribozyme, an expression vector encoding a transdominant negative mutant, an intracellular antibody, a peptide and a small molecule.

38. The method of claim 35, wherein said composition further comprises a pharmaceutically acceptable excipient.

39. A method of treating a disease or disorder associated with a dysfunctional IFN response in a subject in need thereof, wherein said method comprises administering to said subject a therapeutically effective amount of a composition comprising an activator of a regulator of IFNAR1.

40. The method of claim 39, wherein said regulator of IFNAR1 is at least one selected from the group consisting of PERK, PTP1B, and PKD2.

41. The method of claim 39, wherein said activator is at least one selected from the group consisting of an siRNA, a microRNA, an antisense nucleic acid, a ribozyme, an expression vector encoding a transdominant negative mutant, an intracellular antibody, a peptide and a small molecule.

42. The method of claim 39, wherein said composition further comprises a pharmaceutically acceptable excipient.

43. The method of claim 39, wherein said composition is administered in combination with another therapeutic agent.

44. The method of claim 39, wherein said disease is selected from the group consisting of a viral infection, cancer and an autoimmune disease.

45. (canceled)

46. (canceled)
